SEQUENCE LISTING (8 sequences)

SEQ ID NO: 1 - protein, 120 aa, Artificial Sequence
  Amino acid sequences of the L2G7 mature heavy chain and light chain
QVQLQQSGADLMKPGASVKISCKATGYTFSGNWIEWVKQRPGHGLEWIGEILPGSGNTNYNEKFKGKATFTADTSSNTAYMQLSSLTSEDSAVYYCARGGHYYGSSWDYWGQGTTLTVSS

SEQ ID NO: 2 - protein, 120 aa, Artificial Sequence
  Amino acid sequences of the HuL2G7 mature heavy chain and light chain
EVQLVQSGAEVKKPGASVKVSCKVSGYTFSGNWIEWVRQAPGKGLEWIGEILPGSGNTNYNEKFKGKATMTADTSTDTAYMELSSLRSEDTAVYYCARGGHYYGSSWDYWGQGTLVTVSS

SEQ ID NO: 3 - protein, 125 aa, Artificial Sequence
  Amino acid sequence for AAC18323 of the mature heavy chain and light chain
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATPVGRCSSTSCYHPLDYWGQGTLVTVSS

SEQ ID NO: 4 - protein, 108 aa, Artificial Sequence
  Amino acid sequences of the L2G7 mature heavy chain variable regions translated from the cloned cDNAs
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 5 - protein, 108 aa, Artificial Sequence
  Amino acid sequences of the HuL2G7 mature heavy chain variable regions translated from the cloned cDNAs
DIVMTQSPSSLSASVGDRVTITCKASENVVTYVSWYQQKPGKAPKLLIYGASNRYTGVPDRFSGSGSGTDFTLTISSLQPEDFATYYCGQGYSYPYTFGQGTKLEIKR

SEQ ID NO: 6 - protein, 108 aa, Artificial Sequence
  Amino acid sequence for AAC18323 of the mature heavy chain variable regions translated from the cloned cDNAs
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKR

SEQ ID NO: 7 - protein, 469 aa, Artificial Sequence
  Amino acid sequences of the entire HuL2G7 heavy chain and light chain
MDCTWRILFLVAAATGTHAEVQLVQSGAEVKKPGASVKVSCKVSGYTFSGNWIEWVRQAPGKGLEWIGEILPGSGNTNYNEKFKGKATMTADTSTDTAYMELSSLRSEDTAVYYCARGGHYYGSSWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 161 bp, Artificial Sequence
  The first amino acids of the HuL2G7 mature heavy and light chains
maawdthgdvmtssssasvgdrvttckasnvvtyvswykgkakygasnrytgvdrsgsgsgtdttssdatyycggysyytggtkkrtvaasvsdksgtasvvcnnyrakvwkvdnasgnssvtdskdstysssttskadykhkvyacvthgssvtksnrgc

(19) United States
(12) Patent Application Publication
(10) Pub. No.: US 2013/0315895 A1
(43) Pub. Date: Nov. 28, 2013

(54) COMBINATION OF A cMET INHIBITOR AND AN ANTIBODY TO HGF AND/OR cMET

(71) Applicants: Pamela Farrell, San Diego, CA (US); Patrick Vincent, San Diego, CA (US)

(72) Inventors: Pamela Farrell, San Diego, CA (US); Patrick Vincent, San Diego, CA (US)

(73) Assignee: TAKEDA PHARMACEUTICAL COMPANY LIMITED, Osaka (JP)

(21) Appl. No.: 13/703,861

(22) PCT Filed: Jun. 30, 2011
(86) PCT No.: PCT/US11/42622

§ 371 Date: Aug. 2, 2013

Related U.S. Application Data
(60) Provisional application No. 61/360,693, filed on Jul. 1, 2010.

(51) Int. Cl. A61K 39/395; A61K 31/437; A61K 45/06; C07D 487/04; C07D 401/14; A61K 31/4545; C07D 471/04; A61K 31/5025

(52) U.S. Cl. CPC A61K 39/3955; C07D 471/04; A61K 31/437; A61K 31/5025; C07D 487/04; C07D 401/14; A61K 31/4545; A61K 45/06
USPC 424/133.1; 424/158.1; 424/142.1

(57) ABSTRACT

Methods for treating a disease state for which HGF/cMET possesses activity that contributes to the pathology and/or symptomology of the disease state, as well as kits and articles of manufacture for use in practicing these methods.

### FIELD OF THE INVENTION

The present invention relates to methods for treating a disease state for which HGF/cMET possesses activity that contributes to the pathology and/or symptomology of the disease state, as well as kits and articles of manufacture for use in practicing these methods.

### BACKGROUND OF THE INVENTION

HGF/cMET have been reported to play important roles in several aspects of cancer development. Human Hepatocyte Growth Factor (HGF, also known as scatter factor), the ligand for cMET, is a multifunctional heterodimeric polypeptide produced by mesenchymal cells. cMET is a receptor tyrosine kinase and is predominantly expressed on cells of epithelial/endothelial origin resulting in paracrine epithelial-mesenchymal cell signaling (Stoker, M. et al., Nature 327: 239-242 (1987)). Binding of HGF to the extracellular region of cMET activates the intracellular cMET tyrosine kinase activity.

cMET is believed to be involved in protein phosphorylation events that regulate cell proliferation, apoptosis, motility, and dissociation of cell-cell interactions, morphogenesis, angiogenesis, and epithelial-mesenchymal transition. Misregulation of cMET can lead to unregulated cell proliferation and survival. cMET is thought to be a key regulator of invasive growth, cancer tumorgenesis, and progression to metastasis (Trusolino, T. and Comoglio, P. Nature Reviews Cancer: 2: 289-300 (2002)). cMET gene amplification, alteration, mutation, and protein over expression or activation of cMET through autocrine or paracrine mechanisms have been detected in a wide variety of carcinomas. For example, in human gastric cancer tissue, cMET has been found to be over expressed and amplified (Smolen, G. A., et al., PNAS 103: 2316-2321, (2006)). In human glioblastomas and carcinomas of lung, thyroid and breast, cMET has been found to be activated as a result of increased HGF levels and autocrine signaling (Birchmeier, C. et al. Rev. Mol. Cell. Biol. 4: 915-925, (2003)). In human lung cancer tissue, cMET signaling has been found to be upregulated as a mechanism of drug resistance (Engelman, J. A., et al. Science 316: 1049-1043, (2007)). Activating mutations in cMET, although not as common, have been reported in sporadic and hereditary papillary renal carcinomas, head and neck squamous carcinomas as well as gastric and lung cancers. Furthermore, increased expression, the most common cMET alteration found in a wide variety of human tumors (including but not limited to renal, ovarian, hepatocellular, non-small cell lung, bone, liver metastasis of colon, oral squamous cell, esophageal, gastric, pancreatic, and prostatic cancers) correlates with poor prognosis (Benvenuti, S, and Comoglio, P. M., J. Cell. Physiol. 213: 316-325, (2007)).

HGF has been shown to stimulate angiogenesis, morphogenesis and motogenesis, as well as the growth and scattering of various cell types (Bussolino et al., J. Cell. Biol. 119: 629, 1992; Zarnegar and Michalopoulos, J. Cell. Biol. 129:1177, 1995; Matsumoto et al., Ciba. Found. Symp. 212:198, 1997; Birchmeier and Gherardi, Trends Cell. Biol. 8:404, 1998; Xin et al., Am. J. Pathol. 158:1 111, 2001).

In addition to regulating a variety of normal cellular functions, HGF and its receptor c-Met have been shown to be involved in the initiation, invasion and metastasis of tumors (Jeffers et al., J. MoI. Med. 74:505, 1996; Comoglio and Trusolino, J. Clin. Invest. 109:857, 2002). HGF/cMet are coexpressed, often over-expressed, on various human solid tumors including tumors derived from lung, colon, rectum, stomach, kidney, ovary, skin, multiple myeloma and thyroid tissue (Prat et al., Int. J. Cancer 49:323, 1991; Chan et al., Oncogene 2:593, 1988; Weidner et al., Am. J. Respir. Cell. MoI. Biol. 8:229, 1993; Derksen et al., Blood 99:1405, 2002). HGF acts as an autocrine (Rong et al., Proc. Natl. Acad. Sci. USA 9 1:4731, 1994; Koochekpour et al., Cancer Res. 57:5391, 1997) and paracrine growth factor (Weidner et al., Am. J. Respir. Cell. Mol. Biol. 8:229, 1993) and anti-apoptotic regulator (Gao et al., J. Biol. Chem. 276:47257, 2001) for these tumors.

There is a continued need to find new therapeutic agents to treat human diseases. HGF and cMET are attractive targets for the discovery of new therapeutics due to the important roles of both HGF and cMET in cancer and other diseases.

### SUMMARY OF THE INVENTION

The present invention relates to methods for treating a disease state for which HGF/cMET possesses activity that contributes to the pathology and/or symptomology of the disease state, as well as kits and articles of manufacture for use in practicing these methods. In particular, the present invention relates to the combination of an antibody that inhibits the HGF/cMET signaling pathway and a cMET inhibitor. The present invention also relates to the combination of an anti-HGF antibody and a cMET inhibitor, which is an especially attractive therapeutic modality which has not been explored. Further, the present invention relates to the combination of an anti-cMET antibody and a cMET inhibitor.

In one of its aspects, the present invention relates to methods for treating a disease state for which HGF/cMET possesses activity that contributes to the pathology and/or symptomology of the disease state. In one embodiment, the method comprises administering a cMET inhibitor, or a pharmaceutically acceptable salt thereof, and an antibody that inhibits the HGF/cMET signaling pathway to a patient. In one particular variation, the disease state is cancer including, but not limited to, hepatocellular carcinoma, brain cancer, glioblastoma and pancreatic cancer.

In one variation of the above embodiments and variations, the antibody binds to the cMET receptor. In another variation of the above embodiments and variations, the antibody binds to a cMET ligand. In still another variation of the above embodiments and variations, the antibody binds to HGF. In yet another variation of the above embodiments and variations, the antibody binds to human HGF. In a further variation of the above embodiments and variations, the antibody is a chimeric L2G7 monoclonal antibody. In still a further variation of the above embodiments and variations, the antibody is a humanized L2G7 monoclonal antibody. In yet a further variation of the above embodiments and variations, the antibody is a human L2G7 monoclonal antibody. In yet a further variation of the above embodiments and variations, the antibody is an antibody which competes for binding with the L2G7 antibody.

In another variation of the above embodiments and variations, the antibody is selected from the group consisting of:
OA-5d5;
AMG 102;
AV-299 (SCH 900706);
L2G7 and other antibodies as described in WO 2005/107800;
humanized L2G7 and other antibodies as described in WO 2007/115049;
an antibody as described in WO 2005/017107;
an antibody as described in WO 2007/143090 and/or WO 2007/143098;
an antibody as described in WO 2001/034650;
an antibody as described in US 2005/0118643;
an antibody as described in WO 2005/017107;
an antibody as described in US 2007/0092520;
an antibody as described in U.S. Pat. No. 7,494,650; and
an antibody as described in U.S. Pat. No. 7,220,410.

In a further variation of the above embodiments and variations, the cMET inhibitor has the formula

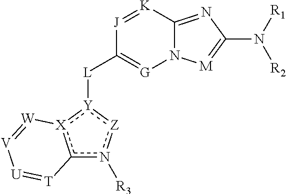

or a pharmaceutically acceptable salt thereof, wherein
G is selected from the group consisting of CR₄ and N;
J is selected from the group consisting of CR₅ and N;
K is selected from the group consisting of CR₆ and N;
M is selected from the group consisting of CR₇ and N;
L is absent or a linker providing 1, 2, 3, 4, 5 or 6 atom separation between the rings to which L is attached, wherein the atoms of the linker providing the separation are selected from the group consisting of carbon, oxygen, nitrogen, and sulfur;
T is selected from the group consisting of CR₈ and N;
U is selected from the group consisting of CR₉ and N;
V is selected from the group consisting of CR₁₀ and N;
W is selected from the group consisting of CR₁₁ and N;
X is selected from the group consisting of CR₁₂ and N;
Y is selected from the group consisting of CR₁₃ and N;
Z is selected from the group consisting of CR₁₄R₁₅ and NR₁₆;
R₁ is selected from the group consisting of hydrogen, carbonyloxy, (C₁₋₁₀)alkoxy, (C₄₋₁₂)aryloxy, hetero(C₁₋₁₀)aryloxy, carbonyl, oxycarbonyl, aminocarbonyl, (C₁₋₁₀)alkyl carbonyl, (C₃₋₁₂)cycloalkyl(C₁₋₅)carbonyl, hetero(C₃₋₁₂)cycloalkyl(C₁₋₁₀)carbonyl, aryl(C₁₋₁₀)carbonyl, hetero(C₁₋₁₀)aryl(C₁₋₅)carbonyl, (C₉₋₁₂)bicycloaryl(C₁₋₅)carbonyl, hetero(C₈₋₁₂)bicycloaryl(C₁₋₅)carbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁ has the formula

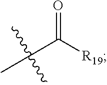

R₂ is hydrogen or a substituent convertible in vivo to hydrogen;
R₃ is selected from the group consisting of hydrogen, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₃ is absent when the nitrogen to which it is bound forms part of a double bond;
R₄ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₅ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₆ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₇ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₈ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₉ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, amido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, amino(C₁₋₁₀)alkyl, amido(C₁₋₁₀)alkylamino(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, carbonyl(C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₀ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, amido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, amino(C₁₋₁₀)alkyl, amido(C₁₋₁₀)alkylamino(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, carbonyl(C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₁ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₂ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₂ is absent when the carbon to which it is bound forms part of a double bond;
R₁₃ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₃ is absent when the carbon to which it is bound forms part of a double bond;
R₁₄ and R₁₅ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₅ is absent when the carbon to which it is bound forms part of a double bond;
R₁₆ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₆ is absent when the nitrogen to which it is bound forms part of a double bond; and
R₁₉ selected from the group consisting of hydrogen, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of the above embodiments and variations, the cMET inhibitor has the formula

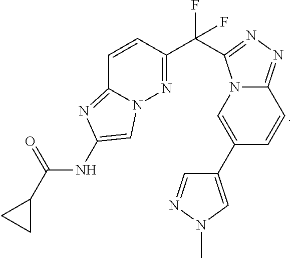 [SMILES: Cn1cc(-c2ccc3nnc(C(F)(F)c4ccc5nc(NC(=O)C6CC6)cn5n4)n3c2)cn1]

In yet a further variation of the above embodiments and variations, the cMET inhibitor has the formula

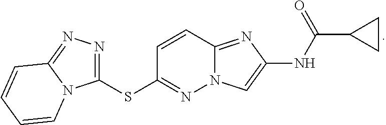 [SMILES: O=C(Nc1cn2nc(Sc3nnc4ccccn34)ccc2n1)C1CC1]

In another variation of the above embodiments and variations, the cMET inhibitor has the formula

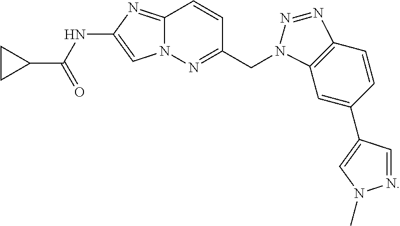 [SMILES: Cn1cc(-c2ccc3nnn(Cc4ccc5nc(NC(=O)C6CC6)cn5n4)c3c2)cn1]

In still another variation of the above embodiments and variations, the cMET inhibitor has the formula:

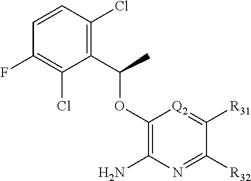

wherein
Q₂ is N or CR₄₂;
R₃₁ is selected from the group consisting of hydrogen, halogen, (C₆₋₁₂)aryl, 5-12 membered heteroaryl, (C₃₋₁₂)cycloalkyl, 3-12 membered heteroalicyclic, O—(CR₃₆R₃₇)_{q}R₃₄, COR₃₄; C(O)OR₃₄, CN, NO₂, S(O)₁R₃₄, SO₂NR₃₄R₃₅; NR₃₄C(O)R₃₅, C(NR₃₆)NR₃₄R₃₅, (C₁₋₈)alkyl, (C₂₋₈)alkenyl and (C₂₋₈)alkynyl, and each hydrogen in R₃₁ is optionally substituted by one or more R₃₃ groups;
R₃₂ is hydrogen, halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, O(CR₃₆R₃₇)_{q}R₃₄, NR₃₄C(O)R₃₅, (CR₃₆R₃₇)_{q}C(O)OR₃₄, (CR₃₆R₃₇)_{q}NCR₃₄R₃₅, C(═NR₃₆)NR₃₄R₃₅, NR₃₄C(O)NR₃₅R₃₆, NR₃₄S(O)_{P}R₃₅ or C(O)NR₃₄R₃₅, and each hydrogen in R₃₂ is optionally substituted by R₃₈;
each R₃₃ is independently halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, O(CR₃₆R₃₇)_{q}R₃₄, NR₃₄C(O)R₃₅, (CR₃₆R₃₇)_{q}C(O)OR₃₄, (CR₃₆R₃₇)_{q}OR₃₄, (CR₃₆R₃₇)_{q}C(O)NR₃₄R₃₅, (CR₃₆R₃₇)_{q}NCR₃₄R₃₅, C(═NR₃₆)NR₃₄R₃₅, NR₃₄C(O)NR₃₅R₃₆, NR₃₄S(O)_{P}R₃₅ or C(O)NR₃₄R₃₅, each hydrogen in R₃₃ is optionally substituted by R₃₈, and R₃₃ groups on adjacent atoms may combine to form a (C₆₋₁₂)aryl, 5-12 membered heteroaryl, (C₃₋₁₂)cycloalkyl or 3-12 membered heteroalicyclic group;
each R₃₄, R₃₅, R₃₆ and R₃₇ is independently hydrogen, halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl; or any two of R₃₄, R₃₅, R₃₆ and R₃₇ bound to the same nitrogen atom may, together with the nitrogen to which they are bound, be combined to form a 3 to 12 membered heteroalicyclic or 5-12 membered heteroqaryl group optionally containing 1 to 3 additional heteroatoms selected from N, O and S; or any two of R₃₄, R₃₅, R₃₆ and R₃₇ bound to the same carbon atom may be combined to form a (C₃₋₁₂)cycloalkyl, (C₆₋₁₂)aryl, 3-12 membered heteroalicyclic or 5-12 memebered heteroaryl group; and each hydrogen in R₃₄, R₃₅, R₃₆ and R₃₇ is optionally substituted by R₃₈;
each R₃₈ is independently halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, NH₂, CN, OH, O—(C₁₋₁₂)alkyl, O—(CH₂)_{q}(C₃₋₁₂)cycloalkyl, O—(CH₂)_{q}(C₆₋₁₂)aryl, O—(CH₂)_{q}(3-12 membered heteroalicyclic) or O—(CH₂)_{q}(5-12 membered heteroaryl); and each hydrogen in R₃₈ is optionally substituted by R₄₁;
each R₃₉ and R₄₀ is independently hydrogen, halogen, (C₁₋₁₂)alkyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, NR₃₄C(O)R₃₅, (CR₃₆R₃₇)_{q}C(O)OR₃₄, (CR₃₆R₃₇)_{q}NCR₃₄R₃₅, NR₃₄C(O)NR₃₅R₃₆, NR₃₄S(O)ₚR₃₅ or C(O)NR₃₄R₃₅; R₃₉ or R₄₀ may combine with a ring atom of A or a substitutent of A to form a (C₃₋₁₂)cycloalkyl, 3-12 memebered heteroalicyclic, (C₆₋₁₂)aryl or 5-12 memebered heteroaryl ring fused to A; and each hydrogen in R₃₉ and R₄₀ is optionally substituted by R₃₃;
each R₄₁ is independently halogen, (C₁₋₁₂)alkyl, (C₁₋₁₂)alkoxy, (C₃₋₁₂)cycloalkyl, (C₆₋₁₂)aryl, 3-12 membered heteroalicyclic, 5-12 membered heteroaryl, O—(C₁₋₁₂)alkyl, O—(CH2)₁(C₃₋₁₂)cycloalkyl, O—(CH₂)_{q}(C₆₋₁₂)aryl, O—(CH₂)_{q}(3-12 membered heteroalicyclic, O—(CH₂)_{q}(5-12 membered heteroaryl or CN, and each hydrogen in R₄₁ is optionally substituted by halogen, OH, CN, (C₁₋₁₂)alkyl which may be partially or fully halogenated, O—(C₁₋₁₂)alkyl which may be partially or fully halogenated, CO, SO or SO2;
R₄₂ is hydrogen, halogen (C₁₋₁₂)alkyl, (C₂₋₁₂)alkenyl, (C₂₋₁₂)alkynyl, (C₃₋₁₂)cycloalkyl, (C₆₋₁₂)aryl, 3-12 memebered heteroalicyclic, 5-12 memebered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, O—C(O)R₃₄, O—(CR₃₆R₃₇)_{q}R₃₄, NR₃₄C(O)R₃₅, (CR₃₆R₃₇)_{q}C(O)OR₃₄, (CR₃₆R₃₇)_{q}NCR₃₄R₃₅, C(═NR₃₆)NR₃₄R₃₅, NR₃₄C(O)NR₃₅R₃₆, NR₃₄S(O)ₚR₃₅ or C(O)NR₃₄R₃₅, and each hydrogen in R₄₂ is optionally substituted by R₃₃;
each R₄₃ is independently halogen, (C₁₋₁₂)alkyl, (C₂₋₁₂)aleknyl, (C₂₋₁₂)alkynyl, (C₃₋₁₂)cycloalkyl, (C₆₋₁₂)aryl, 3-12 membered hertoalicyclic, 5-12 memebered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, O—C(O)R₃₄, O—(CR₃₆R₃₇)_{q}R₃₄, NR₃₄C(O)R₃₅, (CR₃₆R₃₇)_{q}C(O)OR₃₄, (CR₃₆R₃₇)_{q}OR₃₄, (CR₃₆R₃₇)_{q}C(O)NR₃₄R₃₅, (CR₃₆R₃₇)_{q}NCR₃₄R₃₅, C(═NR₃₆)NR₃₄R₃₅, NR₃₄C(O)NR₃₅R₃₆, NR₃₄S(O)ₚR₃₅, C(O)NR₃₄R₃₅, (CR₃₆R₃₇)_{q}(3-12 memebered heteroalicyclic), (CR₃₆R₃₇)_{q}(C₃₋₁₂)cycloalkyl, (CR₃₆R₃₇)_{q}(C₆₋₁₂)aryl, (CR₃₆R₃₇)_{q}(5-12 membered heteroaryl, (CR₃₆R₃₇)_{q}C(O)NR₃₄R₃₅ or (CR₃₆R₃₇)_{q}C(O)R₃₄, R₄₃ groups on adjacent atoms may combine to form a (C₆₋₁₂)aryl, 5-12 memebered heteroaryl, (C₃₋₁₂)cycloalkyl or 3-12 memebered heteroalicyclic group, and each hydrogen in R₄₃ is optionally substituted by R₃₃;
each 1 is independently 0, 1 or 2;
each q is independently 0, 1, 2, 3 or 4; and
each p is independently 1 or 2,
   or a pharmaceutically acceptable salt thereof.

In yet another variation of the above embodiments and variations, the cMET inhibitor has the formula:

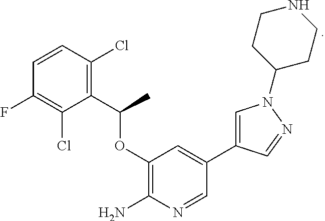 [SMILES: C[C@@H](Oc1cc(-c2cnn(C3CCNCC3)c2)cnc1N)c1c(Cl)ccc(F)c1Cl]

In a further variation of the above embodiments and variations, the cMET inhibitor has the formula:

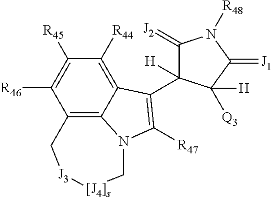

wherein
R₄₄, R₄₅ and R₄₆ are independently selected from the group consisting of H, F, Cl, Br, I, NR₅OR₅₁, (C₁₋₆)alkyl, (C₁₋₆) substituted alkyl, (C₃₋₉)cycloalkyl, (C₃₋₉) substituted cycloalkyl, O—(C₁₋₆)alkyl, O—(C₃₋₉)cycloalkyl, O—(C₃₋₉) substituted cycloalkyl, aryl, heteroaryl and heterocyclyl;
R₄₇ is selected from the group consisting of H, (C₁₋₄)alkyl and (C₁₋₄) substituted alkyl;
R₄₈ is selected from the group consisting of H, (C₁₋₆)alkyl, CH₂R₄₉, CONHR₅₂, COR₅₃ and SO₂R₅₄;
R₄₉ is selected from the group consisting of O—P(═O)(OH)₂, O—P(═O)(OH)(O—(C₁₋₆)alkyl), O—P(═O)(O—(C₁₋₆)alkyl)₂, O—P(═O)(OH)(O—(CH₂)phenyl), O—P(═O)(O—(CH₂)phenyl)₂, a carboxylic acid group, an amino carboxylic acid group and a peptide;
R₅₀ and R₅₁ are independently selected from the group consisting of H and (C₁₋₆)alkyl;
R₅₂, R₅₃ and R₅₄ are independently selected from the group consisting of H, NHR₅₅, (C_{—}_{¹⁻⁶})alkyl, (C_{—}_{¹⁻⁶}) substituted alkyl, (C₃₋₉)cycloalkyl, (C₃₋₉) substituted cycloalkyl, aryl, heteroaryl and heterocyclyl;
Q₃ is selected from the group consisting of indolyl, substituted indolyl, aryl, heteroaryl, heterocyclyl and alkyl;
J₁ and J₂ are independently selected from the group consisting of O, S, H₂, where R₄₇ is (C₁₋₄)alkyl or (C₁₋₄) substituted alkyl when both J₁ and J₂ are O, and R₄₈ is H, (C₁₋₆)alkyl or CH₂R₄₉ when both J₁ and J₂ are not H₂;
J₃ is selected from the group consisting of —CH₂—, —NR₅₅—, S, O and a bond;
R₅₅ is selected from the group consisting of H, (C₁₋₆)alkyl, (C₁₋₆) substituted alkyl, (C₃₋₉)cycloalkyl, (C₃₋₉) substituted cycloalkyl, O—(C₁₋₆)alkyl, C(═O)—O—(C₁₋₆)alkyl and C(═O)—O—(C₁₋₆) substituted alkyl;
J₄ is selected from the group consisting of —CH₂—, CO and a bond; and
s is 0, 1 or 2.

In yet a further variation of the above embodiments and variations, the cMET inhibitor is selected from the group consisting of:
K-252a;
SU-11274;
PHA-665752 and other cMET inhibitors described in WO 2002/096361;
AM7;
AMG-208 and other cMet inhibitors described in WO 2009/091374;
JNJ-38877605 and other cMet inhibitors described in WO 2007/075567;
MK-2461 and other cMet inhibitors described in WO 2007/002254 and/or WO 2007/002258;
PF-04217903 and other cMet inhibitors described in WO 2007/132308;
BMS 777607;
GSK 136089 (also known as XL-880 and Foretinib) and other cMET inhibitors described in WO 2005/030140;
BMS 907351 (also known as XL-184);
EMD 1214063;
LY 2801653;
ARQ 197;
MK 8033;
PF 2341066 and other cMET inhibitors described in WO 2006/021881;
MGCD 265;
E 7050;
MP 470;
SGX 523;
cMet inhibitors described in Kirin J. J. Cui, Inhibitors targeting hepatocyte growth factor receptor and their potential therapeutic applications. Expert Opin. Ther. Patents 2007; 17:1035-45;
cMet inhibitors described in WO 2008/103277;
cMet inhibitors described in WO 2008/008310;
cMet inhibitors described in WO 2007/138472;
cMet inhibitors described in WO 2008/008539;
cMet inhibitors described in WO 2009/007390;
cMet inhibitors described in WO 2009/053737;
cMet inhibitors described in WO 2009/024825;
cMet inhibitors described in WO 2008/071451;
cMet inhibitors described in WO 2007/130468;
cMet inhibitors described in WO 2008/051547;
cMet inhibitors described in WO 2008/053157;
cMet inhibitors described in WO 2008/017361; WO 2008/145242; WO 2008/145243; WO 2008/148449; WO 2009/007074; WO 2009/006959; WO 2009/024221; WO 2009/030333; and/or WO 2009/083076;
cMet inhibitors described in WO 2009/093049;
cMet inhibitors described in US 2008/039457;
cMet inhibitors described in WO 2007/149427;
cMet inhibitors described in WO 2007/050309;
cMet inhibitors described in WO 2009/056692;
cMet inhibitors described in WO 2009/087305;
cMet inhibitors described in US 2009/197864;
cMet inhibitors described in US 2009/197862;
cMet inhibitors described in US 2009/156594;
cMet inhibitors described in WO 2008/124849;
cMet inhibitors described in WO 2008/067119;
cMet inhibitors described in WO 2007/064797;
cMet inhibitors described in WO 2009/045992;
cMet inhibitors described in WO 2008/088881;
cMet inhibitors described in WO 2007/081978;
cMet inhibitors described in WO 2008/079294;
cMet inhibitors described in WO 2008/079291;
cMet inhibitors described in WO 2008/086014;
cMet inhibitors described in WO 2009/033084;
cMet inhibitors described in WO 2007/059202;
cMet inhibitors described in US 2009/170896;
cMet inhibitors described in WO 2009/077874 and/or WO 2007/023768;
cMet inhibitors described in WO 2008/049855;
cMet inhibitors described in WO 2009/026717; and
cMet inhibitors described in WO 2008/046216.

In another variation of the above embodiments and variations, the method further comprises the step of administering one or more additional therapeutic agents. In one particular variation, the one or more additional therapeutic agents comprise a Hedgehog inhibitor. In another particular variation, the one or more additional therapeutic agents comprise an EGFR antagonist. In still another particular variation, the one or more additional therapeutic agents comprise a PTEN agonist. In still another particular variation, the one or more additional therapeutic agents comprise a nucleoside analogue. In still another particular variation, the one or more additional therapeutic agents comprise a PDGFR antagonist. In still another particular variation, the one or more additional therapeutic agents comprise a VEGFR antagonist. In still another particular variation, the one or more additional therapeutic agents comprise a c-KIT antagonist. In still another particular variation, the one or more additional therapeutic agents comprise a FLT3 inhibitor.

The invention is also directed to kits and other articles of manufacture for treating disease states associated with HGF/cMET. In one embodiment, a kit is provided that comprises the cMET inhibitor, or a pharmaceutically acceptable salt thereof; the antibody that inhibits the HGF/cMET signaling pathway; and instructions. The kit may optionally further include the one or more additional therapeutic agents. The instructions may indicate the disease state for which the kit is to be used, storage information, dosing information and/or instructions regarding how to administer the cMET inhibitor, antibody and/or additional therapeutic agent or agents. The kit may also comprise packaging materials. The packaging material may comprise a container for housing the contents of the kit. The kit may also optionally comprise additional components, such as syringes for administration of the contents of the kit. The kit may comprise the cMET inhibitor, antibody and/or additional therapeutic agent or agents in single or multiple dose forms.

In another embodiment, an article of manufacture is provided that comprises the cMET inhibitor, or a pharmaceutically acceptable salt thereof; the antibody that inhibits the HGF/cMET signaling pathway; and packaging materials. The article of manufacture may optionally further include the one or more additional therapeutic agents. The packaging material may comprise a container for housing the contents of the article of manufacture. The container may optionally comprise a label indicating the disease state for which the article is to be used, storage information, dosing information and/or instructions regarding how to administer the cMET inhibitor, antibody and/or additional therapeutic agent or agents. The kit may also optionally comprise additional components, such as syringes for administration of the composition. The article may comprise the cMET inhibitor, antibody and/or additional therapeutic agent or agents in single or multiple dose forms.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well known in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound comprising the formula,” “compound having the formula” and “compound of the formula” is intended to encompass the compound and all pharmaceutically acceptable ionized forms and solvates, all possible stereoisomers, and all possible resonance forms and tautomers unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to inhibiting cMET and thus may themselves be prodrugs for another compound. Such prodrugs of another compound may or may not themselves independently have HGF/cMET inhibitory activity.

All references cited herein, including patent applications and publications, are hereby incorporated by reference in their entireties.

The L2G7 hybridoma has been deposited with the American Type Culture Collection, P.O. Box 1549 Manassas, Va. 20108, as ATCC Number PTA-5162 under the Budapest Treaty.

### BRIEF DESCRIPTION OF THE DRAWINGS

FIG. 1. Amino acid sequences of the L2G7 mature heavy chain (A) and light chain (B) variable regions translated from the cloned cDNAs. Amino acid sequences of the HuL2G7 heavy chain (A) and light chain (B) mature variable regions are shown aligned with L2G7 and acceptor V regions. The CDRs are underlined in the L2G7 sequences, and the amino acids substituted with mouse L2G7 amino acids are underlined in the HuL2G7 sequences, with the initial amino acid H 1E double-underlined. The Kabat numbering system is used.

FIG. 2. Amino acid sequences of the entire HuL2G7 heavy chain (A) and light chain (B). The first amino acids of the mature heavy and light chains (i.e., after cleavage of the signal sequences) are double underlined and labeled with the number 1; these amino acids are also the first amino acids of the mature V regions. In the heavy chain, the first amino acids of the CH1, hinge, CH2 and CH3 regions are underlined, and in the light chain, the first amino acid of the C_{κ}; region is underlined.

FIG. 3. Graph of mean tumor growth in the in vivo evaluation of Compounds 3 and 45, alone and in combination with Anti-HGF mAb HuL2G7, in Human U87MG Glioblastoma Implanted Nude Mice. G1 is the results for Vehicle 1/Vehicle 2. G2 is the results for Compound 45 (136.05)/Vehicle 2. G3 is the results for Compound 3 (200)/Vehicle 2. G4 is the results for Vehicle 1/HuL2G7 (10), iv; qwk×2. G5 is the results for Compound 45 (136.05)/HuL2G7 (10). G6 is the results for Compound 3 (200)/HuL2G7 (10).

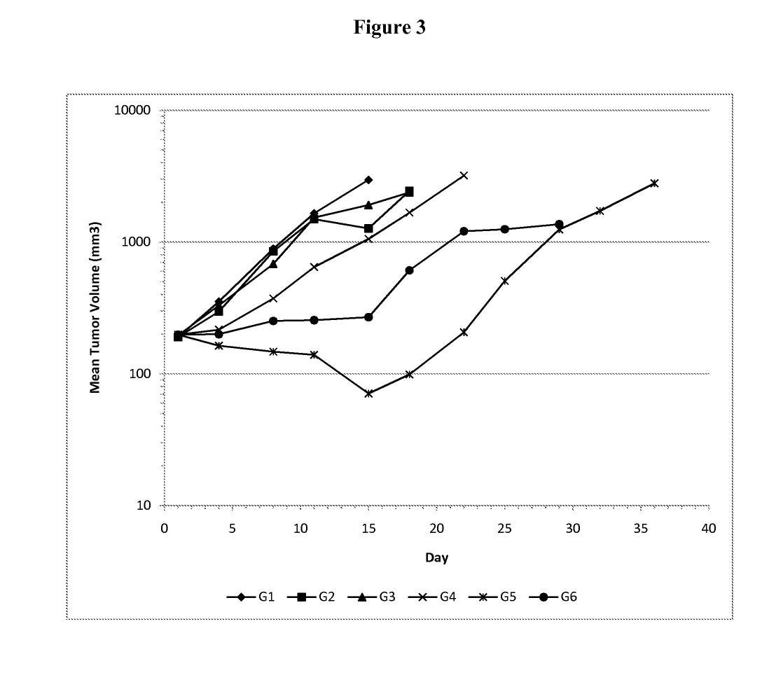

### DEFINITIONS

Unless otherwise stated, the following terms used in the specification and claims shall have the following meanings for the purposes of this Application.

It is noted that, as used in the specification and the appended claims, the singular forms “a,” “an” and “the” include plural referents unless the context clearly dictates otherwise. Further, definitions of standard chemistry terms may be found in reference works, including Carey and Sundberg “ADVANCED ORGANIC CHEMISTRY 4^{TH} ED.” Vols. A (2000) and B (2001), Plenum Press, New York. Also, unless otherwise indicated, conventional methods of mass spectroscopy, NMR, HPLC, protein chemistry, biochemistry, recombinant DNA techniques and pharmacology, within the skill of the art are employed.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with (C₃₋₈) rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkenyl” means a straight or branched, carbon chain that contains at least one carbon-carbon double bond (—CR═CR′— or CR═CR′R″, wherein R, R′ and R″ are each independently hydrogen or further substituents). Examples of alkenyl include vinyl, allyl, isopropenyl, pentenyl, hexenyl, heptenyl, 1-propenyl, 2-butenyl, 2-methyl-2-butenyl, and the like. In particular embodiments, “alkenyl,” either alone or represented along with another radical, can be a (C₂₋₂₀)alkenyl, a (C₂₋₁₅)alkenyl, a (C₂₋₁₀)alkenyl, a (C₂₋₅)alkenyl or a (C₂₋₃)alkenyl. Alternatively, “alkenyl,” either alone or represented along with another radical, can be a (C₂)alkenyl, a (C₃)alkenyl or a (C₄)alkenyl.

“Alkoxy” means an oxygen moiety having a further alkyl substituent. The alkoxy groups of the present invention can be optionally substituted.

“Alkyl” represented by itself means a straight or branched, saturated or unsaturated, aliphatic radical having a chain of carbon atoms, optionally with one or more of the carbon atoms being replaced with oxygen (See “oxaalkyl”), a carbonyl group (See “oxoalkyl”), sulfur (See “thioalkyl”), and/or nitrogen (See “azaalkyl”). Preferably, the term “alkyl” means a straight or branched, saturated or unsaturated, aliphatic radical having a chain of carbon atoms. (C_{X})alkyl and (C_{X-Y})alkyl are typically used where X and Y indicate the number of carbon atoms in the chain. For example, (C₁₋₆)alkyl includes alkyls that have a chain of between 1 and 6 carbons (e.g., methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl and the like) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenethyl, 1-phenylethyl, 3-phenylpropyl, 2-thienylmethyl, 2-pyridinylmethyl and the like). In particular embodiments, “alkyl,” either alone or represented along with another radical, can be a (C₁₋₂₀)alkyl, a (C₁₋₁₅)alkyl, a (C₁₋₁₀)alkyl, a (C₁₋₅)alkyl or a (C₁₋₃)alkyl. Alternatively, “alkyl,” either alone or represented along with another radical, can be a (C₁)alkyl, a (C₂)alkyl or a (C₃)alkyl.

“Alkylene”, unless indicated otherwise, means a straight or branched, saturated or unsaturated, aliphatic, divalent radical. (C_{X})alkylene and (C_{X-Y})alkylene are typically used where X and Y indicate the number of carbon atoms in the chain. For example, (C₁₋₆)alkylene includes methylene (—CH₂—), ethylene (—CH₂CH₂—), trimethylene (—CH₂CH₂CH₂—), tetramethylene (—CH₂CH₂CH₂CH₂—) 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like. In particular embodiments, “alkylene,” either alone or represented along with another radical, can be a (C₁₋₂₀)alkylene, a (C₁₋₁₅)alkylene, a (C₁₋₁₀)alkylene, a (C₁₋₅)alkylene or a (C₁₋₃)alkylene. Alternatively, “alkylene,” either alone or represented along with another radical, can be a (C₁)alkylene, a (C₂)alkylene or a (C₃)alkylene.

“Alkylidene” means a straight or branched, saturated or unsaturated, aliphatic radical connected to the parent molecule by a double bond. (C_{X})alkylidene and (C_{X-Y}) alkylidene are typically used where X and Y indicate the number of carbon atoms in the chain. For example, (C₁₋₆)alkylidene includes methylene (═CH₂), ethylidene (═CHCH₃), isopropylidene (═C(CH₃)₂), propylidene (═CHCH₂CH₃), allylidene (═CH—CH═CH₂), and the like. In particular embodiments, “alkylidene,” either alone or represented along with another radical, can be a (C₁₋₂₀)alkylidene, a (C₁₋₁₅)alkylidene, a (C₁₋₁₀)alkylidene, a (C₁₋₅)alkylidene or a (C₁₋₃)alkylidene. Alternatively, “alkylidene,” either alone or represented along with another radical, can be a (C₁)alkylidene, a (C₂)alkylidene or a (C₃)alkylidene.

“Alkynyl” means a straight or branched, carbon chain that contains at least one carbon-carbon triple bond (—C≡C— or —C≡CR, wherein R is hydrogen or a further substituent). Examples of alkynyl include ethynyl, propargyl, 3-methyl-1-pentynyl, 2-heptynyl and the like. In particular embodiments, “alkynyl,” either alone or represented along with another radical, can be a (C₂₋₂₀)alkynyl, a (C₂₋₁₅)alkynyl, a (C₂₋₁₀)alkynyl, a (C₂₋₅)alkynyl or a (C₂₋₃)alkynyl. Alternatively, “alkynyl,” either alone or represented along with another radical, can be a (C₂)alkynyl, a (C₃)alkynyl or a (C₄)alkynyl.

“Amido” means the radical C(═O)—NR—, C(═O)—NRR′, —NR—C(═O)— and/or —NR—C(═O)R′, wherein each R and R′ are independently hydrogen or a further substituent.

“Amino” means a nitrogen moiety having two further substituents where, for example, a hydrogen or carbon atom is attached to the nitrogen. For example, representative amino groups include —NH₂, —NHCH₃, —N(CH₃)₂, —NH((C₁₋₁₀)alkyl), —N((C₁₋₁₀) alkyl)₂, —NH(aryl), —NH(heteroaryl), —N(aryl)₂, —N(heteroaryl)₂, and the like. Optionally, the two substituents together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (See “heteroaryl”).

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. (C_{X})aryl and (C_{X-Y})aryl are typically used where X and Y indicate the number of carbon atoms in the ring. In particular embodiments, “aryl,” either alone or represented along with another radical, can be a (C₃₋₁₄)aryl, a (C₃₋₁₀)aryl, a (C₃₋₇)aryl, a (C₈₋₁₀)aryl or a (C₅₋₇)aryl. Alternatively, “aryl,” either alone or represented along with another radical, can be a (C₅)aryl, a (C₆)aryl, a (C₇)aryl, a (C₈)aryl, a (C₉)aryl or a (C₁₀)aryl.

“Azaalkyl” means an alkyl, as defined above, except where one or more of the carbon atoms forming the alkyl chain are replaced with substituted or unsubstituted nitrogen atoms (—NR— or —NRR′, wherein R and R′ are each independently hydrogen or further substituents). For example, a (C₁₋₁₀)azaalkyl refers to a chain comprising between 1 and 10 carbons and one or more nitrogen atoms.

“Bicycloalkyl” means a saturated or partially unsaturated fused, spiro or bridged bicyclic ring assembly. In particular embodiments, “bicycloalkyl,” either alone or represented along with another radical, can be a (C₄₋₁₅)bicycloalkyl, a (C₄₋₁₀)bicycloalkyl, a (C₆₋₁₀)bicycloalkyl or a (C₈₋₁₀)bicycloalkyl. Alternatively, “bicycloalkyl,” either alone or represented along with another radical, can be a (C₈)bicycloalkyl, a (C₉)bicycloalkyl or a (C₁₀)bicycloalkyl.

“Bicycloaryl” means a fused, spiro or bridged bicyclic ring assembly wherein at least one of the rings comprising the assembly is aromatic. (C_{X})bicycloaryl and (C_{X-Y}) bicycloaryl are typically used where X and Y indicate the number of carbon atoms in the bicyclic ring assembly and directly attached to the ring. In particular embodiments, “bicycloaryl,” either alone or represented along with another radical, can be a (a (C₄₋₁₅)bicycloaryl, a (C₄₋₁₀)bicycloaryl, a (C₆₋₁₀)bicycloaryl or a (C₈₋₁₀)bicycloaryl. Alternatively, “bicycloaryl,” either alone or represented along with another radical, can be a (C₈)bicycloaryl, a (C₉)bicycloaryl or a (C₁₀)bicycloaryl.

“Bridging ring” and “bridged ring” as used herein refer to a ring that is bonded to another ring to form a compound having a bicyclic or polycyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRR′, wherein R and R′ are each independently hydrogen or further substituents.

“Carbocycle” means a ring consisting of carbon atoms.

“Carbonyl” means the radical C(═O)— and/or C(═O)R, wherein R is hydrogen or a further substituent. It is noted that the carbonyl radical may be further substituted with a variety of substituents to form different carbonyl groups including acids, acid halides, aldehydes, amides, esters, and ketones.

“Carboxy” means the radical C(═O)—O— and/or C(═O)—OR, wherein R is hydrogen or a further substituent. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

As used herein, “cMet” is synonymous with “c-Met”, “MET”, “Met”, “heptaocyte growth factor receptor” and other designations known to those skilled in the art.

“Cyano” means the radical —CN.

“Cycloalkyl” means a non-aromatic, saturated or partially unsaturated, monocyclic, bicyclic or polycyclic ring assembly. (C_{X})cycloalkyl and (C_{X-Y})cycloalkyl are typically used where X and Y indicate the number of carbon atoms in the ring assembly. For example, (C₃₋₁₀)cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like. In particular embodiments, “cycloalkyl,” either alone or represented along with another radical, can be a (C₃₋₁₄)cycloalkyl, a (C₃₋₁₀)cycloalkyl, a (C₃₋₇)cycloalkyl, a (C₈₋₁₀)cycloalkyl or a (C₅₋₇)cycloalkyl. Alternatively, “cycloalkyl,” either alone or represented along with another radical, can be a (C₅)cycloalkyl, a (C₆)cycloalkyl, a (C₇)cycloalkyl, a (C₈)cycloalkyl, a (C₉)cycloalkyl or a (C₁₀)cycloalkyl.

“Cycloalkylene” means a divalent, saturated or partially unsaturated, monocyclic, bicyclic or polycyclic ring assembly. (C_{X})cycloalkylene and (C_{X-Y})cycloalkylene are typically used where X and Y indicate the number of carbon atoms in the ring assembly. In particular embodiments, “cycloalkylene,” either alone or represented along with another radical, can be a (C₃₋₁₄)cycloalkylene, a (C₃₋₁₀)cycloalkylene, a (C₃₋₇)cycloalkylene, a (C₈₋₁₀)cycloalkylene or a (C₅₋₇)cycloalkylene. Alternatively, “cycloalkylene,” either alone or represented along with another radical, can be a (C₅)cycloalkylene, a (C₆)cycloalkylene, a (C₇)cycloalkylene, a (C₈)cycloalkylene, a (C₉)cycloalkylene or a (C₁₀)cycloalkylene.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a ring that is bonded to another ring to form a compound having a bicyclic structure where the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems may be saturated, partially saturated, carbocyclics, heterocyclics, aromatics, heteroaromatics, and the like.

“Halo” means fluoro, chloro, bromo or iodo.

“Heteroalkyl” means alkyl, as defined in this Application, provided that one or more of the atoms within the alkyl chain is a heteroatom. In particular embodiments, “heteroalkyl,” either alone or represented along with another radical, can be a hetero(C₁₋₂₀)alkyl, a hetero(C₁₋₁₅)alkyl, a hetero(C₁₋₁₀)alkyl, a hetero(C₁₋₅)alkyl, a hetero(C₁₋₃)alkyl or a hetero(C₁₋₂)alkyl. Alternatively, “heteroalkyl,” either alone or represented along with another radical, can be a hetero(C₁)alkyl, a hetero(C₂)alkyl or a hetero(C₃)alkyl.

“Heteroaryl” means a monocyclic, bicyclic or polycyclic aromatic group wherein at least one ring atom is a heteroatom and the remaining ring atoms are carbon. Monocyclic heteroaryl groups include, but are not limited to, cyclic aromatic groups having five or six ring atoms, wherein at least one ring atom is a heteroatom and the remaining ring atoms are carbon. The nitrogen atoms can be optionally quaternerized and the sulfur atoms can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, a cycloalkenyl ring, and another monocyclic heteroaryl or heterocycloalkyl ring. These bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups of this invention can be substituted or unsubstituted. In particular embodiments, “heteroaryl,” either alone or represented along with another radical, can be a hetero(C₁₋₁₃)aryl, a hetero(C₂₋₁₃)aryl, a hetero(C₂₋₆)aryl, a hetero(C₃₋₉)aryl or a hetero(C₅₋₉)aryl. Alternatively, “heteroaryl,” either alone or represented along with another radical, can be a hetero(C₃)aryl, a hetero(C₄)aryl, a hetero(C₅)aryl, a hetero(C₆)aryl, a hetero(C₇)aryl, a hetero(C₈)aryl or a hetero(C₉)aryl.

“Heteroatom” refers to an atom that is not a carbon atom. Particular examples of heteroatoms include, but are not limited to, nitrogen, oxygen, and sulfur.

“Heterobicycloalkyl” means bicycloalkyl, as defined in this Application, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like. In particular embodiments, “heterobicycloalkyl,” either alone or represented along with another radical, can be a hetero(C₁₋₁₄)bicycloalkyl, a hetero(C₄₋₁₄)bicycloalkyl, a hetero(C₄₋₉)bicycloalkyl or a hetero(C₅₋₉)bicycloalkyl. Alternatively, “heterobicycloalkyl,” either alone or represented along with another radical, can be a hetero(C₅)bicycloalkyl, hetero(C₆)bicycloalkyl, hetero(C₇)bicycloalkyl, hetero(C₈)bicycloalkyl or a hetero(C₉)bicycloalkyl.

“Heterobicycloaryl” means bicycloaryl, as defined in this Application, provided that one or more of the atoms within the ring is a heteroatom. For example, hetero(C₄₋₁₂)bicycloaryl as used in this Application includes, but is not limited to, 2-amino-4-oxo-3,4-dihydropteridin-6-yl, tetrahydroisoquinolinyl, and the like. In particular embodiments, “heterobicycloaryl,” either alone or represented along with another radical, can be a hetero(C₁₋₁₄)bicycloaryl, a hetero(C₄₋₁₄)bicycloaryl, a hetero(C₄₋₉)bicycloarylor a hetero(C₅₋₉)bicycloaryl. Alternatively, “heterobicycloaryl,” either alone or represented along with another radical, can be a hetero(C₅)bicycloaryl, hetero(C₆)bicycloaryl, hetero(C₇)bicycloaryl, hetero(C₈)bicycloaryl or a hetero(C₉)bicycloaryl.

“Heterocycloalkyl” means cycloalkyl, as defined in this Application, provided that one or more of the atoms forming the ring is a heteroatom selected, independently from N, O, or S, Non-exclusive examples of heterocycloalkyl include piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like. In particular embodiments, “heterocycloalkyl,” either alone or represented along with another radical, can be a hetero(C₁₋₁₃)cycloalkyl, a hetero(C₁₋₉)cycloalkyl, a hetero(C₁₋₆)cycloalkyl, a hetero(C₅₋₉)cycloalkyl or a hetero(C₂₋₆)cycloalkyl. Alternatively, “heterocycloalkyl,” either alone or represented along with another radical, can be a hetero(C₂)cycloalkyl, a hetero(C₃)cycloalkyl, a hetero(C₄)cycloalkyl, a hetero(C₅)cycloalkyl, a hetero(C₆)cycloalkyl, hetero(C₇)cycloalkyl, hetero(C₈)cycloalkyl or a hetero(C₉)cycloalkyl.

“Heterocycloalkylene” means cycloalkylene, as defined in this Application, provided that one or more of the ring member carbon atoms is replaced by a heteroatom. In particular embodiments, “heterocycloalkylene,” either alone or represented along with another radical, can be a hetero(C₁₋₁₃)cycloalkylene, a hetero(C₁₋₉)cycloalkylene, a hetero(C₁₋₆)cycloalkylene, a hetero(C₅₋₉)cycloalkylene or a hetero(C₂₋₆)cycloalkylene. Alternatively, “heterocycloalkylene,” either alone or represented along with another radical, can be a hetero(C₂)cycloalkylene, a hetero(C₃)cycloalkylene, a hetero(C₄)cycloalkylene, a hetero(C₅)cycloalkylene, a hetero(C₆)cycloalkylene, hetero(C₇)cycloalkylene, hetero(C₈)cycloalkylene or a hetero(C₉)cycloalkylene.

“Hydroxy” means the radical —OH.

“IC₅₀” means the molar concentration of an inhibitor that produces 50% inhibition of the target enzyme.

“Imino” means the radical CR(═NR′) and/or C(═NR′)—, wherein R and R′ are each independently hydrogen or a further substituent.

“Isomers” means compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

Moiety and linker providing “X atom separation” between two other moieties mean that the chain of atoms directly linking the two other moieties is X atoms in length. When X is given as a range (e.g., X₁-X₂), then the chain of atoms is at least X₁ and not more than X₂ atoms in length. It is understood that the chain of atoms can be formed from a combination of atoms including, for example, carbon, nitrogen, sulfur and oxygen atoms. Further, each atom can optionally be bound to one or more substituents, as valencies allow. In addition, the chain of atoms can form part of a ring. Accordingly, in one embodiment, a moiety providing X atom separation between two other moieties (R and R′) can be represented by R-(L)ₓ-R′ where each L is independently selected from the group consisting of CR″R′″, NR″, O, S, CO, CS, C═NR′″, SO, SO₂, and the like, where any two or more of R″, R′″, R″ and R′″ can be taken together to form a substituted or unsubstituted ring.

“Nitro” means the radical —NO₂.

“Oxaalkyl” means an alkyl, as defined above, except where one or more of the carbon atoms forming the alkyl chain are replaced with oxygen atoms (—O— or —OR, wherein R is hydrogen or a further substituent). For example, an oxa(C₁₋₁₀)alkyl refers to a chain comprising between 1 and 10 carbons and one or more oxygen atoms.

“Oxoalkyl” means an alkyl, as defined above, except where one or more of the carbon atoms forming the alkyl chain are replaced with carbonyl groups (—C(═O)— or —C(═O)—R, wherein R is hydrogen or a further substituent). The carbonyl group may be an aldehyde, ketone, ester, amide, acid, or acid halide. For example, an oxo(C₁₋₁₀)alkyl refers to a chain comprising between 1 and 10 carbon atoms and one or more carbonyl groups.

“Oxy” means the radical O— or —OR, wherein R is hydrogen or a further substituent. Accordingly, it is noted that the oxy radical may be further substituted with a variety of substituents to form different oxy groups including hydroxy, alkoxy, aryloxy, heteroaryloxy or carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

The term “pharmaceutically acceptable salt” refers to salts of pharmaceutically acceptable organic acids and bases or inorganic acids and bases. Such salts are well known in the art and include those described in Journal of Pharmaceutical Science, 66, 2-19 (1977). Examples are the hydrochloride and mesylate salts.

“Polycyclic ring” includes bicyclic and multi-cyclic rings. The individual rings comprising the polycyclic ring can be fused, spiro or bridging rings.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

“Protected derivatives” means derivatives of inhibitors in which a reactive site or sites are blocked with protecting groups. Protected derivatives are useful in the preparation of inhibitors or in themselves may be active as inhibitors. A comprehensive list of suitable protecting groups can be found in T. W. Greene, Protecting Groups in Organic Synthesis, 3rd edition, John Wiley & Sons, Inc. 1999.

“Ring” and “ring assembly” means a carbocyclic or a heterocyclic system and includes aromatic and non-aromatic systems. The system can be monocyclic, bicyclic or polycyclic. In addition, for bicyclic and polycyclic systems, the individual rings comprising the polycyclic ring can be fused, spiro or bridging rings.

“Subject” and “patient” include humans, non-human mammals (e.g., dogs, cats, rabbits, cattle, horses, sheep, goats, swine, deer, and the like) and non-mammals (e.g., birds, and the like).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl[(CH₃)₃C—OCO—], benzyloxycarbonyl, p-methoxybenzyloxycarbonyl, vinyloxycarbonyl, β-(p-toluenesulfonyl)ethoxycarbonyl, and the like. Examples of suitable amino acid residues include amino acid residues per se and amino acid residues that are protected with a protecting group. Suitable amino acid residues include, but are not limited to, residues of Gly (glycine), Ala (alanine; CH₃CH(NH₂)CO—), Arg (arginine), Asn (asparagine), Asp (aspartic acid), Cys (cysteine), Glu (glutamic acid), His (histidine), Ile (isoleucine), Leu (leucine; (CH₃)₂CHCH₂CH(NH₂)CO—), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and 13-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like. Suitable peptide residues include peptide residues comprising two to five, and optionally two to three, of the aforesaid amino acid residues. Examples of such peptide residues include, but are not limited to, residues of such peptides as Ala-Ala[CH₃CH(NH₂)CO—NHCH(CH₃)CO—], Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [(CH₃)₃C—OCO—], and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl).

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical SO— and/or —SO—R, wherein R is hydrogen or a further substituent. It is noted that the sulfinyl radical may be further substituted with a variety of substituents to form different sulfinyl groups including sulfinic acids, sulfinamides, sulfinyl esters, and sulfoxides.

“Sulfonyl” means the radical —SO₂— and/or —SO₂—R, wherein R is hydrogen or a further substituent. It is noted that the sulfonyl radical may be further substituted with a variety of substituents to form different sulfonyl groups including sulfonic acids, sulfonamides, sulfonate esters, and sulfones.

“Therapeutically effective amount” and “pharmaceutically effective amount” mean that amount which, when administered to an animal for treating a disease, is sufficient to effect such treatment for the disease.

“Thio” denotes replacement of an oxygen by a sulfur and includes, but is not limited to, —SR, —S— and ═S containing groups.

“Thioalkyl” means an alkyl, as defined above, except where one or more of the carbon atoms forming the alkyl chain are replaced with sulfur atoms (—S— or S—R, wherein R is hydrogen or a further substituent). For example, a thio(C₁₋₁₀)alkyl refers to a chain comprising between 1 and 10 carbons and one or more sulfur atoms.

“Thiocarbonyl” means the radical C(═S)— and/or C(═S)—R, wherein R is hydrogen or a further substituent. It is noted that the thiocarbonyl radical may be further substituted with a variety of substituents to form different thiocarbonyl groups including thioacids, thioamides, thioesters, and thioketones.

“Treatment” or “treating” means any administration of a compound of the present invention and includes:

(1) preventing the disease from occurring in an animal which may be predisposed to the disease but does not yet experience or display the pathology or symptomololgy of the disease,

(2) inhibiting the disease in an animal that is experiencing or displaying the pathology or symptomology of the diseased (i.e., arresting further development of the pathology and/or symptomology), or

(3) ameliorating the disease in an animal that is experiencing or displaying the pathology or symptomalogy of the diseased (i.e., reversing the pathology and/or symptomalogy).

It is noted in regard to all of the definitions provided herein that the definitions should be interpreted as being open ended in the sense that further substituents beyond those specified may be included. Hence, a C₁ alkyl indicates that there is one carbon atom but does not indicate what the substituents on the carbon atom are. Hence, a (C₁)alkyl comprises methyl (i.e., —CH₃) as well as —CRR′R″ where R, R′, and R″ may each independently be hydrogen or a further substituent where the atom attached to the carbon is a heteroatom or cyano. Hence, CF₃, CH₂OH and CH₂CN, for example, are all (C₁)alkyls. Similarly, terms such as alkylamino and the like comprise dialkylamino and the like.

A compound having a formula that is represented with a dashed bond is intended to include the formulae optionally having zero, one or more double bonds, as exemplified and shown below:

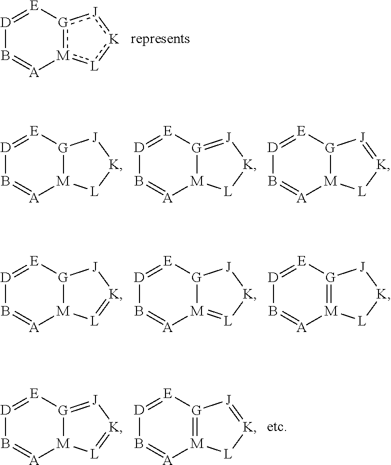

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

### DETAILED DESCRIPTION OF THE INVENTION

The present invention relates to methods of treating conditions mediated by HGF/cMET. The present invention also relates to kits and articles of manufacture comprising such compounds.

cMET belongs to the phosphoryl transferase family of enzymes that transfer phosphorous-containing groups from one substrate to another. By the conventions set forth by the Nomenclature Committee of the International Union of Biochemistry and Molecular Biology (IUBMB) enzymes of this type have Enzyme Commission (EC) numbers starting with 2.7.-.-(See, Bairoch A., The ENZYME database in Nucleic Acids Res. 28:204-305 (2000)). Kinases are a class of enzymes that function in the catalysis of phosphoryl transfer. The protein kinases constitute one of the largest subfamilies of structurally related phosphoryl transferases and are responsible for the control of a wide variety of cellular signal transduction processes. (See, Hardie, G. and Hanks, S. (1995) The Protein Kinase Facts Book, I and II, Academic Press, San Diego, Calif.).

Disregulation of cMET is implicated in such diseases as cancers (including carcinomas (e.g., bladder, breast, cervical, cholangiocarcinoma, colorectal, esophageal, gastric, head and neck, kidney, liver, lung, nasopharygeal, ovarian, pancreatic, prostate and thyroid); musculoskeletal sarcomas (e.g., osteosarcoma, synovial sarcoma, and rhabdomyosarcoma); soft tissue sarcomas (e.g., MFH/fibrosrcoma, leiomyosarcoma, and Kaposi's sarcoma); hematopoietic malignancies (e.g., multiple myeloma, lymphomas, adult T cell leukemia, acute myelogenous leukemia, and chronic myeloid leukemia); and other neoplasms (e.g., glioblastomas, astrocytomas, melanoma, mesothelioma, and Wilm's tumor)); and proliferative diseases (e.g., myeloproliferative disorders, atherosclerosis, and fibrosis of the lung).

It is noted that the pharmaceutical compositions of the present invention may also possess inhibitory activity for other receptor tyrosine kinase family members and thus may be used to address disease states associated with these other family members. In particular, the pharmaceutical compositions of the present invention may be used to modulate the activity of other proteins in the Met subfamily (e.g., Ron and Sea).

### cMET Inhibitors

In one embodiment, the cMET inhibitor has the formula as described in International Patent Application No. PCT/US2009/053913, which is hereby incorporated by reference in its entirety. In particular, the cMet inhibitor has the formula:

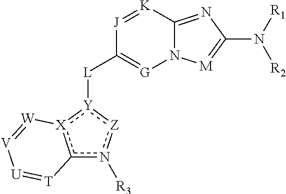

or pharmaceutically acceptable salt thereof, wherein
G is selected from the group consisting of CR₄ and N;
J is selected from the group consisting of CR₅ and N;
K is selected from the group consisting of CR₆ and N;
M is selected from the group consisting of CR₇ and N;
L is absent or a linker providing 1, 2, 3, 4, 5 or 6 atom separation between the rings to which L is attached, wherein the atoms of the linker providing the separation are selected from the group consisting of carbon, oxygen, nitrogen, and sulfur;
T is selected from the group consisting of CR₈ and N;
U is selected from the group consisting of CR₉ and N;
V is selected from the group consisting of CR₁₀ and N;
W is selected from the group consisting of CR₁₁ and N;
X is selected from the group consisting of CR₁₂ and N;
Y is selected from the group consisting of CR₁₃ and N;
Z is selected from the group consisting of CR₁₄R₁₅ and NR₁₆;
R₁ is selected from the group consisting of hydrogen, carbonyloxy, (C₁₋₁₀)alkoxy, (C₄₋₁₂)aryloxy, hetero(C₁₋₁₀)aryloxy, carbonyl, oxycarbonyl, aminocarbonyl, (C₁₋₁₀)alkyl carbonyl, (C₃₋₁₂)cycloalkyl(C₁₋₅)carbonyl, hetero(C₃₋₁₂)cycloalkyl(C₁₋₁₀)carbonyl, aryl(C₁₋₁₀)carbonyl, hetero(C₁₋₁₀)aryl(C₁₋₅)carbonyl, (C₉₋₁₂)bicycloaryl(C₁₋₅)carbonyl, hetero(C₈₋₁₂)bicycloaryl(C₁₋₅)carbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁ has the formula

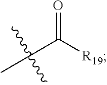

R₂ is hydrogen or a substituent convertible in vivo to hydrogen;
R₃ is selected from the group consisting of hydrogen, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₃ is absent when the nitrogen to which it is bound forms part of a double bond;
R₄ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₅ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₆ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₇ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₈ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₉ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, amido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, amino(C₁₋₁₀)alkyl, amido(C₁₋₁₀)alkylamino(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, carbonyl(C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₀ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, amido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, amino(C₁₋₁₀)alkyl, amido(C₁₋₁₀)alkylamino(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, carbonyl(C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₁ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₂ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₂ is absent when the carbon to which it is bound forms part of a double bond;
R₁₃ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₃ is absent when the carbon to which it is bound forms part of a double bond;
R₁₄ and R₁₅ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₅ is absent when the carbon to which it is bound forms part of a double bond;
R₁₆ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₆ is absent when the nitrogen to which it is bound forms part of a double bond; and
R₁₉ selected from the group consisting of hydrogen, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

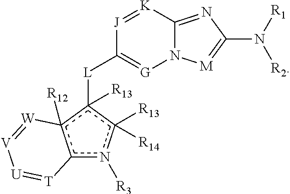

In another embodiment, the cMET inhibitor has the formula:

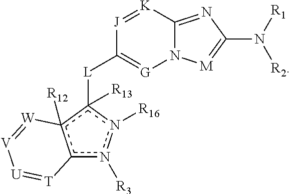

In another embodiment, the cMET inhibitor has the formula:

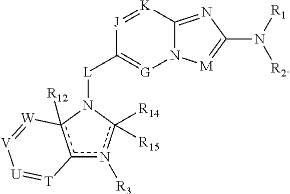

In another embodiment, the cMET inhibitor has the formula:

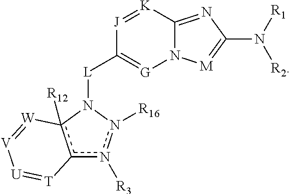

In another embodiment, the cMET inhibitor has the formula:

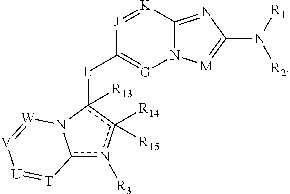

In another embodiment, the cMET inhibitor has the formula:

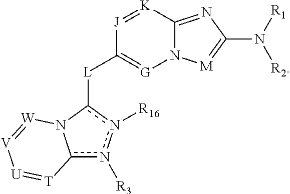

In another embodiment, the cMET inhibitor has the formula:

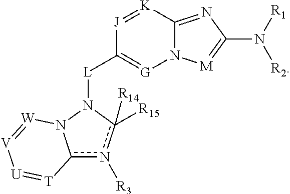

In another embodiment, the cMET inhibitor has the formula:

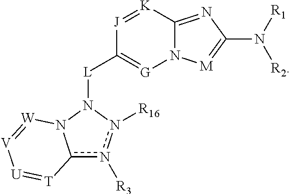

In another embodiment, the cMET inhibitor has the formula:

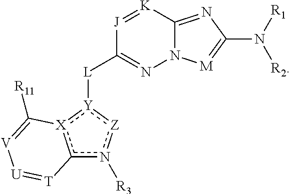

In another embodiment, the cMET inhibitor has the formula:

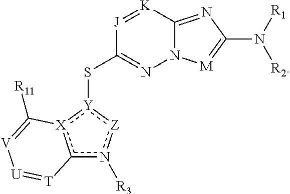

In another embodiment, the cMET inhibitor has the formula:

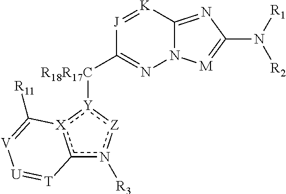

wherein R₁₇ and R₁₈ are each independently elected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

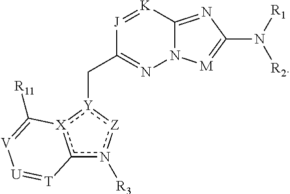

In another embodiment, the cMET inhibitor has the formula:

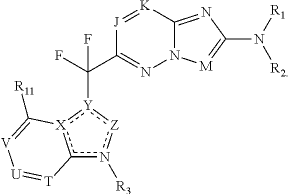

In another embodiment, the cMET inhibitor has the formula:

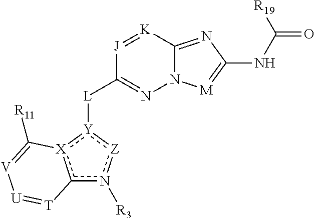

wherein R₁₉ selected from the group consisting of hydrogen, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

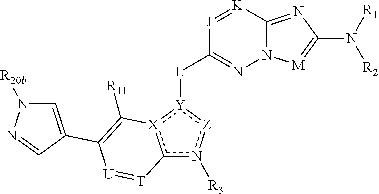

wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

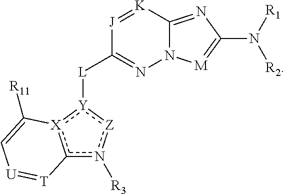

In another embodiment, the cMET inhibitor has the formula:

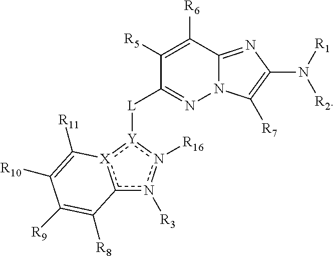

In another embodiment, the cMET inhibitor has the formula:

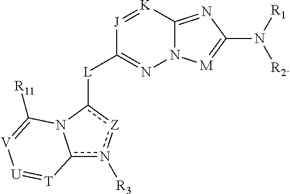

In another embodiment, the cMET inhibitor has the formula:

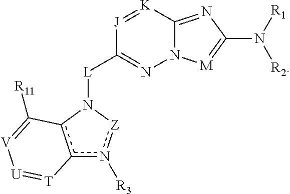

In another embodiment, the cMET inhibitor has the formula:

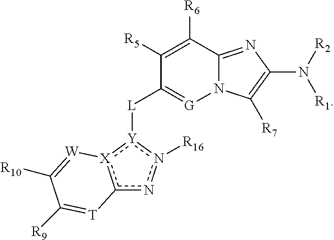

In another embodiment, the cMET inhibitor has the formula:

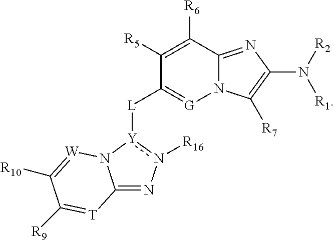

In another embodiment, the cMET inhibitor has the formula:

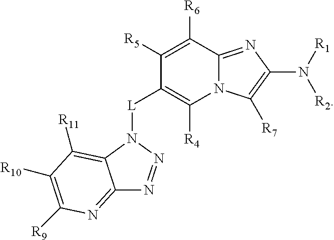

In another embodiment, the cMET inhibitor has the formula:

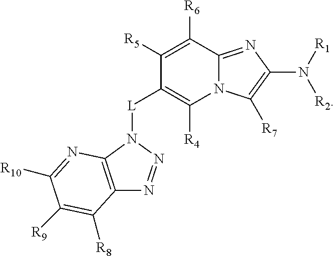

In another embodiment, the cMET inhibitor has the formula:

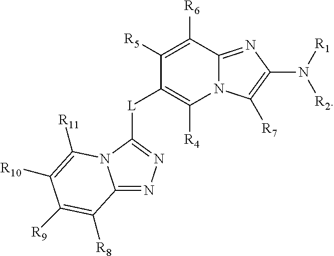

In another embodiment, the cMET inhibitor has the formula:

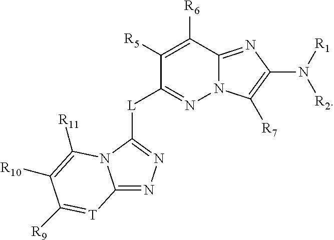

In another embodiment, the cMET inhibitor has the formula:

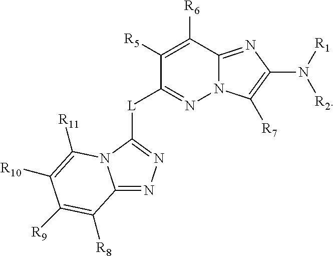

In another embodiment, the cMET inhibitor has the formula:

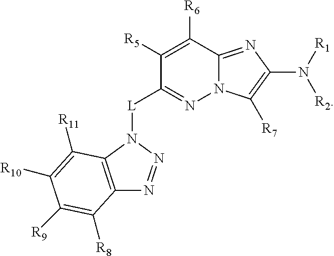

In another embodiment, the cMET inhibitor has the formula:

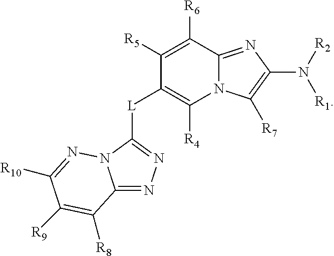

In another embodiment, the cMET inhibitor has the formula:

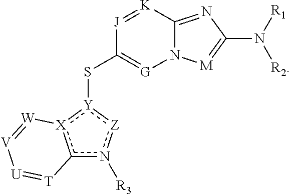

In another embodiment, the cMET inhibitor has the formula:

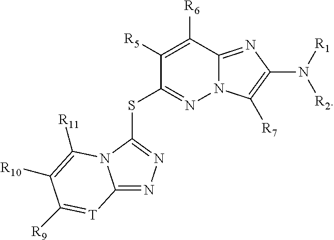

In another embodiment, the cMET inhibitor has the formula:

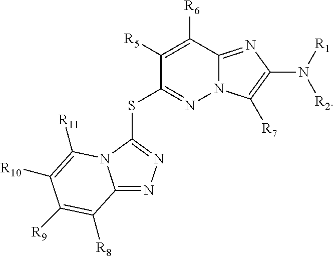

In another embodiment, the cMET inhibitor has the formula:

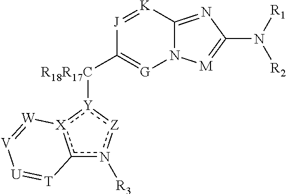

wherein R₁₇ and R₁₈ are each independently elected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

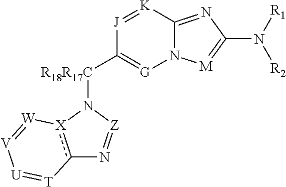

wherein R₁₇ and R₁₈ are each independently elected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

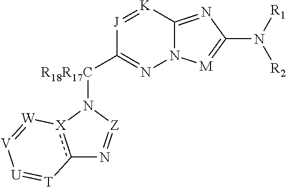

wherein R₁₇ and R₁₈ are each independently elected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

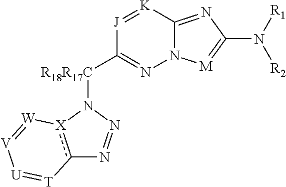

wherein R₁₇ and R₁₈ are each independently elected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

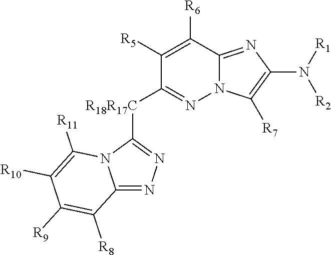

wherein R₁₇ and R₁₈ are each independently elected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

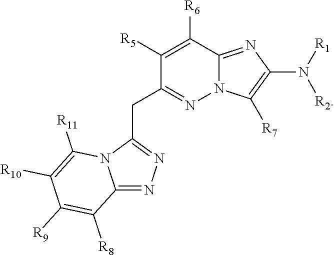

In another embodiment, the cMET inhibitor has the formula:

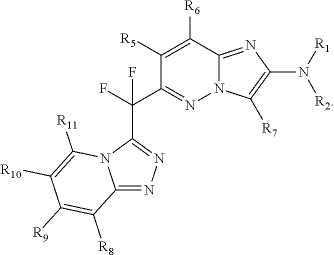

In another embodiment, the cMET inhibitor has the formula:

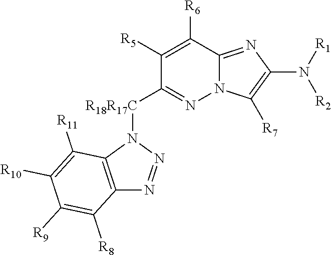

wherein R₁₇ and R₁₈ are each independently elected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another embodiment, the cMET inhibitor has the formula:

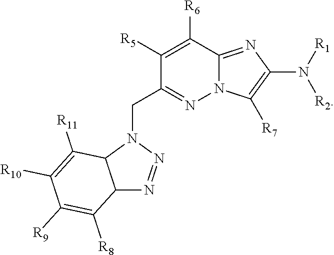

In one variation of each of the above embodiments, G is CR₄. In another variation of each of the above embodiments, G is N.

In still another variation of each of the above embodiments and variations, J is CR₅. In yet another variation of each of the above embodiments and variations, J is N.

In a further variation of each of the above embodiments and variations, K is CR₆. In still a further variation of each of the above embodiments and variations, K is N.

In yet a further variation of each of the above embodiments and variations, M is CR₇. In another variation of each of the above embodiments and variations, M is N.

In another variation of each of the above embodiments and variations,
L is a linker selected from the group consisting of —(CR₂₇R₂₈)ᵣ—, —CO—, —CS—, —C(═NR₂₉)—, —NR₃₀—, —O—, —S—, —SO—, —SO₂— and combinations thereof;
r is selected from the group consisting of 1, 2 and 3;
R₂₇ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, amido, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₂₈ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, amido, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₂₉ is selected from the group consisting of hydrogen, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
R₃₀ is selected from the group consisting of hydrogen, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In still another variation of each of the above embodiments and variations, L is a substituted or unsubstituted (C₁₋₅)alkyl. In yet another variation of each of the above embodiments and variations, L is —CH₂—. In a further variation of each of the above embodiments and variations, L is —CH(CH₃)—. In still a further variation of each of the above embodiments and variations, L is —C(CH₃)₂—. In yet a further variation of each of the above embodiments and variations, L is CF₂—. In a further variation of each of the above embodiments and variations, L is —S—. In another variation of each of the above embodiments and variations, L is —SO—. In still another variation of each of the above embodiments and variations, L is —SO₂—. In yet another variation of each of the above embodiments and variations, L is —CO—. In another variation of each of the above embodiments and variations, L is —O—. In still another variation of each of the above embodiments and variations, L is —NH—. In yet another variation of each of the above embodiments and variations, L is —CH₂—. In a further variation of each of the above embodiments and variations, L is —CO—NH—. In still a further variation of each of the above embodiments and variations, L is —NH—CO—. In yet a further variation of each of the above embodiments and variations, L is —SO₂—NH—. In another variation of each of the above embodiments and variations, L is —NH—SO₂—. In still another variation of each of the above embodiments and variations, L is —NH—NH—. In yet another variation of each of the above embodiments and variations, L is —CO—O—. In a further variation of each of the above embodiments and variations, L is —O—CO—.

In still a further variation of each of the above embodiments and variations, T is CR₈. In yet a further variation of each of the above embodiments and variations, T is N.

In another variation of each of the above embodiments and variations, U is CR₉. In still another variation of each of the above embodiments and variations, U is N.

In yet another variation of each of the above embodiments and variations, V is CR₁₀. In a further variation of each of the above embodiments and variations, V is N.

In still a further variation of each of the above embodiments and variations, W is CR₁₁. In yet a further variation of each of the above embodiments and variations, W is N.

In another variation of each of the above embodiments and variations, X is CR₁₂. In still another variation of each of the above embodiments and variations, X is N.

In yet another variation of each of the above embodiments and variations, Y is CR₁₃. In a further variation of each of the above embodiments and variations, Y is N.

In still a further variation of each of the above embodiments and variations, Z is CR₁₄R₁₅. In yet a further variation of each of the above embodiments and variations, Z is NR₁₆.

In another variation of each of the above embodiments and variations, T, Y and Z are each N. In still another variation of each of the above embodiments and variations, G is CR₄, J is CR₅, K is CR₆, M is CR₇, T is CR₈, U is CR₉, V is CR₁₀, W is CR₁₁, X is CR₁₂ and Y is CR₁₃. In yet another variation of each of the above embodiments and variations, G is CR₄, J is CR₅, K is CR₆, M is CR₇, T is CR₈, U is CR₉, V is CR₁₀, W is CR₁₁ and Y is CR₁₃. In a further variation of each of the above embodiments and variations, G, J, K, M, U, V, W, and X are each CH. In still a further variation of each of the above embodiments and variations, W, Y and Z are each N. In yet a further variation of each of the above embodiments and variations, G, J, K, M, T, U, V, and X are each CH. In another variation of each of the above embodiments and variations, G, X and Z are each N. In still another variation of each of the above embodiments and variations, J, K, M, T, U, V, W and Y are each CH. In yet another variation of each of the above embodiments and variations, X and Z are each N. In a further variation of each of the above embodiments and variations, G, J, K, M, T, U, V, W and Y are each CH. In still a further variation of each of the above embodiments and variations, T is CR₈, U is CR₉, V is CR₁₀, W is CR₁₁, X is CR₁₂, Y is N and Z is CR₁₄R₁₅. In yet a further variation of each of the above embodiments and variations, T is CR₈, U is CR₉, V is CR₁₀, W is CR₁₁, X is N, Y is CR₁₃ and Z is NR₁₆. In another variation of each of the above embodiments and variations, G is N, J is CR₅, K is CR₆ and M is CR₇. In a further variation of each of the above embodiments and variations, J is CR₅, K is CR₆, T is CR₈, U is CR₉, V is CR₁₀ and Z is NR₁₆. In still a further variation of each of the above embodiments and variations, one and only one of G and W is N. In another variation of each of the above embodiments and variations, G is N and W is CR₁₁. In still another variation of each of the above embodiments and variations, G is CR₄ and W is N. In yet a further variation of each of the above embodiments and variations, one and only one of W and Z is N. In another variation of each of the above embodiments and variations, one and only one of W and X is N. In still another variation of each of the above embodiments and variations, one and only one of W, X and Z is N. In a further variation of each of the above embodiments and variations, G is N; and J, K, M, T, U, V and W are each CH. In still a further variation of each of the above embodiments and variations, G is N, J; K, M, T, U and W are each CH; and V is CR₁₀. In still a further variation of each of the above embodiments and variations, G is N, J; K, M, T, U and W are each CH; and V is CR₁₀, wherein R₁₀ is a substituted or unsubstituted hetero(C₁₋₁₀)aryl.

In a further variation of each of the above embodiments and variations, CR₅, CR₆, CR₇, CR₈ and CR₉ are each hydrogen.

In a further variation of each of the above embodiments and variations, R₁ is selected from the group consisting of hydrogen, carbonyl, oxycarbonyl, aminocarbonyl, (C₁₋₁₀)alkylcarbonyl, (C₃₋₁₂)cycloalkyl(C₁₋₅)carbonyl, hetero(C₃₋₁₂)cycloalkyl(C₁₋₁₀)carbonyl, aryl(C₁₋₁₀)carbonyl, hetero(C₁₋₁₀)aryl(C₁₋₅)carbonyl, (C₉₋₁₂)bicycloaryl(C₁₋₅)carbonyl, hetero(C₈₋₁₂)bicycloaryl(C₁₋₅)carbonyl, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;

In yet another variation of each of the above embodiments and variations, R₁ is hydrogen. In a further variation of each of the above embodiments and variations, R₁ has the formula

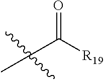

wherein R₁₉ selected from the group consisting of hydrogen, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₂ is hydrogen. In another variation of each of the above embodiments and variations, R₂ is halo. In another variation of each of the above embodiments and variations, R₂ is a substituted or unsubstituted (C₁₋₃)alkyl. In still another variation of each of the above embodiments and variations, R₂ is methyl.

In yet a further variation of each of the above embodiments and variations, R₃ is absent. In still a further variation of each of the above embodiments and variations, R₃ is hydrogen. In another variation of each of the above embodiments and variations, R₃ is a substituted or unsubstituted (C₁₋₃)alkyl. In still another variation of each of the above embodiments and variations, R₃ is methyl.

In still a further variation of each of the above embodiments and variations, R₄ is hydrogen. In another variation of each of the above embodiments and variations, R₄ is halo. In another variation of each of the above embodiments and variations, R₄ is a substituted or unsubstituted (C₁₋₃)alkyl. In still another variation of each of the above embodiments and variations, R₄ is methyl.

In still a further variation of each of the above embodiments and variations, R₅ is hydrogen. In another variation of each of the above embodiments and variations, R₅ is halo. In another variation of each of the above embodiments and variations, R₅ is a substituted or unsubstituted (C₁₋₃)alkyl. In still another variation of each of the above embodiments and variations, R₅ is methyl.

In still a further variation of each of the above embodiments and variations, R₆ is hydrogen. In another variation of each of the above embodiments and variations, R₆ is halo. In another variation of each of the above embodiments and variations, R₆ is a substituted or unsubstituted (C₁₋₃)alkyl. In still another variation of each of the above embodiments and variations, R₆ is methyl.

In still a further variation of each of the above embodiments and variations, R₇ is hydrogen. In another variation of each of the above embodiments and variations, R₇ is halo. In another variation of each of the above embodiments and variations, R₇ is a substituted or unsubstituted (C₁₋₃)alkyl. In still another variation of each of the above embodiments and variations, R₇ is methyl.

In still a further variation of each of the above embodiments and variations, R₈ is hydrogen. In another variation of each of the above embodiments and variations, R₈ is halo. In another variation of each of the above embodiments and variations, R₈ is a substituted or unsubstituted (C₁₋₃)alkyl. In still another variation of each of the above embodiments and variations, R₈ is methyl.

In yet a further variation of each of the above embodiments and variations, R₉ has the formula —C(═O)—NHR₂₀ₐ wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another variation of each of the above embodiments and variations, R₉ has the formula —((C₁₋₃)alkyl)-NHR₂₀ₐ wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still another variation of each of the above embodiments and variations, R₉ has the formula —CH₂—NHR₂₀ₐ wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₉ has the formula —NHR₂₀ₐ wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, wherein R₉ has the formula —NH—C(═O)R₂₀ₐ wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₉ is a substituted or unsubstituted (C₁₋₃)alkoxy. In another variation of each of the above embodiments and variations, R₉ is hydrogen. In another variation of each of the above embodiments and variations, R₉ is halo. In a further variation of each of the above embodiments and variations, R₉ is a substituted or unsubstituted (C₁₋₃)alkyl. In still another variation of each of the above embodiments and variations, R₉ is methyl. In another variation of each of the above embodiments and variations, R₉ is —CF₃.

In still another variation of each of the above embodiments and variations, R₉ has the formula

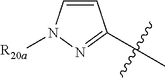

wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₉ has the formula

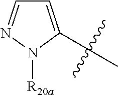

wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₉ has the formula

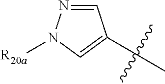

wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₉ has the formula

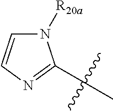

wherein R₂₀ₐ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet a further variation of each of the above embodiments and variations, R₉ has the formula

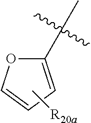

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another variation of each of the above embodiments and variations, R₉ has the formula

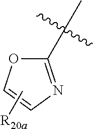

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still another variation of each of the above embodiments and variations, R₉ has the formula

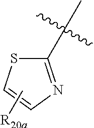

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₉ has the formula

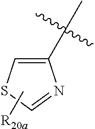

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₉ has the formula

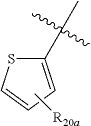

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₉ has the formula

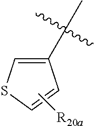

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet a further variation of each of the above embodiments and variations, R₉ has the formula

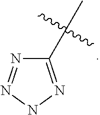 [SMILES: *c1nn[nH]n1]

In another variation of each of the above embodiments and variations, R₉ has the formula

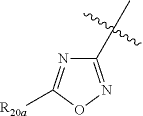

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still another variation of each of the above embodiments and variations, R₉ has the formula

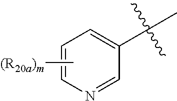

wherein
m is selected from the group consisting of 0, 1, 2, 3 and 4; and
R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₉ has the formula

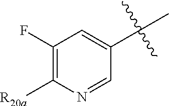

wherein R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₉ has the formula

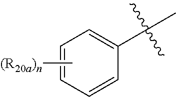

wherein
n is selected from the group consisting of 0, 1, 2, 3, 4 and 5; and
R₂₀ₐ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₉ has a formula selected from the group consisting of

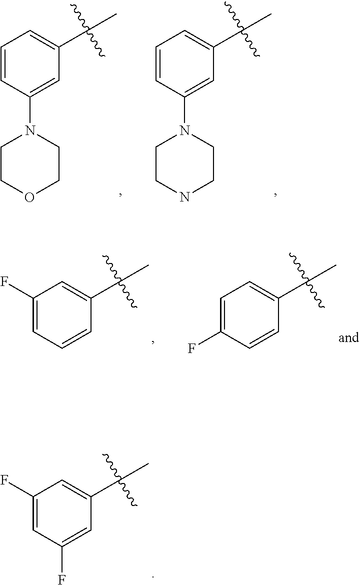 [SMILES: *c1cccc(N2CCOCC2)c1 ; *c1cccc(N2CCNCC2)c1 ; *c1cccc(F)c1 ; *c1ccc(F)cc1 ; *c1cc(F)cc(F)c1]

In yet a further variation of each of the above embodiments and variations, R₉ has a formula selected from the group consisting of

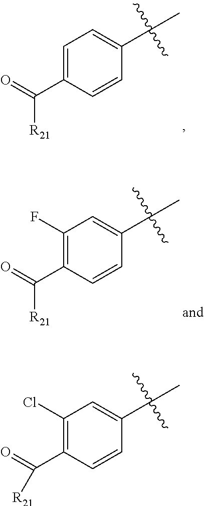

wherein R₂₁ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still another variation of each of the above embodiments and variations, R₁₀ has the formula —C(═O)—NHR_{20b} wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₁₀ has the formula —((C₁₋₃)alkyl)-NHR_{20b} wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₁₀ has the formula —CH₂—NHR_{20b} wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₁₀ has the formula —NHR_{20b} wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet a further variation of each of the above embodiments and variations, R₁₀ has the formula —NH—C(═O)R_{20b} wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₁₀ is hydrogen. In still another variation of each of the above embodiments and variations, R₁₀ is halo. In a further variation of each of the above embodiments and variations, R₁₀ is selected from the group consisting of Cl, Br and I. In still a further variation of each of the above embodiments and variations, R₁₀ is a substituted or unsubstituted (C₁₋₃)alkyl. In a further variation of each of the above embodiments and variations, R₁₀ is methyl. In still a further variation of each of the above embodiments and variations, R₁₀ is —CF₃. In another variation of each of the above embodiments and variations, R₁₀ is a substituted or unsubstituted (C₁₋₃)alkoxy. In a further variation of each of the above embodiments and variations, R₁₀ is cyano.

In yet a further variation of each of the above embodiments and variations, R₁₀ has the formula

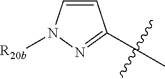

wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another variation of each of the above embodiments and variations, R₁₀ has the formula

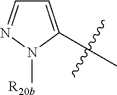

wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still another variation of each of the above embodiments and variations, R₁₀ has the formula

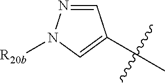

wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₁₀ has the formula

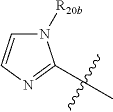

wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₁₀ has the formula

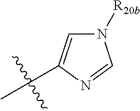

wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₁₀ has the formula

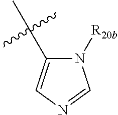

wherein R_{20b} selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another variation of each of the above embodiments and variations, R₁₀ is a substituted or unsubstituted hetero(C₁₋₁₀)aryl. In still another variation of each of the above embodiments and variations, R₁₀ is a substituted or unsubstituted hetero(C₁₋₅)aryl. In yet another variation of each of the above embodiments and variations, R₁₀ is a substituted or unsubstituted hetero(C₃₋₁₂)cycloalkyl. In a further variation of each of the above embodiments and variations, R₁₀ is a substituted or unsubstituted hetero(C₃₋₆)cycloalkyl.

In a further variation of each of the above embodiments and variations, R₁₀ is a substituted 5-membered heteroaryl group having 1-3 heteroatoms, wherein the heteroaryl ring has at least one oxo group in the ring or at least one hydroxy substituent. In one particular variation, the heteroaryl group has 1-3 nitrogen atoms.

In a further variation of each of the above embodiments and variations, R₁₀ is a substituted 5-membered heterocycloalkyl group having 1-3 heteroatoms, wherein the heterocycloalkyl ring has at least one oxo group in the ring or at least one hydroxy substituent. In one particular variation, the heterocycloalkyl group has 1-3 nitrogen atoms.

In a further variation of each of the above embodiments and variations, R₁₀ is a substituted 6-membered heteroaryl group having 1-3 heteroatoms, wherein the heteroaryl ring has at least one oxo group in the ring or at least one hydroxy substituent. In one particular variation, the heteroaryl group has 1-3 nitrogen atoms.

In a further variation of each of the above embodiments and variations, R₁₀ is a substituted 6-membered heterocycloalkyl group having 1-3 heteroatoms, wherein the heterocycloalkyl ring has at least one oxo group in the ring or at least one hydroxy substituent. In one particular variation, the heterocycloalkyl group has 1-3 nitrogen atoms.

In yet another variation of each of the above embodiments and variations, R₁₀ has the formula

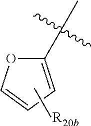

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₁₀ has the formula

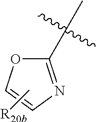

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₁₀ has the formula

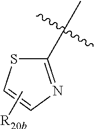

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet a further variation of each of the above embodiments and variations, R₁₀ has the formula

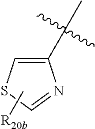

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another variation of each of the above embodiments and variations, R₁₀ has the formula

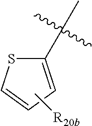

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still another variation of each of the above embodiments and variations, R₁₀ has the formula

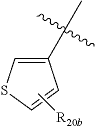

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet another variation of each of the above embodiments and variations, R₁₀ has the formula

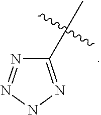 [SMILES: *c1nn[nH]n1]

In a further variation of each of the above embodiments and variations, R₁₀ has the formula

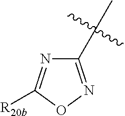

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₁₀ has the formula

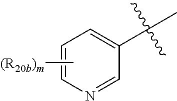

wherein
m is selected from the group consisting of 0, 1, 2, 3 and 4; and
R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In yet a further variation of each of the above embodiments and variations, R₁₀ has the formula

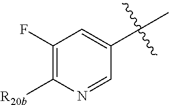

wherein R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another variation of each of the above embodiments and variations, R₁₀ has the formula

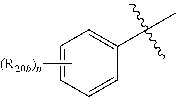

wherein
n is selected from the group consisting of 0, 1, 2, 3, 4 and 5; and
R_{20b} is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still another variation of each of the above embodiments and variations, R₁₀ has a formula selected from the group consisting of

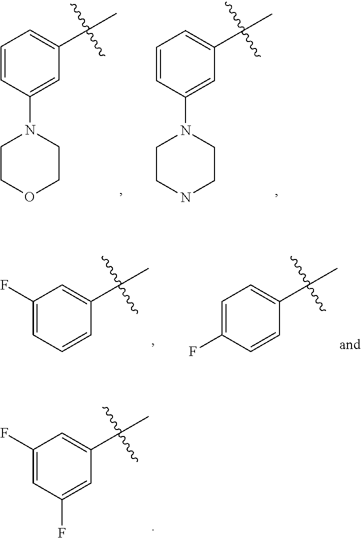 [SMILES: *c1cccc(N2CCOCC2)c1 ; *c1cccc(N2CCNCC2)c1 ; *c1cccc(F)c1 ; *c1ccc(F)cc1 ; *c1cc(F)cc(F)c1]

In yet another variation of each of the above embodiments and variations, R₁₀ has a formula selected from the group consisting of

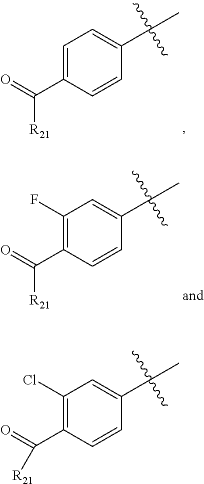

wherein R₂₁ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₁₁ is hydrogen. In still a further variation of each of the above embodiments and variations, R₁₁ is halo. In yet a further variation of each of the above embodiments and variations, R₁₁ is a substituted or unsubstituted (C₁₋₃)alkyl. In another variation of each of the above embodiments and variations, R₁₁ is methyl.

In yet a further variation of each of the above embodiments and variations, R₁₂ is absent. In a further variation of each of the above embodiments and variations, R₁₂ is hydrogen. In still a further variation of each of the above embodiments and variations, R₁₂ is halo. In yet a further variation of each of the above embodiments and variations, R₁₂ is a substituted or unsubstituted (C₁₋₃)alkyl. In another variation of each of the above embodiments and variations, R₁₂ is methyl.

In still another variation of each of the above embodiments and variations, R₁₃ is absent. In a further variation of each of the above embodiments and variations, R₁₃ is hydrogen. In still a further variation of each of the above embodiments and variations, R₁₃ is halo. In yet a further variation of each of the above embodiments and variations, R₁₃ is a substituted or unsubstituted (C₁₋₃)alkyl. In another variation of each of the above embodiments and variations, R₁₃ is methyl.

In a further variation of each of the above embodiments and variations, R₁₄ is hydrogen. In still a further variation of each of the above embodiments and variations, R₁₄ is halo. In yet a further variation of each of the above embodiments and variations, R₁₄ is a substituted or unsubstituted (C₁₋₃)alkyl. In another variation of each of the above embodiments and variations, R₁₄ is methyl.

In still a further variation of each of the above embodiments and variations, R₁₅ is absent. In a further variation of each of the above embodiments and variations, R₁₅ is hydrogen. In still a further variation of each of the above embodiments and variations, R₁₅ is halo. In yet a further variation of each of the above embodiments and variations, R₁₅ is a substituted or unsubstituted (C₁₋₃)alkyl. In another variation of each of the above embodiments and variations, R₁₅ is methyl.

In yet a further variation of each of the above embodiments and variations, R₁₆ is absent. In a further variation of each of the above embodiments and variations, R₁₆ is hydrogen. In still a further variation of each of the above embodiments and variations, R₁₆ is halo. In yet a further variation of each of the above embodiments and variations, R₁₆ is a substituted or unsubstituted (C₁₋₃)alkyl. In another variation of each of the above embodiments and variations, R₁₆ is methyl.

In a further variation of each of the above embodiments and variations, R₁₇ is hydrogen. In still a further variation of each of the above embodiments and variations, R₁₇ is halo. In another variation of each of the above embodiments and variations, R₁₇ is fluoro. In yet a further variation of each of the above embodiments and variations, R₁₇ is a substituted or unsubstituted (C₁₋₃)alkyl. In another variation of each of the above embodiments and variations, R₁₇ is methyl. In one variation of each of the above embodiments and variations containing R₁₇, R₁₇ is unsubstituted.

In a further variation of each of the above embodiments and variations, R₁₈ is hydrogen. In still a further variation of each of the above embodiments and variations, R₁₈ is halo. In another variation of each of the above embodiments and variations, R₁₈ is fluoro. In yet a further variation of each of the above embodiments and variations, R₁₈ is a substituted or unsubstituted (C₁₋₃)alkyl. In another variation of each of the above embodiments and variations, R₁₈ is methyl. In one variation of each of the above embodiments and variations containing R₁₈, R₁₈ is unsubstituted.

In another variation of each of the above embodiments and variations, R₁₉ is selected from the group consisting of hydrogen, amino, (C₁₋₁₀)alkylamino, sulfonamido, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In another variation of each of the above embodiments and variations, R₁₉ is a substituted or unsubstituted (C₁₋₅)alkyl. In still another variation of each of the above embodiments and variations, R₁₉ is methyl. In yet another variation of each of the above embodiments and variations, R₁₉ is trifluoromethyl. In a further variation of each of the above embodiments and variations, R₁₉ is isopropyl. In still a further variation of each of the above embodiments and variations, R₁₉ is butyl. In yet a further variation of each of the above embodiments and variations, R₁₉ is a substituted or unsubstituted (C₃₋₆)cycloalkyl. In another variation of each of the above embodiments and variations, R₁₉ is cyclopropyl. In still another variation of each of the above embodiments and variations, R₁₉ is cyclopentyl. In another variation of each of the above embodiments and variations, R₁₉ is a substituted or unsubstituted hetero(C₃₋₁₂)cycloalkyl. In another variation of each of the above embodiments and variations, R₁₉ is a substituted or unsubstituted (C₄₋₁₂)aryl. In another variation of each of the above embodiments and variations, R₁₉ is a substituted or unsubstituted hetero(C₄₋₁₀)aryl.

In yet another variation of each of the above embodiments and variations, R₁₉ has the formula

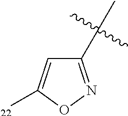

wherein R₂₂ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In a further variation of each of the above embodiments and variations, R₁₉ has the formula

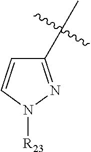

wherein R₂₃ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In still a further variation of each of the above embodiments and variations, R₁₉ has the formula

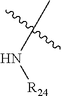

wherein R₂₄ selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In one variation of each of the above embodiments and variations containing R₁₉, R₁₉ is unsubstituted.

In a further variation of each of the above embodiments and variations, R₂₀ₐ is selected from the group consisting of hydrogen, carbonyl, oxycarbonyl, aminocarbonyl, (C₁₋₁₀)alkylcarbonyl, (C₃₋₁₂)cycloalkyl(C₁₋₅)carbonyl, hetero(C₃₋₁₂)cycloalkyl(C₁₋₁₀)carbonyl, aryl(C₁₋₁₀)carbonyl, hetero(C₁₋₁₀)aryl(C₁₋₅)carbonyl, (C₉₋₁₂)bicycloaryl(C₁₋₅)carbonyl, hetero(C₈₋₁₂)bicycloaryl(C₁₋₅)carbonyl, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;

In yet a further variation of each of the above embodiments and variations, R₂₀ₐ is hydrogen. In another variation of each of the above embodiments and variations, R₂₀ₐ is a substituted or unsubstituted (C₁₋₅)alkyl. In still another variation of each of the above embodiments and variations, R₂₀ₐ is methyl. In yet another variation of each of the above embodiments and variations, R₂₀ₐ is ethyl. In a further variation of each of the above embodiments and variations, R₂₀ₐ is propyl. In still a further variation of each of the above embodiments and variations, R₂₀ₐ is a substituted or unsubstituted aryl. In yet a further variation of each of the above embodiments and variations, R₂₀ₐ is a substituted or unsubstituted phenyl. In another variation of each of the above embodiments and variations, R₂₀ₐ is a substituted or unsubstituted (C₃₋₁₂)cycloalkyl. In still another variation of each of the above embodiments and variations, R₂₀ₐ is a substituted or unsubstituted cyclohexyl. In yet another variation of each of the above embodiments and variations, R₂₀ₐ is a substituted or unsubstituted hetero(C₄₋₁₀)aryl. In a further variation of each of the above embodiments and variations, R₂₀ₐ is a substituted or unsubstituted hydroxy(C₁₋₆)alkyl. In still a further variation of each of the above embodiments and variations, R₂₀ₐ is hydroxyethyl. In yet a further variation of each of the above embodiments and variations, R₂₀ₐ is halo. In another variation of each of the above embodiments and variations, R₂₀ₐ is fluoro. In still another variation of each of the above embodiments and variations, R₂₀ₐ is a substituted or unsubstituted hetero(C₃₋₆)cycloalkyl(C₁₋₄alkyl.

In yet another variation of each of the above embodiments and variations, R₂₀ₐ has the formula

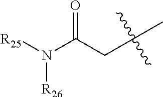

wherein R₂₅ and R₂₆ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In one variation of each of the above embodiments and variations containing R₂₀ₐ, R₂₀ₐ is unsubstituted.

In a further variation of each of the above embodiments and variations, R_{20b} is selected from the group consisting of hydrogen, carbonyl, oxycarbonyl, aminocarbonyl, (C₁₋₁₀)alkylcarbonyl, (C₃₋₁₂)cycloalkyl(C₁₋₅)carbonyl, hetero(C₃₋₁₂)cycloalkyl(C₁₋₁₀)carbonyl, aryl(C₁₋₁₀)carbonyl, hetero(C₁₋₁₀)aryl(C₁₋₅)carbonyl, (C₉₋₁₂)bicycloaryl(C₁₋₅)carbonyl, hetero(C₈₋₁₂)bicycloaryl(C₁₋₅)carbonyl, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;

In yet a further variation of each of the above embodiments and variations, R_{20b} is hydrogen. In another variation of each of the above embodiments and variations, R_{20b} is a substituted or unsubstituted (C₁₋₅)alkyl. In still another variation of each of the above embodiments and variations, R_{20b} is methyl. In yet another variation of each of the above embodiments and variations, R_{20b} is ethyl. In a further variation of each of the above embodiments and variations, R_{20b} is propyl. In still a further variation of each of the above embodiments and variations, R_{20b} is a substituted or unsubstituted aryl. In yet a further variation of each of the above embodiments and variations, R_{20b} is a substituted or unsubstituted phenyl. In another variation of each of the above embodiments and variations, R_{20b} is a substituted or unsubstituted (C₃₋₁₂)cycloalkyl. In still another variation of each of the above embodiments and variations, R_{20b} is a substituted or unsubstituted cyclohexyl. In yet another variation of each of the above embodiments and variations, R_{20b} is a substituted or unsubstituted hetero(C₄₋₁₀)aryl. In a further variation of each of the above embodiments and variations, R_{20b} is a substituted or unsubstituted hydroxy(C₁₋₆)alkyl. In still a further variation of each of the above embodiments and variations, R_{20b} is hydroxyethyl. In yet a further variation of each of the above embodiments and variations, R_{20b} is halo. In another variation of each of the above embodiments and variations, R_{20b} is fluoro. In still another variation of each of the above embodiments and variations, R_{20b} is a substituted or unsubstituted hetero(C₃₋₆)cycloalkyl(C₁₋₄alkyl.

In yet another variation of each of the above embodiments and variations, R_{20b} has the formula

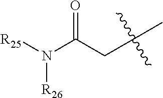

wherein R₂₅ and R₂₆ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

In one variation of each of the above embodiments and variations containing R_{20b}, R_{20b} is unsubstituted.

In a further variation of each of the above embodiments and variations, R₂₁ is a substituted or unsubstituted (C₁₋₅)alkylamino. In still a further variation of each of the above embodiments and variations, R₂₁ is CH₃NH—. In one variation of each of the above embodiments and variations containing R₂₁, R₂₁ is unsubstituted.

In yet a further variation of each of the above embodiments and variations, R₂₂ is a substituted or unsubstituted (C₁₋₃)alkyl. In another variation of each of the above embodiments and variations, R₂₂ is methyl. In one variation of each of the above embodiments and variations containing R₂₂, R₂₂ is unsubstituted.

In still another variation of each of the above embodiments and variations, R₂₃ is a substituted or unsubstituted (C₁₋₃)alkyl. In yet another variation of each of the above embodiments and variations, R₂₃ is methyl. In one variation of each of the above embodiments and variations containing R₂₃, R₂₃ is unsubstituted.

In a further variation of each of the above embodiments and variations, R₂₄ is a substituted or unsubstituted (C₁₋₅)alkyl. In still a further variation of each of the above embodiments and variations, R₂₄ is isopropyl. In yet a further variation of each of the above embodiments and variations, R₂₄ is tert-butyl. In another variation of each of the above embodiments and variations, R₂₄ is a substituted or unsubstituted (C₃₋₆)cycloalkyl. In still another variation of each of the above embodiments and variations, R₂₄ is cyclopropyl. In yet another variation of each of the above embodiments and variations, R₂₄ is cyclopentyl. In another variation of each of the above embodiments and variations, R₂₄ is a substituted or unsubstituted aryl. In a further variation of each of the above embodiments and variations, R₂₄ is a substituted or unsubstituted phenyl. In still a further variation of each of the above embodiments and variations, R₂₄ is a substituted or unsubstituted hetero(C₃₋₁₂)cycloalkyl. In yet a further variation of each of the above embodiments and variations, R₂₄ is a substituted or unsubstituted pyrrolidinyl. In another variation of each of the above embodiments and variations, R₂₄ is a substituted or unsubstituted piperidinyl. In one variation of each of the above embodiments and variations containing R₂₄, R₂₄ is unsubstituted.

In still another variation of each of the above embodiments and variations, R₂₅ is hydrogen. In still a further variation of each of the above embodiments and variations, R₂₅ is halo. In yet a further variation of each of the above embodiments and variations, R₂₅ is a substituted or unsubstituted (C₁₋₃)alkyl. In another variation of each of the above embodiments and variations, R₂₅ is methyl. In one variation of each of the above embodiments and variations containing R₂₅, R₂₅ is unsubstituted.

In yet another variation of each of the above embodiments and variations, R₂₆ is hydrogen. In still a further variation of each of the above embodiments and variations, R₂₆ is halo. In yet a further variation of each of the above embodiments and variations, R₂₆ is a substituted or unsubstituted (C₁₋₃)alkyl. In another variation of each of the above embodiments and variations, R₂₆ is methyl. In one variation of each of the above embodiments and variations containing R₂₆, R₂₆ is unsubstituted.

In yet another variation of each of the above embodiments and variations, R₂₇ is hydrogen. In a further variation of each of the above embodiments and variations, R₂₇ is halo. In still a further variation of each of the above embodiments and variations, R₂₇ is a substituted or unsubstituted (C₁₋₃)alkyl.

In yet another variation of each of the above embodiments and variations, R₂₈ is hydrogen. In a further variation of each of the above embodiments and variations, R₂₈ is halo. In still a further variation of each of the above embodiments and variations, R₂₈ is a substituted or unsubstituted (C₁₋₃)alkyl.

In yet a further variation of each of the above embodiments and variations, R₂₉ is hydrogen. In still another variation of each of the above embodiments and variations, R₂₉ is a substituted or unsubstituted (C₁₋₃)alkyl.

In another variation of each of the above embodiments and variations, R₃₀ is selected from the group consisting of hydrogen, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted. In yet a further variation of each of the above embodiments and variations, R₃₀ is hydrogen. In still another variation of each of the above embodiments and variations, R₃₀ is a substituted or unsubstituted (C₁₋₃)alkyl.

In a further variation of each of the above embodiments and variations, n is 1. In still a further variation of each of the above embodiments and variations, n is 2. In still another variation of each of the above embodiments and variations, r is 1. In yet another variation of each of the above embodiments and variations, r is 2.

In yet a further variation of each of the above embodiments and variations, m is 1. In another variation of each of the above embodiments and variations, m is 2.

In another embodiment, the cMET inhibitor has the formula:

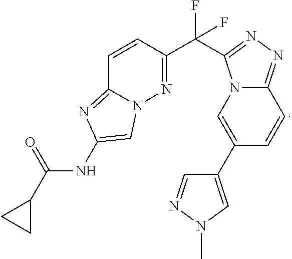 [SMILES: Cn1cc(-c2ccc3nnc(C(F)(F)c4ccc5nc(NC(=O)C6CC6)cn5n4)n3c2)cn1]

In another embodiment, the cMET inhibitor has the formula:

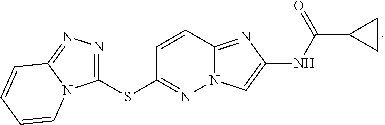 [SMILES: O=C(Nc1cn2nc(Sc3nnc4ccccn34)ccc2n1)C1CC1]

In another embodiment, the cMET inhibitor has the formula:

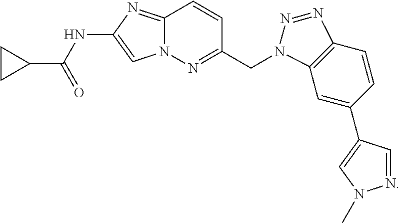 [SMILES: Cn1cc(-c2ccc3nnn(Cc4ccc5nc(NC(=O)C6CC6)cn5n4)c3c2)cn1]

In another embodiment, the cMET inhibitor is as described in WO 2006/021884, which is hereby incorporated by reference in its entirety. In particular, the cMet inhibitor has the formula:

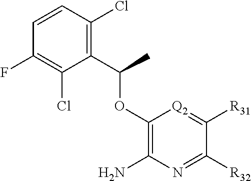

wherein
Q₂ is N or CR₄₂;
R₃₁ is selected from the group consisting of hydrogen, halogen, (C₆₋₁₂)aryl, 5-12 membered heteroaryl, (C₃₋₁₂)cycloalkyl, 3-12 membered heteroalicyclic, O—(CR₃₆R₃₇)_{q}R₃₄, COR₃₄, C(O)OR₃₄, CN, NO₂, S(O)₁R₃₄, SO₂NR₃₄R₃₅, NR₃₄C(O)R₃₅, C(═NR₃₆)NR₃₄R₃₅, (C₁₋₈)alkyl, (C₂₋₈)alkenyl and (C₂₋₈)alkynyl, and each hydrogen in R₃₁ is optionally substituted by one or more R₃₃ groups;
R₃₂ is hydrogen, halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, O(CR₃₆R₃₇)_{q}R₃₄, NR₃₄C(O)R₃₅, (CR₃₆R₃₇)_{q}C(O)OR₃₄, (CR₃₆R₃₇)_{q}NCR₃₄R₃₅, C(═NR₃₆)NR₃₄R₃₅, NR₃₄C(O)NR₃₅R₃₆, NR₃₄S(O)_{P}R₃₅ or C(O)NR₃₄R₃₅, and each hydrogen in R₃₂ is optionally substituted by R₃₈;
each R₃₃ is independently halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, O(CR₃₆R₃₇)_{q}R₃₄, NR₃₄C(O)R₃₅, (CR₃₆R₃₇)_{q}C(O)OR₃₄, (CR₃₆R₃₇)_{q}OR₃₄, (CR₃₆R₃₇)_{q}C(O)NR₃₄R₃₅, (CR₃₆R₃₇)_{q}NCR₃₄R₃₅, C(═NR₃₆)NR₃₄R₃₅, NR₃₄C(O)NR₃₅R₃₆, NR₃₄S(O)_{P}R₃₅ or C(O)NR₃₄R₃₅, each hydrogen in R₃₃ is optionally substituted by R₃₈, and
R₃₃ groups on adjacent atoms may combine to form a (C₆₋₁₂)aryl, 5-12 membered heteroaryl, (C₃₋₁₂)cycloalkyl or 3-12 membered heteroalicyclic group;
each R₃₄, R₃₅, R₃₆ and R₃₇ is independently hydrogen, halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl; or any two of R₃₄, R₃₅, R₃₆ and R₃₇ bound to the same nitrogen atom may, together with the nitrogen to which they are bound, be combined to form a 3 to 12 membered heteroalicyclic or 5-12 membered heteroqaryl group optionally containing 1 to 3 additional heteroatoms selected from N, O and S; or any two of R₃₄, R₃₅, R₃₆ and R₃₇ bound to the same carbon atom may be combined to form a (C₃₋₁₂)cycloalkyl, (C₆₋₁₂)aryl, 3-12 membered heteroalicyclic or 5-12 memebered heteroaryl group; and each hydrogen in R₃₄, R₃₅, R₃₆ and R₃₇ is optionally substituted by R₃₈;
each R₃₈ is independently halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, NH₂, CN, OH, O—(C₁₋₁₂)alkyl, O—(CH₂)_{q}(C₃₋₁₂)cycloalkyl, O—(CH₂)_{q}(C₆₋₁₂)aryl, O—(CH₂)_{q}(3-12 membered heteroalicyclic) or O—(CH₂)_{q}(5-12 membered heteroaryl); and each hydrogen in R₃₈ is optionally substituted by R₄₁;
each R₃₉ and R₄₀ is independently hydrogen, halogen, (C₁₋₁₂)alkyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, NR₃₄C(O)R₃₅, (CR₃₆R₃₇)_{q}C(O)OR₃₄, (CR₃₆R₃₇)_{q}NCR₃₄R₃₅, NR₃₄C(O)NR₃₅R₃₆, NR₃₄S(O)ₚR₃₅ or C(O)NR₃₄R₃₅; R₃₉ or R₄₀ may combine with a ring atom of A or a substitutent of A to form a (C₃₋₁₂)cycloalkyl, 3-12 memebered heteroalicyclic, (C₆₋₁₂)aryl or 5-12 memebered heteroaryl ring fused to A; and each hydrogen in R₃₉ and R₄₀ is optionally substituted by R₃₃;
each R₄₁ is independently halogen, (C₁₋₁₂)alkyl, (C₁₋₁₂)alkoxy, (C₃₋₁₂)cycloalkyl, (C₆₋₁₂)aryl, 3-12 membered heteroalicyclic, 5-12 membered heteroaryl, O—(C₁₋₁₂)alkyl, O—(CH₂)₁(C₃₋₁₂)cycloalkyl, O—(CH₂)_{q}(C₆₋₁₂)aryl, O—(CH₂)_{q}(3-12 membered heteroalicyclic, O—(CH₂)_{q}(5-12 membered heteroaryl or CN, and each hydrogen in R₄₁ is optionally substituted by halogen, OH, CN, (C₁₋₁₂)alkyl which may be partially or fully halogenated, O—(C₁₋₁₂)alkyl which may be partially or fully halogenated, CO, SO or SO₂;
R₄₂ is hydrogen, halogen (C₁₋₁₂)alkyl, (C₂₋₁₂)alkenyl, (C₂₋₁₂)alkynyl, (C₃₋₁₂)cycloalkyl, (C₆₋₁₂)aryl, 3-12 memebered heteroalicyclic, 5-12 memebered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, O—C(O)R₃₄, O—(CR₃₆R₃₇)_{q}R₃₄, NR₃₄C(O)R₃₅, (CR₃₆R₃₇)_{q}C(O)OR₃₄, (CR₃₆R₃₇)_{q}NCR₃₄R₃₅, C(═NR₃₆)NR₃₄R₃₅, NR₃₄C(O)NR₃₅R₃₆, NR₃₄S(O)ₚR₃₅ or C(O)NR₃₄R₃₅, and each hydrogen in R₄₂ is optionally substituted by R₃₃;
each R₄₃ is independently halogen, (C₁₋₁₂)alkyl, (C₂₋₁₂)aleknyl, (C₂₋₁₂)alkynyl, (C₃₋₁₂)cycloalkyl, (C₆₋₁₂)aryl, 3-12 membered hertoalicyclic, 5-12 memebered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, O—(CR₃₆R₃₇)_{q}R₃₄, NR₃₄C(O)R₃₅, (CR₃₆R₃₇)_{q}C(O)OR₃₄, (CR₃₆R₃₇)_{q}OR₃₄, (CR₃₆R₃₇)_{q}C(O)NR₃₄R₃₅, (CR₃₆R₃₇)_{q}NCR₃₄R₃₅, C(NR₃₆)NR₃₄R₃₅, NR₃₄C(O)NR₃₅R₃₆, NR₃₄S(O)ₚR₃₅, C(O)NR₃₄R₃₅, (CR₃₆R₃₇)_{q}(3-12 memebered heteroalicyclic), (CR₃₆R₃₇)_{q}(C₃₋₁₂)cycloalkyl, (CR₃₆R₃₇)_{q}(C₆₋₁₂)aryl, (CR₃₆R₃₇)_{q}(5-12 membered heteroaryl, (CR₃₆R₃₇)_{q}C(O)NR₃₄R₃₅ or (CR₃₆R₃₇)_{q}C(O)R₃₄; R₄₃ groups on adjacent atoms may combine to form a (C₆₋₁₂)aryl, 5-12 memebered heteroaryl, (C₃₋₁₂)cycloalkyl or 3-12 memebered heteroalicyclic group, and each hydrogen in R₄₃ is optionally substituted by R₃₃;
each 1 is independently 0, 1 or 2;
each q is independently 0, 1, 2, 3 or 4; and
each p is independently 1 or 2,
   or a pharmaceutically acceptable salt thereof.

In another embodiment, the cMET inhibitor has the formula:

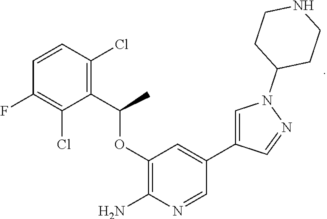 [SMILES: C[C@@H](Oc1cc(-c2cnn(C3CCNCC3)c2)cnc1N)c1c(Cl)ccc(F)c1Cl]

In still another embodiment, the cMET inhibitor is as described in WO 2009/002806, which is hereby incorporated by reference in its entirety. In particular, the cMet inhibitor has the formula:

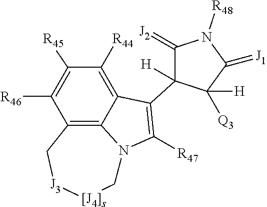

wherein
R₄₄, R₄₅ and R₄₆ are independently selected from the group consisting of H, F, Cl, Br, I, NR₅₀R₅₁, (C₁₋₆)alkyl, (C₁₋₆) substituted alkyl, (C₃₋₉)cycloalkyl, (C₃₋₉) substituted cycloalkyl, O—(C₁₋₆)alkyl, O—(C₃₋₉)cycloalkyl, O—(C₃₋₉) substituted cycloalkyl, aryl, heteroaryl and heterocyclyl;
R₄₇ is selected from the group consisting of H, (C₁₋₄)alkyl and (C₁₋₄) substituted alkyl;
R₄₈ is selected from the group consisting of H, (C₁₋₆)alkyl, CH₂R₄₉, CONHR₅₂, COR₅₃ and SO₂R₅₄;
R₄₉ is selected from the group consisting of O—P(═O)(OH)₂, O—P(═O)(OH)(O—(C₁₋₆)alkyl), O—P(═O)(O—(C₁₋₆)alkyl)₂, O—P(═O)(OH)(O—(CH₂)phenyl), O—P(═O)(O—(CH₂)phenyl)₂, a carboxylic acid group, an amino carboxylic acid group and a peptide;
R₅₀ and R₅₁ are independently selected from the group consisting of H and (C₁₋₆)alkyl;
R₅₂, R₅₃ and R₅₄ are independently selected from the group consisting of H, NHR₅₅, (C_{—}_{¹⁻⁶})alkyl, (C_{—}_{¹⁻⁶}) substituted alkyl, (C₃₋₉)cycloalkyl, (C₃₋₉) substituted cycloalkyl, aryl, heteroaryl and heterocyclyl;
Q₃ is selected from the group consisting of indolyl, substituted indolyl, aryl, heteroaryl, heterocyclyl and alkyl;
J₁ and J₂ are independently selected from the group consisting of O, S, H₂, where R₄₇ is (C₁₋₄)alkyl or (C₁₋₄) substituted alkyl when both J₁ and J₂ are O, and R₄₈ is H, (C₁₋₆)alkyl or CH₂R₄₉ when both J₁ and J₂ are not H₂;
J₃ is selected from the group consisting of —CH₂—, —NR₅₅—, S, O and a bond;
R₅₅ is selected from the group consisting of H, (C₁₋₆)alkyl, (C₁₋₆) substituted alkyl, (C₃₋₉)cycloalkyl, (C₃₋₉) substituted cycloalkyl, O—(C₁₋₆)alkyl, C(═O)—O—(C₁₋₆)alkyl and C(═O)—O—(C₁₋₆) substituted alkyl;
J₄ is selected from the group consisting of —CH₂—, CO and a bond; and
s is 0, 1 or 2.

In another embodiment, the cMET inhibitor has the formula:

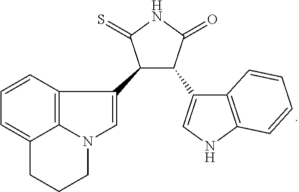 [SMILES: O=C1NC(=S)[C@H](c2cn3c4c(cccc24)CCC3)[C@H]1c1c[nH]c2ccccc12]

In yet another embodiment, the cMET inhibitor is selected from the group of cMet inhibitors described in Expert Opin. Ther. Patents (2010) 20(2), 159-177. In particular, the cMet inhibitor can be selected from the group consisting of:
K-252a (Schiering et al., Crystal structure of the tyrosine kinase domain of the hepatocyte growth factor receptor c-Met and its complex with the microbial alkaloid K-252a. Proc. Nat. Acad. Sci. USA 2003; 100:12654-99.);
SU-11274 (Sattler et al., A novel small molecule Met inhibitor induces apoptosis in cells transformed by the oncogenic TPR-MET tyrosine kinase. Cancer Res. 2003; 63:5462-69.);
PHA-665752 (Christensen et al., A selective small molecule inhibitor of c-Met kinase inhibits c-Met dependent phenotypes in vitro and exhibits cytoreductive antitumor activity in vivo. Cancer Res. 2009; 63:7345-55.) and other cMET inhibitors described in WO 2002/096361, which is hereby incorporated by reference in its entirety;
AM7 (Bellon et al., c-Met inhibitors with novel binding mode show activity against several hereditary papillary renal cell carcinoma-related mutations. J. Biol. Chem. 2008; 283:2675-83.);
AMG-208 (Amgen) and other cMet inhibitors described in WO 2009/091374, which is hereby incorporated by reference in its entirety;
JNJ-38877605 (Johnson & Johnson) and other cMet inhibitors described in WO 2007/075567, which is hereby incorporated by reference in its entirety;
MK-2461 (Merck) and other cMet inhibitors described in WO 2007/002254 and/or WO 2007/002258, which are hereby incorporated by reference in their entireties;
PF-04217903 (Pfizer) and other cMet inhibitors described in WO 2007/132308, which is hereby incorporated by reference in its entirety;
BMS 777607 (BMS);
GSK 136089 (also known as XL-880 and Foretinib) and other cMET inhibitors described in WO 2005/030140, which is hereby incorporated by reference in its entirety;
BMS 907351 (also known as XL-184);
EMD 1214063;
LY 2801653;
ARQ 197 (Arqule);
MK 8033 (Merck);
PF 2341066 (Pfizer) and other cMET inhibitors described in WO 2006/021881, which is hereby incorporated by reference in its entirety;
MGCD 265 (MethylGene);
E 7050 (Eisai);
MP 470 (SuperGen);
SGX 523 (Lilly);
cMet inhibitors described in Kirin J. J. Cui, Inhibitors targeting hepatocyte growth factor receptor and their potential therapeutic applications. Expert Opin. Ther. Patents 2007; 17:1035-45;
cMet inhibitors described in WO 2008/103277, which is hereby incorporated by reference in its entirety;
cMet inhibitors described in WO 2008/008310, which is hereby incorporated by reference in its entirety;
cMet inhibitors described in WO 2007/138472, which is hereby incorporated by reference in its entirety;
cMet inhibitors described in WO 2008/008539, which is hereby incorporated by reference in its entirety;
cMet inhibitors described in WO 2009/007390, which is hereby incorporated by reference in its entirety;
cMet inhibitors described in WO 2009/053737, which is hereby incorporated by reference in its entirety;
cMet inhibitors described in WO 2009/024825, which is hereby incorporated by reference in its entirety;
cMet inhibitors described in WO 2008/071451, which is hereby incorporated by reference in its entirety;
cMet inhibitors described in WO 2007/130468, which is hereby incorporated by reference in its entirety;
cMet inhibitors described in WO 2008/051547, which is hereby incorporated by reference in its entirety;
cMet inhibitors described in WO 2008/053157, which is hereby incorporated by reference in its entirety;
cMet inhibitors described in WO 2008/017361; WO 2008/145242; WO 2008/145243; WO 2008/148449; WO 2009/007074; WO 2009/006959; WO 2009/024221; WO 2009/030333; and/or WO 2009/083076, which are hereby incorporated by reference in their entireties;
cMet inhibitors described in WO 2009/093049, which is hereby incorporated by reference in its entirety;
cMet inhibitors described in US 2008/039457, which is hereby incorporated by reference in its entirety;
cMet inhibitors described in WO 2007/149427, which is hereby incorporated by reference in its entirety;
cMet inhibitors described in WO 2007/050309, which is hereby incorporated by reference in its entirety;
cMet inhibitors described in WO 2009/056692, which is hereby incorporated by reference in its entirety;
cMet inhibitors described in WO 2009/087305, which is hereby incorporated by reference in its entirety;
cMet inhibitors described in US 2009/197864, which is hereby incorporated by reference in its entirety;
cMet inhibitors described in US 2009/197862, which is hereby incorporated by reference in its entirety;
cMet inhibitors described in US 2009/156594, which is hereby incorporated by reference in its entirety;
cMet inhibitors described in WO 2008/124849, which is hereby incorporated by reference in its entirety;
cMet inhibitors described in WO 2008/067119, which is hereby incorporated by reference in its entirety;
cMet inhibitors described in WO 2007/064797, which is hereby incorporated by reference in its entirety;
cMet inhibitors described in WO 2009/045992, which is hereby incorporated by reference in its entirety;
cMet inhibitors described in WO 2008/088881, which is hereby incorporated by reference in its entirety;
cMet inhibitors described in WO 2007/081978, which is hereby incorporated by reference in its entirety;
cMet inhibitors described in WO 2008/079294, which is hereby incorporated by reference in its entirety;
cMet inhibitors described in WO 2008/079291, which is hereby incorporated by reference in its entirety;
cMet inhibitors described in WO 2008/086014, which is hereby incorporated by reference in its entirety;
cMet inhibitors described in WO 2009/033084, which is hereby incorporated by reference in its entirety;
cMet inhibitors described in WO 2007/059202, which is hereby incorporated by reference in its entirety;
cMet inhibitors described in US 2009/170896, which is hereby incorporated by reference in its entirety;
cMet inhibitors described in WO 2009/077874 and/or WO 2007/023768, which are hereby incorporated by reference in their entireties;
cMet inhibitors described in WO 2008/049855, which is hereby incorporated by reference in its entirety;
cMet inhibitors described in WO 2009/026717, which is hereby incorporated by reference in its entirety; and
cMet inhibitors described in WO 2008/046216, which is hereby incorporated by reference in its entirety.

### Antibody that Inhibits the HGF/SF-MET Signaling Pathway

Antibodies are very large, complex molecules (molecular weight of 150,000 or about 1320 amino acids) with intricate internal structure. A natural antibody molecule contains two identical pairs of polypeptide chains, each pair having one light chain and one heavy chain; hence the fundamental structural unit of an antibody is a tetramer. Each light chain and heavy chain in turn consists of two regions: a variable (“V”) region involved in binding the target antigen, and a constant (“C”) region that interacts with other components of the immune system. The light and heavy chain variable regions fold up together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which locks onto the target antigen. The position and length of the CDRs have been precisely defined. See, Kabat, E. et al., Sequences of Proteins of Immunological Interest, U.S. Department of Health and Human Services, 1983, 1987, which are herein incorporated by reference in their entireties. The part of a variable region not contained in the CDRs is called the framework, which forms the environment for the CDRs. In each chain, the three CDRs are interspersed with four framework sections in this order: FR1, CDR1, FR2, CDR2, FR3, CDR3, FR4.

Amino acids from the variable regions of the mature heavy and light chains of immunoglobulins are designated Hx and Lx respectively, where x is a number designating the position of an amino acid according to the scheme of Kabat et al. Kabat et al. lists many amino acid sequences for antibodies for each subgroup, and lists the most commonly occurring amino acid for each residue position in that subgroup to generate a consensus sequence. Kabat et al. uses a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat et al. by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Moreover, any two antibody sequences can be uniquely aligned, for example to determine percent identity, by using the Kabat numbering system so that each amino acid in one antibody sequence is aligned with the amino acid in the other sequence that has the same Kabat number. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

A monoclonal antibody (mAb) is a single molecular species of antibody and therefore does not encompass polyclonal antibodies produced by injecting an animal (such as a rodent, rabbit or goat) with an antigen, and extracting serum from the animal. A humanized antibody is a genetically engineered (monoclonal) antibody in which the CDRs from a “donor antibody” (e.g., an antibody from a mouse, rat, hamster or other similar species) are grafted onto a human antibody (“acceptor antibody”). Humanized antibodies can also be made with less than the complete CDRs from a mouse antibody (See, e.g., Pascalis et al., J. Immunol. 169:3076, 2002). Most commonly, the first heavy chain hypervariable loop H1, as defined by Chothia & Lesk, J. MoI. Biol. 196:901-917, 1987, from the donor antibody is also transferred to the humanized antibody. Thus, a humanized antibody is an antibody having CDRs from a donor antibody and variable region frameworks and constant regions from human antibodies. The light and heavy chain acceptor frameworks may be from the same or different human antibodies and may each be a composite of two or more human antibody frameworks; or alternatively, may be a consensus sequence of a set of human frameworks (e.g., a subgroup of human antibodies as defined in Kabat et al.), i.e., a sequence having the most commonly occurring amino acid in the set at each position. In addition, to retain high binding affinity, at least one of two additional structural elements can be employed. See, Queen et al., U.S. Pat. Nos. 5,530,101 and 5,585,089, each of which is incorporated herein by reference in its entirety.

In the first structural element, the framework of the heavy chain variable region of the humanized antibody is chosen to have high sequence identity (at least 65%) with the framework of the heavy chain variable region of the donor antibody, by suitably selecting the acceptor antibody from among the many known human antibodies. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are generally chosen on the basis of their ability to interact with the CDRs. For example, the replaced amino acids can be adjacent to a CDR in the donor antibody sequence or within 4-6 angstroms of a CDR in the humanized antibody as measured in 3-dimensional space.

On the other hand, since humanized mAbs must originate with a non-human donor mAb, humanized mAbs do not encompass essentially human mAbs made by isolating nucleic acids encoding variable regions from a human and selecting them using phage display methods (see, e.g., Dower et al., WO 91/17271; McCafferty et al., WO 92/001047; Winter, WO 92/20791; and Winter, FEBS Lett. 23.92, 1998, each of which is hereby incorporated by reference in its entirety) or by using transgenic mice (see, e.g., Lonberg et al., WO 93/12227; Kucherlapati WO 91/10741, and Burgess et al., WO 2005/027107, each of which is hereby incorporated by reference in its entirety).

The epitope of a mAb is the region of its antigen to which the mAb binds. Two antibodies bind to the same or overlapping epitope if each competitively inhibits (i.e., blocks) binding of the other to the antigen. That is, a 1×, 5×, 10×, 20× or 100× excess of one antibody inhibits binding of the other by at least 50% but preferably 75%, 90% or even 99% as measured in a competitive binding assay (see, e.g., Junghans et al., Cancer Res. 50:1495, 1990). Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

In one embodiment, the antibody that inhibits the HGF/SF-MET signaling pathway binds HGF. In one variation, the antibody preferably binds to human HGF (RefSeq Accession Number NM_{—}000601, which is hereby incorporated by reference in its entirety).

A monoclonal antibody (mAb) that binds HGF (i.e., an anti-HGF mAb) is said to neutralize HGF, or be neutralizing, if the binding partially or completely inhibits one or more biological activities of HGF (i.e., when the mAb is used as a single agent). Among the biological properties of HGF that a neutralizing antibody may inhibit are the ability of HGF to bind to its cMet receptor, to cause the scattering of certain cell lines such as Madin-Darby canine kidney (MDCK) cells; to stimulate proliferation of (i.e., be mitogenic for) certain cells including hepatocytes, 4 MBr-5 monkey epithelial cells, and various human tumor cells, or to stimulate angiogenesis, for example as measured by stimulation of human vascular endothelial cell (HWEC) proliferation or tube formation or by induction of blood vessels when applied to the chick embryo chorioallantoic membrane (CAM). Antibodies of the invention preferably bind to human HGF, i.e., to the protein encoded by the RefSeq sequence with Accession Number NM_{—}000601, which is hereby incorporated by reference in its entirety.

A neutralizing mAb of the invention at a concentration of, e.g., 0.01, 0.1, 0.5, 1, 2, 5, 10, 20 or 50 g/ml will inhibit a biological function of HGF (e.g., stimulation of proliferation or scattering) by about at least 50% but preferably 75%, more preferably by: 90% or 95% or even 99%, and most preferably approximately 100% (essentially completely) as assayed by methods described in US2004/0208876, which is incorporated herein by reference, or by methods known in the art. Inhibition is considered complete if the level of activity is within the margin of error for a negative control lacking HGF. Typically, the extent of inhibition is measured when the amount of HGF used is just sufficient to fully stimulate the biological activity, or is 0.05, 0.1, 0.5, 1, 3 or 10 g/ml.

Preferably, at least 50%, 75%, 90%, or 95% or essentially complete inhibition will be achieved when the molar ratio of antibody to HGF is 0.5×, 1×, 2×, 3×, 5× or 10×. Preferably, the mAb will be neutralizing, i e, inhibit the biological activity, when used as a single agent, but possibly 2 mAbs will be needed together to give inhibition. Most preferably, the mAb will neutralize not just one but several of the biological activities listed above; for purposes herein, an anti-HGF mAb that used as a single agent neutralizes all the biological activities of HGF will be called “fully neutralizing”, and such mAbs are most preferable. MAbs of the invention will preferably be specific for HGF, that is they will not bind, or only bind to a much lesser extent (e.g., Kₐ at least ten-fold less), proteins that are related to HGF such as fibroblast growth factor (FGF) and vascular endothelial growth factor (VEGF). Preferred antibodies lack agonistic activity toward HGF. That is, the antibodies block interaction of HGF with cMet without stimulating cells bearing HGF directly. MAbs of the invention typically have a binding affinity (Kₐ) for HGF of at least 10⁷ M⁻¹ but preferably 10⁸ M⁻¹ or higher, and most preferably 10⁹ M⁻¹ or higher or even 10¹⁰ M⁻¹ or higher.

MAbs of the invention include anti-HGF antibodies in their natural tetrameric form (2 light chains and 2 heavy chains) and may be of any of the known isotypes IgG, IgA, IgM, IgD and IgE and their subtypes, i.e., human IgG1, IgG2, IgG3, IgG4 and mouse IgG1, IgG2a, IgG2b, and IgG3. The mAbs of the invention are also meant to include fragments of antibodies such as Fv, Fab and F(ab′)2; bifunctional hybrid antibodies (e.g., Lanzavecchia et al., Eur. J. hnmunol. 17:105, 1987), single-chain antibodies (Huston et al., Proc. Natl. Acad.; Sci. USA 85:5879, 1988; Bird et al., Science 242:423, 1988), and antibodies with altered constant regions (e.g., U.S. Pat. No. 5,624,821). The mAbs may be of animal (e.g., mouse, rat, hamster or chicken) origin, or they may be genetically engineered. Rodent mAbs are made by standard methods well-known in the art, comprising multiple immunization with HGF in appropriate adjuvant i.p., i.v., or into the footpad, followed by extraction of spleen or lymph node cells and fusion with a suitable immortalized cell line, and then selection for hybridomas that produce antibody binding to HGF, as described in, for example, US2004/0208876, which is incorporated herein by reference. Chimeric and humanized mAbs, made by art-known methods mentioned supra, are preferred embodiments of the invention. Human antibodies made, e.g., by phage display or transgenic mice methods are also preferred (see e.g., Dower et al., McCafferty et al., Winter, Lonberg et al., Kucherlapati' supra). More generally, human-like, reduced immunogenicity and genetically engineered antibodies as defined herein are all preferred.

The neutralizing anti-HGF mAbs L1H, L2C7 and L2G7 mAbs are examples of the invention, with L2G7 a preferred example. Neutralizing mAbs with the same or overlapping epitope as any of these mAbs, e.g., as L2G7, provide other examples. A chimeric or humanized form of L2G7 is an especially preferred embodiment. A mAb (including chimeric, humanized and human antibodies) that competes with L2G7 for binding to HGF and neutralizes HGF in at least one, and preferably all, in vitro or in vivo assays described herein is also preferred. MAbs that are 90%, 95%, 99% or 100% identical (determined by aligning antibody sequences according to the Kabat convention) to L2G7 in amino acid sequence, at least in the CDRs are included in the invention. Preferably such antibodies differ from L2G7 by a small number of functionally inconsequential amino acid substitutions (e.g., conservative substitutions), deletions, or insertions. Preferably such antibodies retain the functional properties of L2G7, i.e., such antibodies neutralize HGF in at least one, and preferably all, in vitro or in vivo assays described herein. For purposes of classifying amino acids substitutions as conservative or nonconservative, amino acids may be grouped as follows: Group I (hydrophobic side chains): Met, Ala, Val, Leu, Ile; Group II (neutral hydrophilic side chains): Cys, Ser. Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe. Conservative substitutions involve substitutions between amino acids in the same class. Non-conservative substitutions constitute exchanging a member of one of these classes for a member of another.

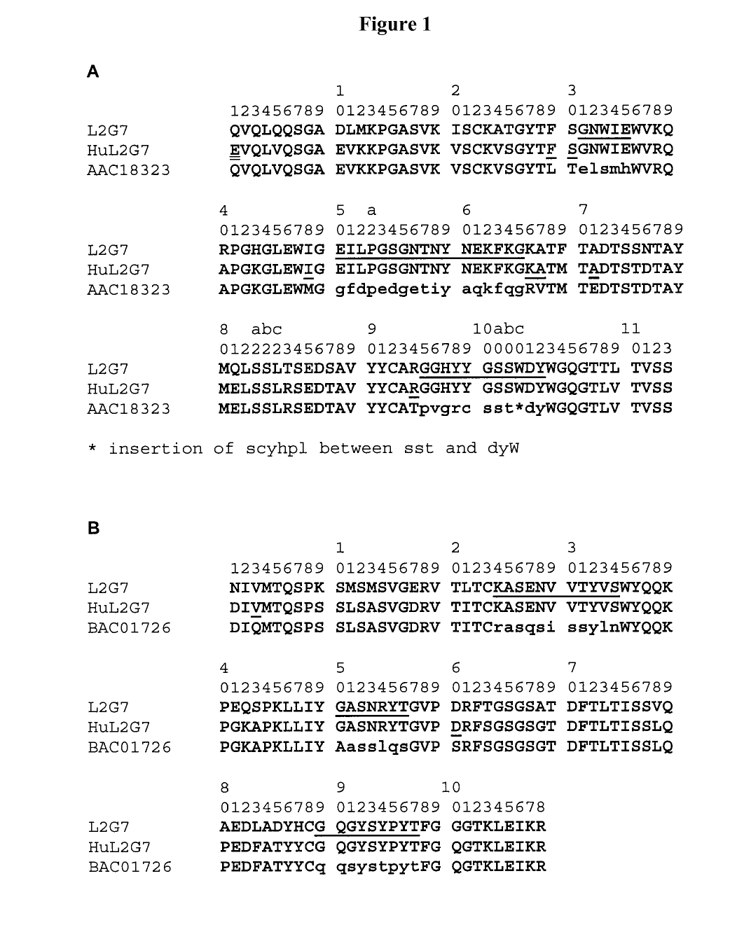

Humanized mAbs useful in the present invention include humanized forms of the mouse L2G7 mAb. The sequences of the mature heavy and light chain variable regions of the mouse L2G7 mAb are shown in FIGS. 1A and 1B respectively. Hence, humanized forms of the L2G7 mAb encompass most or all of the CDR amino acids from these sequences in human variable region frameworks (including single, composite or consensus sequence human frameworks). For example, some humanized antibodies include three intact CDRs from the L2G7 heavy chain and three intact CDRs from the light chain. Other humanized antibodies include at least one intact CDR from the L2G7 heavy chain and at least one intact CDR from the L2G7 light chain. Some humanized antibodies include at least one CDR in which some residues are from the corresponding CDR of L2G7 and the others are from a CDR of a human antibody, preferably the same human antibody as supplies the variable region framework containing the CDR.

In some humanized antibodies useful in the present invention, at least 1, 3, 5 or all positions selected from the group H29, H30, H48, H66, H67, H71, H94, L3, and L60 are occupied by an amino acid present at the corresponding position by Kabat numbering in the mouse L2G7 antibody. In the human acceptor variable region frameworks used in the generation of HuL2G7, as described in US2008/0019974, which is incorporated herein by reference, all of these positions are occupied by human residues differing from the amino acid present at the corresponding position in the mouse L2G7 antibody. Thus, it is preferable to substitute all or most positions selected from the group. If other human variable region frameworks are used, some of the positions may be occupied by amino acids that are the same in the human variable region framework and the mouse L2G7 antibody. Accordingly, substitution is not performed at such positions but can be performed at other positions differing between the human variable region framework and mouse L2G7 antibody in accordance with the rules of Queen, U.S. Pat. No. 5,530,101 and U.S. Pat. No. 5,585,089. Regardless of the choice of human variable region framework, substitution of other amino acids besides those specified in the above group is also possible as discussed below.

However, in general neither the heavy chain variable region framework nor the light chain variable region framework of the humanized antibody includes more than ten or twelve substitutions resulting in residues not present in the acceptor human variable region framework (including human consensus variable region frameworks and composite human variable region frameworks).

Any constant regions present in the humanized antibodies useful in the present invention are human or essentially so, having no more than ten, and preferably two or fewer substitutions relative to a natural human constant region. Some substitutions are advantageous in increasing the half-life of an antibody and/or its affinity for FcγRn. Other substitutions, usually conservative substitutions, as discussed below, are neutral in effect.

Exemplified humanized forms of L2G7 include mature heavy and light chain variable regions having the sequences shown in FIGS. 1A and 1B respectively. Other preferred forms of humanized L2G7 include mature heavy and light chain variable regions having sequences at least 90%, 95%, 98% or 99% identical to these sequences (when aligned according to Kabat numbering, supra), and/or differ from them by a small number (typically involving no more than 5 or 10 amino acids) of functionally inconsequential substitutions, deletions and/or insertions. For example, the first amino acid of the heavy chain may be either Glu or Gln. The substitutions are usually conservative. Substitutions relative to the V regions of HuL2G7, as described in FIGS. 1A and 1B and in US2008/0019974, incorporated herein by reference, are preferably avoided at positions H29, H30, H48, H66, H67, H71, H94, L3, and L60, where amino acids from mouse L2G7 were included due to the interaction of these positions with CDRs. Substitutions preferably occur in variable region framework positions, but can also occur in CDR regions. If a CDR region is substituted, it is preferable to replace a mouse amino acid with an amino acid from the corresponding position (Kabat numbering) of a human antibody, preferably the same human antibody that supplies the acceptor variable region frameworks.

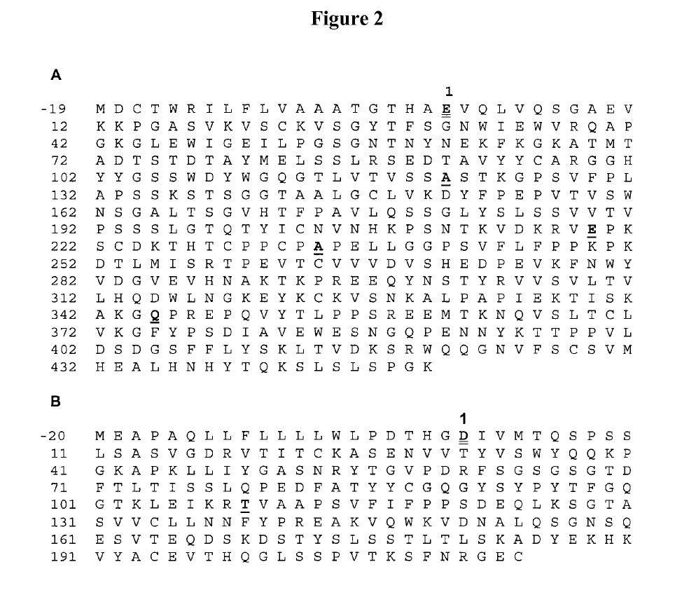

Usually, the humanized L2G7 mAbs are of the IgG1, IgG2, IgG3 or IgG4 isotype with a kappa light chain. An IgG1 mAb having the variable regions of FIGS. 1A and 1B respectively combined with complete human gamma-1 and kappa constant region is designated HuL2G7. The complete heavy and light chains of HuL2G7 are respectively shown in FIGS. 2A and 2B. Only the mature parts of these sequences beginning at the positions indicated by the number 1 actually constitute HuL2G7, as the preceding signal peptides are cleaved off before or during antibody secretion.

Variants of HuL2G7 retaining similar binding characteristics to HuL2G7 can be obtained by mutagenesis followed by mass selection using the phage display methods discussed above. Variants are initially selected for specific binding to HGF, optionally in competition with HuL2G7 or mouse L2G7. Variants having the same or similar binding characteristics as the exemplified antibody can then be tested functionally.

Preferred humanized L2G7 mAbs are neutralizing or fully neutralizing against HGF as defined supra. Preferably, for some, most or all biological properties of HGF measured (e.g., binding to Met, stimulation of proliferation of Mv 1 Lu or HUVEC cells), the neutralizing activity of the humanized mAb is within 3-fold, more preferably within 2-fold or 1.5-fold, and most preferably indistinguishable from (i.e., to within experimental error), the neutralizing activity of L2G7 itself. That is, no more than 3-fold, 2-fold, 1.5-fold or the same amount of humanized mAb relative to L2G7 is needed to obtain the same extent of inhibition of the biological property (for example, as measured by IC₅₀'s). Preferably, the affinity for HGF of the humanized mAbs is also within 3-fold, 2-fold or essentially indistinguishable from that of L2G7. Similarly, in xenograft mouse models (e.g., using a human glioma cell line such as U87), the humanized mAbs preferably inhibit tumor growth within 3-fold, 2-fold or indistinguishably from the mouse L2G7 mAb. Indeed, preferably only a 40, 20 or even 10 μg dose of humanized mAb administered twice per week completely inhibits growth of U87 tumor xenografts.

Native mAbs of the invention may be produced from their hybridomas. Genetically engineered mAbs, e.g., chimeric or humanized mAbs, may be expressed by a variety of art known methods. For example, genes encoding their light and heavy chain V regions may be synthesized from overlapping oligonucleotides and inserted together with available C regions into expression vectors (e.g., commercially available from Invitrogen) that provide the necessary regulatory regions, e.g., promoters, enhancers, poly A sites, etc. Use of the CMV promoter-enhancer is preferred. The expression vectors may then be transfected using various well-known methods such as lipofection or electroporation into a variety of mammalian cell lines such as CHO or non-producing myelomas including Sp2/0 and NS0, and cells expressing the antibodies selected by appropriate antibiotic selection. See, e.g., U.S. Pat. No. 5,530,101. Larger amounts of antibody may be produced by growing the cells in commercially available bioreactors.

Once expressed, the mAbs or other antibodies of the invention may be purified according to standard procedures of the art such as microfiltration, ultrafiltration, protein A or G affinity chromatography, size exclusion chromatography, anion exchange chromatography, cation exchange chromatography and/or other forms of affinity chromatography based on organic dyes or the like. Substantially pure antibodies of at least about 90 or 95% homogeneity are preferred, and 98% or 99% or more homogeneity most preferred, for pharmaceutical uses.

In particular, the antibody can be selected from the group consisting of:
OA-5d5;
AMG 102 (Amgen);
AV299 (SCH 900706 (Schering);
L2G7 and other antibodies as described in WO 2005/107800, which is hereby incorporated by reference in its entirety;
humanized L2G7 (HuL2G7) and other antibodies as described in WO 2007/115049, which is hereby incorporated by reference in its entirety;
an antibody as described in WO 2005/017107, which is hereby incorporated by reference in its entirety;
an antibody as described in WO 2007/143090 and/or WO 2007/143098, which are hereby incorporated by reference in their entireties;
an antibody as described in WO 2001/034650, which is hereby incorporated by reference in its entirety;
an antibody as described in US 2005/0118643, which is hereby incorporated by reference in its entirety;
an antibody as described in WO 2005/017107, which is hereby incorporated by reference in its entirety;
an antibody as described in US 2007/0092520, which is hereby incorporated by reference in its entirety;
an antibody as described in U.S. Pat. No. 7,494,650, which is hereby incorporated by reference in its entirety; and
an antibody as described in U.S. Pat. No. 7,220,410, which is hereby incorporated by reference in its entirety.

In another embodiment, the antibody that inhibits the HGF/SF-MET signaling pathway binds with cMET. In particular, such antibody can be selected from the group consisting of:
LA480;
an antibody as described in WO 2005/007193; U.S. Pat. No. 5,646,036; U.S. Pat. No. 5,686,292; U.S. Pat. No. 6,207,152; U.S. Pat. No. 6,214,344; U.S. Pat. No. 6,099,841; and/or WO 1992/020792, which are hereby incorporated by reference in their entireties; and
an antibody as described in WO 2009/007427, which is hereby incorporated in its entirety.

### One or More Additional Therapeutic Agents

A wide variety of therapeutic agents may have a therapeutic additive or synergistic effect with the cMET inhibitors and antibodies useful with the present invention. Combination therapies that comprise a cMET inhibitor, an antibody useful with the present invention and one or more optional other therapeutic agents can be used, for example, to: 1) enhance the therapeutic effect(s) of the methods of the present invention and/or the one or more other therapeutic agents; 2) reduce the side effects exhibited by the methods of the present invention and/or the one or more other therapeutic agents; and/or 3) reduce the effective dose of the cMET inhibitor and/or antibody useful with the present invention and/or the one or more optional other therapeutic agents. For example, such therapeutic agents may additively or synergistically combine with the cMET inhibitor and/or antibody useful with present invention to inhibit undesirable cell growth, such as inappropriate cell growth resulting in undesirable benign conditions or tumor growth.

Examples of therapeutic agents that may be used in combination with the cMET inhibitors and antibodies useful with the present invention include, but are not limited to, anti-proliferative agents, anticancer agents, alkylating agents, antibiotic agents, antimetabolic agents, hormonal agents, plant-derived agents, and biologic agents.

Alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. Combination therapy including an inhibitor of the present invention and an alkylating agent may have therapeutic synergistic effects on cancer and reduce sides affects associated with these chemotherapeutic agents.

Antibiotic agents are a group of drugs that produced in a manner similar to antibiotics as a modification of natural products. Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. Combination therapy including an inhibitor of the present invention and an antibiotic agent may have therapeutic synergistic effects on cancer and reduce sides affects associated with these chemotherapeutic agents.

Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Combination therapy including an inhibitor of the present invention and a antimetabolic agent may have therapeutic synergistic effects on cancer and reduce sides affects associated with these chemotherapeutic agents.

Hormonal agents are a group of drugs that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, and flutamide), aromatase inhibitors (e.g., aminoglutethimide, anastrozole and tetrazole), ketoconazole, goserelin acetate, leuprolide, megestrol acetate and mifepristone. Combination therapy including an inhibitor of the present invention and a hormonal agent may have therapeutic synergistic effects on cancer and reduce sides affects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), and taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including an inhibitor of the present invention and a plant-derived agent may have therapeutic synergistic effects on cancer and reduce sides affects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy including an inhibitor of the present invention and a biologic agent may have therapeutic synergistic effects on cancer, enhance the patient's immune responses to tumorigenic signals, and reduce potential sides affects associated with this chemotherapeutic agent.

Cytokines possess profound immunomodulatory activity. Some cytokines such as interleukin-2 (IL-2, aldesleukin) and interferon have demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. Examples of interleukins that may be used in conjunction with inhibitors of the present invention include, but are not limited to, interleukin 2 (IL-2), and interleukin 4 (IL-4), interleukin 12 (IL-12).

Interferons □include more than 23 related subtypes with overlapping activities, all of the IFN subtypes within the scope of the present invention. IFN has demonstrated activity against many solid and hematologic malignancies, the later appearing to be particularly sensitive.

Other cytokines that may be used in conjunction with the inhibitors of the present invention include those cytokines that exert profound effects on hematopoiesis and immune functions. Examples of such cytokines include, but are not limited to erythropoietin, granulocyte-CSF (filgrastin), and granulocyte, macrophage-CSF (sargramostim). These cytokines may be used in conjunction with an inhibitor of the present invention to reduce chemotherapy-induced myelopoietic toxicity.

Other immuno-modulating agents other than cytokines may also be used in conjunction with the inhibitors of the present invention to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including an inhibitor of the present invention and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and malignant CD20⁺ pre-B and mature B cells. RITUXAN® is used as single agent for the treatment of patients with relapsed or refractory low-grade or follicular, CD20+, B cell non-Hodgkin's lymphoma. Combination therapy including an inhibitor of the present invention and RITUXAN® may have therapeutic synergistic effects not only on lymphoma, but also on other forms or types of malignant tumors.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth—cancer. Examples of the tumor suppressor genes include, but are not limited to, DPC-4, NF-1, NF-2, RB, p53, WT1, BRCA1, and BRCA2.

DPC-4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. The tumor suppressor gene can be transferred into the tumor cells where it exerts its tumor suppressing functions. Combination therapy including an inhibitor of the present invention and a tumor suppressor may have therapeutic synergistic effects on patients suffering from various forms of cancers.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to the tumor cell, TAAs provide targets for the immune system to recognize and cause their destruction. Example of TAAs include, but are not limited to gangliosides (GM2), prostate specific antigen (PSA), alpha-fetoprotein (AFP), carcinoembryonic antigen (CEA) (produced by colon cancers and other adenocarcinomas, e.g. breast, lung, gastric, and pancreas cancer s), melanoma associated antigens (MART-1, gp100, MAGE 1,3 tyrosinase), papillomavirus E6 and E7 fragments, whole cells or portions/lysates of antologous tumor cells and allogeneic tumor cells.

An adjuvant may be used to augment the immune response to TAAs. Examples of adjuvants include, but are not limited to, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan, a chemotherapeutic agent which is believe to reduce tumor-induced suppression when given in low doses.

In another embodiment, the cMET inhibitors and antibodies useful with the present invention are used in combination with a Hedgehog inhibitor. In one particular embodiment, the Hedgehog inhibitor is selected from the group of Hedgehog inhibitors described in WO 2009/126840, which is hereby incorporated by reference in its entirety.

In still another embodiment, the cMET inhibitors and antibodies useful with the present invention are used in combination with an EGF inhibitor. In one particular embodiment, the EGF inhibitor is selected from the group of EGF inhibitors described in WO 2009/126834, which is hereby incorporated by reference in its entirety.

In yet another embodiment, the cMET inhibitors and antibodies useful with the present invention are used in combination with a PTEN agonist. In one particular embodiment, the PTEN agonist is selected from the group of PTEN agonists described in WO 2009/126842, which is hereby incorporated by reference in its entirety.

In yet another embodiment, the cMet inhibitors and antibodies useful with the present invention are used in combination with a nucleoside analogue, a PDGFR antagonist, a VEGFR antagonist, a c-KIT antagonist, or a FLT3 inhibitor.

In a further embodiment, the cMET inhibitors and antibodies useful with the present invention are used in combination with a MEK inhibitor. In one particular embodiment, the MEK inhibitor is selected from the group of MEK inhibitor s described in WO 08/079,814, which is hereby incorporated by reference in its entirety. In another particular embodiment, the MEK inhibitor has the formula:

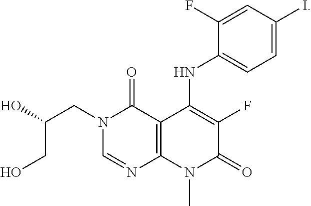 [SMILES: Cn1c(=O)c(F)c(Nc2ccc(I)cc2F)c2c(=O)n(C[C@@H](O)CO)cnc21]

In still a further embodiment, the cMET inhibitors and antibodies useful with the present invention are used in combination with a SYK inhibitor. In a further embodiment, the cMET inhibitors and antibodies useful with the present invention are used in combination with a PI3K inhibitor. In yet a further embodiment, the cMET inhibitors and antibodies useful with the present invention are used in combination with an mTOR inhibitor. In another embodiment, the cMET inhibitors and antibodies useful with the present invention are used in combination with a PI3K/mTOR inhibitor.

### Pharmaceutical Compositions

The cMET inhibitor, antibody and/or one or more additional therapeutic agents are optionally formulated as part of one or more pharmaceutical compositions comprising one or more of the cMET inhibitor, antibody and additional pharmaceutical agents, together with a pharmaceutically acceptable excipient, wherein the proportion and nature of the excipient or excipients are determined by the properties of the therapeutic agent or agents comprising the composition, the chosen route of administration and standard pharmaceutical practice.

The term “pharmaceutically acceptable excipient” refers to those typically used in preparing pharmaceutical compositions and should be pharmaceutically pure and non-toxic in the amounts used. They generally are a solid, semi-solid, or liquid material which can serve as a vehicle or medium for the active ingredient. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

### Administration

The cMET inhibitor, antibody and/or one or more additional therapeutic agents can be administered at the same time and/or sequentially. For example, the cMET inhibitor, either alone or together with the one or more additional therapeutic agents, can be administered before, during or after administration of the antibody.

Further, the dosing regimens for the cMET inhibitor, the antibody and the one or more additional therapeutic agents can be separately selected. For example, the cMET inhibitor, either alone or together with the one or more additional therapeutic agents, can be dosed daily, while the antibody can be administered weekly. Administration of the cMET inhibitor, antibody and/or one or more additional therapeutic agents may be coextensive, that is, occurring during overlapping periods of time.

In effecting treatment of a patient in need of such treatment, administration of the cMET inhibitor, antibody and/or one or more additional therapeutic agents can be accomplished using any of a variety of routes that make the active ingredient bioavailable. For example, the cMET inhibitor, antibody and/or one or more additional therapeutic agents can be administered by oral and parenteral routes, more particularly by inhalation, subcutaneously, intramuscularly, intravenously, transdermally, intranasally, rectally, vaginally, occularly, topically, sublingually, transbuccally, intraperitoneally, intravenously, intraarterially, transdermally, intraadiposally, intrathecally and via local delivery for example by catheter or stent. For example, the cMET inhibitor, either alone or together with the one or more additional therapeutic agents, can be administered orally, while the antibody can be administered intravenously.

One skilled in the art can readily select the proper form and route of administration depending upon the particular characteristics of the active ingredient or ingredients selected, the disorder or condition to be treated, the stage of the disorder or condition, and other relevant circumstances. Pharmaceutical compositions comprising the cMET inhibitor, antibody and/or one or more additional therapeutic agents may be administered to the patient, for example, in the form of liquid formulations (such as solutions and suspensions that are adapted for oral or parenteral administration), tablets, capsules, cachets, papers, lozenges, wafers, elixirs, ointments, transdermal patches, aerosols, inhalants, suppositories and reconstituted lyophilized powders.

Pharmaceutical compositions comprising the mAb can be administered to a patient by any suitable route, especially parentally by intravenous infusion or bolus injection, intramuscularly or subcutaneously. Intravenous infusion can be given over as little as 15 minutes, but more often for 30 minutes, or over 1, 2 or even 3 hours. The mAb can also be injected directly into the site of disease (e.g., a tumor), or encapsulated into carrying agents such as liposomes. The dose given will be sufficient to alleviate the condition being treated (“therapeutically effective dose”) and is likely to be 0.1 to 5 mg/kg body weight, for example 1, 2, 3 or 4 mg/kg, but may be as high as 10 mg/kg or even 15 or 20 mg/kg. A fixed unit dose may also be given, for example, 50, 100, 200, 500 or 1000 mg, or the dose may be based on the patient's surface area, e.g., 100 mg/m². Usually between 1 and 8 doses, (e.g., 1, 2, 3, 4, 5, 6, 7 or 8) are administered to treat cancer, but 10, 20 or more doses may be given. The mAb can be administered daily, biweekly, weekly, every other week, monthly or at some other interval, depending, e.g. on the half-life of the mAb, for 1 week, 2 weeks, 4 weeks, 8 weeks, 3-6 months or longer. Repeated courses of treatment are also possible, as in chronic administration.

Pharmaceutical compositions comprising the cMET inhibitor and/or one or more additional therapeutic agents are preferably formulated in a unit dosage form, each dosage typically containing from about 0.5 mg to about 200 mg each of the cMET inhibitor, antibody and/or one or more additional therapeutic agents. The term “unit dosage form” refers to a physically discrete unit suitable as a single dosage, each unit containing a predetermined quantity of active ingredient, in association with a suitable pharmaceutical excipient, by which one or more is used throughout the dosing regime to produce the desired therapeutic effect.

Pharmaceutical compositions comprising the cMET inhibitor, antibody and/or one or more additional therapeutic agents can be prepared in a manner well known in the pharmaceutical art. The amount of the cMET inhibitor, antibody and/or one or more additional therapeutic agents may be varied depending upon its particular form and may conveniently be between 1% to about 70% of the weight of the unit dosage form.

### EXAMPLES

### Preparation of Select cMET Inhibitors

Various methods can be used for synthesizing select cMET inhibitors useful in the present invention. Representative methods for synthesizing these compounds are provided in the Examples. It is noted, however, that the compounds may also be synthesized by other synthetic routes that others may devise.

It will be readily recognized that certain cMET inhibitors useful in the present invention have atoms with linkages to other atoms that confer a particular stereochemistry to the compound (e.g., chiral centers). It is recognized that synthesis of compounds may result in the creation of mixtures of different stereoisomers (i.e., enantiomers and diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) and can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

cMET inhibitors useful in the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound with a suitable carbamylating agent (e.g., 1,1-acyloxyalkylcarbonochloridate, para-nitrophenyl carbonate, or the like).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

cMET inhibitors useful in the present invention may be conveniently prepared, or formed during the process of the invention, as solvates (e.g., hydrates). Hydrates of the compounds may be conveniently prepared by recrystallization from an aqueous/organic solvent mixture, using organic solvents such as dioxin, tetrahydrofuran or methanol.

cMET inhibitors useful in the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification.

All references to ether or Et₂O are to diethyl ether; and brine refers to a saturated aqueous solution of NaCl. Unless otherwise indicated, all temperatures are expressed in ° C. (degrees Centigrade). All reactions are conducted under an inert atmosphere at RT unless otherwise noted.

¹H NMR spectra were recorded on a Bruker Avance 400. Chemical shifts are expressed in parts per million (ppm). Coupling constants are in units of Hertz (Hz). Splitting patterns describe apparent multiplicities and are designated as s (singlet), d (doublet), t (triplet), q (quartet), m (multiplet), br (broad).

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60E-254), visualized with UV light, 5% ethanolic phosphomolybdic acid, Ninhydrin or p-anisaldehyde solution. Flash column chromatography was performed on silica gel (230-400 mesh, Merck).

The starting materials and reagents used in preparing these compounds are either available from commercial suppliers such as the Aldrich Chemical Company (Milwaukee, Wis.), Bachem (Torrance, Calif.), Sigma (St. Louis, Mo.), or may be prepared by methods well known to a person of ordinary skill in the art, following procedures described in such standard references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The entire disclosures of all documents cited throughout this application are incorporated herein by reference.

Synthetic Schemes for Select cMET Inhibitors

cMET inhibitors useful in the present invention may be synthesized according to the reaction schemes shown below. Other reaction schemes could be readily devised by those skilled in the art. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

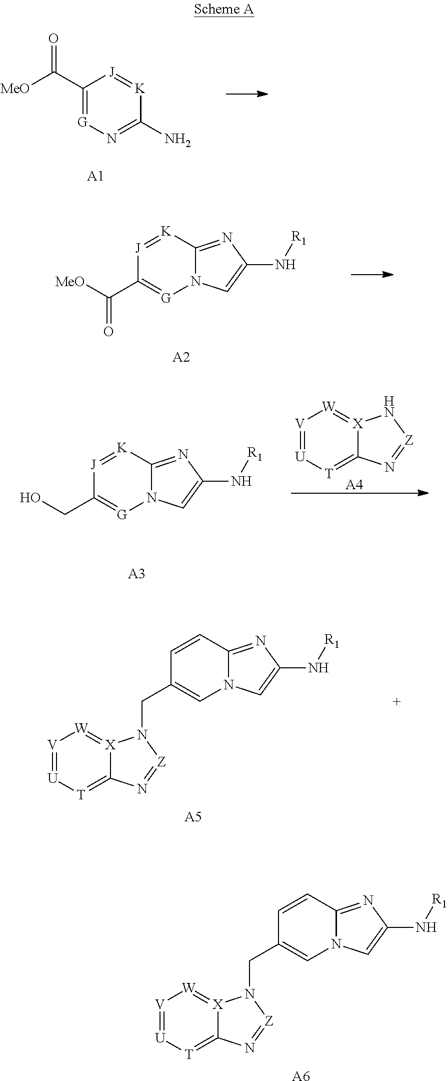

Compound A2 can be prepared starting from compound A1 in three steps following an analogous method to that described in U.S. Pat. No. 6,358,971, which is incorporated by reference herein in its entirety. Reduction of the ester can be accomplished in the presence of a reducing agent such as LAH or NaBH₄ in THF or dioxane at 0-80° C. for 1-8 hrs to provide alcohol (A3). Standard Mitsunobu coupling of A3 and A4 in the presence of triphenylphosphine and an azodicarboxalate, such as DEAD or DIAD, at 0-80° C. for 1-24 hrs can be used to provide compounds A5 and A6. Compounds A5 and A6 can be separated using any of a variety of techniques known in the art including, for example, preparative LCMS.

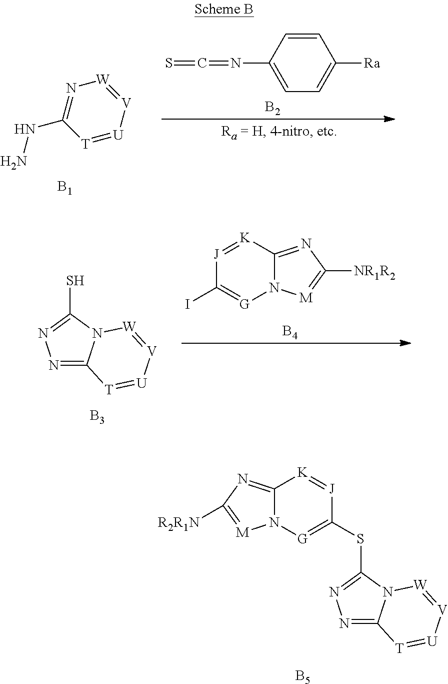

Compound B1 is treated with compound B2 (e.g., phenylisothiocyanate or p-nitrophenylisothiocyanate) in DME at 100-120° C. in a microwave for 1-8 hrs to provide thiol (B3). A palladium mediated coupling of compound B3 and B4 in DME with Pd₂(dba)₃ and 4,5-bis(phenylphosphino)-9,9-dimethylxanthene at 100-120° C. in a microwave for 1-8 hrs can be used to provide compound B5.

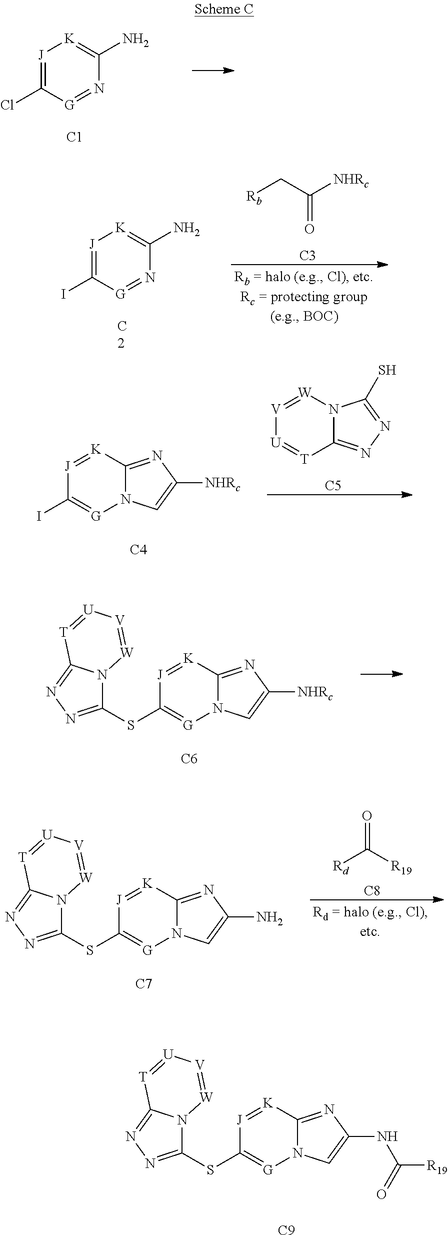

Aniline (C1) is heated in HI at 100° C. for 18 hrs to provide compound C2. Aniline (C2) is treated with acetamide (C3) and a base such as sodium hydrogen phosphate in a polar solvent (eg. DMA, DMF, or DMSO) and heated at 100-120° C. in a microwave for 1-8 hrs to afford cyclized product C4. Palladium mediated coupling of C4 and C5 can be accomplished in DME with Pd₂(dba)₃ and 4,5-bis(phenylphosphino)-9,9-dimethylxanthene at 100-120° C. in a microwave for 1-8 hrs to provide compound C6. Removal of the R, group from C6 can be accomplished in a solvent such as THF or dioxane or an alcohol such as MeOH, EtOH, or iPrOH in the presence of an acid such as HCl or TFA at 0-100° C. for 1-24 hrs to provide compound C7. Compound C7 is treated with the appropriate acylchloride in a solvent (e.g., DCM, THF, or CHCl₃) in the presence of a base (e.g., TEA, DIEA, or pyridine) at 0-80° C. for 1-24 hrs to provide compound C9.

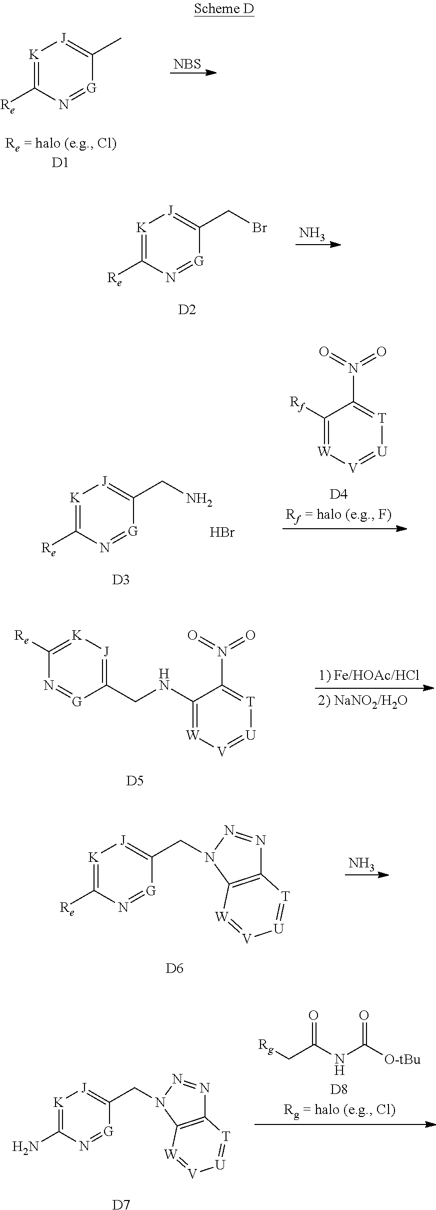

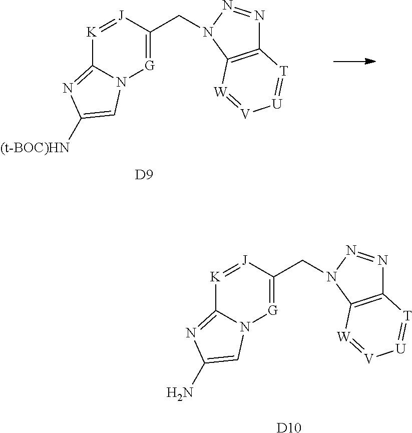

Compound D1 is brominated under standard conditions such as NBS in the presence of a peroxide, AIBN, or 200-400 w light in CCl₄ at reflux (e.g., for 1-8 hrs). Displacement of bromide (D2) with ammonia in an alcoholic solvent (e.g., MeOH, EtOH, or i-PrOH) at 0-80° C. for 1-24 hrs provides compound D3. The substitution reaction with compounds D3 and D4 is accomplished in a solvent (e.g., DMF, DMA, or EtOH) in the presence of a base (e.g., TEA, DIEA, or pyridine) at 0-80° C. for 1-24 hrs to provide the arylnitro D5. Reduction of the arylnitro (D5) is accomplished under standard conditions using a palladium catalyst such as 10% Pd/C or metal (e.g., Fe or Zn) in acidic medium. Subsequent ring closure of the resulting aniline to the triazole is accomplished in the presence of aqueous NaNO₂ to provide compound D6. Treatment of compound D6 in ammonia saturated solvent (e.g., EtOH or i-PrOH in a sealed vessel) at 100-150° C. for 1-4 days provides aniline D7. Aniline (D7) is treated with acetamide (D8) and a base such as sodium hydrogen phosphate in a polar solvent (e.g., DMA, DMF, or DMSO) and heated at 100-120° C. in a microwave for 1-8 hrs to afford cyclized product D9. Treatment of compound D9 with acid (e.g., HCl or TFA) provides compound D10.

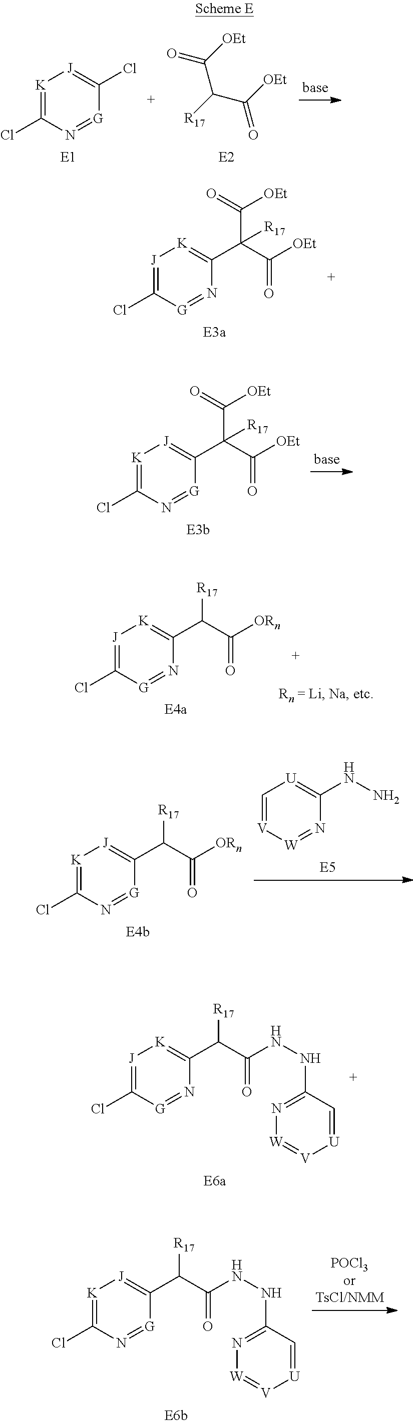

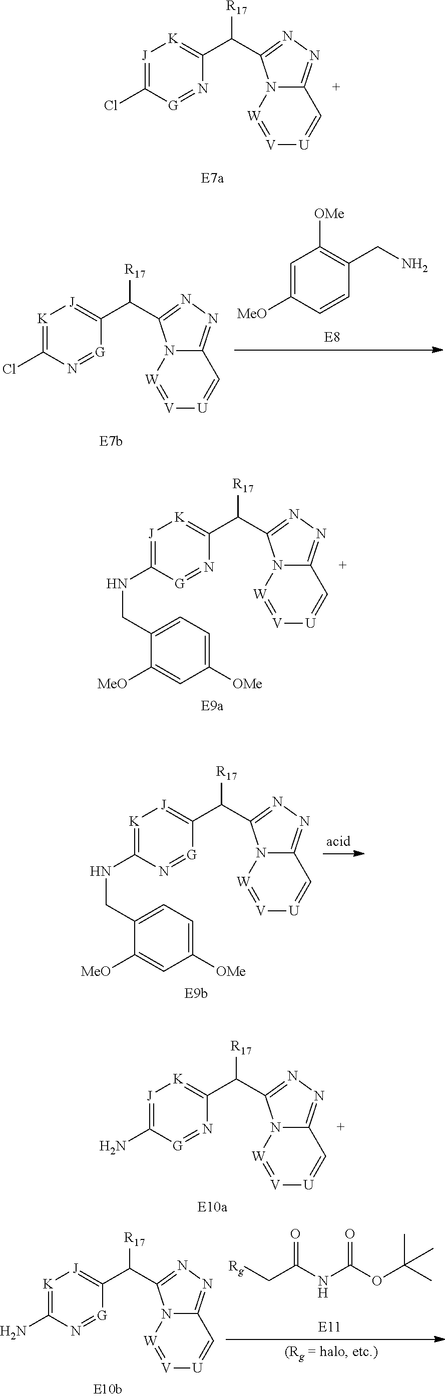

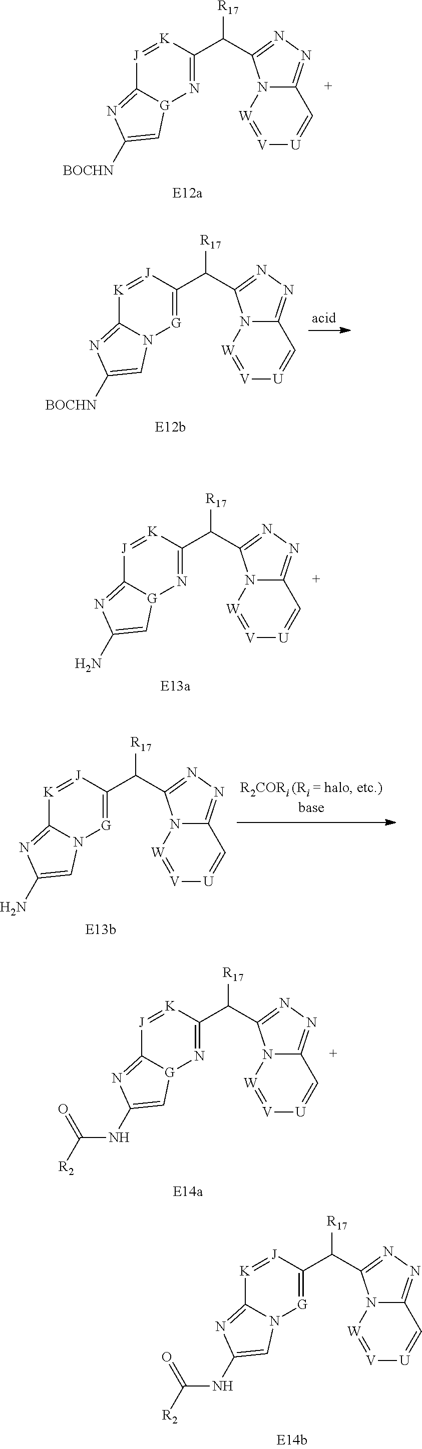

Compounds E3a and E3b can be prepared starting from compounds E1 and E2 by treating compound E2 with a base such as NaH in a solvent such as THF or dioxane at −78-0° C. and subsequent treatment with compound E1. Compound E3a and E3b are optionally separated prior to further use. Treatment of compound E3a and/or E3b with a base such as LiOH or NaOH at −20-75° C. for 1-8 hrs provides compounds E4a and/or E4b as the lithium or sodium salt, respectively. Treatment of compounds E4a and/or E4b with compound E5 in the presence of EDC or DCC for 1-24 hrs provides compounds E6a and/or E6b. Treatment of compounds E6a and/or E6b with POCl₃ at 80-160° C. provides compounds E7a and/or E7b. Alternatively, compounds E7a and/or E7b can be prepared by treating compounds E6a and/or E6b with TsCl/N-methylmorpholine at ambient temperature to 100° C. for 30 min to 8 hrs. Coupling of compound E8 and compounds E7a and/or E7b is achieved in a solvent such as THF, Dioxane, or DMF at 25-100° C. for 2-24 hrs in the presence of a co-base such as DIEA or Et₃N. Removal of the dimethoxy benzyl group from compounds E9a and/or E9b is achieved by treating compounds E9a and/or E9b with an acid such as TFA at 25-75° C. for 2-24 hrs to provide compounds E10a and/or E10b. Treatment of compounds E10a and/or E10b with compound E11 in the presence of a base such as Na₂HPO₄ in a solvent such as DMA at 120° C. provides compounds E12a and/or E12b. Treatment of compounds E12a and/or E12b with an acid such as TFA or HCl provides compounds E13a and/or E13b. Treatment of compounds E13a and/or E13b with an acid halide in the presence of a co-base such as DIEA or Et₃N at 25-75° C. in a solvent such as DCM, THF, dioxane or DMF provides compounds E14a and/or E14b.

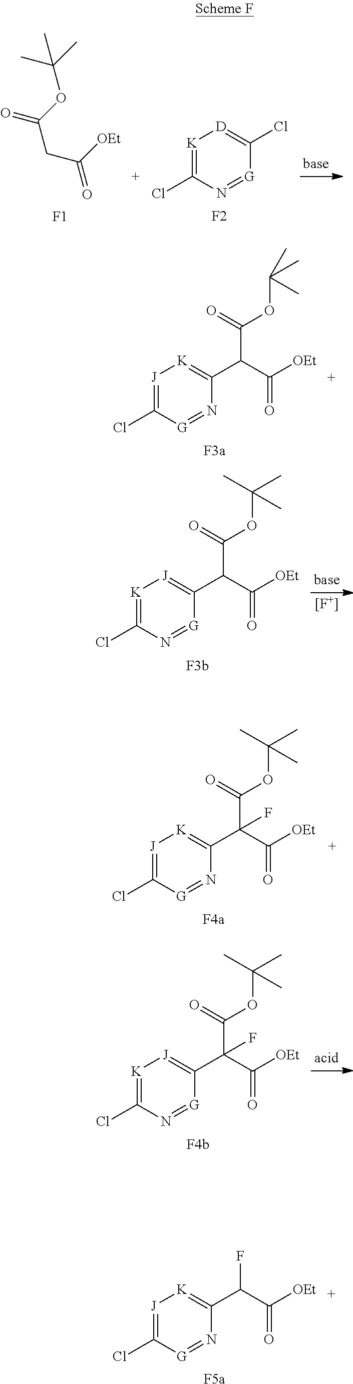

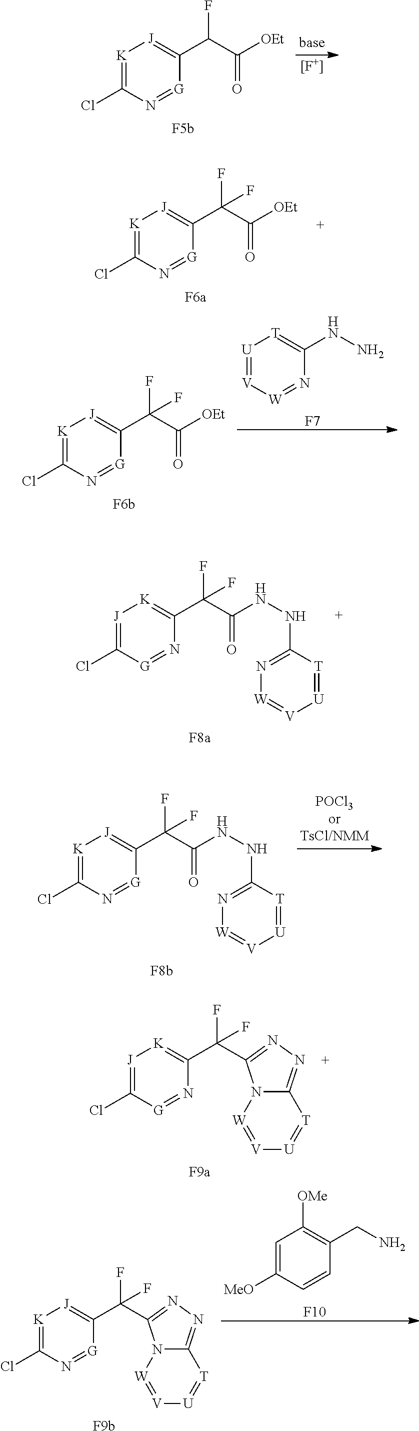

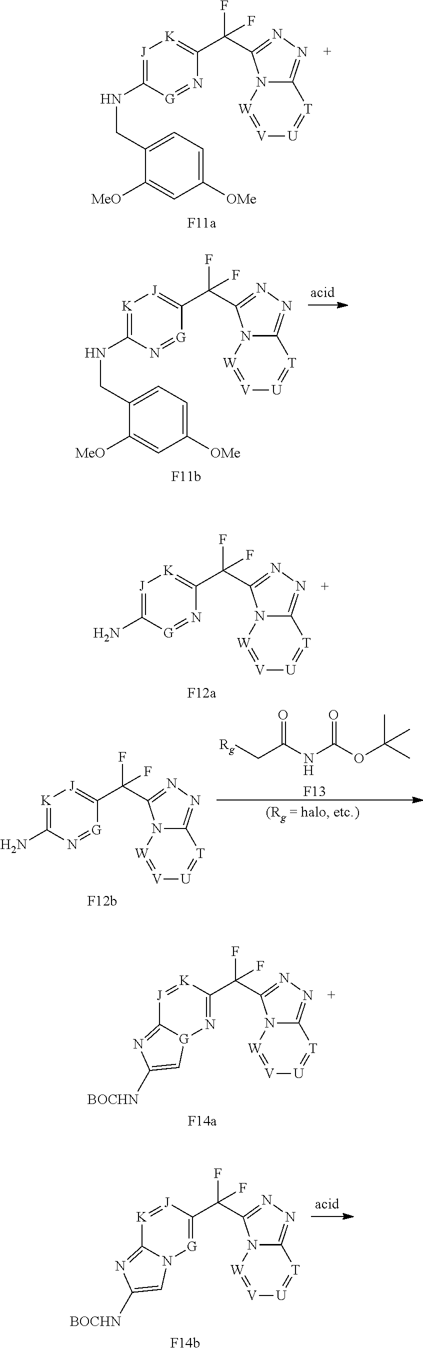

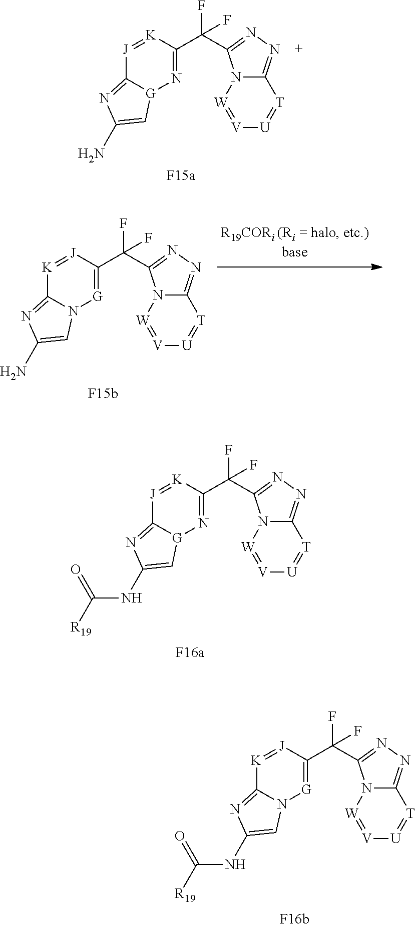

Compounds F3a and F3b can be prepared starting from compounds F1 and F2 by treating compound F1 with a base such as NaH in a solvent such as THF or dioxane at −78-0° C. and subsequent treatment with compound F2. Compound F3a and F3b are optionally separated prior to further use. Treatment of compounds F3a and/or F3b with a base such as NaH and a fluoride ion source provides compounds F4a and/or F4b. Subsequent treatment of compounds F4a and/or F4b with an acid such as TFA or HCl in a solvent such as THF, DCM, or dioxane at 0-75° C. for 1-8 hrs provides compounds F5a and/or F5b. Treatment of compounds F5a and/or F5b with a base such as NaH and a fluoride ion source provides compounds F6a and/or F6b. Treatment of compounds F6a and/or F6b with compound F7 in the presence of base such as TEA or DIEA in solvents such as MeOH or EtOH for 1-24 hrs provides compounds F8a and/or F8b. Treatment of compounds F8a and/or F8b with POCl₃ at 80-160° C. provides compounds F9a and/or F9b. Alternatively, compounds F9a and/or F9b can be prepared by treating compounds F8a and/or F8b with TsCl/N-methylmorpholine at ambient temperature to 100° C. for 30 min to 8 hrs. Coupling of compounds F9a and/or F9b with compound F10 is achieved in a solvent such as THF, Dioxane, or DMF at 25-100° C. for 2-24 hrs in the presence of a co-base such as DIEA or Et₃N. Removal of the dimethoxy benzyl group from compounds F11a and/or F 11b is achieved by treating compounds F11a and/or F11b with an acid such as TFA at 25-75° C. for 2-24 hrs to provide compounds F12a and/or F12b. Treatment of compounds F12a and/or F12b with compound F13 in the presence of a base such as Na₂HPO₄ in a solvent such as DMA at 120° C. provides compounds F14a and/or F14b. Treatment of compounds F14a and/or F14b with an acid such as TFA or HCl provides compounds F15a and/or F15b. Treatment of compounds F15a and/or F15b with an acid halide in the presence of a co-base such as DIEA or Et₃N at 25-75° C. in a solvent such as DCM, THF, dioxane or DMF provides compounds F16a and/or F16b.

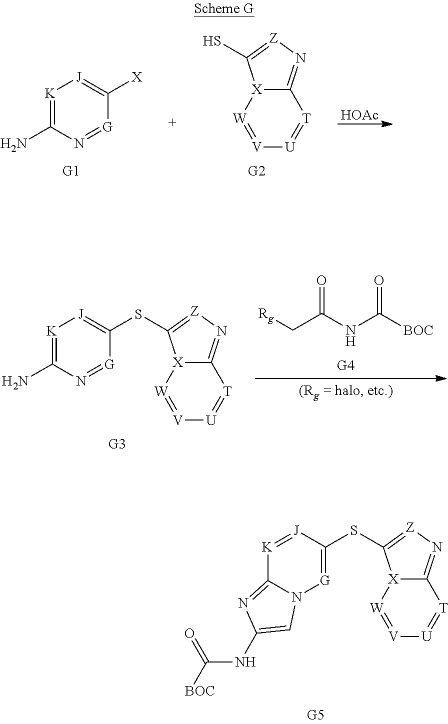

Heating compound G1 and G2 in HOAc at 50-100° C. for 2-24 hrs provides compound G3. Treatment of compound G3 with compound G4 in the presence of a base such as Na₂HPO₄ in a solvent such as DMA at 120° C. provides compound G5.

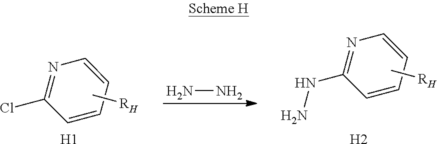

A solution of H1 (5 mmol; wherein R_{H} is R₈, R₉, R₁₀ or R₁₁) and hydrazine (30 mmol) in isopropanol or EtOH (5 mL) is heated under microwave conditions at 60-160° C. for 2-5 h dependent on substrates. The solid product is filtered, washed with water and dried under high vacuum. Excess hydrazine can optionally be removed by concentrating the mixture and co-evaporating it with MeOH and Et₃N. The product can be suspended in ether, filtered and dried under high vacuum (e.g., overnight).

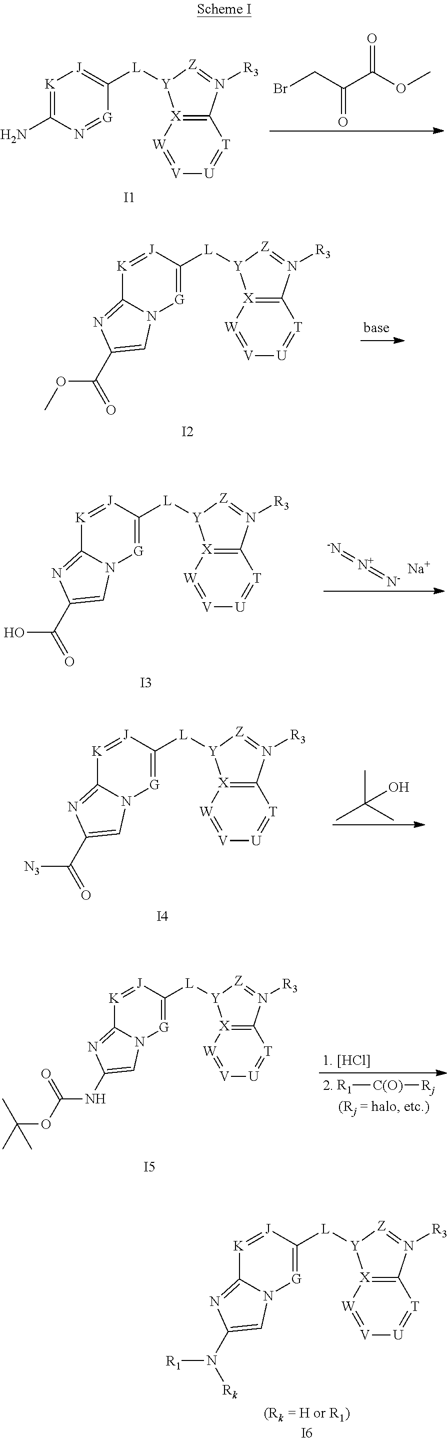

Compound I1 is treated with methyl 3-bromo-2-oxopropanoate in the presence of a base such as NaHCO₃, Na₂CO₃, sodium phosphate (mono, di, or tri-basic), or K₂CO₃ in a solvent such as dioxane or THF and heated at 50-100° C. for 1-18 hrs to provide compound of structure 12. Compound I2 is saponified with a base such as NaOH, KOH, or LiOH in a solvent such as MeOH, EtOH, or alcohol water mixtures with stirring for 1-18 hrs at 25-100° C. to provide compound of structure 13. Compound I3 can be treated with sodium azide and PyBOB in the presence of a base such as TEA, DIEA, or pyridine in an anhydrous solvent such as DMA or DMF and stirred for 1-18 hrs at ambient temperature to provide compound 14. Compound I4 is stirred in t-butanol for 1-18 hrs at 25120° C. to provide compound I5. Compound I5 is treated with an acid such as TFA or HCl in a solvent such as DCM or dioxane and stirred for 1-18 hrs at 25-100° C. This deprotected product is then treated with an acylchloride in the presence of a base such as TEA, DIEA, or pyridine (which gives rise to a bis acylated product) or without a base (which gives rise to a mono acylated product) in a solvent such as DCM, THF, dioxane, or DMF and stirred for 1-18 hrs at 25-100° C. to yield compounds of structure 16.

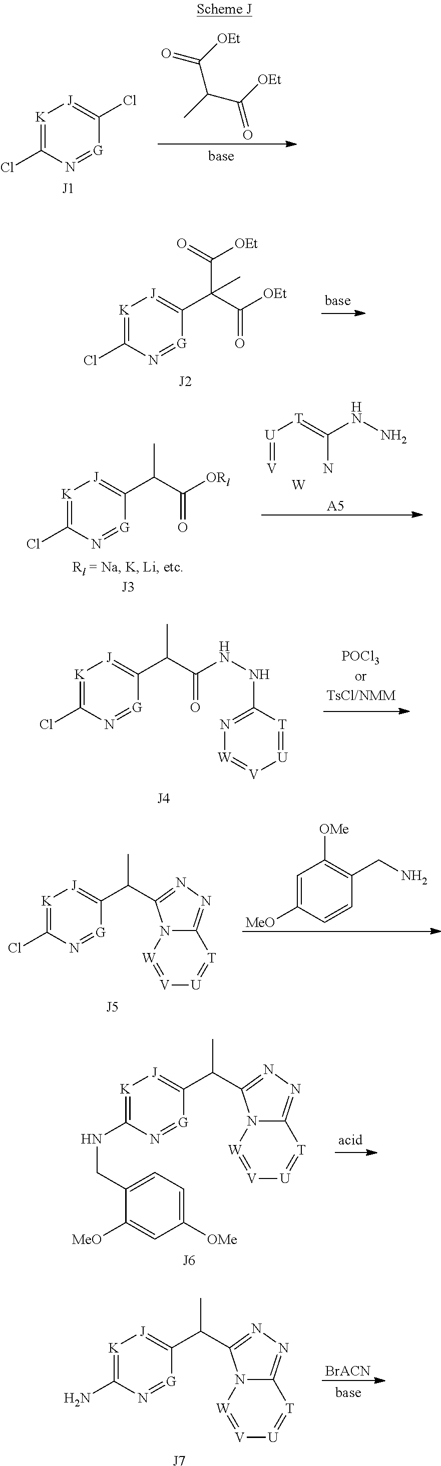

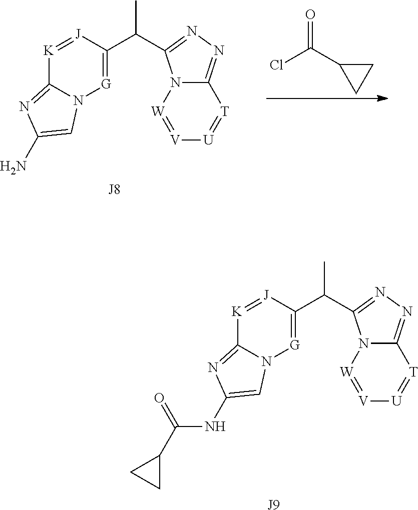

To a mixture of a base such as NaH or potassium t-butoxide in a solvent such as dioxane or THF is added diethyl 2-methylmalonate at −78-0° C. The mixture is allowed to warm to ambient temperature over 1-4 hrs. To this is added J1 portion-wise and the reaction is then stirred for 1-18 hrs at 25-100° C. to give compounds of structure J2. Compound J2 is saponified with a base such as NaOH, KOH, or LiOH in a solvent such as MeOH, EtOH, or alcohol water mixtures with stirring for 1-18 hrs at 25-100° C. to provide compound of structure J3 as the appropriate salt. Compound J3 and A5 in the presence of coupling reagents such as EDCI and HoBT or PyBOB in a solvent such as DMF or DMA are stirred for 1-8 hrs at 25-100° C. to provide compounds of structure J4. Compound J4 is heated in phosphorous oxychloride for 1-18 hrs at 80-160° C. to give compounds of structure J5. Alternatively, compound J5 can be prepared by treating compound J4 with TsCl/N-methylmorpholine at ambient temperature to 100° C. for 30 min to 8 hrs. Compound J5 is treated with 2,4-dimethoxybenzylamine in the presence of a base such as NaHCO₃, Na₂CO₃, sodium phosphate (mono, di, or tri-basic), or K₂CO₃ and in a solvent such as IPA and is heated in a microwave for 1-24 hrs at 75-150° C. to give compounds of structure J6. Compound J6 is treated with an acid such as TFA or HCl in a solvent such as DCM or dioxane for 1-18 hrs at 25-100° C. to give compounds of structure J7. Compound J7 is treated with bromoacetonitrile in the presence of a base such as NaHCO₃, Na₂CO₃, sodium phosphate (mono, di, or tri-basic), or K₂CO₃ and in a solvent such as IPA for 1-18 hrs at 100° C. in a sealed tube to provide compounds of structure J8. Compound J8 is treated with cyclopropanecarbonyl chloride in the presence of a base such as TEA, DIEA, or pyridine or in the absence of a base and in a solvent such as DCM, THF, or dioxane for 1-8 hrs at ambient temperature to provide compounds of structure J9.

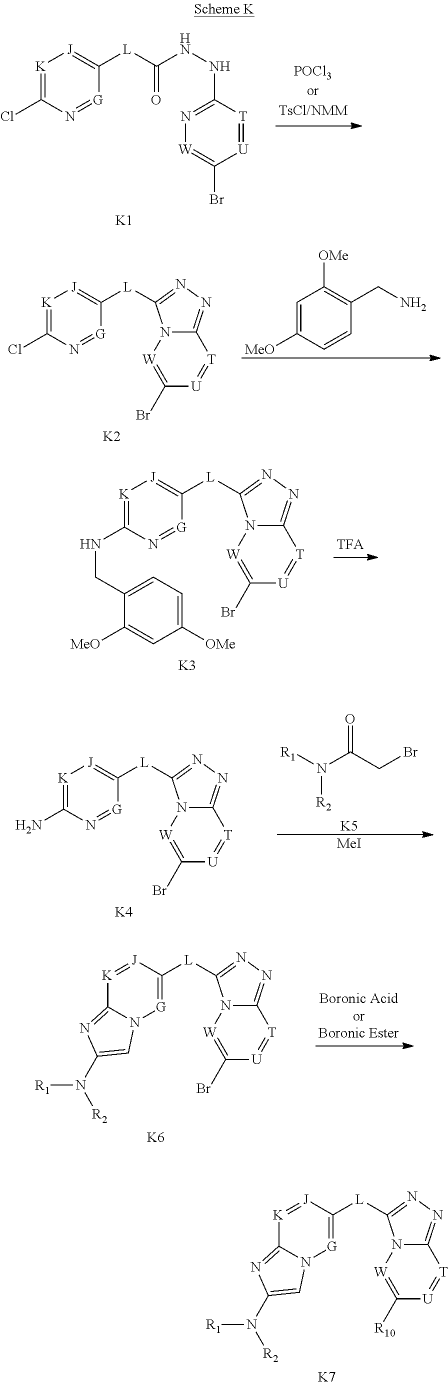

Compound K1 is heated in phosphorous oxychloride for 1-18 hrs at 80-160° C. to give compounds of structure K2. Alternatively, compound K2 can be prepared by treating compound K1 with TsCl/N-methylmorpholine at ambient temperature to 100° C. for 30 min to 8 hrs. Compound K2 is treated with 2,4-dimethoxybenzylamine in the presence of a base such as NaHCO₃, Na₂CO₃, sodium phosphate (mono, di, or tri-basic), or K₂CO₃ and in a solvent such as IPA and is heated in a microwave for 1-24 hrs at 75-150° C. to give compounds of structure K3. Compound K3 is treated with an acid such as TFA or HCl in a solvent such as DCM or dioxane for 1-18 hrs at 25-100° C. to give compounds of structure K4. Compounds of structure K4 is heated with compounds of structure K5 in the presence of KI and a base such as sodium hydrogenphosphate and in a solvent such as DMA or DME for 1-24 hrs at 50-120° C. to provide compounds of structure K6. Compound K6 is treated with the appropriate boronic acid or boronic ester in the presence of a catalyst such as tetrakis(triphenylphosphine)palladium(0) and a base such as Cs₂CO₃ (aq) or K₂CO₃ (aq) in a solvent such as dioxane, THF, or DME for 15 min 8 hrs at 50-120° C. to give compounds of structure K7.

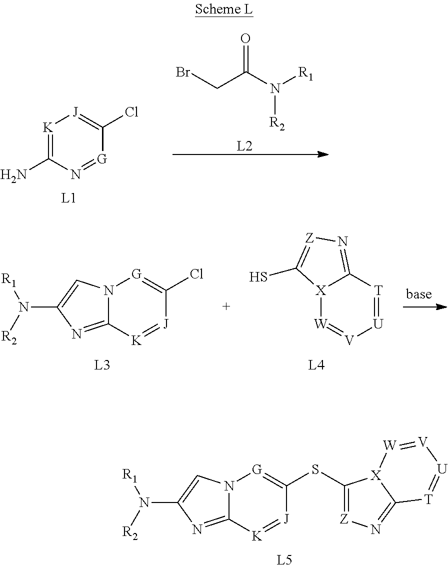

Compound L1 is treated with compound L2 and a base (eg., K₂HPO₄) in a polar solvent (eg., DMA, DMF or DMSO) and heated at 65-85° C. to provide compound L3. Compound L3 was reacted with Compound L4 and a base (eg., K₂CO₃) in a polar solvent (eg., DMA, DMF or DMSO) at 130-160° C. and atmospheric pressure to 125 psi N₂ to provide compound L5.

Chiral components can be separated and purified using any of a variety of techniques known to those skilled in the art. For example, chiral components can be purified using supercritical fluid chromatography (SFC). In one particular variation, chiral analytical SFC/MS analyses are conducted using a Berger analytical SFC system (AutoChem, Newark, Del.) which consists of a Berger SFC dual pump fluid control module with a Berger FCM 1100/1200 supercritical fluid pump and FCM 1200 modifier fluid pump, a Berger TCM 2000 oven, and an Alcott 718 autosampler. The integrated system can be controlled by BI-SFC Chemstation software version 3.4. Detection can be accomplished with a Watrers ZQ 2000 detector operated in positive mode with an ESI interface and a scan range from 200-800 Da with 0.5 second per scan. Chromatographic separations can be performed on a ChiralPak AD-H, ChiralPak AS-H, ChiralCel OD-H, or ChiralCel OJ-H column (5μ, 4.6×250 mm; Chiral Technologies, Inc. West Chester, Pa.) with 10 to 40% methanol as the modifier and with or without ammonium acetate (10 mM). Any of a variety of flow rates can be utilized including, for example, 1.5 or 3.5 mL/min with an inlet pressure set at 100 bar. Additionally, a variety of sample injection conditions can be used including, for example, sample injections of either 5 or 10 μL in methanol at 0.1 mg/mL in concentration.

In another variation, preparative chiral separations are performed using a Berger MultiGram II SFC purification system. For example, samples can be loaded onto a ChiralPak AD column (21×250 mm, 10 μl. In particular variations, the flow rate for separation can be 70 mL/min, the injection volume up to 2 mL, and the inlet pressure set at 130 bar. Stacked injections can be applied to increase the efficiency.

In each of the above reaction procedures or schemes, the various substituents may be selected from among the various substituents otherwise taught herein.

Descriptions of the syntheses of particular compounds according to the present invention based on the above reaction scheme are set forth herein.

Examples of cMET Inhibitors

The present invention is further exemplified, but not limited by, the following examples that describe the synthesis of particular cMET inhibitors useful in the present invention.

### Compounds 1 and 2: N-(6-((3H-[1,2,3]triazolo[4,5-b]pyridin-3-yl)methyl)imidazo[1,2-a]pyridin-2-yl)-2,2,2-trifluoro acetamide and N-(6-((1H-[1,2,3]triazolo[4,5-b]pyridin-1-yl)methyl)imidazo-[1,2-a]pyridin-2-yl)-2,2,2-trifluoroacetamide

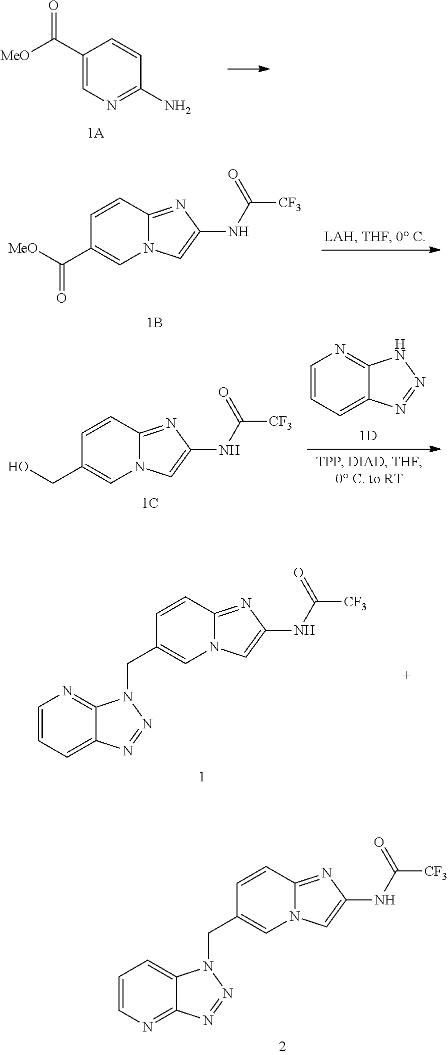

### Methyl 2-(2,2,2-trifluoroacetamido)imidazo[1,2-a]pyridine-6-carboxylate (1B)

Compound 1B was prepared starting from compound 1A in three steps following an analogous method to that described in U.S. Pat. No. 6,358,971, which is incorporated by reference herein in its entirety. Specifically, a mixture of methyl 6-aminonicotinate (1A, 3 g, 19.7 mmol) and tosylchloride (4.5 g, 23.6 mmol) in pyridine (40 mL) was heated at 80° C. for 16 hrs. The reaction was cooled to room temperature and pyridine was removed in vacuo. The resulting residue was diluted with water and allowed to stir for 10 min. The resulting solid was filtered and dried under vacuum to provide methyl 6-(4-methylphenylsulfonamido)nicotinate which was used without further purification. To a suspension of methyl 6-(4-methylphenylsulfonamido) nicotinate (5.3 g, 17.3 mmol) in anhydrous DMF was sequentially added DIEA (3.31 mL, 19.03 mmol) and then 2-bromoacetamide (2.63 g, 19.03 mmol). The reaction was stirred at ambient temperature for 24 hrs and then poured into water. The resulting solid was filtered and dried under vacuum to provide methyl 1-(2-amino-2-oxoethyl)-6-(4-methylphenylsulfonamido)-1,6-dihydropyridine-3-carboxylate which was used without further purification. To a suspension of methyl 1-(2-amino-2-oxoethyl)-6-(4-methylphenylsulfonamido)-1,6-dihydropyridine-3-carboxylate (1.0 g, 2.8 mmol) in DCM (20 mL) was added TFAA (8.0 mL, 57.5 mmol) dropwise at ambient temperature. The reaction was refluxed for 2 hrs, cooled to ambient temperature, and then concentrated in vacuo. The resulting residue was suspended in saturated sodium bicarbonate and stirred for 15 min. The resulting solid was filtered and dried under vacuum to provide methyl 2-(2,2,2-trifluoroacetamido)imidazo[1,2-a]pyridine-6-carboxylate (1B) which was used without further purification.

### 2,2,2-Trifluoro-N-(6-(hydroxymethyl)imidazo[1,2-a]pyridin-2-yl)acetamide (1C)

To a stirred solution of compound 1B (470 mg, 1.64 mmol) in anhydrous THF (20 mL) were portion wise added lithium aluminum hydride (155 mg, 4.09 mmol) at 0° C. After being stirred for 1 h at 0° C., the reaction was quenched with 0.16 mL of water, followed by 0.16 mL of 15% NaOH and 0.48 mL of water. The heterogenous reaction mixture was stirred for 0.5 h at room temperature and then filtered through celite. Residue was washed with THF. Filtrate and washings were concentrated and the crude residue was purified by column chromatography to furnish compound 1C (220 mg, 52%). ¹H NMR (400 MHz, DMSO-d₆) δ ppm 12.41 (s, 1H), 8.52 (s, 1H), 8.21 (s, 1H), 7.47 (d, J=9.2 Hz, 1H), 7.25 (d, J=9.6 Hz, 1H), 5.36 (t, J=5.6 Hz, 1H) 4.48 (d, J=5.6 Hz, 2H) ESI-MS: m/z 260.2 (M+H)⁺.

### N-(6-((3H-[1,2,3]triazolo[4,5-b]pyridin-3-yl)methyl)imidazo[1,2-a]pyridin-2-yl)-2,2,2-trifluoroacetamide and N-(6-((1H-[1,2,3]triazolo[4,5-b]pyridin-1-yl)methyl)imidazo-[1,2-a]pyridin-2-yl)-2,2,2-trifluoroacetamide (1 and 2)

To a stirred solution of compound 1C (30 mg, 0.12 mmol) in anhydrous THF (3.0 mL) were sequentially added compound 1D (28 mg, 0.23 mmol) and triphenyl phosphine (61 mg, 0.23 mmol). The reaction mixture was cooled to 0° C., and to it diisopropyl-azodicarboxylate (0.05 mL, 0.23 mmol) was added in drop wise manner. After the addition was over, stirring continued for another 0.5 h at 0° C. and then for 12 h at room temperature. Solvents were removed in vacuum and the residue was purified by preparative HPLC to provide compounds 1 and 2 as TFA salts. ESI-MS for both compounds: m/z 362.1 (M+H)⁺.

### Compound 3: N-(6-([1,2,4]triazolo[4,3-a]pyridin-3-ylthio)imidazo[1,2-b]pyridazin-2-yl)cyclo-propanecarboxamide

### Method A

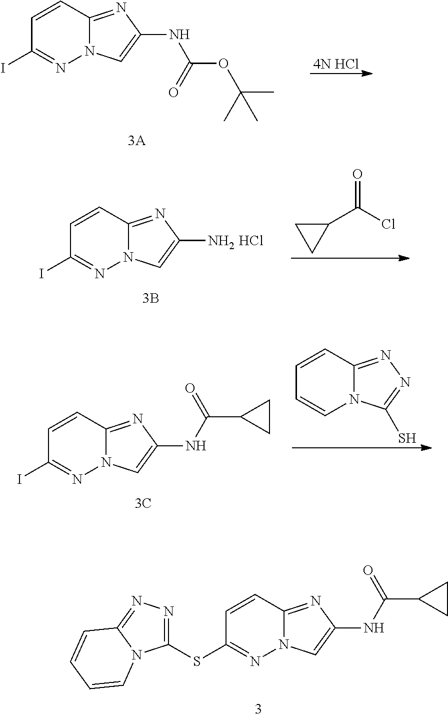

### 6-iodoimidazo[1,2-b]pyridazin-2-amine hydrochloride (3B)

Compound 3B was synthesized according to a procedure analogous to that described in International Patent Publication No. WO 2008/06192 (Takeda Pharmaceutical Company Limited), which is incorporated by reference herein in its entirety. Specifically, tert-butyl 6-iodoimidazo[1,2-b]pyridazin-2-ylcarbamate (3A, 5.9 g, 16.38 mmol) was dissolved in 4N HCl in dioxane (60 mL) and stirred at ambient temperature for 4 hours. Ether (140 mL) was added to the reaction and a brown precipitate formed. The precipitate was filtered and washed with ether to give 6-iodoimidazo[1,2-b]pyridazin-2-amine hydrochloride in quantitative yield. This material was used without purification. ESI-MS: m/z 261.0 (M+H)⁺.

### N-(6-iodoimidazo[1,2-b]pyridazin-2-yl)cyclopropanecarboxamide (3C)

Compound 3C was synthesized according to a procedure analogous to that described in International Patent Publication No. WO 2008/06192 (Takeda Pharmaceutical Company Limited), which is incorporated by reference herein in its entirety. 6-Iodoimidazo[1,2-b]pyridazin-2-amine hydrochloride (4.45 g, 17.11 mmol) and cyclopropanecarbonyl chloride (1.35 mL, 18.82 mmol) were dissolved in DMA (85 mL) and stirred for 3 hours at ambient temperature. The reaction mixture was then poured into water (400 mL) resulting in the formation of a brown precipitate. The solid was filtered and dried under vacuum to give N-(6-iodoimidazo[1,2-b]pyridazin-2-yl)cyclopropanecarboxamide (4.2 g, 77%). ¹H NMR (400 MHz, DMSO-d₆) δ=11.21 (s, 1H), 8.26-8.17 (m, 1H), 7.74 (d, J=9.3 Hz, 1H), 7.49 (d, J=9.1 Hz, 1H), 2.07-1.87 (m, 1H), 0.97-0.77 (m, 4H) ESI-MS: m/z 329.1 (M+H)⁺.

### N-(6-([1,2,4]triazolo[4,3-a]pyridin-3-ylthio)imidazo[1,2-b]pyridazin-2-yl)cyclopropanecarboxamide (3)

N-(6-iodoimidazo[1,2-b]pyridazin-2-yl)cyclopropanecarboxamide (0.5 g, 1.52 mmol), [1,2,4]triazolo[4,3-a]pyridine-3-thiol (0.23 g, 1.52 mmol), Tris(dibenzylideneacetone)dipalladium(0) (84 mg, 0.09 mmol), 9,9-Dimethyl-4,5-bis(diphenylphosphino)xanthene (106 mg g, 0.18 mmol), and DIEA (0.531 mL, 3.05 mmol) were dissolved in DME (15.2 mL) and heated in a microwave at 120° C. for 30 min. The reaction mixture was concentrated to dryness, reconstituted in DMSO, and purified by preparative LCMS to give N-(6-([1,2,4]triazolo[4,3-a]pyridin-3-ylthio)imidazo[1,2-b]pyridazin-2-yl)cyclopropanecarboxamide (1 g, 43%) as a TFA salt. ¹H NMR (400 MHz, DMSO-d₆) δ=11.17 (s, 1H), 8.57-8.44 (m, 1H), 8.03-7.94 (m, 1H), 7.96-7.88 (m, 2H), 7.59 (ddd, J=1.0, 6.6, 9.3 Hz, 1H), 7.22-7.11 (m, 1H), 7.11-7.03 (m, 1H), 1.97-1.84 (m, 1H), 0.83-0.73 (m, 4H) ESI-MS: m/z 352.3 (M+H)⁺. MP 193-195° C.

### Method B

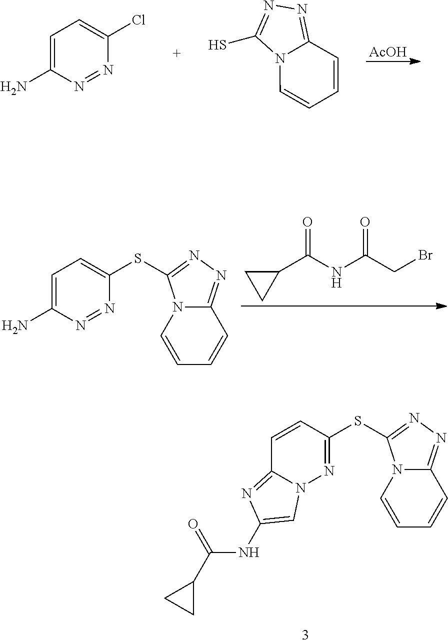

### 6-([1,2,4]Triazolo[4,3-a]pyridin-3-ylsulfanyl)-pyridazin-3-ylamine

A reaction mixture of [1,2,4]triazolo[4,3-a]pyridine-3-thiol (8.49 g, 55.0 mmol) and 6-chloropyridazin-3-amine (6.82 g, 50 mmol) in acetic acid (100 mL) was heated at 80° C. for 20 hrs. The reaction was stripped to dryness via rotary evaporation and the resulting material was suspended in H₂O (100 mL). To this suspension, solid Na₂CO₃ was added in portions until pH 10 was achieved and then the mixture was sonicated. The resulting solid was collected by filtration and rinsed thoroughly with water followed by ethyl ether. The solid was dried in vacuum over P₂O₅ to provide the title compound, 6-([1,2,4]Triazolo[4,3-a]pyridin-3-ylsulfanyl)-pyridazin-3-ylamine, as a white power (11.0 g). ¹H NMR (400 MHz, DMSO-d₆) δ ppm 7.15-7.25 (m, 1H) 7.53-7.58 (m, 1H) 7.61 (ddd, J=9.22, 6.69, 1.26 Hz, 1H) 7.84-7.90 (m, 1H) 7.99 (dt, J=9.09, 1.01 Hz, 1H) 8.51 (dt, J=7.01, 1.04 Hz, 1H) 8.79 (br. s., 2H). ESI-MS: m/z 553.2 (M+H)⁺.

### N-(6-([1,2,4]Triazolo[4,3-a]pyridin-3-ylthio)inidazo[1,2-b]pyridazin-2-yl)cyclopropanecarboxamide

A mixture of 6-([1,2,4]triazolo[4,3-a]pyridin-3-ylthio)pyridazin-3-amine (2.443 g, 10 mmol), N-(2-bromoacetyl)cyclopropanecarboxamide (3.09 g, 15.00 mmol), potassium hydrogenphosphate (5.23 g, 30.0 mmol) and potassium iodide (0.830 g, 5.00 mmol) in DMA (100 mL) was stirred at 120° C. for 2 hrs. Additional amounts of N-(2-bromoacetyl)cyclopropanecarboxamide (2.06 g, 10 mmol) and potassium hydrogenphosphate (1.74 g, 10 mmol) were added to the mixture and the reaction was stirred at 120° C. for another 2 hrs. The reaction was then stirred at room temperature overnight. The reaction mixture was again stirred at 140° C. for 3 hrs and then cooled to room temperature. The resulting solid was filtered off and rinsed with DMA. The filtrate was concentrated to 100 mL and then poured into water (300 mL). The resulting precipitate was collected by filtration and rinsed thoroughly with water. The solid material was re-suspended in 10% MeOH/CH2Cl2 (100 mL), sonicated, refluxed for 30 min and cooled to room temperature. The MeOH/CH2Cl2 solution was filtered through a silica plug, rinsed with 10% MeOH/CH2Cl2 (200 mL). To above organic solution, activated charcoal (0.5 g) was added, the solution was refluxed for 30 min, and then stirred at room temperature overnight. The charcoal was filtered through Celite, and the filtrate was concentrated to dryness to provide an off white solid. This solid was refluxed in MeOH (10 mL) for 30 min. and cooled to room temperature. The resulting off white solid was collected by filtration, rinsed with MeOH (2 mL) and dried in vacuum to provide the title compound, N-(6-([1,2,4]Triazolo[4,3-a]pyridin-3-ylthio)imidazo[1,2-b]pyridazin-2-yl)cyclopropanecarboxamide (1.2 g). ¹H NMR (400 MHz, DMSO-d₆) δ=11.17 (s, 1H), 8.57-8.44 (m, 1H), 8.03-7.94 (m, 1H), 7.96-7.88 (m, 2H), 7.59 (ddd, J=1.0, 6.6, 9.3 Hz, 1H), 7.22-7.11 (m, 1H), 7.11-7.03 (m, 1H), 1.97-1.84 (m, 1H), 0.83-0.73 (m, 4H) ESI-MS: m/z 352.3 (M+H)⁺. MP 193-195° C.

### Method C

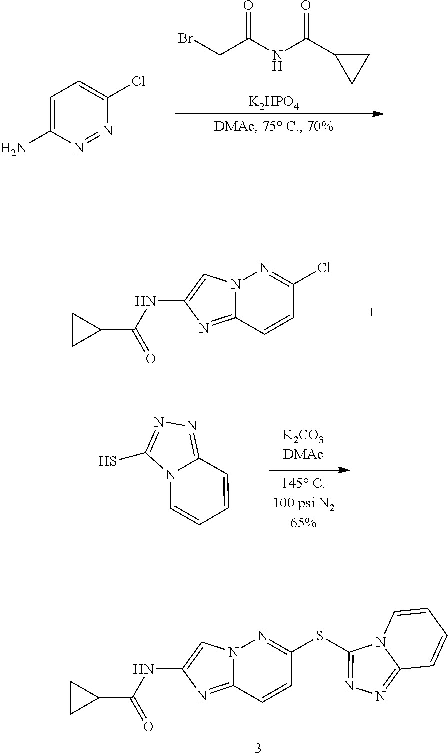

6-Chloropyridazin-3-amine, N-(2-bromoacetyl)cyclopropanecarboxamide, and potassium hydrogenphosphate were mixed in DMA at 75° C. to provide N-(6-chloroimidazo[1,2-b]pyridazin-2-yl)cyclopropanecarboxamide. N-(6-chloroimidazo[1,2-b]pyridazin-2-yl)cyclopropanecarboxamide was reacted with [1,2,4]triazolo[4,3-a]pyridine-3-thiol and K₂CO₃ in DMA at 145° C. and 100 psi N₂ to provide N-(6-([1,2,4]triazolo[4,3-a]pyridin-3-ylthio)imidazo[1,2-b]pyridazin-2-yl)cyclo-propanecarboxamide.

### Compound 4: N-(6-(6-bromo-[1,2,4]triazolo[4,3-a]pyridin-3-ylthio)imidazo[1,2-b]pyridazin-2-yl)cyclopropanecarboxamide

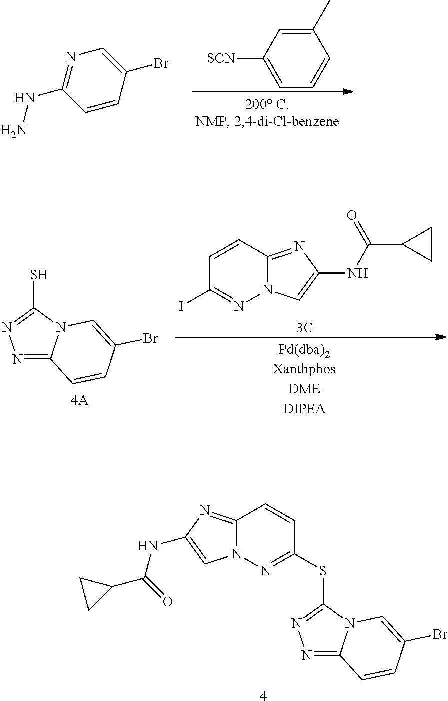

### 6-bromo-[1,2,4]triazolo[4,3-a]pyridine-3-thiol (4A)

To the suspended solution of 5-Bromo-2-hydrazinylpyridine (1.85 g, 10 mmol) in a mixture of 1,4-dichlorobenzene and NMP (5:1, 10 ml) was added isothiocyanato-3-methylbenzene (1.5 g 10 mmol). The mixture was stirred at rt for 5 min, 70° C. for 15 min, and then heated under microwave condition at 200° C. for 1.5 h. The mixture was poured into Ether (20 mL). Solid was filtered and the washed with ether to give title product.

### N-(6-(6-bromo-[1,2,4]triazolo[4,3-a]pyridin-3-ylthio)imidazo[1,2-b]pyridazin-2-yl)cyclopropanecarboxamide (4)

To a sealed tube packed with N-(6-iodoimidazo[1,2-b]pyridazin-2-yl)cyclopropanecarboxamide (100 mg, 0.3 mmol), compound 4A, Pd(dba)₂ (3%/mol), xanthphos (5%/mol) and DIPEA (1.5 mmol) was added DME (2 mL). After degas under vacuum, the mixture was heated under microwave condition at 125° C. for 30 min. The mixture was purified by LCMS to give the title compound as a TFA salt. ¹H NMR (400 MHz, CDCl₃-CD₃OD 10:1) δ ppm 8.5 (s, 1H), 8.1 (s, 1H), 7.8 (d, J=9.8 Hz, 1H), 7.7 (d, J=9.2 Hz, 2H), 7.60 (d, J=9.6 Hz, 1H), 7.1 (d, J=9.36 Hz, 1H), 1.7 (m, 1H), 1.05 (m, 2H), 0.90 (m, 2H) ESI-MS: m/z 430.2 (M+H)⁺.

### Compound 5: N-(6-(6-(1-methyl-1H-pyrazol-4-yl)-[1,2,4]triazolo[4,3-a]pyridin-3-ylthio)-imidazo[1,2-b]pyridazin-2-yl)cyclopropanecarboxamide

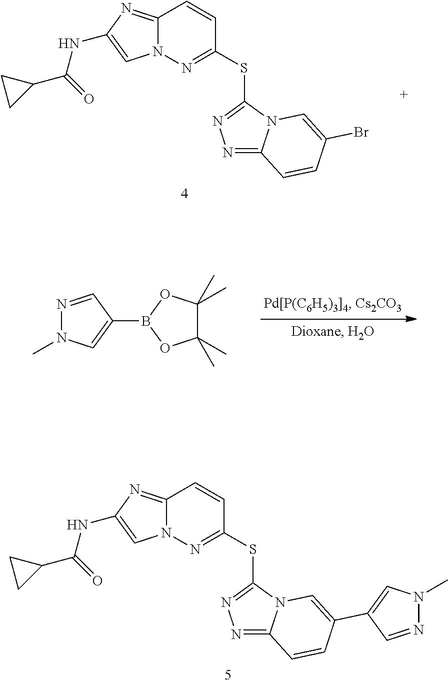

A of mixture Compound 4 (15 mg, 0.35 mmol), 1-methyl-4-(4,4,5,5-tetramethyl-1,3,2-dioxaborolan-2-yl)-1H-pyrazole (11 mg, 0.52 mmol), Cs₂CO₃ (350 mg), and tetrakis(triphenylphosphine)palladium(0) (i.e., Pd[P(C₆H₅)₃]₄) (1%/mol) in dioxane-H₂O (20:1, 1.5 mL) was heated under micro wave condition at 140° C. for 30 min. The mixture was purified by LCMS to give title compound as a TFA salt. ¹H NMR (400 MHz, CDCl₃-CD₃OD 10:1) δ ppm 8.33 (s, 1H), 8.14 (s, 1H), 7.75 (s, 1H), 7.70 (s, 1H), 7.67 (d, J=9.5 Hz, 1H), 7.56 (dd, J=9.4 and 1.6 Hz, 1H), 7.06 (d, J=9.4 Hz, 1H), 3.96 (s, 3H), 1.67 (m, 1H), 1.06 (m, 2H), 0.90 (m, 2H) ESI-MS: m/z 432.2 (M+H)⁺.

### Compound 6: N-(6-(6-cyano-[1,2,4]triazolo[4,3-a]pyridin-3-ylthio)imidazo[1,2-b]pyridazin-2-yl)cyclopropanecarboxamide

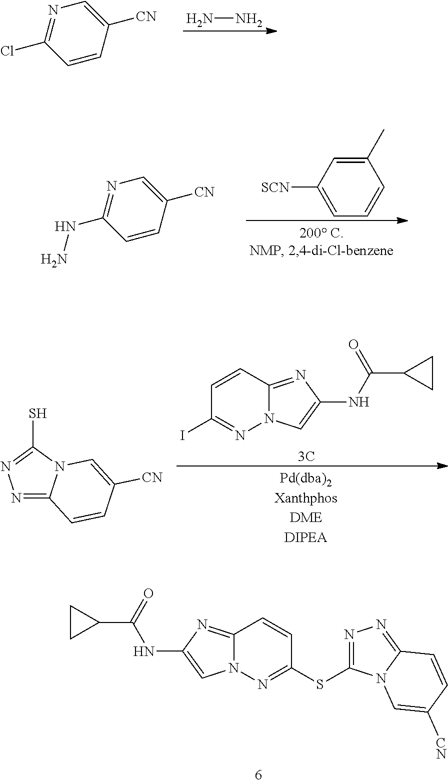

To a suspended mixture of 6-chloronicotinonitrile (7 g, 50.8 mmol) in EtOH (50 mL), was added NH₂NH₂ (10 g, 310.2 mmol). The mixture was stirred at room temperature for 10 min, and then 60° C. for 5 h. The mixture was cooled to room temperature. The solids were filtered out, washed with water and dried under high vacuum to give 6-hydrazinylnicotinonitrile (3.5 g). Compound 6 was prepared from 6-hydrazinylnicotinonitrile following the procedure of the synthesis of compound 4. ¹H NMR (400 MHz, CDCl₃-CD₃OD 10:1) δ ppm 11.37 (s, 1H), 8.70 (t, J=1.2 Hz, 1H), 8.14 (s, 1H), 8.11 (dd, J=1.04 and 9.52 Hz, 1H), 7.96 (dd, J=1.12 and 9.52 Hz, 1H), 7.61 (dd, J=1.52 and 9.48 Hz, 1H), 7.38 (d, J=9.0 Hz, 1H), 1.77 (m, 1H), 1.06 (m, 2H), 0.90 (m, 2H) ESI-MS: m/z 377.2 (M+H)⁺.

### Compound 7: tert-butyl 6-(6-chloro-[1,2,4]triazolo[4,3-a]pyridin-3-ylthio)imidazo[1,2-b]pyridazin-2-ylcarbamate

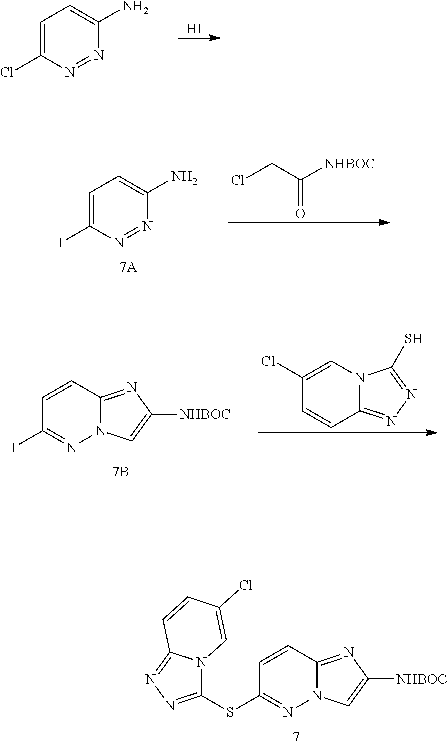

### 6-iodopyridazin-3-amine (7A)

Compound 7A was synthesized according to a procedure analogous to that described in International Patent Publication No. WO 2007/30366 (Smithkline Beecham Corporation), which is incorporated by reference herein in its entirety. Specifically, 6-chloropyridazin-3-amine (10.0 g, 77.2 mmol) was mixed with hydroiodic acid (78 mL, aqueous 57%) and heated to 100° C. overnight. Ethyl acetate (50 mL) was added after the reaction was cooled to room temperature. The resulting mixture was sonicated and filtered. The filter cake was washed with copious amounts of ethyl acetate. The crude material was then dried under high vacuum to give 6-iodopyridazin-3-amine (7A; 24 g, 89%) as bright yellow solid, which was used without further purification. ¹H NMR (400 MHz, DMSO-d₆) δ ppm 8.36-8.08 (m, 1H), 7.95 (d, J=9.6 Hz, 1H), 7.12-7.03 (m, 1H). ESI-MS: m/z 221.9 (M+H)⁺.

### Tert-butyl 6-iodoimidazo[1,2-b]pyridazin-2-ylcarbamate (7B)

A mixture containing compound 7A (2.5 g, 11.3 mmol), tert-butyl 2-chloroacetylcarbamate (2.4 g, 12.4 mmol), sodium hydrogen phosphate (3.2 g, 22.6 mmol) and DMA (23 mL) was heated at 120° C. for two hours. The mixture was cooled to room temperature, poured into a flask containing 400 mL of water and sonicated. The solids were filtered, rinsed with water then dried under vacuum. The solids were then taken up in 600 mL ethyl acetate and filtered over short plug of silica gel. The filtrate was concentrated down to give compound 7B (1.6 g, 39%), as a dark green solid, which was used without further purification. ¹H NMR (400 MHz, DMSO-d₆) δ ppm 10.21 (br. s., 1H), 8.06-8.00 (m, 1H), 7.69 (d, J=9.9 Hz, 1H), 7.46 (d, J=9.3 Hz, 1H), 1.48 (s, 9H) ESI-MS: m/z 361.1 (M+H)⁺.

### Tert-butyl 6-(6-chloro-[1,2,4]triazolo[4,3-a]pyridin-3-ylthio)imidazo[1,2-b]pyridazin-2-ylcarbamate (7)

Combined compound 7B (50 mg, 0.14 mmol), 6-chloro-[1,2,4]triazolo[4,3-a]pyridine-3-thiol (28 mg, 0.15 mmol), Tris(dibenzylideneacetone)dipalladium(0) (11 mg, 0.01 mmol), 9,9-Dimethyl-4,5-bis(diphenylphosphino)xanthene (14 mg, 0.025 mmol), DIEA (48 uL, 0.28 mmol) and DME (1.4 mL) and heated in a microwave unit at high absorbance, 120° C. for 30 min. The resulting crude material was purified by silica gel using 5% MeOH in DCM. The cleanest fractions were concentrated to give compound 7 (0.06 g; quantitative yield). ¹H NMR (400 MHz, DMSO-d₆) δ ppm 10.18 (br. s., 1H), 8.86-8.82 (m, 1H), 8.11-8.05 (m, 1H), 7.89 (d, J=9.3 Hz, 1H), 7.73 (s, 1H), 7.66 (dd, J=1.9, 9.7 Hz, 1H), 7.09 (d, J=9.6 Hz, 1H), 1.48-1.41 (m, 9H) ESI-MS: m/z 418.2.0 (M+H)⁺.

### Compound 8: 6-(6-chloro-[1,2,4]triazolo[4,3-a]pyridin-3-ylthio)imidazo[1,2-b]pyridazin-2-amine

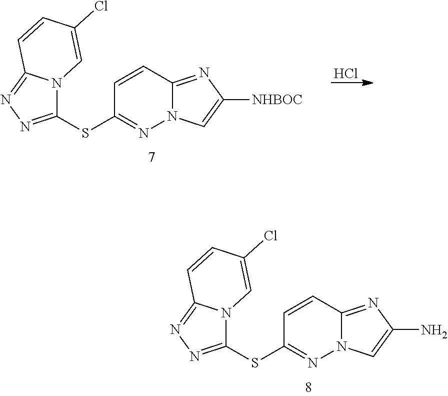

Compound 7 (0.15 g, 0.42 mmol) and 4N HCl in dioxane (2 mL) was combined and stirred at RT for 2 hours to yield compound 8. The crude reaction mixture was concentrated and used without purification. ESI-MS: m/z 318.2 (M+H)⁺.

### Compound 9: N-(6-(6-chloro-[1,2,4]triazolo[4,3-a]pyridin-3-ylthio)imidazo[1,2-b]pyridazin-2-yl)cyclopropanecarboxamide

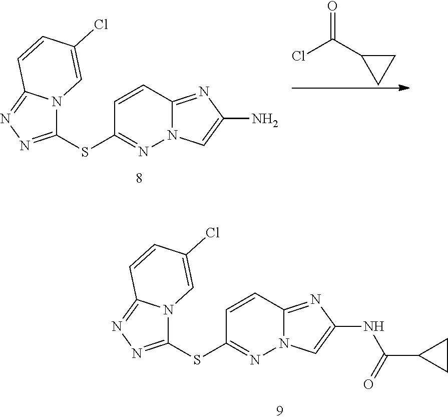 [SMILES: Nc1cn2nc(Sc3nnc4ccc(Cl)cn34)ccc2n1 ; O=C(Cl)C1CC1 ; O=C(Nc1cn2nc(Sc3nnc4ccc(Cl)cn34)ccc2n1)C1CC1]

Compound 8 (0.13 g, 0.41 mmol), cyclopropanecarbonyl chloride (112 uL, 1.23 mmol), triethylamine (690 mL, 4.9 mmol) and DCE (2 mL) were combined and stirred for 2 hours to yield compound 9. The reaction mixture was concentrated and purified by preparative LC. ESI-MS: m/z 386.3 (M+H)⁺.

### Compound 10: 6-(6-Bromo-benzotriazol-1-ylmethyl)-imidazo[1,2-b]pyridazin-2-ylamine hydrochloride

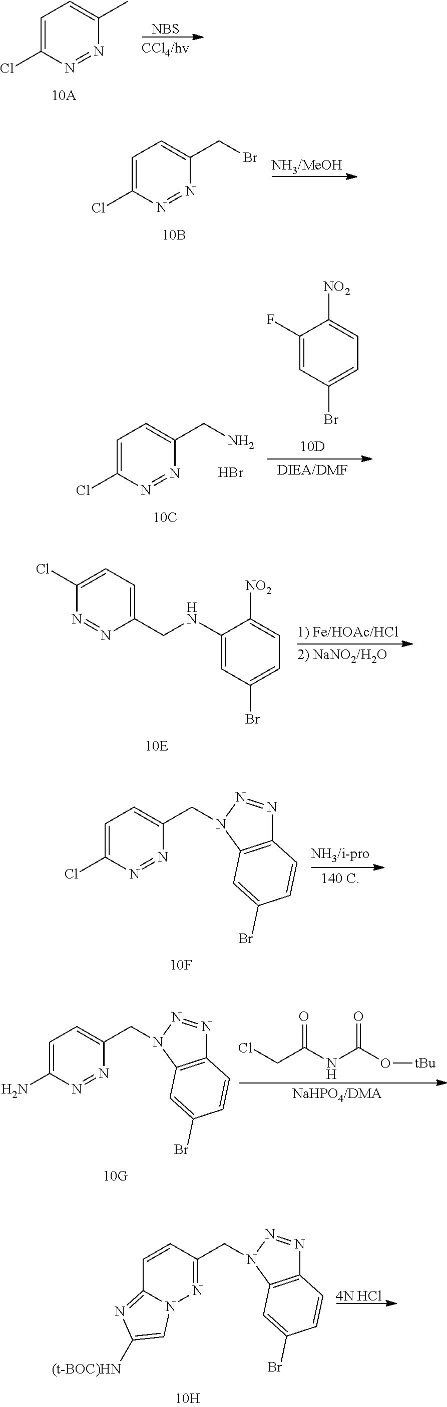

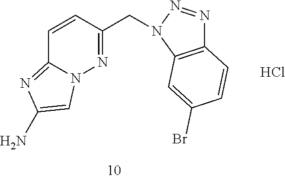 [SMILES: Nc1cn2nc(Cn3nnc4ccc(Br)cc43)ccc2n1 ; Cl]

### 3-Bromomethyl-6-chloro-pyridazine (10B)

A solution of 3-Chloro-6-methyl-pyridazine 10A (5.12 g, 40 mmol) and NBS (8.90 g, 50 mmol) in CCl₄ (300 mL) was refluxed under light (200 w) for 4 hours. The reaction mixture was cooled to room temperature and filtered. The solid residue was washed thoroughly with Cl₄C and filtered. The filtrates was combined, concentrated to small volume and loaded on to a silica gel column. The column was eluted with 50% hexane/ethyl acetate to give 2.2 g of desired product (10B) which was dried in vacuum and used immediately in next step. ESI-MS: m/z 206.9 (M+H)⁺.

### C-(6-Chloro-pyridazin-3-yl)-methylamine Hydrobromide (10C)

A solution of compound 10B (2.08 g, 10 mmol) in methanol (50 mL) was added into a pre-saturated ammonia/methanol solution (200 mL) at 0° C. The reaction solution was sealed and stirred at room temperature overnight. The methanol solution was concentrated and dried in vacuum to give 2.2 g of crude product (10C) as hydrobromide salt, which was used in next step without further purification. ESI-MS: m/z 144.1 (M+H)⁺.

### (5-Bromo-2-nitro-phenyl)-(6-chloro-pyridazin-3-ylmethyl)-amine (10E)

A solution of compound 10C (1.00 g, 4.46 mmol) and 4-bromo-2-fluoro-1-nitro-benzene (10D) (0.97 g, 4.40 mmol) in DMF (50 mL) with DIEA (1.30 g, 10 mmol) was stirred at 65° C. for two hours. The reaction was poured into ice/water and the solid precipitate was collected by filtration, washed with water and dried in vacuum over P₂O₅ overnight to give 1.5 g of desired product (10E). ESI-MS: m/z 342.9 (M+H)⁺.

### 6-Bromo-1-(6-chloro-pyridazin-3-ylmethyl)-1H-benzotriazole (10F)

To a solution of compound 10E (1.40 g, 4.08 mmol) in HOAc/HCl (50/5 mL) was added iron power (2.2 g, 40 mmol). The reaction mixture was stirred at 50° C. for 30 minutes, then cooled to room temperature and filtered. A solution of NaNO₂ (0.35 g, 5 mmol) in water (2 mL) was then added drop wise into above acid solution at 0° C. The reaction solution was stirred for one hour and concentrated to dryness under reduced pressure. The resulting residue was sonicated in ethyl acetate/NaHCO₃ solution and the precipitate was filtered off and rinsed thoroughly with ethyl acetate. The organic solution was separated and dried with MgSO₄, filtered and concentrated to give 1.2 g of desired product (10F). ESI-MS: m/z 323.9 (M+H)⁺.

### 6-(6-Bromo-benzotriazol-1-ylmethyl)-pyridazin-3-ylamine (10G)

To a suspension of compound 10F (1.2 g, 3.7 mmole) in isopropanol (15 mL) in a stainless pressure tube, an ammonia gas was bubbled through at −78° C. for 5 minutes. The pressure tube was sealed and heated in an oil bath at 140° C. for three days. The reaction solution was then re-cooled, transferred to a round bottle, concentrated and dried in vacuum to give 1.3 g of crude product (10G), which was used in next step without further purification. ESI-MS: m/z 305.1 (M+H)⁺.

### [6-(6-Bromo-benzotriazol-1-ylmethyl)-imidazo[1,2-b]pyridazin-2-yl]-carbamic acid tert-butyl ester (10H)

A mixture of compound 10G (1.0 g, 3.28 mmol), (2-chloro-acetyl)-carbamic acid tert-butyl ester (1.0 g, 5.0 mmol) and Na₂HPO₄ (1.4 g, 10 mmol) in DMA (50 mL) was stirred at 135° C. for four hours. Solvent was removed under reduced pressure. The residue was sonicated in ethyl acetate/water, and the precipitate was filtered off. Ethyl acetate solution was separated and concentrated and loaded on silica gel. The silica column was eluted with hexane/ethyl acetate (1/2) to give 0.45 g of desired product (10H). ESI-MS: m/z 444.1 (M+H)⁺.

### 6-(6-Bromo-benzotriazol-1-ylmethyl)-imidazo[1,2-b]pyridazin-2-ylamine Hydrochloride (10)

A solution of compound 10H (0.44 g, 1.0 mmol) in 4N HCl/dioxane (10 mL) was stirred at room temperature for 60 minutes, concentrated and dried in vacuum to give 0.3 g of product (10) as a hydrochloride salt. ESI-MS: m/z 344.1 (M+H)⁺.

### Compound 11: Cyclopropanecarboxylic acid [6-(6-bromo-benzotriazol-1-ylmethyl)-imidazo[1,2-b]pyridazin-2-yl]-amide

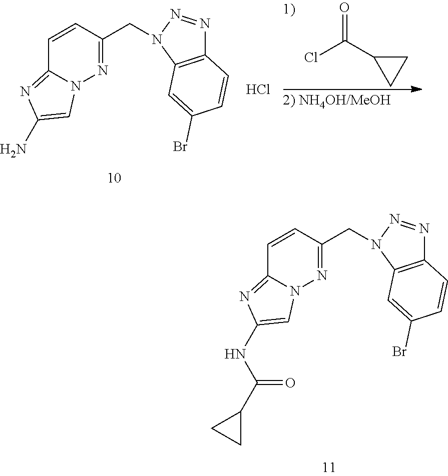 [SMILES: Nc1cn2nc(Cn3nnc4ccc(Br)cc43)ccc2n1 ; Cl ; O=C(Cl)C1CC1 ; O=C(Nc1cn2nc(Cn3nnc4ccc(Br)cc43)ccc2n1)C1CC1]

To a solution of compound 10 (0.34 g, 1 mmol) in CH₂Cl₂ (25 mL) was added DIEA (0.31 g, 3 mmol), followed by cyclopropanecarbonyl chloride (0.23 g, 2.2 mmol). The reaction solution was stirred at room temperature for 60 minutes and concentrated. The residue was re-dissolved in methanol (10 mL) and ammonium hydroxide (0.5 mL) was added. The reaction solution was stirred for 30 minutes and concentrated. The residue was dissolved in ethyl acetate and washed with 5% citric acid and then saturated NaHCO₃. The organic phase was dried with MgSO₄ and concentrated to give 0.25 g of desired product (11). ESI-MS: m/z 412.1 (M+H)⁺.

### Compound 12: N-(6-((6-methyl-1H-benzo[d][1,2,3]triazol-1-yl)methyl)imidazo[1,2-b]pyridazin-2-yl)cyclopropanecarboxamide

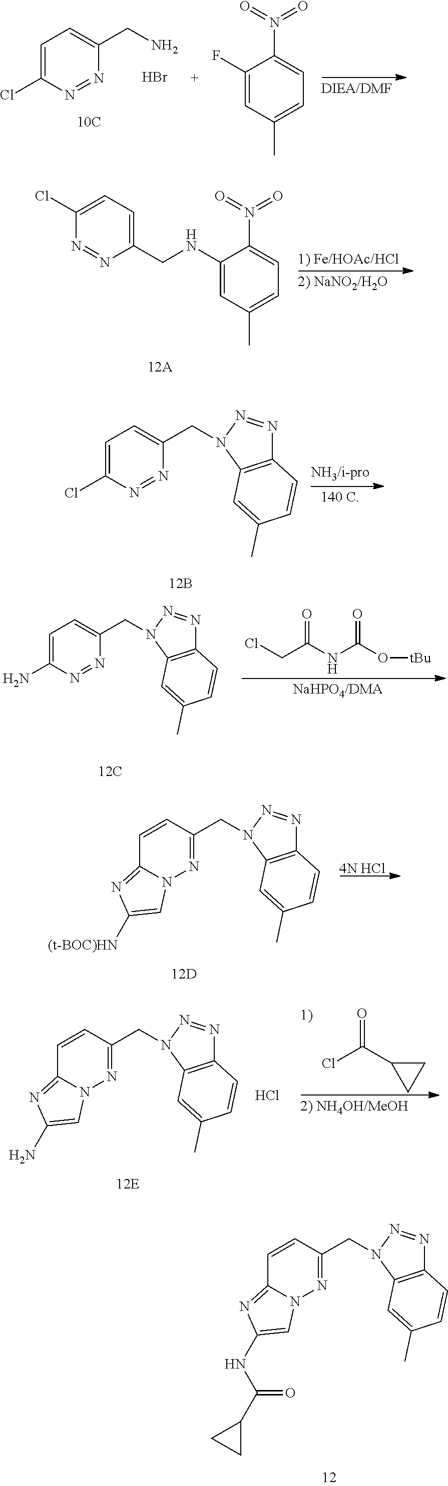

Compound 12E was prepared using a similar procedure to that described in connection with compound 10, except that 2-fluoro-4-methyl-1-nitro-benzene was used in place of compound 10D. Compound 12 was then prepared from compound 12E using the method described in connection with compound 11. ¹H NMR (DMSO-d₆): δ ppm 11.20 (s, 1H), 8.20 (s, 1H), 7.96 (m, 2H), 7.68 (s, 1H), 7.28 (d, 1H), 7.10 (d, 1H), 6.14 (s, 2H), 2.46 (s, 3H), 1.94 (m, 1H), 0.80 (m, 4H).

### Compound 13: Cyclopropanecarboxylic acid 6-[6-(1-methyl-1H-pyrazol-4-yl)-benzotriazol-1-ylmethyl]-imidazo[1,2-b]pyridazin-2-yl}-amide

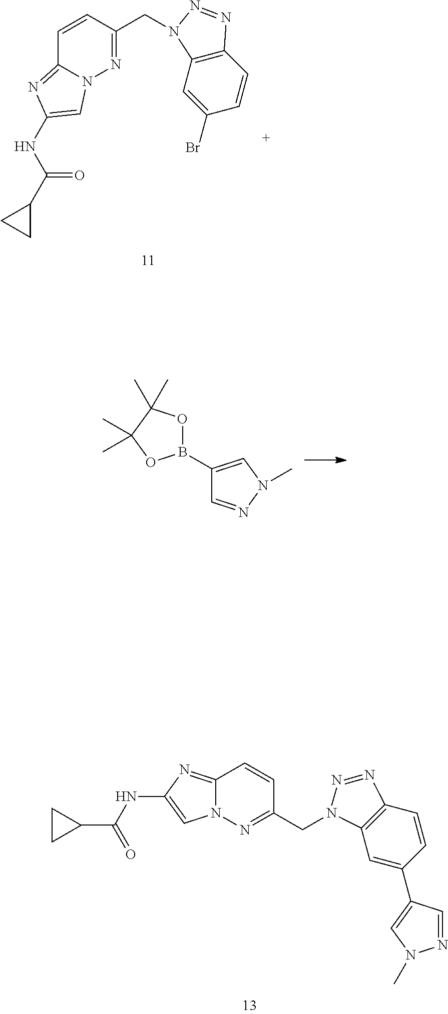

A mixture of compound 11 (20 mg, 0.05 mmol), 1-methylpyrazole-4-boronic acid pinacol ester (20 mg, 0.1 mmol) and catalytic amount of Pd(dppf)Cl₂ in a NaHCO₃ saturated dioxane/H2O (2/1) solution (0.5 mL) was heated using a microwave oven at 120° C. for 30 minutes. After preparative LCMS purification, 11 mg of the desired product was obtained as the TFA salt. ¹H NMR (DMSO-d₆): δ ppm 11.40 (s, 1H), 8.32 (s, 1H), 8.16 (s, 1H), 8.08 (s, 1H), 8.04 (d, 1H), 7.96 (m, 2H), 7.64 (m, 1H), 7.14 (d, 1H), 6.16 (s, 2H), 3.90 (s, 3H), 1.94 (m, 1H), 0.80 (m, 4H) ESI-MS: m/z 414.2 (M+H)⁺.

In a similar manner as compound 13, compounds 14-24 were synthesized and purified from compound 11 and the corresponding boronic acid or ester.

| Compound | Structure | LC/MS | NMR |
|---|---|---|---|
| 14 | 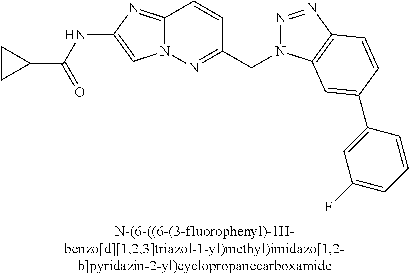 [SMILES: O=C(Nc1cn2nc(Cn3nnc4ccc(-c5cccc(F)c5)cc43)ccc2n1)C1CC1] | 427 | ¹H NMR (400 MHz, DMSO-d₆) δ □ ppm 11.17 (s, 1H), 8.40-8.28 (m, 1 H), 8.21-8.11 (m, 1H), 7.97 (d, J = 9.3 Hz, 1H), 7.86-7.78 (m, 1H), 7.69- 7.45 (m, 4H), 7.31-7.21 (m, 1H), 7.16 (d, J = 9.3 Hz, 1H), 6.30-6.17 (m, 2 H), 1.92 (quin, J = 6.1 Hz, 1H), 0.86-0.75 (m, 4H) |
| 15 | 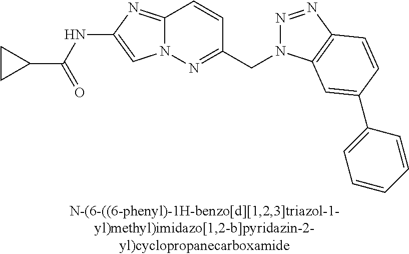 [SMILES: O=C(Nc1cn2nc(Cn3nnc4ccc(-c5ccccc5)cc43)ccc2n1)C1CC1] | 409 | ¹H NMR (400 MHz, DMSO-d₆) δ ppm 11.18 (s, 1H), 8.24 (s, 1H), 8.19- 8.13 (m, 2H), 7.97 (d, J = 9.3 Hz, 1H), 7.81-7.73 (m, 3H), 7.52 (t, J = 7.6 Hz, 2H), 7.45-7.38 (m, 1 H), 7.16 (d, J = 9.1 Hz, 1 H), 6.28-6.19 (m, 2H), 1.92 (quin, J = 6.1 Hz, 1 H), 0.85-0.78 (m, 4H) |
| 16 | 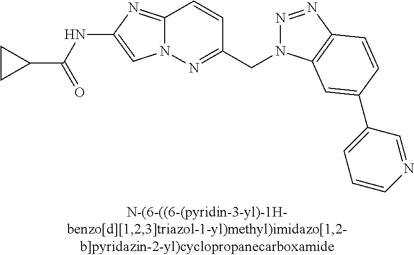 [SMILES: O=C(Nc1cn2nc(Cn3nnc4ccc(-c5cccnc5)cc43)ccc2n1)C1CC1] | 410 | - - - |
| 17 | 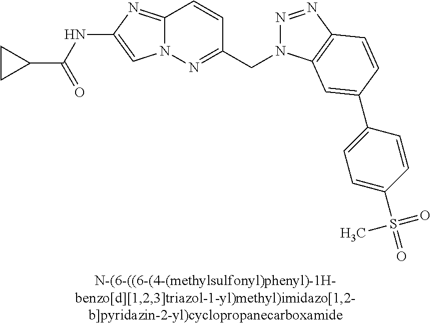 [SMILES: CS(=O)(=O)c1ccc(-c2ccc3nnn(Cc4ccc5nc(NC(=O)C6CC6)cn5n4)c3c2)cc1] | 487 | ¹H NMR (400 MHz, DMSO-d₆) δ ppm 11.18 (s, 1H), 8.39 (s, 1H), 8.25- 8.20 (m, 1H), 8.14 (s, 1 H), 8.09-8.03 (m, 4H), 7.98 (d, J = 9.9 Hz, 1H), 7.86-7.80 (m, 1H), 7.17 (d, J = 9.3 Hz, 1H), 6.27 (s, 2H), 3.28 (s, 3H), 1.98- 1.85 (m, 1H), 0.85-0.77 (m, 4H) |
| 18 | 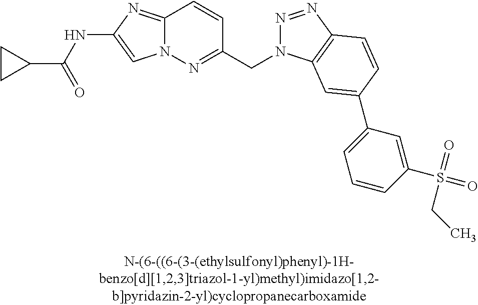 [SMILES: CCS(=O)(=O)c1cccc(-c2ccc3nnn(Cc4ccc5nc(NC(=O)C6CC6)cn5n4)c3c2)c1] | 501 | ¹H NMR (400 MHz, DMSO-d6) δ ppm 11.17 (s, 1H), 8.39 (s, 1H), 8.28- 8.20 (m, 2H), 8.13 (s, 2 H), 7.95 (dd, J = 8.7, 14.8 Hz, 3H), 7.90-7.79 (m, 2 H), 7.15 (d, J = 9.3 Hz, 1 H), 6.28 (s, 2H), 1.92 (d, J = 5.3 Hz, 1H), 1.14 (t, J = 7.2 Hz, 4H), 0.88-0.76 (m, 4H) |
| 19 | 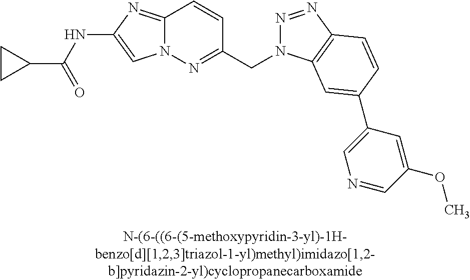 [SMILES: COc1cncc(-c2ccc3nnn(Cc4ccc5nc(NC(=O)C6CC6)cn5n4)c3c2)c1] | 440 | ¹H NMR (400 MHz, DMSO-d₆) δ ppm 11.18 (s, 1H), 8.64 (d, J = 1.8 Hz, 1H), 8.47-8.36 (m, 2 H), 8.21 (d, J = 8.8 Hz, 1 H), 8.14 (s, 1H), 7.98 (d, J = 9.3 Hz, 1H), 7.91-7.79 (m, 2H), 7.17 (d, J = 9.3 Hz, 1H), 6.26 (s, 2H), 3.97-3.93 (m, 3H), 2.02- 1.86 (m, 1H), 0.83-0.78 (m, 4H) |
| 20 | 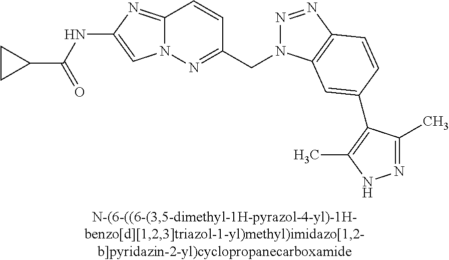 [SMILES: Cc1n[nH]c(C)c1-c1ccc2nnn(Cc3ccc4nc(NC(=O)C5CC5)cn4n3)c2c1] | 427 | ¹H NMR (400 MHz, DMSO-d₆) δ ppm 11.20 (s, 1H), 8.17 (s, 1H), 8.08 (d, J = 8.6 Hz, 1H), 7.99 (d, J = 9.3 Hz, 1H), 7.71 (s, 1H), 7.44-7.35 (m, 1 H), 7.17 (d, J = 9.3 Hz, 1 H), 6.20 (s, 2H), 2.26- 2.16 (m, 6H), 2.02-1.88 (m, 1H), 0.90-0.77 (m, 4 H) |
| 21 | 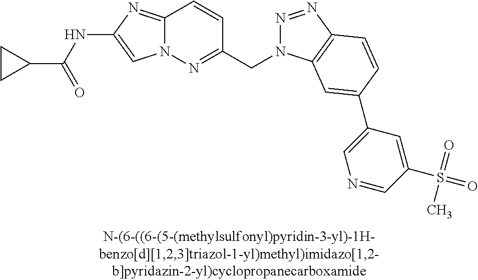 [SMILES: CS(=O)(=O)c1cncc(-c2ccc3nnn(Cc4ccc5nc(NC(=O)C6CC6)cn5n4)c3c2)c1] | 488 | ¹H NMR (400 MHz, DMSO-d₆) δ ppm 11.17 (s, 1H), 9.35 (d, J = 2.0 Hz, 1H), 9.19-9.04 (m, 1 H), 8.70-8.65 (m, 1H), 8.54 (s, 1H), 8.30-8.25 (m, 1H), 8.18-8.10 (m, 1 H), 8.00-7.91 (m, 2H), 7.17 (d, J = 9.3 Hz, 1H), 6.29 (s, 2H), 3.42 (s, 3H), 1.96-1.86 (m, 1H), 0.85- 0.77 (m, 4H) |
| 22 | 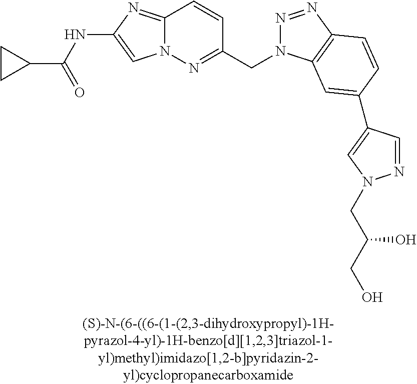 [SMILES: O=C(Nc1cn2nc(Cn3nnc4ccc(-c5cnn(C[C@H](O)CO)c5)cc43)ccc2n1)C1CC1] | 474 | ¹H NMR (400 MHz, DMSO-d₆) δ ppm 11.18 (s, 1H), 8.30 (s, 1H), 8.25 (s, 1H), 8.14 (d, 2H), 8.00 (m, 3H), 7.70 (d, 1H), 7.20 (d, 1H), 6.16 (s, 2H), 4.26 (m, 1H), 4.10 (m, 1H), 3.34 (m, 1H), 1.94 (m, 1H), 0.83 (m, 4H). |
| 23 | 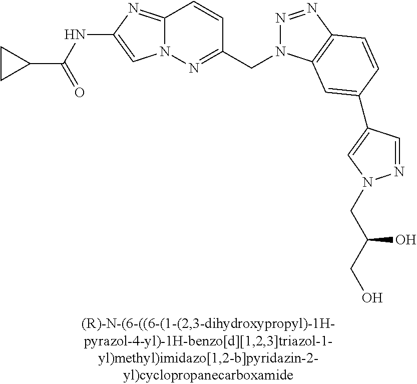 [SMILES: O=C(Nc1cn2nc(Cn3nnc4ccc(-c5cnn(C[C@@H](O)CO)c5)cc43)ccc2n1)C1CC1] | 474 | ¹H NMR (400 MHz, DMSO-d₆) δ ppm 11.18 (s, 1H), 8.30 (s, 1H), 8.25 (s, 1H), 8.14 (d, 2H), 8.00 (m, 3H), 7.70 (d, 1H), 7.20 (d, 1H), 6.16 (s, 2H), 4.26 (m, 1H), 4.10 (m, 1H), 3.34 (m, 1H), 1.94 (m, 1H), 0.83 (m, 4H) |
| 24 ^{a} | 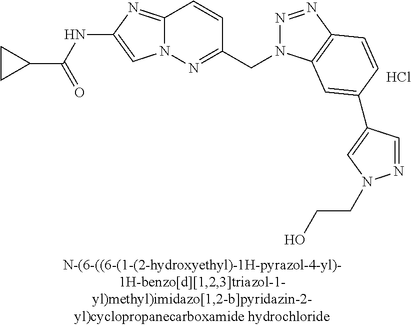 [SMILES: O=C(Nc1cn2nc(Cn3nnc4ccc(-c5cnn(CCO)c5)cc43)ccc2n1)C1CC1 ; Cl] | 444 | ¹H NMR (DMSO-d₆): δ 11.21 (s, 1H), 8.30 (s, 1H), 8.20 (s, 1H), 8.12 (s, 1H), 8.02 (m, 3H), 7.70 (d, 1H), 7.14 (d, 1H), 6.16 (s, 2H), 4.17 (t, 2H), 3.77 (m, 2H), 1.94 (m, 1H), 0.83 (m, 4H) |

^{a} Melting point: >200° C.

### Compound 13: N-(6-((6-(1-methyl-1H-pyrazol-4-yl)-1H-benzo[d][1,2,3]triazol-1-yl)methyl)imidazo[1,2-b]pyridazin-2-yl)cyclopropanecarboxamide

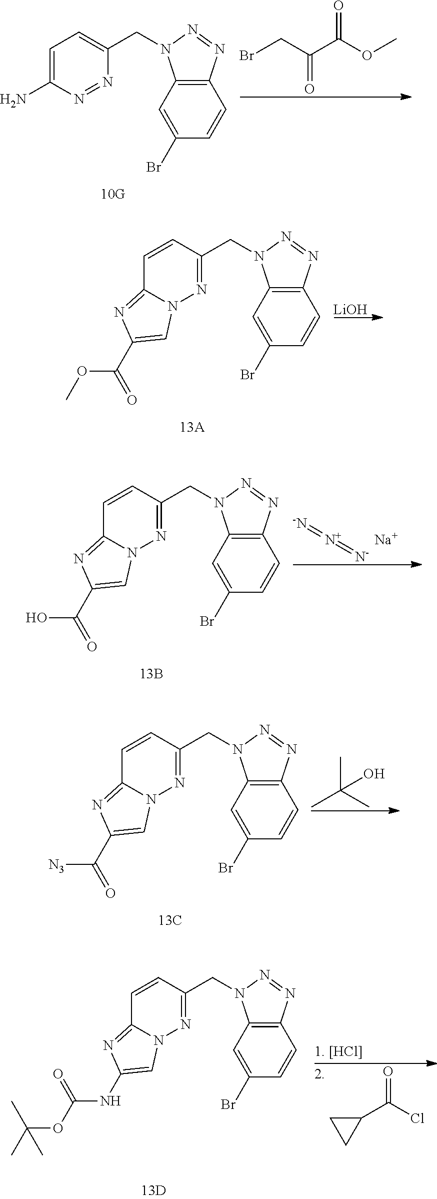

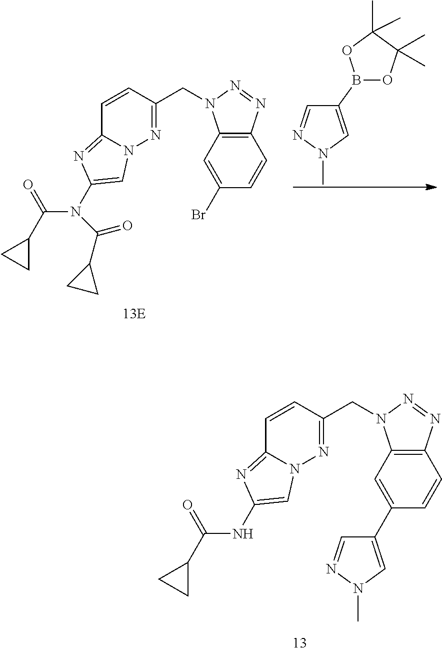

### Ethyl 6-((6-bromo-1H-benzo[d][1,2,3]triazol-1-yl)methyl)imidazo[1,2-b]pyridazine-2-carboxylate

A mixture of 6-((6-bromo-1H-benzo[d][1,2,3]triazol-1-yl)methyl)pyridazin-3-amine 10G (4.8 g, 15.73 mmol), ethyl 3-bromo-2-oxopropanoate (4.60 g, 23.60 mmol) and NaHCO₃ (4.0 g) in dioxane was heated at 60° C. for 1 hr. Additional ethyl 3-bromo-2-oxopropanoate (1.5 g, 7.87 mmol, 0.5 eq) was added and stirred at 60° C. for an additional hour. The reaction mixture was filtered and rinsed with dioxane. 4-Methylbenzenesulfonic acid (2.71 g, 15.73 mmol) was added to the filtrate, and the reaction was heated at 75° C. for 2 hrs. The reaction was evaporated to dryness via rotary evaporation and the resulting residue was dissolved in EtOAc. The organic phase was washed with saturated NaHCO₃ followed by 0.1 N NaOH (3×150 mL). The solution was dried with MgSO₄, filtered, concentrated to dryness and purified by MPLC (10% MeOH/CH₂Cl₂) to provide the title compound, ethyl 6-((6-bromo-1H-benzo[d][1,2,3]triazol-1-yl)methyl)imidazo[1,2-b]pyridazine-2-carboxylate 13A (4.7 g, 11.6 mmol, 50%). ¹H NMR (400 MHz, DMSO-d₆) δ ppm 1.30 (t, J=7.07 Hz, 3H) 4.30 (q, J=7.07 Hz, 2H) 6.26 (s, 2H) 7.33 (d, J=9.60 Hz, 1H) 7.59 (dd, J=8.72, 1.64 Hz, 1H) 8.08 (d, J=8.84 Hz, 1H) 8.23 (d, J=9.60 Hz, 1H) 8.31 (d, J=1.26 Hz, 1H) 8.77 (s, 1H). MS: m/z 401.2 (M+H)⁺.

### 6-((6-bromo-1H-benzo[d][1,2,3]triazol-1-yl)methyl)imidazo[1,2-b]pyridazine-2-carboxylic acid

To a solution of ethyl 6-((6-bromo-1H-benzo[d][1,2,3]triazol-1-yl)methyl)imidazo[1,2-b]pyridazine-2-carboxylate 13A (5 g, 12.46 mmol) in 10% H₂O/MeOH (200 mL), LiOH (0.597 g, 24.92 mmol) was added. The reaction was stirred at ambient temperature for 18 hrs and then concentrated to remove the MeOH. H₂O (100 mL) was added and the pH was adjusted to 4 with concentrated HCl. The resulting solid was collected by filtration, rinsed with water followed by EtOAc, and dried in vacuum over P₂O₅ to provide the title compound, 6-((6-bromo-1H-benzo[d][1,2,3]triazol-1-yl)methyl)imidazo[1,2-b]pyridazine-2-carboxylic acid 13B (3.6 g 9.65 mmol, 77%). The filtrate was extracted with EtOAc (3×150 mL) and the organics were combined, dried with MgSO₄, filtered and concentrated to dryness to give an additional 0.6 g of product. ¹H NMR (400 MHz, DMSO-d₆) δ ppm 6.24 (s, 2H) 7.24 (d, J=9.35 Hz, 1H) 7.58 (dd, J=8.84, 1.77 Hz, 1H) 8.04-8.14 (m, 2H) 8.32 (d, J=1.01 Hz, 1H) 8.47 (s, 1H). MS: m/z 373.2 (M+H)⁺.

### 6-((6-bromo-1H-benzo[d][1,2,3]triazol-1-yl)methyl)imidazo[1,2-b]pyridazine-2-carbonyl azide

To a stirred solution of 6-((6-bromo-1H-benzo[d][1,2,3]triazol-1-yl)methyl)imidazo[1,2-b]pyridazine-2-carboxylic acid 13B (3.6 g, 9.65 mmol), N-ethyl-N-isopropylpropan-2-amine (7.48 g, 57.9 mmol) and sodium azide (6.27 g, 96 mmol) in anhydrous DMF (60 mL), PyBOP (6.02 g, 11.58 mmol) was added in portions at ambient temperature over 5 min. The reaction was stirred for another 30 min and then poured into a EtOAc/H₂O (100/300 mL) mixture and shaken well. The resulting precipitate was filtered, rinsed with H₂O followed by EtOAc, and dried in vacuum over P₂O₅ for 18 hrs to provide the title compound, 6-((6-bromo-1H-benzo[d][1,2,3]triazol-1-yl)methyl)imidazo[1,2-b]pyridazine-2-carbonyl azide 13C (2.1 g, 5.28 mmol, 55%). The EtOAc solution washed with 5% citric acid (2×100 mL), NaHCO₃, dried with MgSO₄, filtered and concentrated to dryness to give additional crude product (1.6 g of 80% pure material). ¹H NMR (400 MHz, DMSO-d₆) δ ppm 6.28 (s, 2H) 7.37 (d, J=9.60 Hz, 1H) 7.59 (dd, J=8.84, 1.77 Hz, 1H) 8.09 (d, J=8.84 Hz, 1H) 8.26 (d, J=10.10 Hz, 1H) 8.32 (d, J=1.26 Hz, 1H) 8.92 (s, 1H). MS: m/z 398.2 (M+H)⁺.

### tert-Butyl 6-((6-bromo-1H-benzo[d][1,2,3]triazol-1-yl)methyl)imidazo[1,2-b]pyridazin-2-ylcarbamate

A suspension of 6-((6-bromo-1H-benzo[d][1,2,3]triazol-1-yl)methyl)imidazo[1,2-b]pyridazine-2-carbonyl azide 13C (1 g, 2.51 mmol) in 2-methylpropan-2-ol (50 mL, 753 mmol) was heated at 100° C. in a microwave for 3 hrs. The reaction was repeated once. The reactions were combined and the solvent was removed via rotary evaporation. The resulting residue was suspended in EtOAc/H₂O and sonicated. The resulting solid was collected by filtration, rinsed with H₂O followed by EtOAc, and dried in vacuum over P₂O₅ to provide the title compound, tert-Butyl 6-((6-bromo-1H-benzo[d][1,2,3]triazol-1-yl)methyl)imidazo[1,2-b]pyridazin-2-ylcarbamate 13D (1.7 g, 3.83 mmol, 77%). ¹H NMR (400 MHz, DMSO-d₆) δ ppm 1.47 (s, 9H) 6.18 (s, 2H) 7.13 (d, J=9.35 Hz, 1H) 7.58 (dd, J=8.72, 1.64 Hz, 1H) 7.94 (d, J=9.85 Hz, 2H) 8.07 (d, J=8.84 Hz, 1H) 8.31 (d, J=1.77 Hz, 1H) 10.24 (br, 1H). MS: m/z 444.2 (M+H)⁺.

### N-(6-((6-bromo-1H-benzo[d][1,2,3]triazol-1-yl)methyl)imidazo[1,2-b]pyridazin-2-yl)-N-(cyclopropanecarbonyl)cyclopropanecarboxamide: A mixture of tert-butyl 6-((6-bromo-1H-benzo[d][1,2,3]triazol-1-yl)methyl)imidazo[1,2-b]pyridazin-2-ylcarbamate 13D (1.7 g, 3.83 mmol) in 4N HCl/dioxane (30 mL) was stirred at ambient temperature for 2 hrs. The solvent was removed via rotary evaporation, and the resulting residue was dried in vacuum for 18 hrs to yield the 6-((6-bromo-1H-benzo[d][1,2,3]triazol-1-yl)methyl)imidazo[1,2-b]pyridazin-2-amine as an HCl salt. This material was used without further purification. To a solution of 6-((6-bromo-1H-benzo[d][1,2,3]triazol-1-yl)methyl)imidazo[1,2-b]pyridazin-2-amine HCl (1.8 g, 5.23 mmol) in CH₂Cl₂ was added N-ethyl-N-isopropylpropan-2-amine (2.70 g, 20.92 mmol) followed by cyclopropanecarbonyl chloride (1.640 g, 15.69 mmol) at 0° C. The reaction was stirred at ambient temperature for 2 hrs. The reaction was then washed with 5% citric acid solution followed by saturated NaHCO₃, dried with MgSO₄, filtered and concentrated to dryness. The resulting residue was purified by MPLC (5% MeOH/CH₂Cl₂) to provide the title compound, N-(6-((6-bromo-1H-benzo[d][1,2,3]triazol-1-yl)methyl)imidazo[1,2-b]pyridazin-2-yl)-N-(cyclopropanecarbonyl)cyclopropanecarboxamide 13E (2.0 g, 4.1 mmol, 80%). ¹H NMR (400 MHz, DMSO-d₆) δ ppm 0.75-0.86 (m, 8H) 1.93 (m, 2H) 6.19 (s, 2H) 7.16 (d, J=9.35 Hz, 1H) 7.51-7.64 (m, 1H) 7.99 (d, J=9.35 Hz, 1H) 8.01-8.10 (m, 1H) 8.13 (s, 1H) 8.31 (d, J=1.77 Hz, 1H). MS: m/z 480.2 (M+H)⁺.

### N-(6-((6-(1-methyl-1H-pyrazol-4-yl)-1H-benzo[d][1,2,3]triazol-1-yl)methyl)imidazo[1,2-b]pyridazin-2-yl)cyclopropanecarboxamide

A mixture of N-(6-((6-bromo-1H-benzo[d][1,2,3]triazol-1-yl)methyl)imidazo[1,2-b]pyridazin-2-yl)-N-(cyclopropanecarbonyl)cyclopropanecarboxamide 13E (0.7 g, 1.457 mmol), 1-methyl-4-(4,4,5,5-tetramethyl-1,3,2-dioxaborolan-2-yl)-1H-pyrazole (0.303 g, 1.457 mmol) and PdCl₂(dppf)-CH₂Cl₂ (0.06 g, 0.075 mmol) in Na₂CO₃ (2N, 6 mL)/dioxane (12 mL) was heated in a microwave at 110° C. for 45 min. The reaction was filtered and rinsed with EtOAc. This reaction was repeated twice. The organic solutions were combined and washed with 5% citric acid followed by NaHCO₃, dried with MgSO₄, and filtered. MeOH was added to the EtOAc solution to give 5% MeOH (v/v), and the resulting solution was filtered through a short silica column and rinsed thoroughly with 5% MeOH/EtOAc. Activated charcoal (1 g) was added to the eluent and stirred at ambient temperature for 1 hr. The solution was filtered through Celite and concentrated to dryness via rotary evaporation. The resulting material was suspended in EtOAc (50 mL) and sonicated. The resulting solid was collected by filtration and rinsed with EtOAc to provide the title compound, N-(6-((6-(1-methyl-1H-pyrazol-4-yl)-1H-benzo[d][1,2,3]triazol-1-yl)methyl)imidazo[1,2-b]pyridazin-2-yl)cyclopropanecarboxamide 13 (820 mg, 1.98 mmol, 45%). The filtrate was concentrated and reconstituted in 10% MeOH/EtOAc, loaded on a short silica column and eluted with 10% MeOH/EA to give provide additional product (190 mg). ¹H NMR (400 MHz, DMSO-d₆) δ ppm 0.76-0.86 (m, 4H) 1.88-1.97 (m, 1H) 3.88 (s, 3H) 6.15 (s, 2H) 7.12 (d, J=9.35 Hz, 1H) 7.66 (dd, J=8.72, 1.39 Hz, 1H) 7.93-8.01 (m, 2H) 8.04 (d, J=8.84 Hz, 1H) 8.10 (s, 1H) 8.17 (s, 1H) 8.26 (s, 1H) 11.18 (s, 1H). MS: m/z 414.2 (M+H)⁺. MP 250.2-251.6° C.

### Compound 25: N-(6-(6-(prop-1-en-2-yl)-[1,2,4]triazolo[4,3-a]pyridin-3-ylthio)imidazo[1,2-b]pyridazin-2-yl)cyclopropanecarboxamide

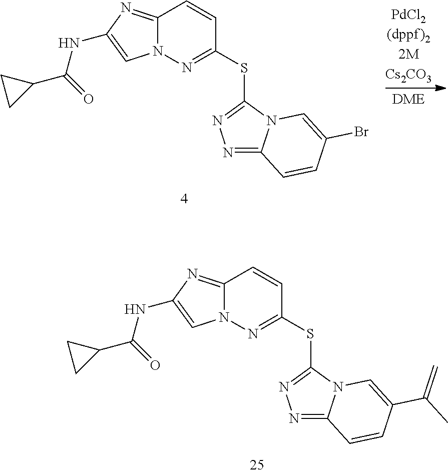 [SMILES: O=C(Nc1cn2nc(Sc3nnc4ccc(Br)cn34)ccc2n1)C1CC1 ; C=C(C)c1ccc2nnc(Sc3ccc4nc(NC(=O)C5CC5)cn4n3)n2c1]

A of mixture Compound 4 (50 mg, 0.10 mmol), 4,4,5,5-tetramethyl-2-(prop-1-en-2-yl)-1,3,2-dioxaborolane (71 mg, 0.43 mmol), Cs₂CO₃ (3 M; 0.11 mL, 0.30 mmol), and PdCl₂(dppf)₂ (4.0 mg, 0.0005 mmol) in DME (1.0 mL) was heated in a microwave at 100° C. for 1 hr. The reaction mixture was then concentrated to dryness via rotary evaporation. The resulting crude material was reconstituted in DMSO (1.0 mL) and purified via preparative mass trigger LCMS using a gradient eluant of 25-50% ACN:0.05% TFA (aq). The collected fractions were combined and the ACN was removed via rotary evaporation. The resulting mixture was lyophilized to provide the TFA salt of the title compound. ¹H NMR (400 MHz, DMSO-d₆) δ ppm 11.17 (s, 1H) 8.31 (s, 1H) 8.14 (s, 1H) 7.95 (s, 1H) 7.91 (d, J=9.5 Hz, 1H) 7.64 (dd, J=9.4 and 1.6 Hz, 1H) 7.05 (d, J=9.4 Hz, 1H) 5.67 (s, 1H) 5.29 (s, 1H) 2.08 (m, 3H) 1.89 (m, 1H) 1.06 (m, 2H) 0.90 (m, 2H) ESI-MS: m/z 391.1 (M+H)⁺.

### Compound 26: N-(6-(6-isopropyl-[1,2,4]triazolo[4,3-a]pyridin-3-ylthio)imidazo[1,2-b]pyridazin-2-yl)cyclopropanecarboxamide

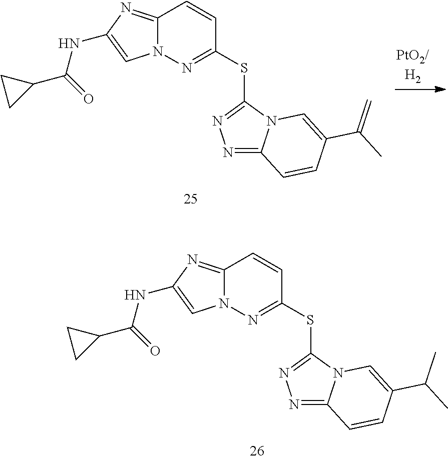 [SMILES: C=C(C)c1ccc2nnc(Sc3ccc4nc(NC(=O)C5CC5)cn4n3)n2c1 ; CC(C)c1ccc2nnc(Sc3ccc4nc(NC(=O)C5CC5)cn4n3)n2c1]

To a solution of N-(6-(6-(prop-1-en-2-yl)-[1,2,4]triazolo[4,3-a]pyridin-3-ylthio)imidazo[1,2-b]pyridazin-2-yl)cyclopropanecarboxamide (150 mg, 0.383 mmol) in ethanol (20 mL) was added platinum (IV) oxide (4.35 mg, 0.019 mmol). The reaction was stirred for 24 hr at 25° C. under 10 psi of H₂. The reaction mixture was then filtered through Celite and the filtrate was concentrated to dryness via rotary evaporation. The resulting crude material was reconstituted in DMSO (1.0 mL) and purified via preparative mass trigger LCMS using a gradient eluant of 25-50% ACN:0.05% TFA (aq). The collected fractions were combined and the ACN was removed via rotary evaporation. The resulting mixture was lyophilized to provide the TFA salt of the title compound (6.0 mg, 0.015 mmol, 3.98% yield) as a yellow solid. ¹H NMR (400 MHz, DMSO-d₆) δ ppm 0.71-0.87 (m, 4H) 1.19 (d, J=6.82 Hz, 6H) 1.79-1.98 (m, 1H) 3.02 (dt, J=13.71, 6.92 Hz, 1H) 7.03 (d, J=9.35 Hz, 1H) 7.62 (dd, J=9.47, 1.64 Hz, 1H) 7.90-7.97 (m, 3H) 8.26 (s, 1H) 11.17 (s, 1H). ESI-MS: m/z 394.3 (M+H)⁺.

### Compound 27: 1-(6-(6-(3-fluorophenyl)-[1,2,4]triazolo[4,3-a]pyridin-3-ylthio)imidazo[1,2-b]pyridazin-2-yl)-3-(2-hydroxyethyl)urea

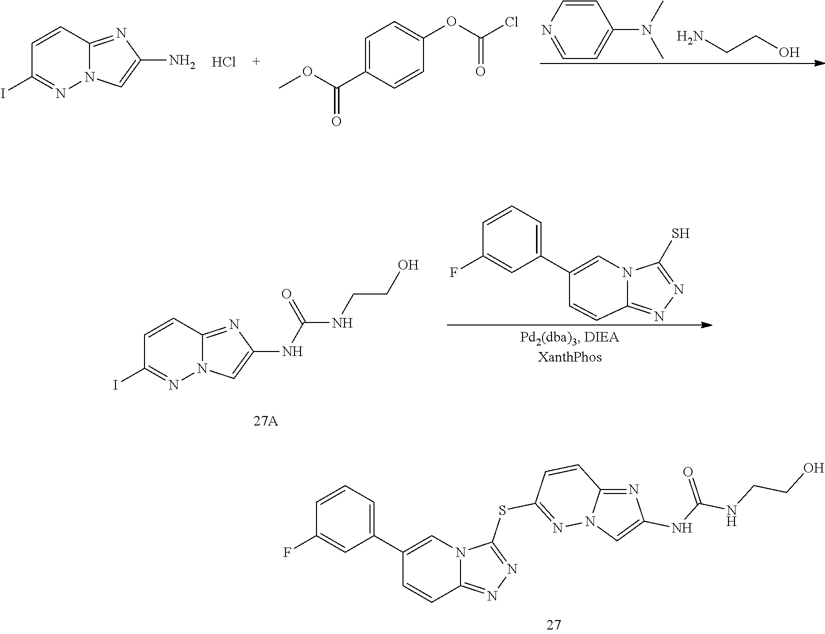

### 1-(2-Hydroxyethyl)-3-(6-iodoimidazo[1,2-b]pyridazin-2-yl)urea

To a solution of 6-iodoimidazo[1,2-b]pyridazin-2-amine hydrochloride (250 mg, 0.843 mmol) and DMAP (227 mg, 1.855 mmol) in DCM (Volume: 5.0 mL) was added methyl 4-(chlorocarbonyloxy)benzoate (199 mg, 0.927 mmol) at 25° C. The reaction was stirred at 25° C. for 1 hr to provide a dark green heterogeneous mixture which was filtered. The solid was resuspended in DCM (Volume: 5.0 mL), treated with ethanolamine (2M in MeOH, 0.843 mL, 1.686 mmol) at room temperature, and then stirred for an additional 1 hr. The reaction was then evaporated to dryness via rotary evaporation, and the resulting residue was suspended in EtOAc (Volume: 10.0 mL). The mixture was filtered, and the resulting black solid was dried under high vacuum and used without further purification.

### 1-(6-(6-(3-Fluorophenyl)-[1,2,4]triazolo[4,3-a]pyridin-3-ylthio)imidazo[1,2-b]pyridazin-2-yl)-3-(2-hydroxyethyl)urea

A mixture of 1-(2-hydroxyethyl)-3-(6-iodoimidazo[1,2-b]pyridazin-2-yl)urea (125 mg, 0.360 mmol), 6-(3-fluorophenyl)-[1,2,4]triazolo[4,3-a]pyridine-3-thiol (88 mg, 0.360 mmol), Pd₂(dba)₃ (19.79 mg, 0.022 mmol), XANTPHOS (25.00 mg, 0.043 mmol), and DIEA (0.126 mL, 0.720 mmol) in DME (Volume: 2.0 mL) was heated in the microwave on high absorbance for 30 min at 120° C. The reaction mixture was then concentrated to dryness via rotary evaporation. The resulting crude material was reconstituted in DMSO (Volume: 1.0 mL) and purified via preparative mass trigger LCMS using a gradient eluant of 20-45% ACN:0.05% TFA (aq). The collected fractions were combined and the ACN was removed via rotary evaporation. The resulting mixture was lyophilized and reconstituted in water:ACN (1:10, 5.0 mL) Two drops of [HCl] were added and the resulting solution was lyophilized to provide the HCl salt of the title compound (20 mg, 0.043 mmol, 11.96% yield) as a brown solid. ¹H NMR (400 MHz, DMSO-d₆) δ ppm 3.14 (q, J=5.47 Hz, 2H), 341 (m, 2H), 6.61 (br. s., 1H) 6.97-7.07 (m, 2H) 7.21-7.32 (m, 1H) 7.53 (td, J=7.96, 6.06 Hz, 1H) 7.62 (d, J=8.34 Hz, 1H) 7.65-7.72 (m, 1H) 7.83 (d, J=9.35 Hz, 1H) 7.99 (dd, J=9.60, 1.77 Hz, 1H) 8.11 (dd, J=9.60, 1.01 Hz, 1H) 8.69-8.93 (m, 1H) 9.17 (s, 1H). ESI-MS: m/z 465.3 (M+H)⁺.

### Compound 28: 2-chloro-4-(3-(2-(cyclopropanecarboxamido)imidazo[1,2-b]pyridazin-6-ylthio)-[1,2,4]triazolo[4,3-a]pyridin-6-yl)-N-methylbenzamide

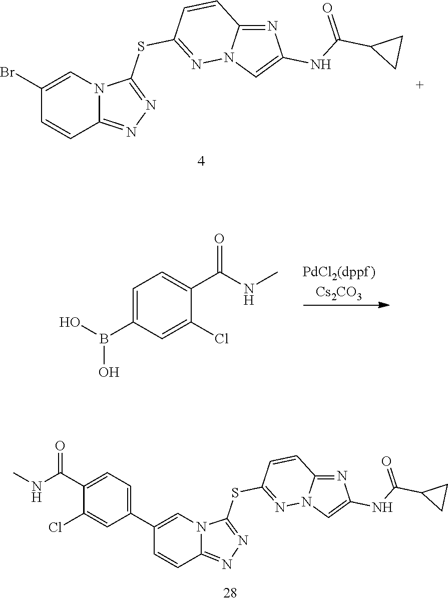 [SMILES: O=C(Nc1cn2nc(Sc3nnc4ccc(Br)cn34)ccc2n1)C1CC1 ; CNC(=O)c1ccc(B(O)O)cc1Cl ; CNC(=O)c1ccc(-c2ccc3nnc(Sc4ccc5nc(NC(=O)C6CC6)cn5n4)n3c2)cc1Cl]

A mixture of N-(6-(6-bromo-[1,2,4]triazolo[4,3-a]pyridin-3-ylthio)imidazo[1,2-b]pyridazin-2-yl)cyclopropanecarboxamide (150 mg, 0.349 mmol), 3-chloro-4-(methylcarbamoyl)phenylboronic acid (112 mg, 0.523 mmol), PdCl₂(dppf) (12.75 mg, 0.017 mmol), and cesium carbonate (0.349 mL, 1.046 mmol) in DME (Volume: 1.0 mL) was heated in a microwave on high absorbance for 1 hr at 100° C. The reaction mixture was then concentrated to dryness via rotary evaporation. The resulting crude material was reconstituted in DMSO (Volume: 1.0 mL) and purified via preparative mass trigger LCMS using a gradient eluant of 20-45% ACN:0.05% TFA (aq). The collected fractions were combined and the ACN was removed via rotary evaporation. The resulting mixture was lyophilized to provide the TFA salt of the titled compound (6.0 mg, 0.012 mmol, 3.32% yield) as a pale yellow solid. ¹H NMR (400 MHz, DMSO-d₆) δ ppm 0.69-0.85 (m, 4H) 1.81-1.96 (m, 1H) 2.75 (d, J=4.55 Hz, 3H) 7.07 (d, J=9.60 Hz, 1H) 7.51 (d, J=8.08 Hz, 1H) 7.79 (dd, J=7.83, 1.77 Hz, 1H) 7.87-7.97 (m, 3H) 8.00 (dd, J=9.60, 1.77 Hz, 1H) 8.11 (dd, J=9.60, 1.01 Hz, 1H) 8.39 (d, J=4.80 Hz, 1H) 8.86 (d, J=1.52 Hz, 1H) 11.15 (s, 1H). ESI-MS: m/z 419.2 (M+H)⁺.

### Compound 29: N-(6-(6-(1-((2-methoxyethoxy)methyl)-1H-pyrazol-4-yl)-[1,2,4]triazolo[4,3-a]pyridin-3-ylthio)imidazo[1,2-b]pyridazin-2-yl)cyclopropanecarboxamide

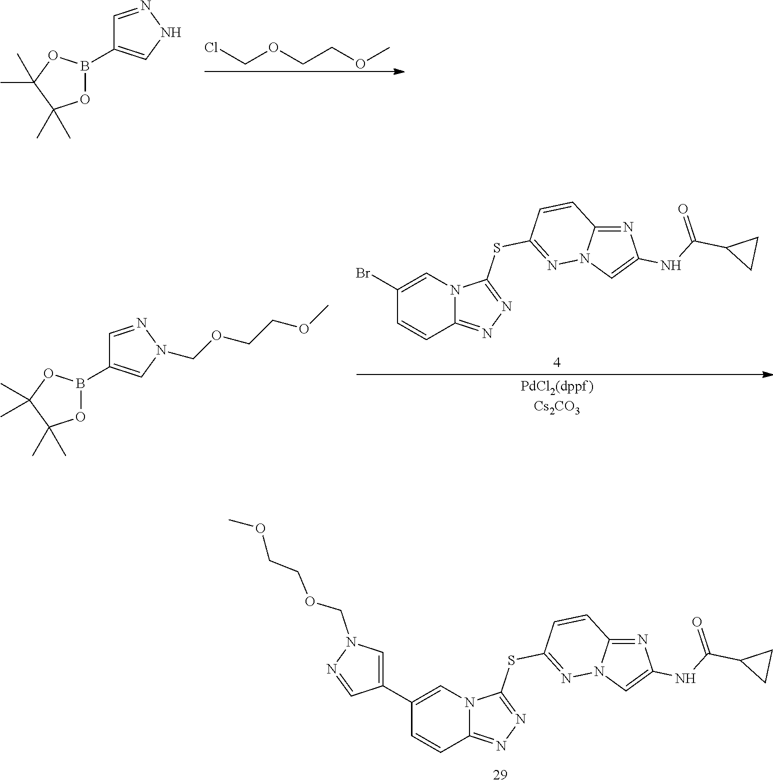

A solution of 4-(4,4,5,5-tetramethyl-1,3,2-dioxaborolan-2-yl)-1H-pyrazole (0.8 g, 4.1 mmol), cesium carbonate (2.0 g, 6.2 mmol), and 1-(chloromethoxy)-2-methoxyethane (0.59 mL, 5.2 mmol) in DMF (14 mL) was heated in a microwave at 90° C. for 1 hr. After the initial heating, additional 1-(chloromethoxy)-2-methoxyethane (0.59 mL) and cesium carbonate (2 g) was added. Heating was repeated for an additional 1 hr. The crude reaction mixtures were then diluted with water (250 mL) and extracted with ethyl acetate (3×50 mL). The title compound was purified by silica gel column using DCM/EtOAc/MeOH (8/1.5/0.5) as eluent to give 1-(2-methoxyethyl)-4-(4,4,5,5-tetramethyl-1,3,2-dioxaborolan-2-yl)-1H-pyrazole (1.6 g) as a light yellow oil. ESI-MS: m/z 283.2 (M+H)⁺.

A mixture of N-(6-(6-bromo-[1,2,4]triazolo[4,3-a]pyridin-3-ylthio)imidazo[1,2-b]pyridazin-2-yl)cyclopropanecarboxamide (100 mg, 0.232 mmol), 1-((2-methoxyethoxy)methyl)-4-(4,4,5,5-tetramethyl-1,3,2-dioxaborolan-2-yl)-1H-pyrazole (328 mg, 1.162 mmol), pdcl2(dppf) (8.50 mg, 0.012 mmol), and cesium carbonate (0.232 mL, 0.697 mmol) in Dioxane (Volume: 1.0 mL) was heated in a microwave on high absorbance for 1 hr at 100° C. The reaction mixture was then concentrated to dryness via rotary evaporation. The resulting crude material was reconstituted in DMSO (Volume: 1.0 mL) and purified via preparative mass trigger LCMS using a gradient eluant of 15-40% ACN:0.05% TFA (aq). The collected fractions were combined and the ACN was removed via rotary evaporation. The resulting mixture was lyophilized to provide the TFA salt of the title compound (8.0 mg, 0.016 mmol, 6.81% yield) as a pale yellow solid. ¹H NMR (400 MHz, DMSO-d₆) δ ppm 0.72-0.78 (m, 4H) 1.90 (m, 1H) 3.20 (m, 3H) 3.38-3.42 (m, 2H) 3.54-3.58 (m, 2H) 5.45 (m, 2H) 7.00 (m, 1H) 7.67 (m, 1H) 7.91-7.96 (m, 3H) 8.04 (m, 1H) 8.59 (m, 1H), 8.76 (m, 1H) 11.16 (s, 1H). ESI-MS: m/z 506.3 (M+H)⁺.

### Compound 30: N-(6-(6-(1-(2-(dimethylamino)-2-oxoethyl)-1H-pyrazol-4-yl)-[1,2,4]triazolo[4,3-a]pyridin-3-ylthio)imidazo[1,2-b]pyridazin-2-yl)cyclopropanecarboxamide

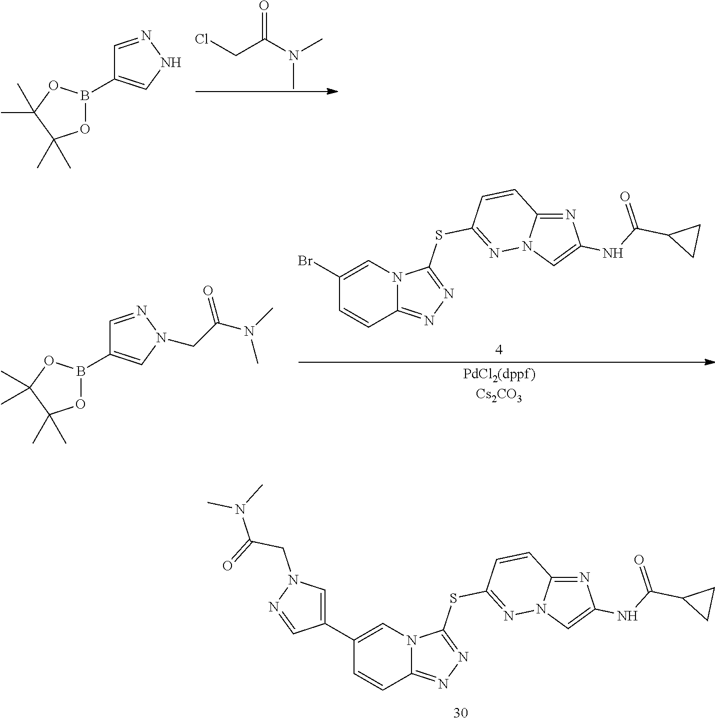

A solution of 4-(4,4,5,5-tetramethyl-1,3,2-dioxaborolan-2-yl)-1H-pyrazole (0.8 g, 4.1 mmol), cesium carbonate (2.0 g, 6.2 mmol), and 2-chloro-N,N-dimethylacetamide (0.47 mL, 4.5 mmol) in DMF (14 mL) was heated in a microwave at 90° C. for 1 hr. The crude reaction mixtures were then diluted with water (300 mL) and extracted with ethyl acetate (3×50 mL). Product was purified by silica gel column using DCM/EtOAc/MeOH (8/1.5/0.5) as eluent to provide N,N-dimethyl-2-(4-(4,4,5,5-tetramethyl-1,3,2-dioxaborolan-2-yl)-1H-pyrazol-1-yl)acetamide (1.3 g) as a light yellow oil. ESI-MS: m/z 280.3 (M+H)⁺.

A mixture of N-(6-(6-bromo-[1,2,4]triazolo[4,3-a]pyridin-3-ylthio)imidazo[1,2-b]pyridazin-2-yl)cyclopropanecarboxamide (100 mg, 0.232 mmol), N,N-dimethyl-2-(4-(4,4,5,5-tetramethyl-1,3,2-dioxaborolan-2-yl)-1H-pyrazol-1-yl)acetamide (324 mg, 1.162 mmol), pdcl2(dppf) (8.50 mg, 0.012 mmol), and cesium carbonate (0.232 mL, 0.697 mmol) in Dioxane (Volume: 1.0 mL) was heated in a microwave on high absorbance for 1 hr at 100° C. The reaction mixture was then concentrated to dryness via rotary evaporation. The resulting crude material was reconstituted in DMSO (Volume: 1.0 mL) and purified via preparative mass trigger LCMS using a gradient eluant of 15-40% ACN:0.05% TFA (aq). The collected fractions were combined and the ACN was removed via rotary evaporation. The resulting mixture was lyophilized to provide the TFA salt of the title compound (4.8 mg, 9.55 μmol, 4.11% yield) as a yellow solid. ¹H NMR (400 MHz, DMSO-d₆) δ ppm 0.79 (d, J=6.06 Hz, 4H) 1.90 (d, J=6.57 Hz, 1H) 2.84 (s, 3H) 3.03 (s, 3H) 5.12 (s, 2H) 7.00 (d, J=9.35 Hz, 1H) 7.87-7.97 (m, 3H) 8.01-8.08 (m, 2H) 8.31 (s, 1H) 8.71 (s, 1H) 11.16 (s, 1H). ESI-MS: m/z 503.3 (M+H)⁺.

### Compound 31: N-(6-(6-(1-(2-methoxyethyl)-1H-pyrazol-4-yl)-[1,2,4]triazolo[4,3-a]pyridin-3-ylthio)imidazo[1,2-b]pyridazin-2-yl)cyclopropanecarboxamide

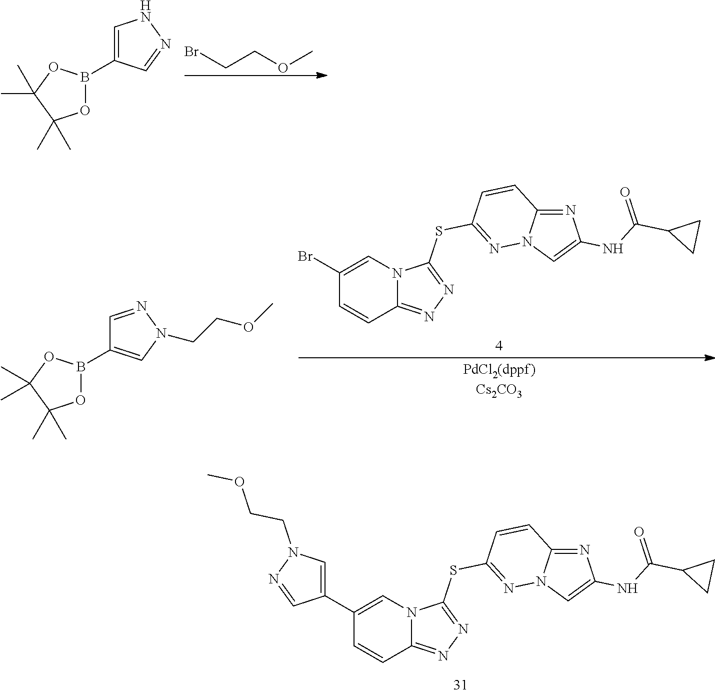

A solution of 4-(4,4,5,5-tetramethyl-1,3,2-dioxaborolan-2-yl)-1H-pyrazole (0.8 g, 4.1 mmol), cesium carbonate (2.0 g, 6.2 mmol), and 1-bromo-2-methoxyethane (0.41 mL, 4.3 mmol) in DMF (14 mL) was heated in a microwave at 90° C. for 1 hr. After the initial heating, additional 1-bromo-2-methoxyethane (0.41 mL) was added to the reaction. Heating was repeated for an additional 1 hr. The crude reaction mixtures were then diluted with water (250 mL) and extracted with ethyl acetate (3×50 mL). Product was purified by silica gel column using DCM/EtOAc/MeOH (8/1.5/0.5) as eluent to give 1-(2-methoxyethyl)-4-(4,4,5,5-tetramethyl-1,3,2-dioxaborolan-2-yl)-1H-pyrazole (1.2 g) as a light yellow oil. ESI-MS: m/z 253.2 (M+H)⁺.

A mixture of N-(6-(6-bromo-[1,2,4]triazolo[4,3-a]pyridin-3-ylthio)imidazo[1,2-b]pyridazin-2-yl)cyclopropanecarboxamide (100 mg, 0.232 mmol), 1-(2-methoxyethyl)-4-(4,4,5,5-tetramethyl-1,3,2-dioxaborolan-2-yl)-1H-pyrazole (410 mg, 1.627 mmol), pdcl2(dppf) (8.50 mg, 0.012 mmol), and cesium carbonate (0.232 mL, 0.697 mmol) in Dioxane (Volume: 1.0 mL) was heated in a microwave on high absorbance for 1 hr at 100° C. The reaction mixture was then concentrated to dryness via rotary evaporation. The resulting crude material was reconstituted in DMSO (Volume: 1.0 mL) and purified via preparative mass trigger LCMS using a gradient eluant of 15-40% ACN:0.05% TFA (aq). The collected fractions were combined and the ACN was removed via rotary evaporation. The resulting mixture was lyophilized to provide the TFA salt of the title compound (6.0 mg, 0.013 mmol, 5.43% yield) as a yellow solid. ¹H NMR (400 MHz, DMSO-d₆) δ ppm 0.69-0.86 (m, 4H) 1.90 (m, 1H) 3.20 (m, 3H) 3.63-3.70 (m, 2H) 4.22-4.27 (m, 2H) 7.00 (m, 1H) 7.57 (m, 1H) 7.86-7.95 (m, 3H) 8.04 (m, 1H) 8.08 (m, 1H) 8.37 (m, 1H), 11.16 (s, 1H). ESI-MS: m/z 476.3 (M+H)⁺.

### Compound 32: N-(6-(6-(1-(2-hydroxyoxyethyl)-1H-pyrazol-4-yl)-[1,2,4]triazolo[4,3-a]pyridin-3-ylthio)imidazo[1,2-b]pyridazin-2-yl)cyclopropanecarboxamide

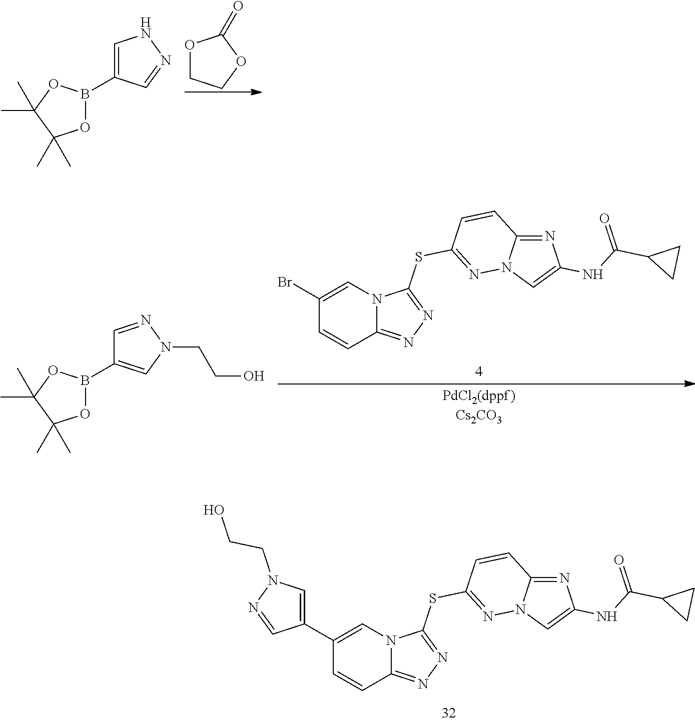

2-(4-(4,4,5,5-tetramethyl-1,3,2-dioxaborolan-2-yl)-1H-pyrazol-1-yl)ethanol was synthesized according to the procedure in PCT Publication No. WO 2008/44022, which is incorporated herein in its entirety. Specifically, 4-(4,4,5,5-tetramethyl-1,3,2-dioxaborolan-2-yl)-1H-pyrazole (5.0 g, 25.8 mmol), 1,3-dioxolan-2-one (2.5 g, 28.3 mmol), and sodium hydroxide (pellets, 1.0 g, 25.8 mmol) were dissolved in DMF (206 mL). The reaction mixture was heated to reflux for 2 hours. Activated charcoal was added after reaction was cooled to ambient temperature and the reaction was stirred for 1 hr and then filtered through Celite. The filter cake was then rinsed with DMF (120 mL), and the filtrate was concentrated to provide 2-(4-(4,4,5,5-tetramethyl-1,3,2-dioxaborolan-2-yl)-1H-pyrazol-1-yl)ethanol as a yellow oil (6 g). The resulting material was used without further purification. ESI-MS: m/z 239.3 (M+H)⁺.

A mixture of N-(6-(6-bromo-[1,2,4]triazolo[4,3-a]pyridin-3-ylthio)imidazo[1,2-b]pyridazin-2-yl)cyclopropanecarboxamide (500 mg, 1.2 mmol), 2-(4-(4,4,5,5-tetramethyl-1,3,2-dioxaborolan-2-yl)-1H-pyrazol-1-yl)ethanol (1.4 g, 5.8 mmol), cesium carbonate (1.14 mL, 3 M solution), and [1,1′-bis(diphenylphosphino)ferrocene]-dichloropalladium(II) (85 mg, 0.12 mmol) in DME (5 mL) was heated in a microwave for 30 min at 100° C. The reaction mixture was then concentrated to dryness via rotary evaporation. The resulting crude material was reconstituted in DMSO (Volume: 1.0 mL) and purified via preparative mass trigger LCMS using a gradient eluant of 10-35% ACN:0.05% TFA (aq). The collected fractions were combined and the ACN was removed via rotary evaporation. The resulting mixture was lyophilized and reconstituted in water:ACN (1:10, 5.0 mL) Two drops of concentrated HCl were added and the resulting solution was lyophilized to provide the HCl salt of the title compound ¹H NMR (400 MHz, DMSO-d₆) δ ppm 11.16 (s, 1H) 8.69 (s, 1H) 8.38 (s, 1H) 8.04-8.08 (m, 1H) 8.02 (d, J=1.01 Hz, 1H) 7.91-7.95 (m, 1H) 7.88-7.91 (m, 2H) 6.99-7.05 (m, 1H) 4.10-4.16 (m, 2H) 3.73 (t, J=5.56 Hz, 2H) 1.90 (quin, J=6.19 Hz, 1H) 0.74-0.84 (m, 4H) ESI-MS: m/z 462.3 (M+H)⁺. MP 182-184° C.

### Compound 33: 3-(2-(cyclopropanecarboxamido)imidazo[1,2-b]pyridazin-6-ylthio)-[1,2,4]triazolo[4,3-a]pyridine-6-carboxylic acid

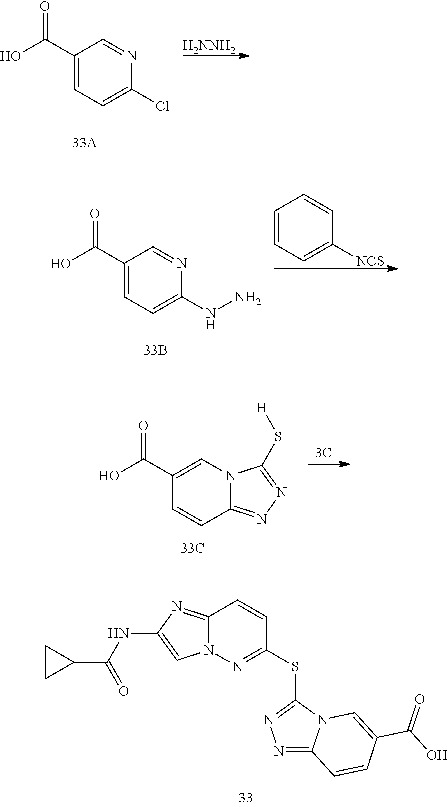

### 3-Mercapto-[1,2,4]triazolo[4,3-a]pyridine-6-carboxylic acid

6-chloronicotinic acid (1.6 g) in MeOH (10 mL) was treated with NH₂NH₂ (2 g) at 140° C. for 2 h. After cooling to −20° C., the solid was filtered to give 560 mg of compound 33B. Compound 33B was treated with isothiocyanatobenzene (141 mg) in 1,2-dichlorobenzene (5 mL) at 100° C. for 10 min, and then 180° C. for 1 h. The product was purified by LCMS to give 3-mercapto-[1,2,4]triazolo[4,3-a]pyridine-6-carboxylic acid (450 mg). ESI-MS: m/z 196.1 (M+H)⁺.

### 3-(2-(Cyclopropanecarboxamido)imidazo[1,2-b]pyridazin-6-ylthio)-[1,2,4]triazolo[4,3-a]pyridine-6-carboxylic acid

Compound 33 was prepared from 3-mercapto-[1,2,4]triazolo[4,3-a]pyridine-6-carboxylic acid following the procedure described in the synthesis of compound 4. ¹H NMR (400 MHz, CDCl₃-CD₃OD 10:1) δ ppm 9.03 (bs, 1H), 8.01 (d, J=9.1 Hz, 1H), 7.87 (d, J=9.6 Hz, 1H), 7.68 (d, J=9.4 Hz, 1H), 7.36 (m, 1H), 7.09 (d, J=9.3 Hz, 1H), 2.84 (m, 1H), 1.72 (m, 1H), 0.97 (m, 2H), 0.84 (m, 2H) ESI-MS: m/z 396.1 (M+H)⁺.

### Compound 34: 3-(2-(cyclopropanecarboxamido)imidazo[1,2-b]pyridazin-6-ylthio)-N-cyclopropyl-[1,2,4]triazolo[4,3-a]pyridine-6-carboxamide

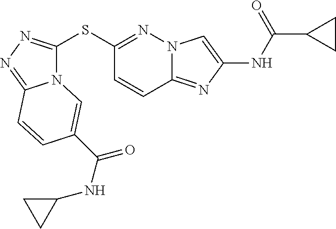 [SMILES: O=C(NC1CC1)c1ccc2nnc(Sc3ccc4nc(NC(=O)C5CC5)cn4n3)n2c1]

To a solution of compound 33 (10 mg), cyclopropanamine (30 mg) and DIEPA (100 uL) in DMF (1 mL) was added HATU (20 mg). The mixture was stirred at rt for 0.5 h and 60° C. for 5 h. The title compound was isolated by preparative LCMS. ¹H NMR (400 MHz, CDCl₃-CD₃OD 10:1) δ ppm 8.95 (bs, 1H), 8.06 (dd, J=9.4, 1.6 Hz, 1H), 7.98 (dd, J=9.6, 1.0 Hz, 1H), 7.93 (s, 1H), 7.87 (d, J=9.4 Hz, 1H), 7.29 (d, J=9.3 Hz, 1H), 1.77 (m, 1H), 1.04 (m, 1H), 0.95 (m, 2H). 0.89 (m, 2H). 0.80 (m, 2H). 0.63 (m, 2H). ESI-MS: m/z 435.1 (M+H)⁺.

### Compound 35: 3-(2-(cyclopropanecarboxamido)imidazo[1,2-b]pyridazin-6-ylthio)-N-isobutyl-[1,2,4]triazolo[4,3-a]pyridine-6-carboxamide

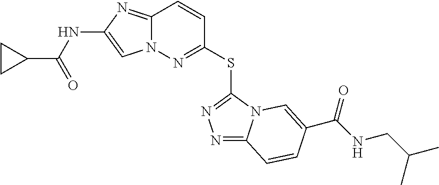 [SMILES: CC(C)CNC(=O)c1ccc2nnc(Sc3ccc4nc(NC(=O)C5CC5)cn4n3)n2c1]

Compound 35 was prepared from 2-methylpropan-1-amine following the procedure described in the synthesis of compound 34. ¹H NMR (400 MHz, CD₃OD) δ ppm 8.88 (bs, 1H), 8.72 (bs, 1H), 7.95-7.88 (m, 3H), 7.15 (bs, 1H), 3.08 (t, J=6.0 Hz, 2H), 1.78 (m, 1H), 1.3-1.15 (m, 3H), 0.85 (m, 2H), 0.82 (d, J=6.8 Hz, 6H) ESI-MS: m/z 451.1 (M+H)⁺.

### Compound 36: N-(6-(6-(4-(piperazin-1-yl)phenyl)-[1,2,4]triazolo[4,3-a]pyridin-3-ylthio)imidazo[1,2-b]pyridazin-2-yl)cyclopropanecarboxamide

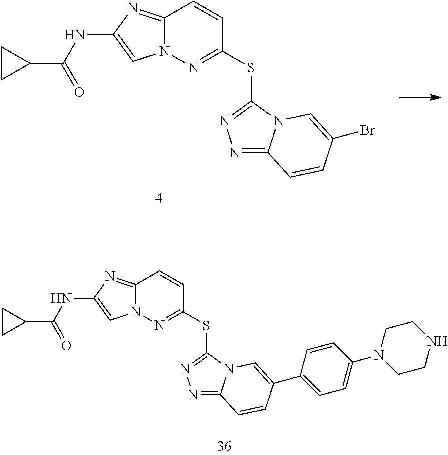 [SMILES: O=C(Nc1cn2nc(Sc3nnc4ccc(Br)cn34)ccc2n1)C1CC1 ; O=C(Nc1cn2nc(Sc3nnc4ccc(-c5ccc(N6CCNCC6)cc5)cn34)ccc2n1)C1CC1]

Compound 36 was prepared from N-(6-(6-bromo-[1,2,4]triazolo[4,3-a]pyridin-3-ylthio)imidazo[1,2-b]pyridazin-2-yl)cyclopropanecarboxamide and tert-butyl 4-(4-(4,4,5,5-tetramethyl-1,3,2-dioxaborolan-2-yl)phenyl)piperazine-1-carboxylate following an analogous procedure to that described in connection with the synthesis of compound 5. The crude product was treated with 4 M HCl-dioxane at 80° C. for 1 h. Purification of the mixture by LCMS gave the title compound as a TFA salt. ¹H NMR (400 MHz, CD₃OD) δ ppm 8.61 (bs, 1H), 7.92-8.0 (m, 2H), 7.8 (bs, 1H), 7.6 (m, 1H), 7.57, (d, J=8.9 Hz, 2H), 7.21 (bs, 1H), 7.08, (d, J=8.8 Hz, 2H), 3.47 (m, 4H), 3.36 (m, 4H), 1.82 (m, 1H), 1.3-1.15 (m, 2H), 0.85 (m, 2H). ESI-MS: m/z 512.2 (M+H)⁺.

### Compound 37: N-(6-(6-(3-hydroxyprop-1-ynyl)-[1,2,4]triazolo[4,3-a]pyridin-3-ylthio)imidazo[1,2-b]pyridazin-2-yl)cyclopropanecarboxamide

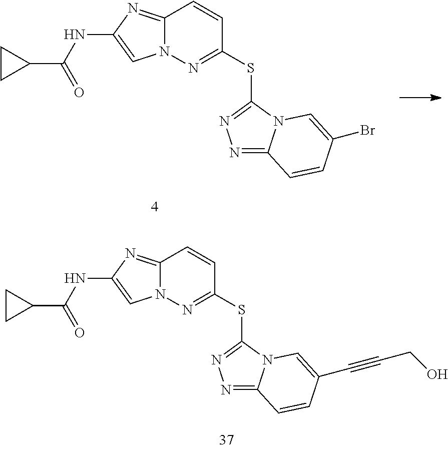 [SMILES: O=C(Nc1cn2nc(Sc3nnc4ccc(Br)cn34)ccc2n1)C1CC1 ; O=C(Nc1cn2nc(Sc3nnc4ccc(C#CCO)cn34)ccc2n1)C1CC1]

A mixture of N-(6-(6-bromo-[1,2,4]triazolo[4,3-a]pyridin-3-ylthio)imidazo[1,2-b]pyridazin-2-yl)cyclopropanecarboxamide (43 mg), prop-2-yn-1-ol (10 mg), Ph₃P (1.1 mg) and tetrakis(triphenylphosphine)palladium(0) (i.e., Pd[P(C₆H₅)₃]₄) (4 mg) in THF (1.5 mL), was stirred at rt for 10 min under N₂, and then CuI (1 mg) was added. The mixture was heated at 60° C. overnight. Purification of the mixture by LCMS gave the title compound as a TFA salt. ¹H NMR (400 MHz, DMSO) δ ppm 11.16 (s, 1H), 8.67 (t, J=1.0 Hz, 1H), 8.00 (dd, J=9.4 and 1.0 Hz, 1H), 7.93 (dd, J=9.3 and 0.7 Hz, 1H), 7.91 (s, 1H), 7.53 (dd, J=9.4 and 1.5 Hz, 1H), 7.12 (d, J=9.4 Hz, 1H), 1.91 (m, 1H), 1.24 (m, 2H), 0.80 (m, 2H). ESI-MS: m/z 406.2 (M+H)⁺.

### Compound 38: N-(6-(6-(2H-tetrazol-5-yl)-[1,2,4]triazolo[4,3-a]pyridin-3-ylthio)imidazo[1,2-b]pyridazin-2-yl)cyclopropanecarboxamide

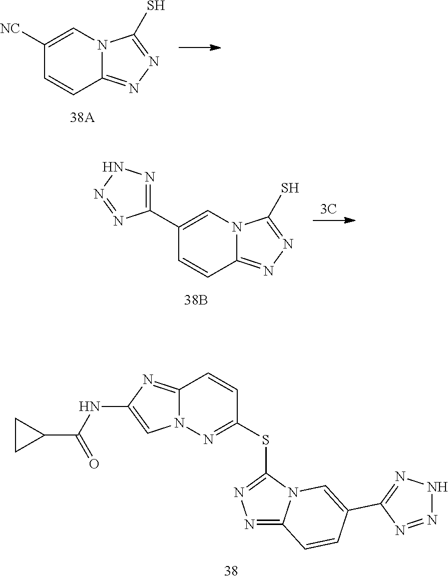 [SMILES: N#Cc1ccc2nnc(S)n2c1 ; Sc1nnc2ccc(-c3nn[nH]n3)cn12 ; O=C(Nc1cn2nc(Sc3nnc4ccc(-c5nn[nH]n5)cn34)ccc2n1)C1CC1]

### 6-(2H-tetrazol-5-yl)[1,2,4]triazolo[4,3-a]pyridine-3-thiol

A mixture of 3-mercapto-[1,2,4]triazolo[4,3-a]pyridine-6-carbonitrile (360 mg, 2 mmol), NaN₃ and NH₄Cl in DMF (10 mL) was heated at 140° C. for 10 h under microwave condition. The product was purified by LCMS to give compound 38B (320 mg). ESI-MS: m/z 220.1 (M+H)⁺.

### 3-(2-(cyclopropanecarboxamido)imidazo[1,2-b]pyridazin-6-ylthio)-N-isobutyl-[1,2,4]triazolo[4,3-a]pyridine-6-carboxamide

Compound 38 was prepared from compound 38B following the procedure described in the synthesis of compound 4. ¹H NMR (400 MHz, DMSO) δ ppm 11.16 (s, 1H), 9.14 (t, J=1.5 Hz, 1H), 8.22 (dd, J=9.6 and 1.0 Hz, 1H), 8.14 (dd, J=9.6 and 0.7 Hz, 1H), 7.95 (s, 1H), 7.94 (d, J=9.4 Hz, 1H), 7.18 (d, J=9.6 Hz, 1H), 1.90 (m, 1H), 0.78 (bd, J=5.8 Hz, 2H). ESI-MS: m/z 419.1 (M+H)⁺.

### Compound 39: N-(6-(6-(5-(methoxymethyl)-1,2,4-oxadiazol-3-yl)-[1,2,4]triazolo[4,3-a]pyridin-3-ylthio)imidazo[1, 2-1)]pyridazin-2-yl)cyclopropanecarboxamide

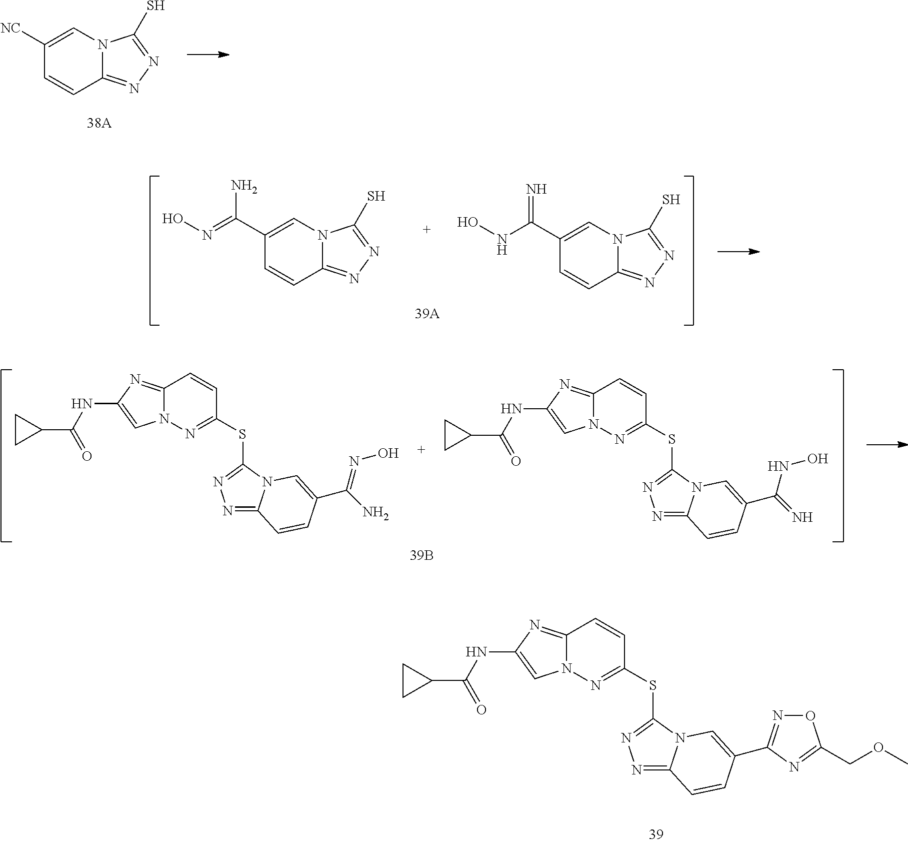

### (Z)—N-hydroxy-3-mercapto-[1,2,4]triazolo[4,3-a]pyridine-6-carboximidamide

A mixture of 3-mercapto-[1,2,4]triazolo[4,3-a]pyridine-6-carbonitrile (390 mg, 2 mmol) and hydroxylamine (50% wt in water, 420 mg) in EtOH (4 mL) was heated at 120° C. for 10 h under microwave condition. The product was purified by LCMS to give compound 39A and its tautomer (128 mg). ESI-MS: m/z 210.0 (M+H)⁺.

### (Z)—N-(6-(6-(N′-hydroxycarbamimidoyl)-[1,2,4]triazolo[4,3-a]pyridin-3-ylthio)imidazo[1,2-b]pyridazin-2-yl)cyclopropanecarboxamide

Compound 39B and its tautomer were prepared from compound 39A and its tautomer following the procedure described in the synthesis of compound 4. ESI-MS: m/z 410.1 (M+H)⁺.

### N-(6-(6-(5-(methoxymethyl)-1,2,4-oxadiazol-3-yl)-[1,2,4]triazolo[4,3-a]pyridin-3-ylthio)imidazo[1,2-b]pyridazin-2-yl)cyclopropanecarboxamide

To a cooled (−10° C.) mixture of compound 39B (30 mg) and DIEPA (200 mL) in THF-DME (1-1, 1.5 mL) was added a solution of 2-methoxyacetyl chloride (11 mg) in THF (0.5 mL). The mixture was stirred at this temperature for 5 min, and then rt for 1 h. The mixture was heated at 140° C. under microwave condition for 30 min, and purified by LCMS to give the title compound as a TFA salt. ¹H NMR (400 MHz, DMSO) δ ppm 11.49 (s, 1H), 9.09 (bs, 1H), 8.19 (dd, J=9.6 and 1.6 Hz, 1H), 8.11-8.12 (m, 2H), 7.92 (d, J=9.3 Hz, 1H), 7.37 (d, J=9.4 Hz, 1H), 4.77 (s, 1H), 3.56 (s, 3H), 1.77 (m, 1H), 1.06 (m, 2H), 0.93 (m, 2H). ESI-MS: m/z 463.1 (M+H)⁺.

### Compound 40: N-(6-(6-(2-hydroxypropan-2-yl)-[1,2,4]triazolo[4,3-a]pyridin-3-ylthio)imidazo[1,2-b]pyridazin-2-yl)cyclopropanecarboxamide

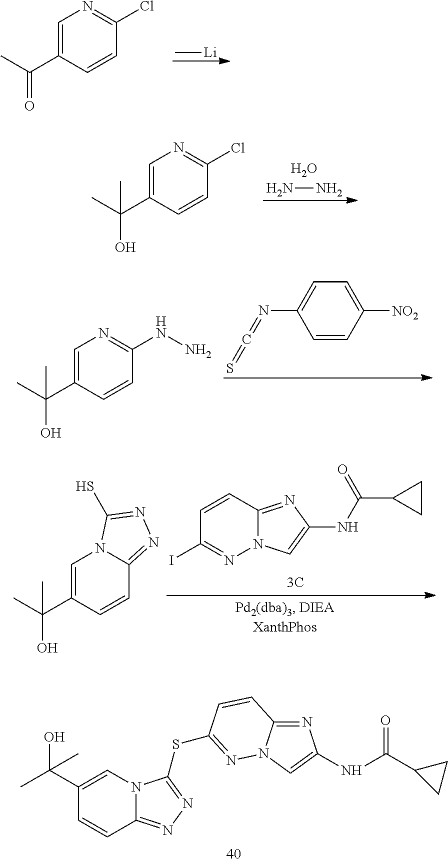

### 2-(6-Chloropyridin-3-yl)propan-2-ol

To a solution of 1-(6-chloropyridin-3-yl)ethanone (2.0 g, 12.85 mmol) in THF (Volume: 25 mL) was added methyllithium (16.07 mL, 25.7 mmol) at 0° C. The reaction was stirred for 1 hr and allowed to warm to room temperature. The resulting deep red solution was quenched with water (Volume: 50 mL), extracted with EtOAc (1×50 mL), and the organic layer was dried over MgSO₄. The organic phase was filtered and the filtrate was evaporated to dryness via rotary evaporation. The resulting material was purified via MPLC (DCM: MeOH, 95:5) to provide 2-(6-chloropyridin-3-yl)propan-2-ol (0.985 g, 5.74 mmol, 44.6% yield) as a dark red solid. ¹H NMR (400 MHz, DMSO-d₆) δ ppm 1.44 (s, 5H) 5.32 (s, 1H) 7.44 (d, J=8.34 Hz, 1H) 7.90 (dd, J=8.34, 2.53 Hz, 1H) 8.49 (d, J=2.53 Hz, 1H). ESI-MS: m/z 172.0 (M+H)⁺.

### 2-(6-Hydrazinylpyridin-3-yl)propan-2-ol: A mixture of 2-(6-chloropyridin-3-yl)propan-2-ol (975 mg, 5.68 mmol), and hydrazine hydrate (5512 μA, 114 mmol) was heated in a microwave on high absorbance for 10 hr at 110° C. The mixture was cooled to room temperature and the hydrazine was removed via rotary evaporation. The resulting yellow residue was used without further work-up.

### 2-(3-Mercapto-[1,2,4]triazolo[4,3-a]pyridin-6-yl)propan-2-ol

A mixture of 2-(6-hydrazinylpyridin-3-yl)propan-2-ol (250 mg, 1.495 mmol) and 1-isothiocyanato-4-nitrobenzene (269 mg, 1.495 mmol) in ACN (Volume: 2.0 mL) was stirred at room temperature for 1 hr. The reaction was diluted with Et₂O (Volume: 5.0 mL), and the resulting solid was filtered. Minimal product was present in the solid. On standing for 1 hr, the filtrate had formed a precipitate. This was filtered to provide 2-(5-(2-hydroxypropan-2-yl)pyridin-2-yl)-N-(4-nitrophenyl)hydrazinecarbothioamide (175 mg, 0.504 mmol, 33.7% yield) as an orange solid. A solution of 2-(5-(2-hydroxypropan-2-yl)pyridin-2-yl)-N-(4-nitrophenyl)hydrazinecarbothioamide (275 mg, 0.792 mmol) in DME (Volume: 2.0 mL) was heated in a microwave on high absorbance for 3 hr at 110° C. The reaction was stripped to dryness onto silica gel via rotary evaporation and purified compound by MPLC with DCM:MeOH (98:2) to provide 2-(3-mercapto-[1,2,4]triazolo[4,3-a]pyridin-6-yl)propan-2-ol.

### N-(6-(6-(2-hydroxypropan-2-yl)[1,2,4]triazolo[4,3-a]pyridin-3-ylthio)imidazo[1,2-b]pyridazin-2-yl)cyclopropanecarboxamide

A mixture of N-(6-iodoimidazo[1,2-b]pyridazin-2-yl)cyclopropanecarboxamide (62.7 mg, 0.191 mmol), Pd₂(dba)₃ (10.50 mg, 0.011 mmol), XANTPHOS (13.27 mg, 0.023 mmol), DIEA (0.067 mL, 0.382 mmol), and 2-(3-mercapto-[1,2,4]triazolo[4,3-a]pyridin-6-yl)propan-2-ol (40 mg, 0.191 mmol) in DME (Volume: 1.5 mL) was heated in a microwave on high absorbance for 30 min at 120° C. The reaction mixture was then concentrated to dryness via rotary evaporation. The resulting crude material was reconstituted in DMSO (Volume: 1.0 mL) and purified via preparative mass trigger LCMS using a gradient eluant of 20-45% ACN:0.05% TFA (aq). The collected fractions were combined and the ACN was removed via rotary evaporation. The resulting mixture was lyophilized to provide the TFA salt of the title compound (3.3 mg, 8.06 μmol, 4.22% yield) as a brown solid. ¹H NMR (400 MHz, DMSO-d₆) δ ppm 0.72-0.85 (m, 3H) 1.43 (s, 4H) 1.91 (m, 1H) 7.04 (d, J=9.35 Hz, 1H) 7.70 (dd, J=9.60, 1.77 Hz, 1H) 7.87-8.00 (m, 2H) 8.32 (d, J=1.52 Hz, 1H) 11.18 (s, 1H). ESI-MS: m/z 410.3 (M+H)⁺.

### Compound 41: N-(6-(6-(trifluoromethyl)-[1,2,4]triazolo[4,3-a]pyridin-3-ylthio)imidazo[1,2-b]pyridazin-2-yl)cyclopropanecarboxamide

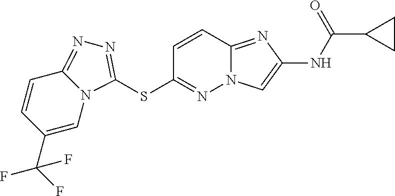 [SMILES: O=C(Nc1cn2nc(Sc3nnc4ccc(C(F)(F)F)cn34)ccc2n1)C1CC1]

The title compound was prepared using an analogous procedure to that described in connection with compound 6. ¹H NMR (400 MHz, DMSO-d₆) δ=11.16 (s, 1H), 9.03 (d, J=1.3 Hz, 1H), 8.25-8.17 (m, 1H), 7.96-7.90 (m, 2H), 7.83 (dd, J=1.5, 9.6 Hz, 1H), 7.17 (d, J=9.6 Hz, 1H), 1.91 (quin, J=6.3 Hz, 1H), 0.84-0.76 (m, 4H) ESI-MS: m/z 420.3 (M+H)⁺.

### Compound 42: N-(6-(6-methyl-[1,2,4]triazolo[4,3-a]pyridin-3-ylthio)imidazo[1,2-b]pyridazin-2-yl)cyclopropanecarboxamide

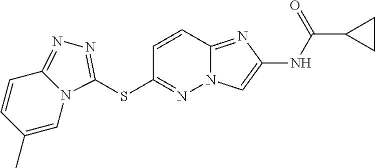 [SMILES: Cc1ccc2nnc(Sc3ccc4nc(NC(=O)C5CC5)cn4n3)n2c1]

The title compound was prepared using an analogous procedure to that described in connection with compound 6. ¹H NMR (400 MHz, DMSO-d₆) δ=11.17 (s, 1H), 8.35 (d, J=1.3 Hz, 1H), 7.96-7.87 (m, 3H), 7.46 (dd, J=1.5, 9.3 Hz, 1H), 7.00 (d, J=9.3 Hz, 1H), 2.34-2.27 (m, 3H), 1.95-1.86 (m, 1H), 0.82-0.75 (m, 4H) ESI-MS: m/z 366.3 (M+H)⁺.

### Compound 43: N-(6-(7-methyl-[1,2,4]triazolo[4,3-a]pyridin-3-ylthio)imidazo[1,2-b]pyridazin-2-yl)cyclopropanecarboxamide

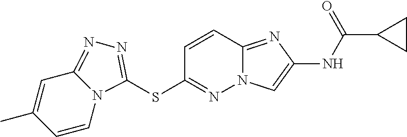 [SMILES: Cc1ccn2c(Sc3ccc4nc(NC(=O)C5CC5)cn4n3)nnc2c1]

The title compound was prepared using an analogous procedure to that described in connection with compound 6. ¹H NMR (400 MHz, DMSO-d₆) δ=11.17 (s, 1H), 8.38 (d, J=7.1 Hz, 1H), 7.96-7.88 (m, 2H), 7.76 (d, J=1.3 Hz, 1H), 7.06 (d, J=9.3 Hz, 1H), 7.00 (dd, J=1.5, 7.1 Hz, 1H), 2.44 (s, 3H), 1.95-1.86 (m, 1H), 0.83-0.73 (m, 4H) ESI-MS: m/z 366.3 (M+H)⁺.

### Compound 44: N-(6-(6-(3-fluorophenyl)-[1,2,4]triazolo[4,3-a]pyridin-3-ylthio)imidazo[1,2-b]pyridazin-2-yl)cyclopropanecarboxamide

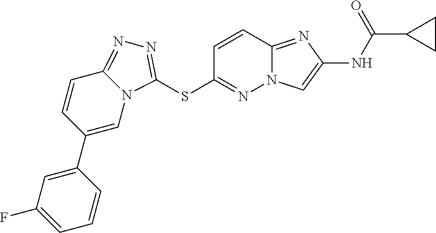 [SMILES: O=C(Nc1cn2nc(Sc3nnc4ccc(-c5cccc(F)c5)cn34)ccc2n1)C1CC1]

The title compound was prepared using an analogous procedure to that described in connection with compound 6. ¹H NMR (400 MHz, DMSO-d₆) δ=11.15 (s, 1H), 8.81 (d, J=1.5 Hz, 1H), 8.11 (dd, J=1.0, 9.6 Hz, 1H), 7.99 (dd, J=1.6, 9.5 Hz, 1H), 7.95-7.89 (m, 1H), 7.68 (dt, J=2.1, 10.4 Hz, 2H), 7.62 (d, J=7.8 Hz, 1H), 7.53 (td, J=6.2, 8.0 Hz, 1H), 7.27 (td, J=2.1, 8.3 Hz, 1H), 7.07 (d, J=9.6 Hz, 1H), 1.89 (quin, J=6.3 Hz, 1H), 0.86-0.74 (m, 4H) ESI-MS: m/z 446.3 (M+H)⁺.

### Compound 45: N-(6-(difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide

### Method A

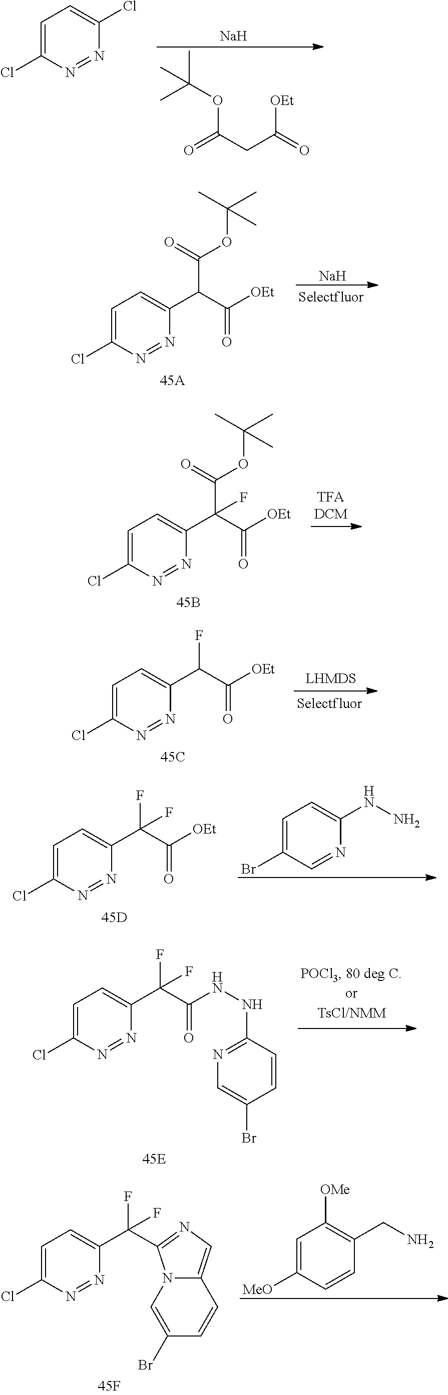

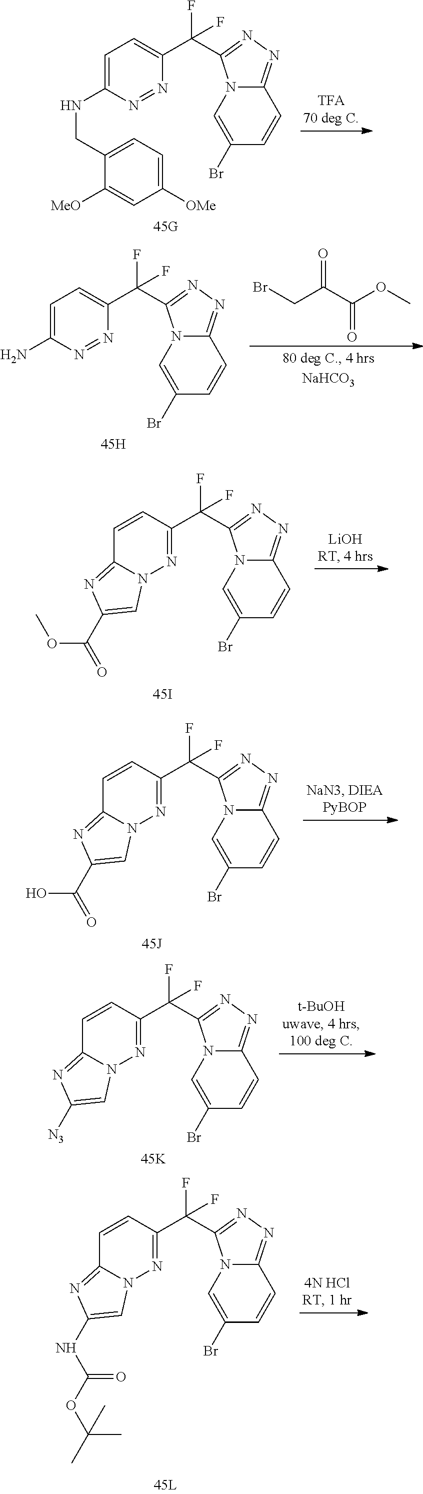

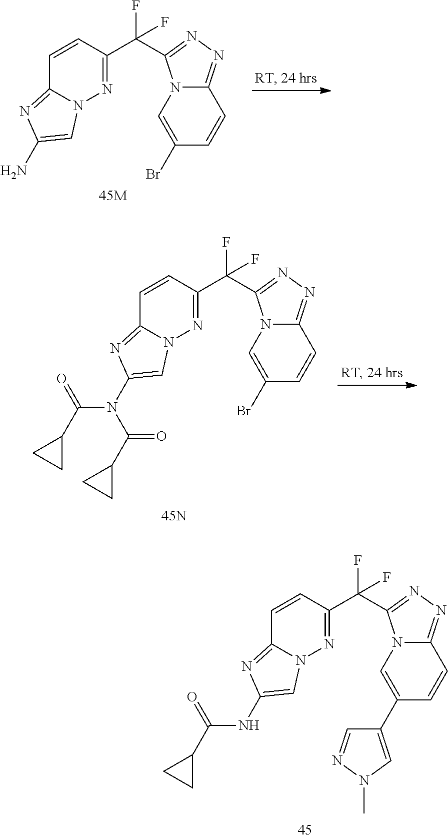 [SMILES: Nc1cn2nc(C(F)(F)c3nnc4ccc(Br)cn34)ccc2n1 ; O=C(C1CC1)N(C(=O)C1CC1)c1cn2nc(C(F)(F)c3nnc4ccc(Br)cn34)ccc2n1 ; Cn1cc(-c2ccc3nnc(C(F)(F)c4ccc5nc(NC(=O)C6CC6)cn5n4)n3c2)cn1]

### 1-tert-Butyl 3-ethyl 2-(6-chloropyridazin-3-yl)malonate

tert-Butyl ethyl malonate (41.3 ml, 211 mmol) was added drop-wise to a suspension of sodium hydride (19.33 g, 483 mmol) in dioxane (1000 mL) at 0° C. The reaction was stirred at 0° C. for 1 hr and then allowed to warm to ambient temperature. 3,6-dichloropyridazine (30 g, 201 mmol) was then added portion wise at 25° C. The reaction was stirred at reflux for 2 hrs and then solvent was removed via rotary evaporation. The resulting residue was dissolved in EtOAc (400 mL), and the organic phase was washed with saturated NaHCO₃ (400 mL), dried over MgSO₄, filtered, and concentrated to dryness via rotary evaporation. This reaction was repeated. The combined crude residues from both batches was purified via MPLC (Hex:EtOAc, 8:2) to provide the title compound, 1-tert-butyl 3-ethyl 2-(6-chloropyridazin-3-yl)malonate (86.5 g, 288 mmol, 71.3%). ¹H NMR (400 MHz, DMSO-d₆) δ ppm 1.12-1.23 (m, 3H) 1.36-1.52 (m, 9H) 4.20 (m, J=10.71, 7.22, 7.22, 3.85, 3.85 Hz, 2H) 5.29 (s, 1H) 7.85 (d, J=8.84 Hz, 1H) 7.99 (d, J=8.84 Hz, 1H). ESI-MS: m/z 301.2 (M+H)⁺.

### 1-tert-Butyl 3-ethyl 2-(6-chloropyridazin-3-yl)-2-fluoromalonate

To a solution of 1-tert-butyl 3-ethyl 2-(6-chloropyridazin-3-yl)malonate 45A (86.5 g, 288 mmol) in THF (2400 mL) was added NaH (12.65 g, 316 mmol). The reaction was stirred at 0° C. for 15 min. A cloudy solution of Selectfluor (112 g, 316 mmol) in DMF (dry, 800 mL) was added drop-wise at 0° C. and then the reaction was allowed to warm to ambient temperature over a 2 hrs. The reaction was then quenched with saturated NH₄Cl (250 mL) and reduced in volume to about 1500 mL. To this mixture, Et₂O (300 mL) and water (50 mL) were added. Layers were separated, and the aqueous layer was extracted with Et₂O (3×300 mL). The combined organic layers were then washed with saturated NaHCO₃ solution (3×150 mL), dried with MgSO₄, filtered, and concentrated to dryness via rotary evaporation. The residue was purified via MPLC (Hex:EtOAc, 8:2) to provide 1-tert-butyl 3-ethyl 2-(6-chloropyridazin-3-yl)-2-fluoromalonate 45B (70.2 g, 220 mmol, 77%). ¹H NMR (400 MHz, DMSO-d₆) δ ppm 1.24 (t, J=7.20 Hz, 3H) 1.46 (s, 9H) 4.28-4.43 (m, 2H) 8.14 (s, 2H). ESI-MS: m/z 319.2 (M+H)⁺.

### Ethyl 2-(6-chloropyridazin-3-yl)-2-fluoroacetate

A solution of 1-tert-butyl 3-ethyl 2-(6-chloropyridazin-3-yl)-2-fluoromalonate 45B (60.2 g, 189 mmol) in 300 mL TFA/DCM (1:1) was stirred at 25° C. for 2 hrs and then concentrated to dryness via rotary evaporation. The resulting residue was dissolved in EtOAc (300 mL), washed with saturated NaHCO₃ solution, dried over MgSO₄, and then concentrated to dryness to give the title compound, ethyl 2-(6-chloropyridazin-3-yl)-2-fluoroacetate 45C (36.6 g, 167 mmol, 89%). The material was used immediately without further purification. ¹H NMR (400 MHz, DMSO-d₆) δ ppm 1.18 (t, J=7.20 Hz, 3H) 4.23 (qd, J=7.12, 4.42 Hz, 2H) 6.43-6.62 (m, 1H) 8.00-8.12 (m, 2H). ESI-MS: m/z 219.0 (M+H)⁺.

### Ethyl 2-(6-chloropyridazin-3-yl)-2,2-difluoroacetate

To a solution of ethyl 2-(6-chloropyridazin-3-yl)-2-fluoroacetate (36.6 g, 167 mmol) in anhydrous THF (500 mL) was added lithium hexamethyldisilazide (201 ml, 201 mmol) drop-wise at −78° C. After 15 minutes, a solution of Selectfluor (71.2 g, 201 mmol) in DMF (183 mL) was added drop-wise. Upon complete addition, the reaction was allowed to warm to ambient temperature over a 30 min period. Saturated NH₄Cl (70 mL) was then added, and THF was removed via rotary evaporation. The resulting residue was diluted with water (500 mL), extracted with Et₂O (3×100 mL), dried with MgSO₄, filtered, and concentrated to dryness. The resulting material was purified via MPLC (Hex:EtOAc, 8:2) to provide ethyl 2-(6-chloropyridazin-3-yl)-2,2-difluoroacetate 45D (20.8 g, 88 mmol, 52.5%). ¹H NMR (400 MHz, DMSO-d₆) δ ppm 1.25 (t, J=7.07 Hz, 3H) 4.38 (q, J=7.07 Hz, 2H) 8.26 (d, J=9.09 Hz, 1H) 8.33 (d, J=8.84 Hz, 1H). ESI-MS: m/z 237.1 (M+H)⁺.

### N′-(5-Bromopyridin-2-yl)-2-(6-chloropyridazin-3-yl)-2,2-difluoroacetohydrazide

To a solution of ethyl 2-(6-chloropyridazin-3-yl)-2,2-difluoroacetate (10.8 g, 45.6 mmol) and 5-bromo-2-hydrazinylpyridine (8.58 g, 45.6 mmol) in ahydrous MeOH (100 mL) was added DIEA (5.90 g, 45.6 mmol). The reaction was stirred at ambient temperature for 18 hrs. The solvent was removed via rotary evaporation and the resulting material was reconstituted in EtOAc. The organic phase was washed with water and concentrated to dryness. The resulting residue was purified by MPLC (EtOAc) to provide the title compound, N′-(5-bromopyridin-2-yl)-2-(6-chloropyridazin-3-yl)-2,2-difluoroacetohydrazide 45E (13.3 g, 35.1 mmol, 77% yield). ¹H NMR (400 MHz, CDCl₃) δ ppm 6.62 (d, J=8.84 Hz, 1H) 7.11 (d, J=9.35 Hz, 1H) 7.47-7.60 (m, 2H) 8.06 (d, J=2.53 Hz, 1H). ESI-MS: m/z 377.9 (M+H)⁺.

### 6-Bromo-3-((6-chloropyridazin-3-yl)difluoromethyl)-[1,2,4]triazolo[4,3-a]pyridine

A mixture of N′-(5-bromopyridin-2-yl)-2-(6-chloropyridazin-3-yl)-2,2-difluoroacetohydrazide (1.0 g, 2.64 mmol) and PCl₅ (1.2 g, 8 mmol) in POCl₃ (40.5 g, 264 mmol) was heated at 140° C. in a sealed tube for 18 hrs. Solvent was removed via rotary evaporation, and the resulting residue was reconstituted in EtOAc. The organic solution was washed with saturated NaHCO₃, dried with MgSO₄, filtered, and concentrated to dryness. The resulting material was purified via MPLC (Hex:EtOAc, 50-100% gradient) to provide the title compound, 6-bromo-3-((6-chloropyridazin-3-yl)difluoromethyl)-[1,2,4]triazolo[4,3-a]pyridine 45F (0.8 g, 2.21 mmol, 84%). ¹H NMR (400 MHz, DMSO-d₆) δ ppm 7.75 (dd, J=9.85, 1.77 Hz, 1H) 8.01 (dd, J=9.85, 1.01 Hz, 1H) 8.31 (d, J=9.09 Hz, 1H) 8.42-8.51 (m, 1H) 8.93 (s, 1H). ESI-MS: m/z 359.9 (M+H)⁺.

6-bromo-3-((6-chloropyridazin-3-yl)difluoromethyl)-[1,2,4]triazolo[4,3-a]pyridine 45F was also prepared by mixing N′-(5-bromopyridin-2-yl)-2-(6-chloropyridazin-3-yl)-2,2-difluoroacetohydrazide (1.514 g, 4 mmol) and 4-methylbenzene-1-sulfonyl chloride (0.839 g, 4.40 mmol) in ethyl acetate (Volume: 25 ml) and cooling the mixture to 10° C. under protection of nitrogen. 4-methylmorpholine (1.142 ml, 10.40 mmol) was added over 2 minutes. The reaction mixture was warmed up to room temperature and stirred for 6 hours. Additional 4-methylmorpholine (0.44 ml, 4 mmol) was added. The reaction mixture was heated at 60° C. for 24 hours to reduce the imidoyl chloride intermediate level to less than 1% by HPLC. The reaction mixture was cooled to room temperature. EtOAc (30 ml) and water (13 ml) was added, and the mixture was stirred for 30 minutes. The aqueous layer was separated. The organic layer was concentrated and the residue was purified by silica gel column to afford 1.28 g of product (89% yield).

### 6-((6-Bromo-[1,2,4]triazolo[4,3-a]pyridin-3-yl)difluoromethyl)-N-(2,4-dimethoxybenzyl)pyridazin-3-amine: A reaction mixture of 6-bromo-3-((6-chloropyridazin-3-yl)difluoromethyl)-[1,2,4]triazolo[4,3-a]pyridine (0.5 g, 1.387 mmol), (2,4-dimethoxyphenyl)methanamine (0.696 g, 4.16 mmol) and NaHCO₃ (0.58 g, 7.0 mmol) in IPA (10 mL) was heated at 140° C. in a microwave for 1 hr. The solvent was removed via rotary evaporation and the resulting residue was reconstituted in EtOAc. The organic solution was washed with water, separated and passed through a short silica plug to provide the title compound, 6-((6-bromo-[1,2,4]triazolo[4,3-a]pyridin-3-yl)difluoromethyl)-N-(2,4-dimethoxybenzyl)pyridazin-3-amine 45G (0.6 g, 1.22 mmol, 88%). ¹H NMR (400 MHz, DMSO-d₆) δ ppm 3.71-3.76 (s, 3H) 3.79 (s, 3H) 3.85-3.89 (m, 2H) 6.46 (dd, J=8.34, 2.53 Hz, 1H) 6.57 (d, J=2.27 Hz, 1H) 6.61-6.66 (m, 1H) 7.08 (d, J=9.35 Hz, 1H) 7.14 (d, J=8.34 Hz, 1H) 7.81 (d, J=9.35 Hz, 1H) 7.99 (dd, J=9.60, 1.01 Hz, 1H) 8.70 (s, 1H). ESI-MS: m/z 491.1 (M+H)⁺.

### 6-((6-Bromo-[1,2,4]triazolo[4,3-a]pyridin-3-yl)difluoromethyl)pyridazin-3-amine

A solution of 6-((6-bromo-[1,2,4]triazolo[4,3-a]pyridin-3-yl)difluoromethyl)-N-(2,4-dimethoxybenzyl)pyridazin-3-amine (16.5 g, 33.6 mmol) and anisole (5.45 g, 50.4 mmol) in TFA (150 mL) was stirred at 70° C. for 30 min. The reaction was concentrated to dryness via rotary evaporation and the resulting residue was sonicated in a solution of Et₂O/NaHCO₃ (pH=7). The resulting solid was collected by filtration, rinsed with water and then Et₂O, and dried in vacuum over P₂O₅ to provide the title compound, 6-((6-bromo-[1,2,4]triazolo[4,3-a]pyridin-3-yl)difluoromethyl)pyridazin-3-amine 45H (11.0 g, 32.2 mmol, 96%). ¹H NMR (400 MHz, DMSO-d₆) δ ppm 7.14 (d, J=9.35 Hz, 1H) 7.71 (dd, J=9.85, 1.77 Hz, 1H) 7.92 (d, J=9.35 Hz, 1H) 7.92 (d, J=10.20 Hz, 1) 8.02 (d, J=10.20, 1 H) 8.74 (s, 1H). ESI-MS: m/z 340.9 (M+H)⁺.

### Methyl 6-((6-bromo-[1,2,4]triazolo[4,3-a]pyridin-3-yl)difluoromethyl)imidazo[1,2-b]pyridazine-2-carboxylate

A reaction mixture of 6-((6-bromo-[1,2,4]triazolo[4,3-a]pyridin-3-yl)difluoromethyl)pyridazin-3-amine (11.0 g, 32.2 mmol), methyl 3-bromo-2-oxopropanoate (11.67 g, 64.5 mmol), and NaHCO₃ (10.84 g) in dioxane (150 mL) was heated at 80° C. for 4 hrs to provide a red reaction mixture. Solids were filtered off, rinsed with dioxane, and the combined filtrates were concentrated via rotary evaporation. The resulting residue was dissolved in EtOAc and washed with 0.1 N NaOH until the red color no longer persisted. The organic phase was then separated and concentrated to dryness. The resulting material was purified via MPLC (5:95, MeOH/EtOAc) to provide the title compound, 45I (4.0 g, 9.45 mmol, 30%). ¹H NMR (400 MHz, DMSO-d₆) δ ppm 3.88 (s, 3H) 7.77 (dd, J=9.73, 1.64 Hz, 1H) 7.85 (d, J=9.60 Hz, 1H) 7.97-8.08 (m, 1H) 8.53 (d, J=9.60 Hz, 1H) 8.98 (d, J=1.01 Hz, 1H) 9.02 (s, 1H). ESI-MS: m/z 423.1 (M+H)⁺.

### 6-((6-Bromo-[1,2,4]triazolo[4,3-a]pyridin-3-yl)difluoromethyl)imidazo[1,2-b]pyridazine-2-carboxylic acid

A solution of 3.methyl 6-((6-bromo-[1,2,4]triazolo[4,3-a]pyridin-3-yl)difluoromethyl)imidazo[1,2-b]pyridazine-2-carboxylate (4.0 g, 9.45 mmol) and LiOH (0.340 g, 14.18 mmol) in 10% H₂O/MeOH (150 mL) was stirred at ambient temperature for 4 hrs. Solvent was removed via rotary evaporation, and the resulting residue was diluted with H₂O (100 mL). The aqueous phase was adjusted to pH=5 with concentrated HCl. The resulting precipitate was collected, rinsed with water, rinsed with EtOAc, and dried under vacuum over P₂O₅ for 18 hrs to provide the title compound, 6-((6-bromo-[1,2,4]triazolo[4,3-a]pyridin-3-yl)difluoromethyl)imidazo[1,2-b]pyridazine-2-carboxylic acid 45J (3.5 g, 8.55 mmol, 90%). ¹H NMR (400 MHz, DMSO-d₆) δ ppm 7.76 (dd, J=9.85, 1.77 Hz, 1H) 7.82 (d, J=9.60 Hz, 1H) 8.02 (d, J=9.60 Hz, 1H) 8.51 (d, J=9.85 Hz, 1H) 8.90 (s, 1H) 8.97 (s, 1H). ESI-MS: m/z 409.0 (M+H)⁺.

### 6-((6-Bromo-[1,2,4]triazolo[4,3-a]pyridin-3-yl)difluoromethyl)imidazo[1,2-b]pyridazine-2-carbonyl azide

To a stirred solution of 6-((6-bromo-[1,2,4]triazolo[4,3-a]pyridin-3-yl)difluoromethyl)imidazo[1,2-b]pyridazine-2-carboxylic acid (3.5 g, 8.55 mmol), N-ethyl-N-isopropylpropan-2-amine (6.63 g, 51.3 mmol) and sodium azide (5.56 g, 86 mmol) in anhyydrous DMF (60 mL), PyBOP (5.34 g, 10.27 mmol) was added in portions at ambient temperature over 5 min. The reaction was stirred for an additional 30 min and diluted with EtOAc (200 mL). The reaction was then poured into a 10% aqueous citric acid (200 mL), and the organic phase was separated and washed with citric acid solution (2×100 mL), saturated NaHCO₃ (3×100 mL), and saturated NaCl solution. The aqueous phases were back extracted with EtOAc (200 mL) and the organic phases were combined, dried with MgSO₄, filtered, and concentrated to dryness via rotary evaporation. The resulting residue was triturated in 1:1 Et₂O/hexane, and the solid was collected by filtration and dried in vacuum over P₂O₅ to provide the title compound, 6-((6-bromo-[1,2,4]triazolo[4,3-a]pyridin-3-yl)difluoromethyl)imidazo[1,2-b]pyridazine-2-carbonyl azide 45K (3.2 g, 7.37 mmol, 92%). ¹H NMR (400 MHz, DMSO-d₆) δ ppm 7.77 (dd, J=9.85, 1.77 Hz, 1H) 7.88 (d, J=9.60 Hz, 1H) 8.00-8.05 (m, 1H) 8.56 (d, J=9.60 Hz, 1H) 8.98 (s, 1H) 9.14 (s, 1H). ESI-MS: m/z 434.0 (M+H)⁺. ESI-MS: m/z 406.0 (M+H)⁺.

### tert-Butyl 6-((6-bromo-[1,2,4]triazolo[4,3-a]pyridin-3-yl)difluoromethyl)imidazo[1,2-b]pyridazin-2-ylcarbamate

6-((6-bromo-[1,2,4]triazolo[4,3-a]pyridin-3-yl)difluoromethyl)imidazo[1,2-b]pyridazine-2-carbonyl azide (3.2 g, 7.37 mmol, divided into 4 portions) in t-butanol (10 mL) was heated in microwave oven at 100° C. for 1 hr. The solvent was removed via rotary evaporation, and the resulting residue was reconstituted in EtOAc, washed with 5% citric acid, and the washed with saturated NaHCO₃. The organic phase was passed through a silica plug, rinsed with EtOAc:MeOH (95:5) and then concentrated to give the title compound, tert-Butyl 6-((6-bromo-[1,2,4]triazolo[4,3-a]pyridin-3-yl)difluoromethyl)imidazo[1,2-b]pyridazin-2-ylcarbamate 45L (2.8 g, 5.83 mmol, 79%), which was used in next step without further purification. ¹H NMR (400 MHz, DMSO-d₆) δ ppm 1.47 (s, 9H) 7.66 (d, J=9.35 Hz, 1H) 7.72-7.78 (m, 1H) 7.96-8.06 (m, 2H) 8.22 (d, J=9.35 Hz, 1H) 8.93 (s, 1H) 10.40 (br, 1H). ESI-MS: m/z 480.1 (M+H)⁺.

### 6-((6-Bromo-[1,2,4]triazolo[4,3-a]pyridin-3-yl)difluoromethyl)imidazo[1,2-b]pyridazin-2-amine

A solution of tert-butyl 6-((6-bromo-[1,2,4]triazolo[4,3-a]pyridin-3-yl)difluoromethyl)imidazo[1,2-b]pyridazin-2-ylcarbamate (2.8 g, 5.83 mmol) in 4N HCl/dioxane (25 mL) was stirred at ambient temperature for 1 hr. The solvent was removed via rotary evaporation and the residue was dried under vacuum to provide the title compound, 6-((6-Bromo-[1,2,4]triazolo[4,3-a]pyridin-3-yl)difluoromethyl)imidazo[1,2-b]pyridazin-2-amine 45M (2.2 g, 5.84 mmol, 92%), as HCl salt. This material was used without further purification. ESI-MS: m/z 380.1 (M+H)⁺.

### N-(6-((6-Bromo-[1,2,4]triazolo[4,3-a]pyridin-3-yl)difluoromethyl)imidazo[1,2-b]pyridazin-2-yl)-N-(cyclopropanecarbonyl)cyclopropanecarboxamide

To a solution of 6-((6-bromo-[1,2,4]triazolo[4,3-a]pyridin-3-yl)difluoromethyl)imidazo[1,2-b]pyridazin-2-amine (2.2 g, 5.84 mmol) and DIEA (4.53 g, 35.0 mmol) in CH₂Cl₂ (250 mL), cyclopropanecarbonyl chloride (1.831 g, 17.52 mmol) was added dropwise at 0° C. The reaction was stirred at ambient temperature for 18 hrs. The reaction was then washed with 5% citric acid (2×100 mL) and then washed with NaHCO₃ (2×100 mL). The organic phase was then dried with MgSO₄, filtered, and concentrated to dryness via rotary evaporation. The resulting material was purified via MPLC (95:5, DCM:MeOH) to provide the title compound, N-(6-((6-Bromo-[1,2,4]triazolo[4,3-a]pyridin-3-yl)difluoromethyl)imidazo[1,2-b]pyridazin-2-yl)-N-(cyclopropanecarbonyl)cyclopropanecarboxamide 45N (2.2 g, 4.27 mmol, 73%). ¹H NMR (400 MHz, DMSO-d₆) δ ppm 0.83-1.03 (m, 8H) 2.05-2.12 (m, 2H) 7.70-7.87 (m, 2H) 7.97-8.06 (m, 1H) 8.48 (d, J=9.60 Hz, 1H) 8.65 (s, 1H) 8.99 (s, 1H). ESI-MS: m/z 516.1 (M+H)⁺.

### N-(6-(Difluoro(6-(1-methyl-1H-pyrazol-4-yl)[1,2,4]triazolo[4,3-a]pyridin-3-yl)methyl)imidazo[1,2-b]pyridazin-2-yl)cyclopropanecarboxamide

A mixture of N-(6-((6-bromo-[1,2,4]triazolo[4,3-a]pyridin-3-yl)difluoromethyl)imidazo[1,2-b]pyridazin-2-yl)-N-(cyclopropanecarbonyl)cyclopropanecarboxamide (0.6 g, 1.16 mmol), 1-methyl-4-(4,4,5,5-tetramethyl-1,3,2-dioxaborolan-2-yl)-1H-pyrazole (0.242 g, 1.162 mmol), and

PdCl₂(dppf):CH₂Cl₂ (0.048 g, 0.058 mmol) in dioxane (10 mL)/2N Na₂CO₃ (5 mL) was heated in a microwave at 110° C. for 30 min. This reaction was repeated 4 times and reaction mixtures were combined. The reaction was diluted with CH₂Cl₂ (500 mL) and washed with H₂O (3×200 mL). The organic phase was separated and concentrated to dryness via rotary evaporation. The resulting residue was suspended in EtOAc and sonicated. The resulting solid was collected by filtration and rinsed with EtOAc. The solid was the dissolved in MeOH/CH₂Cl₂ (5:95, 300 mL) and filtered through a silica plug and rinsed with MeOH/CH₂Cl₂(5:95). Concentrated HCl (0.5 mL) was added to the collected eluent followed by active charcoal. The solution was stirred at ambient temperature for 1 hour and filtered through celite. The solvent was evaporated and the resulting residue was then triturated in EtOAc. The solid was collected by filtration, rinsed with EtOAc and dried in vacuum to provide the title compound, N-(6-(Difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide hydrochloride (0.92 g, 1.78 mmol, 38%). ¹H NMR (400 MHz, DMSO-d₆) δ ppm 0.82 (d, J=6.32 Hz, 4H) 1.91-2.01 (m, 1H) 3.87 (s, 3H) 7.70 (d, J=9.35 Hz, 1H) 7.90 (dd, J=9.60, 1.52 Hz, 1H) 7.98-8.11 (m, 2H) 8.19-8.32 (m, 2H) 8.40 (s, 1H) 8.70 (s, 1H) 11.35 (s, 1H). ESI-MS: m/z 516.1 (M+H)⁺. MP: 195° C. dec.

N-(6-(Difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide hydrochloride was also prepared by dissolving 0.5 g of crude N-(6-(difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide in AcOH (15 ml) at 80° C. to form a clear solution. The solution was cooled to room temperature, Sili-thiourea (commercially available from Silicycle; 0.25 g) was added and the mixture was stirred overnight. The mixture was filtered through celite and 6.5 ml of 1.7M HCl solution in MeOH was added to the AcOH solution. EtOAc (18 ml) was then added slowly to induce crystallization. The mixture was then stirred at rt for 1 hour until a heavy precipitate formed. Additional EtOAc (30 ml) was added and the mixture stirred for 60 min. The solid was then collected via filtration, wash with EtOAc (10 ml) and dried in vacuum at 60° C. over night.

Further, N-(6-(Difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide hydrochloride was prepared by suspending crude N-(6-(difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide (2.2 g; 4.9 mmol) in 333 ml of DCM. 50 ml of 0.2 M HCl in MeOH (10 mmol) was added. The mixture was stirred vigorously for 60 minutes and Sili-Thiourea (commercially available from Silicycle; 1.12 g) was added. The mixture was stirred at room temperature for 14 hours, filtered through Celite and washed with 10% MeOH in DCM (40 ml) to provide a total volume of filtrate of about 420 ml. 15 ml of a 2M HCl MeOH solution was added. The solution was concentrated by rotavap at 225 mbar, 22° C. bath temperature to remove about 325 ml of solvent. The solution was checked for clarity to ensure that solid formation was minimized. 10 ml of a 2M HCl MeOH solution was added. The solution was seeded with 25 mg of N-(6-(difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide hydrochloride salt. The solution was allowed to continue to concentrate at 130 mbar to remove 65 ml solvent in the presence of seed crystal. After the solution turned cloudy, the solution was maintained at room temperature for 40 minutes. The mixture was cooled to 0° C., stirred for 2 hours, and the solids collected by filtration. The solids were dried in vacuum at 60° C. overnight to afford 1.75 g of a pale-yellow solid.

### Method B

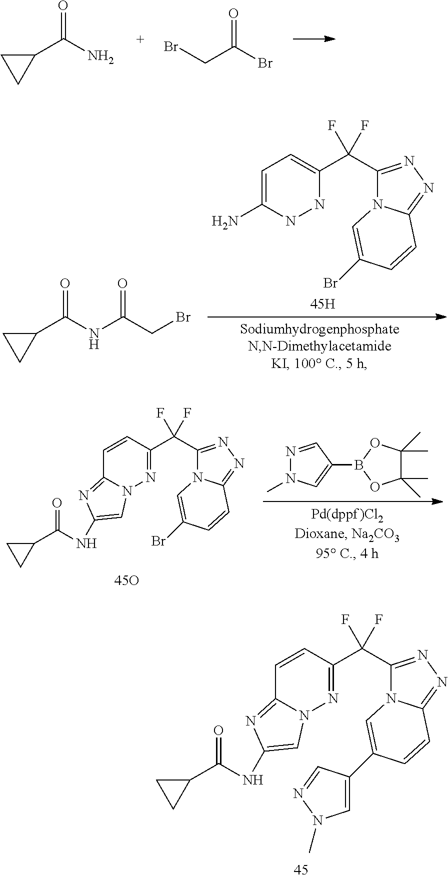

### N-(6-((6-Bromo-[1,2,4]triazolo[4,3-a]pyridin-3-yl)difluoromethyl)imidazo[1,2-b]pyridazin-2-yl)cyclopropanecarboxamide (45O)

Cyclopropanecarboxamide (58.7 g, 690 mmol) in 1,4-Dioxane (1600 ml) was added 2-bromoacetyl bromide (59.9 ml, 690 mmol) at room temperature and stirred for 4 h at 60° C. The reaction mixture was concentrated to dryness. The residue was dissolved in EtOAc and carefully washed with satd. NaHCO₃, water then with brine solution, dried over Na₂SO₄ and concentrated to get N-(2-bromoacetyl)cyclopropanecarboxamide (138 g, 670 mmol, 97% yield) as an off white solid. This material was used without purification. ESI-MS: m/z 208.0 (M+2H)⁺.

To a mixture of 646-bromo-[1,2,4]triazolo[4,3-a]pyridin-3-yl)difluoromethyl)pyridazin-3-amine (21.8 g, 63.9 mmol) and sodium hydrogenphosphate (27.2 g, 192 mmol) in N,N-Dimethylacetamide (250 ml) were added N-(2-bromoacetyl)cyclopropanecarboxamide (19.75 g, 96 mmol) and potassium iodide (10.61 g, 63.9 mmol) at room temperature. The reaction mixture was stirred for 5 h at 100° C. Reaction mixture cooled to room temperature, diluted with EtOAc (1000 ml) and washed with brine solution (5×), dried over Na₂SO₄, volatiles evaporated and the residue was purified by combiflash (2 to 30% MeOH in dichloromethane over 120 min). Product containing fractions were combined and concentrated to get N-(6-((6-bromo-[1,2,4]triazolo[4,3-a]pyridin-3-yl)difluoromethyl)imidazo[1,2-b]pyridazin-2-yl)cyclopropanecarboxamide (13.9 g, 31.0 mmol, 48.5% yield) as an off white solid. ¹H NMR (400 MHz, DMSO-d₆) δ 11.31 (s, 1H), 8.92 (s, 1H), 8.18-8.35 (m, 2H), 7.92-8.06 (m, 1H), 7.61-7.80 (m, 2H), 1.91-2.02 (m, 1H), 0.78-0.89 (m, 4H). ESI-MS: m/z 450.0 (M+2H)⁺.

N-(6-(Difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide (45)

A mixture of N-(6-((6-bromo-[1,2,4]triazolo[4,3-a]pyridin-3-yl)difluoromethyl)imidazo[1,2-b]pyridazin-2-yl)cyclopropanecarboxamide (7.2 g, 16.06 mmol), 1-methyl-4-(4,4,5,5-tetramethyl-1,3,2-dioxaborolan-2-yl)-1H-pyrazole (5.01 g, 24.10 mmol) and PdCl₂(dppf):CH₂Cl₂ (0.655 g, 0.803 mmol) in 1,4-Dioxane:1M Na₂CO₃ (Ratio: 2.1, Volume: 100 ml) was heated at 95° C. for 4 h. The reaction mixture cooled and concentrated to dryness via rotary evaporation, diluted with EtOAc and water. The resulting solid was collected by filtration, rinsed with H₂O followed by EtOAc. The grey color solid dissolved in 20% MeOH in chloroform and treated with activated charcoal for overnight, filtered through a pad of celite and the celite pad rinsed with 20% MeOH in chloroform until no compound detected by HPLC. This solution passed through a silica gel column. The solvent evaporated and the resulting solid rinsed with EtOAc, MeOH, followed by EtOAc and then Et₂O, dried under vacuum to provide N-(6-(difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide (5.13 g, 11.41 mmol, 71.1% yield) as white solid. ¹H NMR (400 MHz, DMSO-d₆) δ 11.35 (s, 1H), 8.71 (s, 1H), 8.41 (s, 1H), 8.19-8.34 (m, 2H), 8.00-8.12 (m, 2H), 7.91 (dd, J=1.64, 9.47 Hz, 1H), 7.71 (d, J=9.35 Hz, 1H), 3.88 (s, 3H), 1.88-2.02 (m, 1H), 0.83 (d, J=6.32 Hz, 4H). ESI-MS: m/z 450.1 (M+H)⁺. MP: 274.9° C.

The bis HCl salt of Compound 45 was prepared as follows. N-(6-(Difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide bis hydrochloride: N-(6-(difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide (2.100 g, 4.67 mmol) in a mixture of MeOH and DCM (Ratio: 1:2, Volume: 100 ml) was added hydrogen chloride (12N, 0.973 ml, 11.68 mmol) at room temperature and stirred for 2 h. Volatiles evaporated to dryness and the solid was rinsed with EtOAc and ether then dried under vacuum to provide N-(6-(difluoro(6-(1-methyl-1H-pyrazol-4-yl)-[1,2,4]triazolo[4,3-a]pyridin-3-yl)methyl)imidazo[1,2-b]pyridazin-2-yl)cyclopropanecarboxamide, 2HCl (2.4 g, 4.59 mmol, 98% yield) as an off white solid. ¹H NMR (400 MHz, DMSO-d₆) δ 12.21 (br. s., 2H), 11.32 (s, 1H), 8.66 (s, 1H), 8.36 (s, 1H), 8.14-8.27 (m, 2H), 7.94-8.08 (m, 2H), 7.87 (dd, J=1.52, 9.60 Hz, 1H), 7.65 (d, J=9.35 Hz, 1H), 3.82 (s, 3H), 1.84-1.99 (m, 1H), 0.77 (d, J=6.32 Hz, 4H). ESI-MS: m/z 450.1 (M+H)⁺. MP: 208° C.

### Compound 46: N-(6-(difluoro(6-(isoxazol-4-yl)-[1,2,4]triazolo[4,3-a]pyridin-3-yl)methyl)imidazo[1,2-b]pyridazin-2-yl)cyclopropanecarboxamide

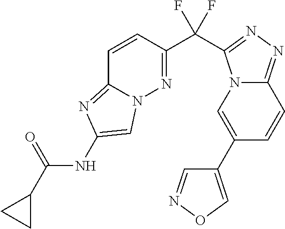 [SMILES: O=C(Nc1cn2nc(C(F)(F)c3nnc4ccc(-c5cnoc5)cn34)ccc2n1)C1CC1]

A mixture of N-(6-((6-bromo-[1,2,4]triazolo[4,3-a]pyridin-3-yl)difluoromethyl)imidazo[1,2-b]pyridazin-2-yl)-N (cyclopropanecarbonyl)cyclopropanecarboxamide (45N, 0.200 g, 0.38 mmol), 4-(4,4,5,5-tetramethyl-1,3,2-dioxaborolan-2-yl)isoxazole (0.1 g, 0.5 mmol) and PdCl₂(dppf):CH₂Cl₂ (5 mg, 0.006 mmol) in Na₂CO₃ (2N, 1 mL)/dioxane (2 mL) was heated at 110° C. in a microwave for 30 min. The reaction mixture was filtered and the solids washed with EtOAc. The filtrate was then washed with saturated NaCl, dried with MgSO₄, filtered, and concentrated to dryness via rotary evaporation. The resulting residue was purified by preparative LCMS. The collected fractions were combined and the resulting mixture was treated with two drops of concentrated HCl. The solution was lyophilized to provide the HCl salt of the title compound, N-(6-(difluoro(6-(isoxazol-4-yl)-[1,2,4]triazolo[4,3-a]pyridin-3-yl)methyl)imidazo[1,2-b]pyridazin-2-yl)cyclopropanecarboxamide (20 mg, 0.04 mmol, 12%). ¹H NMR (400 MHz, DMSO-d₆) δ ppm 0.82 (d, J=6.32 Hz, 4H) 1.91-2.01 (m, 1H) 7.67 (dd, J=9.47, 5.43 Hz, 1H) 7.75 (dd, J=9.73, 1.64 Hz, 1H) 7.84 (dd, J=9.85, 1.77 Hz, 1H) 7.98-8.07 (m, 1H) 8.23-8.34 (m, 2H) 8.46 (s, 1H) 9.02 (s, 1H) 11.36 (d, J=2.27 Hz, 1H). ESI-MS: m/z 437.2 (M+H)⁺.

### Compound 47: N-(6-(difluoro(6-(1-(2-hydroxyethyl)-1H-pyrazol-4-yl)-[1,2,4]triazolo[4,3-a]pyridin-3-yl)methyl)imidazo[1,2-b]pyridazin-2-yl)cyclopropanecarboxamide

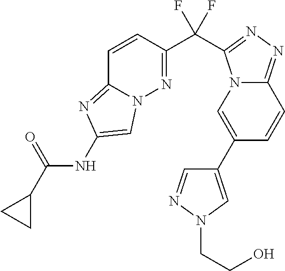 [SMILES: O=C(Nc1cn2nc(C(F)(F)c3nnc4ccc(-c5cnn(CCO)c5)cn34)ccc2n1)C1CC1]

A mixture of N-(6-((6-bromo-[1,2,4]triazolo[4,3-a]pyridin-3-yl)difluoromethyl)imidazo[1,2-b]pyridazin-2-yl)-N (cyclopropanecarbonyl)cyclopropanecarboxamide (45N, 0.067 g, 0.13 mmol), 1-(2-(tetrahydro-2H-pyran-2-yloxy)ethyl)-4-(4,4,5,5-tetramethyl-1,3,2-dioxaborolan-2-yl)-1H-pyrazole (0.042 g, 0.130 mmol) and PdCl₂(dppf) (5 mg, 0.006 mmol) in Na₂CO₃ (2N, 1 mL)/dioxane (2 mL) was heated at 110° C. in a microwave for 30 min. Reaction mixture was filtered and washed with EtOAc. The organic phase was washed with saturated NaCl, dried with MgSO₄, filtered, and concentrated to dryness. The resulting residue was dissolved in 4 N HCl/dioxane and stirred at ambient temperature for 30 min and then concentrated to dryness. The resulting material was purified by preparative LCMS. The collected fractions were combined and the resulting mixture was treated with two drops of concentrated HCl. The solution was lyophilized to provide the HCl salt of the title compound, (18 mg, 0.037 mmol, 29%). ¹H NMR (400 MHz, DMSO-d₆) δ ppm 0.83 (d, J=6.06 Hz, 4H) 1.95 (d, J=5.81 Hz, 1H) 3.77 (t, J=5.43 Hz, 2H) 4.10-4.21 (m, 2H) 7.71 (d, J=9.35 Hz, 1H) 7.87-7.98 (m, 1H) 8.00-8.07 (m, 1H) 8.11 (s, 1H) 8.20-8.33 (m, 2H) 8.44 (s, 1H) 8.73 (s, 1H) 11.35 (s, 1H). ESI-MS: m/z 480.2 (M+H)⁺.

### Compound 48: N-(6-(1-(6-bromo-[1,2,4]triazolo[4,3-a]pyridin-3-yl)ethyl)imidazo[1,2-b]pyridazin-2-yl)cyclopropanecarboxamide

### Method A

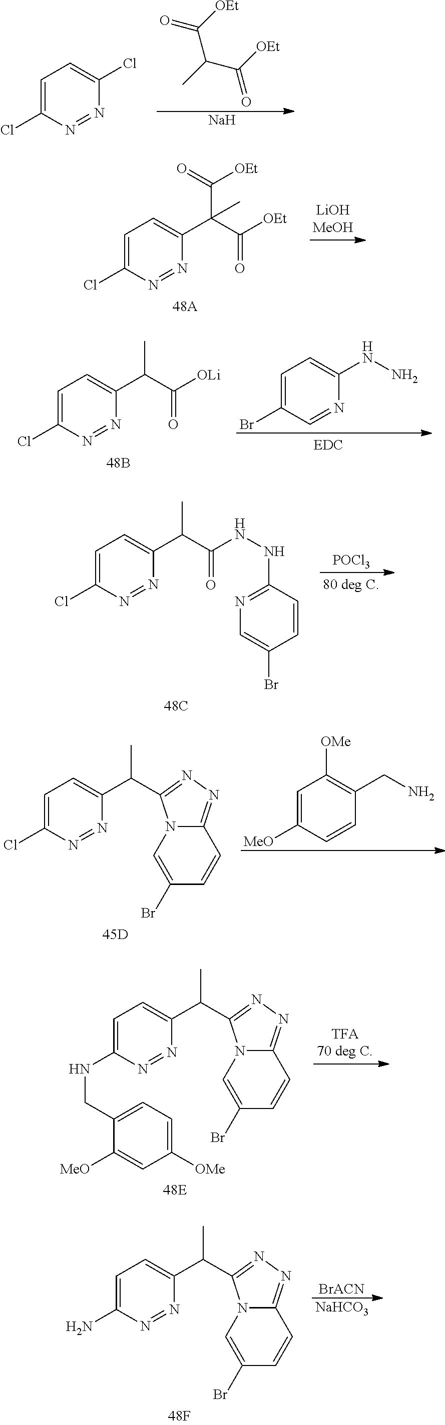

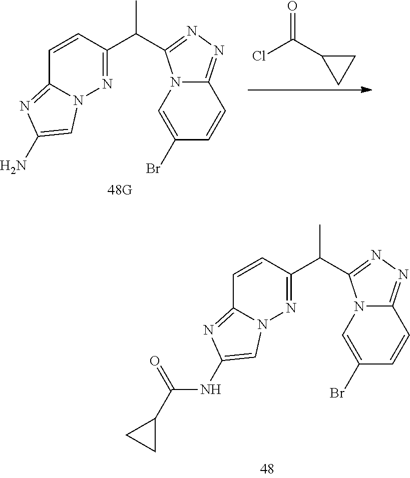

### Diethyl 2-(6-chloropyridazin-3-yl)-2-methylmalonate

To a mixture of NaH (13.42 g, 336 mmol) in dioxane (800 mL) was added diethyl 2-methylmalonate (42.9 ml, 252 mmol) drop-wise at 0° C. The reaction was stirred at 0° C. for 1 hr and allowed to warm to ambient temperature. 3,6-dichloropyridazine (25 g, 168 mmol) was then added portion-wise at 25° C. The reaction was then stirred at reflux for 1 hr. Solvent was removed via rotary evaporation and the resulting residue was dissolved in EtOAc. The organic solution was washed with saturated NaHCO₃ (100 mL) followed by 5% citric acid. The organic layer was then dried with MgSO₄, filtered, and concentrated to dryness. The resulting residue was purified via MPLC (Hex:EtOAc, 3:1) to provide the title compound, diethyl 2-(6-chloropyridazin-3-yl)-2-methylmalonate 48A (12 g, 25% yield) as a yellow oil. ¹H NMR (400 MHz, DMSO-d₆) δ ppm 1.18 (s, 6H) 1.81 (s, 3H) 4.21 (dd, J=7.07, 2.27 Hz, 4H) 8.00 (s, 2H). ESI-MS: m/z 287.1 (M+H)⁺.

### Lithium 2-(6-chloropyridazin-3-yl)propanoate

A solution of diethyl 2-(6-chloropyridazin-3-yl)-2-methylmalonate (19.8 g, 69.1 mmol) and LiOH (3.31 g, 138 mmol) in MeOH:Water (3:1, 200 mL) was stirred at 25° C. for 3 hrs. MeOH was removed from the reaction via rotary evaporation, and the resulting residue was reconstituted in H₂O (300 mL). The aqueous mixture was washed with Et₂O (3×100 mL) and then lyophilized to dryness to provide lithium 2-(6-chloropyridazin-3-yl)propanoate 48B in quantitative yield. The material was used without further work-up. ¹H NMR (400 MHz, DMSO-d₆) δ ppm 1.36 (3H, d, J=7.33 Hz) 3.63 (1H, q, J=7.24 Hz) 7.71 (2H, s). ESI-MS: m/z 187.0 (M+H)⁺.

### N′-(5-Bromopyridin-2-yl)-2-(6-chloropyridazin-3-yl)propanehydrazide

A mixture of lithium 2-(6-chloropyridazin-3-yl)propanoate (7.2 g, 37.4 mmol), 5-bromo-2-hydrazinylpyridine (7.03 g, 37.4 mmol), 1H-benzo[d][1,2,3]triazol-1-ol (5.05 g, 37.4 mmol), and N1-((ethylimino)methylene)-N3,N3-dimethylpropane-1,3-diamine hydrochloride (7.89 g, 41.1 mmol) in DMF (Volume: 320 ml) was stirred for 4 hrs at 25° C. The reaction was poured into H₂O (2 L) and extracted with EtOAc (3×500 mL). The organic phase was dried over MgSO₄, filtered, and evaporated to dryness. The resulting residue was purified by MPLC CHCl₃:MeOH (9:1) to provide the title compound, N′-(5-bromopyridin-2-yl)-2-(6-chloropyridazin-3-yl)propanehydrazide 48C (5.5 g, 15.42 mmol, 41.2% yield). ¹H NMR (400 MHz, DMSO-d₆) δ ppm 1.52 (d, J=7.33 Hz, 3H) 4.17 (d, J=7.07 Hz, 1H) 6.52 (d, J=8.84 Hz, 1H) 7.67 (dd, J=8.84, 2.53 Hz, 1H) 7.87 (d, J=9.09 Hz, 1H) 7.94 (d, J=8.84 Hz, 1H) 8.11 (d, J=1.77 Hz, 1H) 8.66 (d, J=1.52 Hz, 1H) 10.20 (d, J=1.52 Hz, 1H). ESI-MS: m/z 356.1 (M+H)⁺.

### 6-bromo-3-(1-(6-chloropyridazin-3-yl)ethyl)[1,2,4]triazolo[4,3-a]pyridine

A solution of N′-(5-bromopyridin-2-yl)-2-(6-chloropyridazin-3-yl)propanehydrazide (5.0 g, 14.02 mmol) and phosphoryl trichloride (100 mL, 1073 mmol) was stirred at 90° C. for 5 hrs. The reaction was cooled, stripped to dryness, and the resulting material was reconstituted in a mixture of EtOAc and saturated NaHCO₃ (400 mL, 1:1)). The organic phase was isolated, washed with saturated NaHCO₃ (1×100 mL), and dried over MgSO₄. The organic phase was removed via rotary evaporation to provide the title compound, 6-bromo-3-(1-(6-chloropyridazin-3-yl)ethyl)-[1,2,4]triazolo[4,3-a]pyridine 48D (2.2 g, 6.50 mmol, 46.3% yield). This material was used without further purification. ¹H NMR (400 MHz, DMSO-d₆) δ ppm 1.86 (d, J=7.07 Hz, 3H) 5.27 (d, J=7.33 Hz, 1H) 7.49 (dd, J=9.60, 1.77 Hz, 1H) 7.78 (dd, J=9.85, 1.01 Hz, 1H) 7.94 (s, 2H) 8.79 (s, 1H). ESI-MS: m/z 339.1 (M+H)⁺.

### N-(2,4-dimethoxybenzyl)-6-(1-(6-methyl-[1,2,4]triazolo[4,3-a]pyridin-3-yl)ethyl)pyridazin-3-amine

A mixture of 6-bromo-3-(1-(6-chloropyridazin-3-yl)ethyl)-[1,2,4]triazolo[4,3-a]pyridine (650 mg, 1.920 mmol), (2,4-dimethoxyphenyl)methanamine (0.577 mL, 3.84 mmol), and NaHCO₃ (645 mg, 7.68 mmol) in IPA (10.0 mL) was heated in a microwave on high absorbance for 18 hrs at 140° C. The reaction was cooled to ambient temperature, stripped to dryness via rotary evaporation, and reconstituted in EtOAc (25 mL). The insolubles were filtered off and the filtrate was reduced to dryness. The resulting residue was purified via MPLC (DCM:MeOH, 98:2) to provide the title compound, 6-(1-(6-bromo-[1,2,4]triazolo[4,3-a]pyridin-3-yl)ethyl)-N-(2,4-dimethoxybenzyl)pyridazin-3-amine 48E (750 mg, 1.598 mmol, 83% yield) as a yellow solid. ¹H NMR (400 MHz, DMSO-d6) δ ppm 1.77 (d, J=7.07 Hz, 3H) 3.72 (s, 3H) 4.37 (d, J=5.81 Hz, 2H) 4.95 (d, J=7.07 Hz, 1H) 6.44 (dd, J=8.34, 2.53 Hz, 1H) 6.54 (d, J=2.27 Hz, 1H) 6.85 (d, J=9.35 Hz, 1H) 7.03 (s, 1H) 7.12 (d, J=8.34 Hz, 1H) 7.29 (d, J=9.35 Hz, 1H) 7.45 (dd, J=9.73, 1.64 Hz, 1H) 7.69-7.79 (m, 1H) 8.59 (s, 1H). ESI-MS: m/z 469.2 (M+H)⁺.

### 6-(1-(6-bromo-[1,2,4]triazolo[4,3-a]pyridin-3-yl)ethyl)pyridazin-3-amine

A mixture of 6-(1-(6-bromo-[1,2,4]triazolo[4,3-a]pyridin-3-yl)ethyl)-N-(2,4-dimethoxybenzyl)pyridazin-3-amine (750 mg, 1.598 mmol), anisole (0.349 mL, 3.20 mmol), and TFA (1.0 mL, 12.98 mmol) in DCM (5.0 mL) was heated in a microwave on high absorbance for 2 hrs at 75° C. The reaction was stripped to dryness via rotary evaporation, and the resulting oil was treated with Et₂O to produce a solid. The solid was filtered and dried under vacuum to provide the title compound, 6-(1-(6-bromo-[1,2,4]triazolo[4,3-a]pyridin-3-yl)ethyl)pyridazin-3-amine 2,2,2-trifluoroacetate (400 mg, 0.923 mmol, 57.8% yield) as a yellow solid. ¹H NMR 48F (400 MHz, DMSO-d6) δ ppm 1.77 (d, J=7.07 Hz, 3H) 5.05 (d, J=7.07 Hz, 1H) 7.44 (d, J=9.60 Hz, 1H) 7.50 (dd, J=9.73, 1.64 Hz, 1H) 7.73-7.84 (m, 1H) 7.92 (d, J=9.60 Hz, 1H) 8.56 (br. s., 2H) 8.81 (s, 1H). ESI-MS: m/z 319.1 (M+H)⁺.

### 6-(1-(6-bromo-[1,2,4]triazolo[4,3-a]pyridin-3-yl)ethyl)imidazo[1,2-b]pyridazin-2-amine

A mixture of 6-(1-(6-bromo-[1,2,4]triazolo[4,3-a]pyridin-3-yl)ethyl)pyridazin-3-amine (1.0 g, 3.13 mmol), 2-bromoacetonitrile (0.251 ml, 3.76 mmol) and NaHCO₃ (0.053 g) in IPA (10 mL) was heated at 100° C. in a sealed tube for 2 hrs. The solid was filtered off, rinsed with IPA, and stripped to dryness via rotary evaporation. The resulting residue was purified by LCMS and lyophilized as a TFA salt to provide the title compound, 6-(1-(6-bromo-[1,2,4]triazolo[4,3-a]pyridin-3-yl)ethyl)imidazo[1,2-b]pyridazin-2-amine 48G (0.62 mg, 1.73 mmol, 55%). ¹H NMR (400 MHz, DMSO-d₆) δ ppm 1.85 (d, J=7.07 Hz, 3H) 5.17 (d, J=7.07 Hz, 1H) 7.38-7.57 (m, 3H) 7.80 (d, J=9.60 Hz, 1H) 7.99 (d, J=9.09 Hz, 1H) 8.78 (s, 1H). ESI-MS: m/z 358.1 (M+H)⁺.

### N-(6-(1-(6-bromo-[1,2,4]triazolo[4,3-a]pyridin-3-yl)ethyl)imidazo[1,2-b]pyridazin-2-yl)cyclopropanecarboxamide

To a solution of 6-(1-(6-bromo-[1,2,4]triazolo[4,3-a]pyridin-3-yl)ethyl)imidazo[1,2-b]pyridazin-2-amine TFA salt (250 mg, 0.52 mmol) in CH₂Cl₂ was added DIEA (0.65 g, 5.0 mmol) followed by cyclopropanecarbonyl chloride (0.16 g, 1.5 mmol). The reaction was stirred at ambient temperature for 30 min. Solvent was removed via rotary evaporation and the resulting residue was reconstituted in MeOH (10 mL). NH₄OH (0.5 mL) was added and the reaction was stirred at ambient temperature for 1 hr. The reaction was then concentrated to dryness and the resulting material was dissolved in EtOAc, washed with saturated NaHCO₃, dried with MgSO₄, filtered and concentrated to dryness. The crude material was purified via MPLC (5% MeOH/0.1% NH₄OH/EA) to provide the title compound, N-(6-(1-(6-bromo-[1,2,4]triazolo[4,3-a]pyridin-3-yl)ethyl)imidazo[1,2-b]pyridazin-2-yl)cyclopropanecarboxamide 48 (0.22 g, 0.52 mmol, 100%). ¹H NMR (400 MHz, DMSO-d₆) δ ppm 0.73-0.87 (m, 4H) 1.83-1.99 (m, 4H) 5.17 (d, J=7.07 Hz, 1H) 7.38-7.57 (m, 3H) 7.80 (d, J=9.60 Hz, 1H) 7.99 (d, J=9.09 Hz, 1H) 8.78 (s, 1H) 11.14 (s, 1H). ESI-MS: m/z 426.2 (M+H)⁺.

### Method B

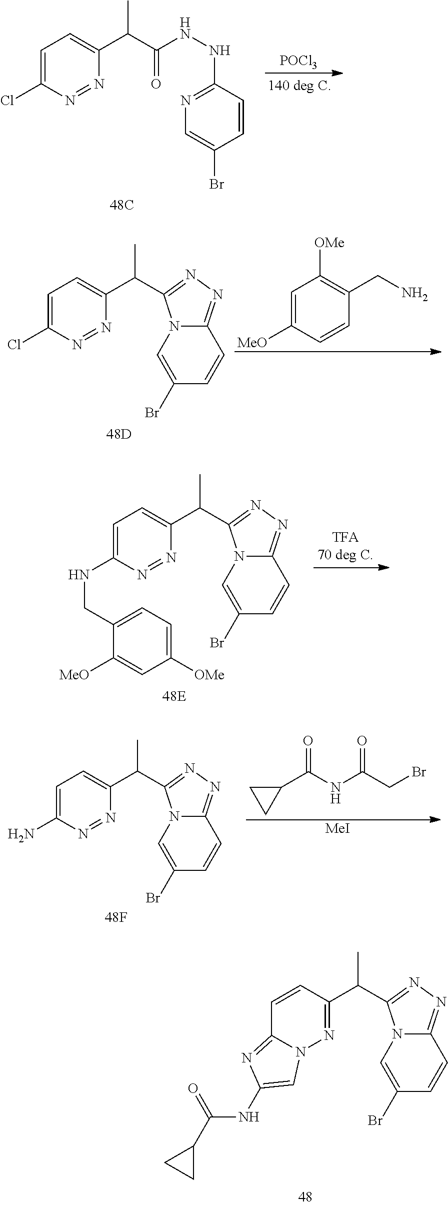

### 6-Bromo-3-(1-(6-chloropyridazin-3-yl)ethyl)-[1,2,4]triazolo[4,3-a]pyridine (48D)

A solution of N′-(5-bromopyridin-2-yl)-2-(6-chloropyridazin-3-yl)propanehydrazide (13 g, 36.5 mmol) in phosphoryl trichloride (84 ml, 901 mmol) was heated in the microwave at 140° C. for 15 min. The reaction was concentrated via rotary evaporation and the resulting mixture was added drop-wise to a mixture of EtOAc:Saturated Bicarbonate (aq) (4:6, 1 L). The organic phase was separated and the aqueous phase was extracted with EtOAc (3×75 mL). The EtOAc solutions were combined and evaporated to dryness. The resulting residue was purified by MPLC (DCM:MeOH, 97:3) to provide the title compound, 6-Bromo-3-(1-(6-chloropyridazin-3-yl)ethyl)-[1,2,4]triazolo[4,3-a]pyridine (48D, 3.3 g, 26.7%). ¹H NMR (400 MHz, DMSO-d₆) δ ppm 1.86 (d, J=7.07 Hz, 3H) 5.27 (d, J=7.33 Hz, 1H) 7.49 (dd, J=9.60, 1.77 Hz, 1H) 7.78 (dd, J=9.85, 1.01 Hz, 1H) 7.94 (s, 2H) 8.79 (s, 1H). ESI-MS: m/z 339.1 (M+H)⁺.

6-(1-(6-Bromo-[1,2,4]triazolo[4,3-a]pyridin-3-yl)ethyl)-N-(2,4-dimethoxybenzyl)pyridazin-3-amine (48E): The title compound was synthesized using an analogous procedure to that described in the preparation of Compound 48E from Method A.

6-(1-(6-Bromo-[1,2,4]triazolo[4,3-a]pyridin-3-yl)ethyl)pyridazin-3-amine (48F): The title compound was synthesized using an analogous procedure to that described in the preparation of Compound 48E from Method A.

N-(6-(1-(6-Bromo-[1,2,4]triazolo[4,3-a]pyridin-3-yl)ethyl)imidazo[1,2-b]pyridazin-2-yl)cyclopropanecarboxamide (48): The title compound was synthesized using an analogous procedure to that described in the preparation of Compound 45O.

### Compound 49: N-(6-(1-(6-(1-methyl-1H-pyrazol-4-yl)[1,2,4]triazolo[4,3-a]pyridin-3-yl)ethyl)imidazo[1,2-b]pyridazin-2-yl)cyclopropanecarboxamide

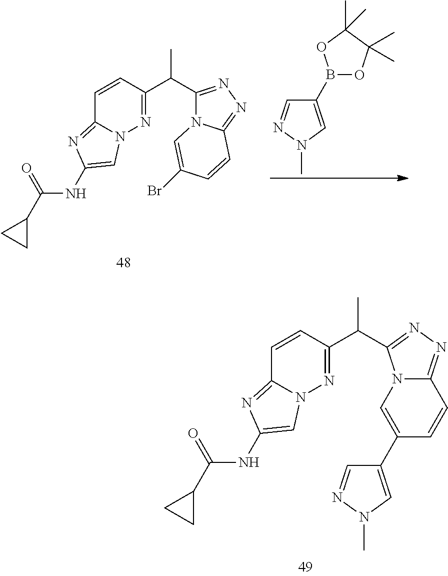 [SMILES: CC(c1ccc2nc(NC(=O)C3CC3)cn2n1)c1nnc2ccc(Br)cn12 ; Cn1cc(B2OC(C)(C)C(C)(C)O2)cn1 ; CC(c1ccc2nc(NC(=O)C3CC3)cn2n1)c1nnc2ccc(-c3cnn(C)c3)cn12]

A mixture of N-(6-(1-(6-bromo-[1,2,4]triazolo[4,3-a]-pyridin-3-yl)imidazol[1,2-b]pyridazin-2-yl)cyclopropanecarboxamide (0.23 g, 0.54 mmol), 1-methyl-4-(4,4,5,5-tetramethyl-1,3,2-dioxaborolan-2-yl)-1H-pyrazole (0.16 g, 0.8 mmol) and PdCl₂(dppf)-CH₂Cl₂ (0.02 g, 0.02 mmol) in Na₂CO₃ (2N, 1 mL)/dioxane (2 mL) was heated in microwave at 110° C. for 45 min. The reaction mixture was filtered, rinsed with EtOAc and concentrated to dryness via rotary evaporation. The resulting material was purified by preparative LCMS. The collected fractions were combined and the resulting mixture was treated with two drops of concentrated HCl. The solution was lyophilized to provide the HCl salt of the title compound, N-(6-(1-(6-(1-methyl-1H-pyrazol-4-yl)-[1,2,4]triazolo[4,3-a]pyridin-3-yl)ethyl)imidazo[1,2-b]pyridazin-2-yl)cyclopropanecarboxamide 49 (0.11 g, 0.25 mmol, 47%). ¹H NMR (400 MHz, DMSO-d₆) δ ppm 0.80 (d, J=6.32 Hz, 4H) 1.84-1.99 (m, 4H) 3.87 (s, 3H) 5.35 (d, J=7.07 Hz, 1H) 7.43 (d, J=9.35 Hz, 1H) 8.02 (d, J=9.35 Hz, 1H) 8.05-8.13 (m, 3H) 8.19 (dd, J=9.60, 1.52 Hz, 1H) 8.38 (s, 1H) 9.01 (s, 1H) 11.18 (s, 1H). MS: m/z 428.2 (M+H)⁺.

### Compound 50: (S)—N-(6-(1-(6-(1-methyl-1H-pyrazol-4-yl)-[1,2,4]triazolo[4,3-a]pyridin-3-yl)ethyl)imidazo[1,2-b]pyridazin-2-yl)cyclopropanecarboxamide

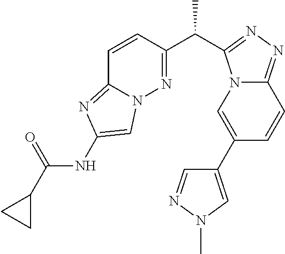 [SMILES: C[C@@H](c1ccc2nc(NC(=O)C3CC3)cn2n1)c1nnc2ccc(-c3cnn(C)c3)cn12]

Compound 50 was obtained from chiral separation of Compound 49 under the following conditions: An SFC/UV system was used with mobile phases of 25% EtOH (plus 10 mM NH₄OAc) in CO₂ on ChiralCel AS-H column (21×250 mm) at a flow rate of 50 mL/min with UV detection at 220 nm. ¹H NMR (400 MHz, DMSO-d₆) δ ppm 0.73-0.88 (m, 3H) 1.81-1.99 (m, 4H) 3.87 (s, 3H) 5.08-5.21 (m, 1H) 7.27 (d, J=9.35 Hz, 1H) 7.63 (dd, J=9.60, 1.52 Hz, 1H) 7.80 (dd, J=9.60, 1.01 Hz, 1H) 7.91-8.00 (m, 2H) 8.12 (s, 1H) 8.23 (s, 1H) 8.62 (s, 1H) 11.13 (s, 1H). MS: m/z 428.2 (M+H)⁺.

### Compound 51: (R)—N-(6-(1-(6-(1-methyl-1H-pyrazol-4-yl)-[1,2,4]triazolo[4,3-a]pyridin-3-yl)ethyl)imidazo[1,2-b]pyridazin-2-yl)cyclopropanecarboxamide

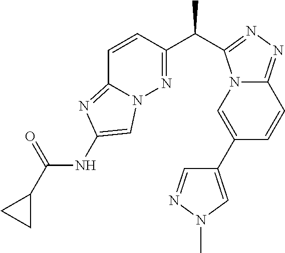 [SMILES: C[C@H](c1ccc2nc(NC(=O)C3CC3)cn2n1)c1nnc2ccc(-c3cnn(C)c3)cn12]

Compound 51 was obtained from chiral separation of Compound 49 under the following conditions: An SFC/UV system was used with mobile phases of 25% EtOH (plus 10 mM NH₄OAc) in CO₂ on ChiralCel AS-H column (21×250 mm) at a flow rate of 50 mL/min with UV detection at 220 nm. ¹H NMR (400 MHz, DMSO-d₆) δ ppm 0.73-0.88 (m, 3H) 1.81-1.99 (m, 4H) 3.87 (s, 3H) 5.08-5.21 (m, 1H) 7.27 (d, J=9.35 Hz, 1 H) 7.63 (dd, J=9.60, 1.52 Hz, 1H) 7.80 (dd, J=9.60, 1.01 Hz, 1H) 7.91-8.00 (m, 2H) 8.12 (s, 1H) 8.23 (s, 1H) 8.62 (s, 1H) 11.13 (s, 1H). MS: m/z 428.2 (M+H)⁺.

### Compound 52: N-(6-(1-([1,2,4]triazolo[4,3-a]pyridin-3-yl)ethyl)imidazo[1,2-b]pyridazin-2-yl)cyclopropanecarboxamide

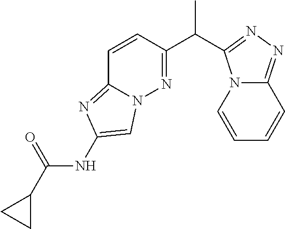 [SMILES: CC(c1ccc2nc(NC(=O)C3CC3)cn2n1)c1nnc2ccccn12]

The title compound was synthesized using an analogous procedure to that described in the preparation of Compound 48 (Method A) using 2-hydrazinylpyridine. ¹H NMR (400 MHz, DMSO-d₆) δ ppm 0.73-0.87 (m, 4H) 1.83-1.99 (m, 4H) 5.04-5.15 (m, 1H) 6.94-7.04 (m, 1H) 7.19 (d, J=9.35 Hz, 1H) 7.38-7.47 (m, 1H) 7.80 (d, J=9.35 Hz, 1H) 7.94 (d, J=9.85 Hz, 1H) 8.10 (s, 1H) 8.35 (d, J=7.07 Hz, 1H) 11.14 (s, 1H). MS: m/z 348.2 (M+H)⁺.

### Compound 53: N-(6-(1-(6-methyl-[1,2,4]triazolo[4,3-a]pyridin-3-yl)ethyl)imidazo[1,2-b]pyridazin-2-yl)cyclopropanecarboxamide

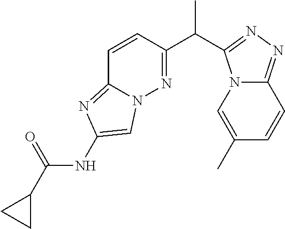 [SMILES: Cc1ccc2nnc(C(C)c3ccc4nc(NC(=O)C5CC5)cn4n3)n2c1]

The title compound was synthesized using an analogous procedure to that described in the preparation of Compound 48 (Method A) using 2-hydrazinyl-5-methylpyridine. ¹H NMR (400 MHz, DMSO-d₆) δ ppm 0.76-0.86 (m, 4H) 1.85 (d, J=7.07 Hz, 3H) 1.92 (m, 1H) 3.56 (s, 3H) 5.02 (d, J=7.07 Hz, 1H) 7.18 (d, J=9.35 Hz, 1H) 7.23-7.31 (m, 1H) 7.70 (d, J=8.84 Hz, 1H) 7.93 (d, J=9.85 Hz, 1H) 8.10 (s, 1H) 8.20 (s, 1H) 11.13 (s, 1H). MS: m/z 362.2 (M+H)⁺.

### Compound 54: (6-(2-([1,2,4]Triazolo[4,3-a]pyridin-3-yl)propan-2-yl)imidazo[1,2-b]pyridazin-2-yl)(cyclopropyl)methanone

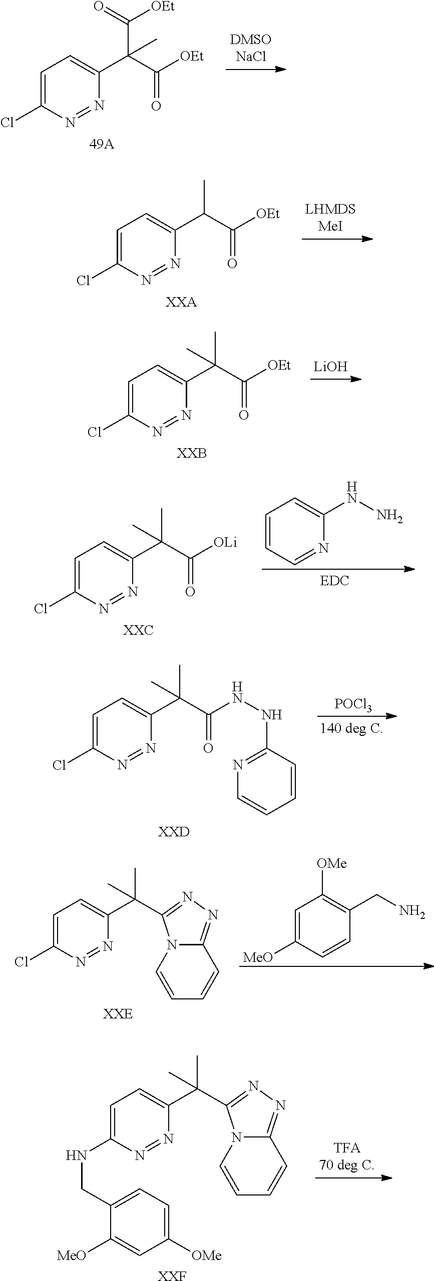

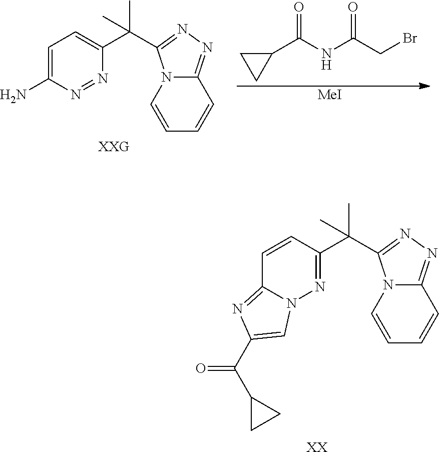

### Ethyl 2-(6-chloropyridazin-3-yl)propanoate (54A)

A solution of diethyl 2-(6-chloropyridazin-3-yl)-2-methylmalonate (11 g, 38.4 mmol) and NaCl (2.69 g, 46.0 mmol) in DMSO (91 mL) and H₂O (1.382 ml) was divided into eight separate microwave vials and each reaction was heated in a microwave at 175° C. for 90 mins on high absorbance. The reactions were combined and then poured into H₂O (300 mL). The aqueous phase was extracted with EtOAc (2×100 mL) and then the organic phase was washed with brine (2×100 mL). The organic phase was dried over MgSO₄, filtered and concentrated to dryness via rotary evaporation. The resulting oil was purified via MPLC (3:1, Hex:EtOAc) to provide the title compound, ethyl 2-(6-chloropyridazin-3-yl)propanoate (54A, 2.9 g, 35%). ₁H NMR (400 MHz, DMSO-d₆) δ ppm 1.13 (s, 3H) 1.49 (d, J=7.33 Hz, 3H) 4.09 (dd, J=7.07, 2.02 Hz, 2H) 4.23 (d, J=7.33 Hz, 1H) 7.83 (d, J=8.84 Hz, 1H) 7.93 (d, J=8.84 Hz, 1H). ESI-MS: m/z 215.0 (M+H)⁺.

### Ethyl 2-(6-chloropyridazin-3-yl)-2-methylpropanoate (54B)

To a solution of ethyl 2-(6-chloropyridazin-3-yl)propanoate (2.8 g, 13.04 mmol) in THF (28 mL) was added lithium hexamethlydisilazide (1.0 molar in THF, 15.65 mL, 15.65 mmol) at −70° C. in dropwise fashion. The reaction was stirred at −70 for 30 min and then iodomethane (0.816 mL, 13.04 mmol) was added. The reaction was stirred for 2 hrs as allowed to warm to room temperature. The reaction was concentrated to dryness via rotary evaporation and the resulting material was purified by MPLC (98:2, DCM:MeOH) to provide the title compound, ethyl 2-(6-chloropyridazin-3-yl)-2-methylpropanoate (54B, 2.82 g, 95%). ¹H NMR (400 MHz, DMSO-d6) δ ppm 1.11 (t, J=7.07 Hz, 3H) 1.58 (s, 6H) 4.08 (q, J=7.07 Hz, 2H) 7.83-7.97 (m, 2H).

### Lithium 2-(6-chloropyridazin-3-yl)-2-methylpropanoate (54C)

The title compound was synthesized using an analogous procedure to that described in the preparation of Compound 48B.

### 2-(6-Chloropyridazin-3-yl)-2-methyl-N′-(pyridin-2-yl)propanehydrazide (54D)

The title compound was synthesized using an analogous procedure to that described in the preparation of Compound 48C using 2-hydrazinylpyridine.

### 3-(2-(6-chloropyridazin-3-yl)propan-2-yl)-[1,2,4]triazolo[4,3-a]pyridine (54E)

A mixture of 2-(6-chloropyridazin-3-yl)-N′-(pyridin-2-yl)propanehydrazide (1.2 g, 4.32 mmol) and phosphoryl trichloride (10.0 mL, 107 mmol) was heated in a microwave on high absorbance for 15 min at 140° C. The resulting reaction was stripped to dryness via rotary evaporation. The resulting residue was reconstituted in EtOAc (50 mL) and washed with saturated bicarbonate (3×50 mL). The organic layer was separated and dried over MgSO₄ and the EtOAc was removed via rotary evaporation to provide the title compound, 3-(2-((6-chloropyridazin-3-yl)propan-2-yl)-[1,2,4]triazolo[4,3-a]pyridine (54E, 0.750 g, 2.74 mmol, 63.4% yield). ₁H NMR (400 MHz, DMSO-d₆) δ ppm 1.94 (6H, s) 6.75-6.82 (1H, m) 7.33 (1H, ddd, J=9.35, 6.57, 1.01 Hz) 7.72-7.82 (2H, m) 7.87-7.99 (2H, m). ESI-MS: m/z 274.1 (M+H)⁺.

### 6-(2-([1,2,4]Triazolo[4,3-a]pyridin-3-yl)propan-2-yl)-N-(2,4-dimethoxybenzyl)pyridazin-3-amine (54F)

The title compound was synthesized using an analogous procedure to that described in the preparation of Compound 48E.

### 6-(2-([1,2,4]Triazolo[4,3-a]pyridin-3-yl)propan-2-yl)pyridazin-3-amine (54G)

The title compound was synthesized using an analogous procedure to that described in the preparation of Compound 48F.

### (6-(2-([1,2,4]Triazolo[4,3-a]pyridin-3-yl)propan-2-yl)imidazo[1,2-b]pyridazin-2-yl)(cyclopropyl)methanone (54)

A mixture of 6-(2-([1,2,4]triazolo[4,3-a]pyridin-3-yl)propan-2-yl)pyridazin-3-amine 2,2,2-trifluoroacetate (50 mg, 0.136 mmol), N-(2-bromoacetyl)cyclopropanecarboxamide (28.0 mg, 0.136 mmol), sodium hydrogenphosphate (57.8 mg, 0.407 mmol), and potassium iodide (22.54 mg, 0.136 mmol) in DMA (Volume: 1160 μA) was heated in a microwave on high absorbance for 1 hr at 100° C. The reaction was purified by preparative LCMS to provide the title compound, (6-(2-([1,2,4]triazolo[4,3-a]pyridin-3-yl)propan-2-yl)imidazo[1,2-b]pyridazin-2-yl)(cyclopropyl)methanone (54, 13 mg, 26%) as a TFA salt. ¹H NMR (400 MHz, DMSO-d₆) δ ppm 0.81 (d, J=6.06 Hz, 4H) 1.87-2.00 (m, 7H) 6.92-6.97 (m, 1H) 7.12 (d, J=9.60 Hz, 1H) 7.55 (dd, J=8.97, 6.69 Hz, 1H) 7.87-8.01 (m, 3H) 8.04 (s, 1H) 11.16 (s, 1H). ESI-MS: m/z 362.1 (M+H)⁺.

### Compound 55: N-(6-(1-(6-(4-fluorophenyl)-[1,2,4]triazolo[4,3-a]pyridin-3-yl)ethyl)imidazo[1,2-b]pyridazin-2-yl)cyclopropanecarboxamide

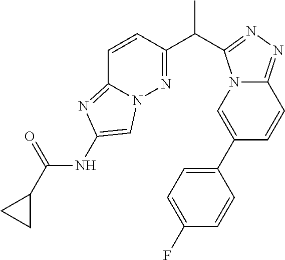 [SMILES: CC(c1ccc2nc(NC(=O)C3CC3)cn2n1)c1nnc2ccc(-c3ccc(F)cc3)cn12]

The title compound was synthesized using an analogous procedure to that described in the preparation of Compound 49 using 4-fluorophenylboronic acid. The crude reaction mixture was purified by preparative LCMS, and the product was isolated as the TFA salt. ¹H NMR (400 MHz, DMSO-d6) δ ppm 0.72-0.88 (m, 4H) 1.81-1.98 (m, 4H) 5.30 (d, J=7.07 Hz, 1H) 7.30 (d, J=9.35 Hz, 1H) 7.32-7.40 (m, 2H) 7.76-7.82 (m, 2H) 7.82-7.88 (m, 1H) 7.95 (t, J=9.09 Hz, 2H) 8.09 (s, 1H) 8.78 (s, 1H) 11.14 (s, 1H). ESI-MS: m/z 442.2 (M+H)⁺.

### Compound 56: N-(6-(1-(6-(4-methoxyphenyl)-[1,2,4]triazolo[4,3-a]pyridin-3-yl)ethyl)imidazo[1,2-b]pyridazin-2-yl)cyclopropanecarboxamide

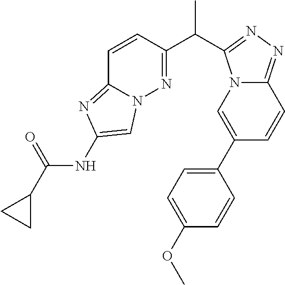 [SMILES: COc1ccc(-c2ccc3nnc(C(C)c4ccc5nc(NC(=O)C6CC6)cn5n4)n3c2)cc1]

The title compound was synthesized using an analogous procedure to that described in the preparation of Compound 49 using 4-methoxyphenylboronic acid. The crude reaction mixture was purified by preparative LCMS, and the product was isolated as the TFA salt. ¹H NMR (400 MHz, DMSO-d6) δ ppm 0.73-0.87 (m, 5H) 1.84-2.00 (m, 5H) 5.30 (d, J=7.33 Hz, 1H) 7.02-7.14 (m, 3H) 7.29 (d, J=9.35 Hz, 1H) 7.61-7.72 (m, 3H) 7.83-8.00 (m, 4H) 8.10 (s, 1H) 8.71 (s, 1H) 11.15 (s, 1H). ESI-MS: m/z 454.2 (M+H)⁺.

### Compound 57: N-(6-(1-(6-(3-cyanophenyl)-[1,2,4]triazolo[4,3-a]pyridin-3-yl)ethyl)imidazo[1,2-b]pyridazin-2-yl)cyclopropanecarboxamide

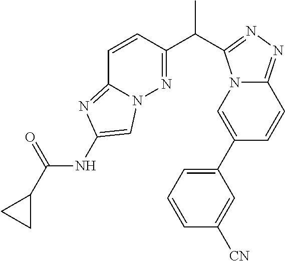 [SMILES: CC(c1ccc2nc(NC(=O)C3CC3)cn2n1)c1nnc2ccc(-c3cccc(C#N)c3)cn12]

The title compound was synthesized using an analogous procedure to that described in the preparation of Compound 49 using 3-cyanophenylboronic acid. The crude reaction mixture was purified by preparative LCMS, and the product was isolated as the TFA salt. ¹H NMR (400 MHz, DMSO-d6) δ ppm 0.71-0.86 (m, 4H) 1.82-1.97 (m, 4H) 5.24-5.37 (m, 1H) 7.31 (d, J=9.60 Hz, 1H) 7.69-7.75 (m, 1H) 7.87-7.99 (m, 4H) 8.05-8.13 (m, 2H) 8.31 (t, J=1.52 Hz, 1H) 8.94 (s, 1H) 11.12 (s, 1H). ESI-MS: m/z 449.2 (M+H)⁺.

### Compound 58: 4-(3-(1-(2-(cyclopropanecarboxamido)imidazo[1,2-b]pyridazin-6-yl)ethyl)-[1,2,4]triazolo[4,3-a]pyridin-6-yl)-2-fluoro-N-methylbenzamide

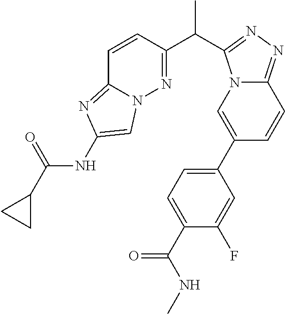 [SMILES: CNC(=O)c1ccc(-c2ccc3nnc(C(C)c4ccc5nc(NC(=O)C6CC6)cn5n4)n3c2)cc1F]

The title compound was synthesized using an analogous procedure to that described in the preparation of Compound 49 using 3-fluoro-4-(methylcarbamoyl)phenylboronic acid. The crude reaction mixture was purified by preparative LCMS, and the product was isolated as the TFA salt. ¹H NMR (400 MHz, DMSO-d6) δ ppm 0.72-0.86 (m, 4H) 1.84-1.98 (m, 4H) 2.80 (d, J=4.55 Hz, 3H) 5.31 (d, J=7.07 Hz, 1H) 7.30 (d, J=9.35 Hz, 1H) 7.66-7.71 (m, 1H) 7.71-7.81 (m, 2H) 7.83-7.88 (m, 1H) 7.89-7.98 (m, 2H) 8.09 (s, 1H) 8.30 (dd, J=4.55, 2.53 Hz, 1H) 8.89 (s, 1H) 11.13 (s, 1H). ESI-MS: m/z 499.2 (M+H)⁺.

### Compound 59: N-(6-(1-(6-(3-(methylsulfonyl)phenyl)-[1,2,4]triazolo[4,3-a]pyridin-3-yl)ethyl)imidazo[1,2-b]pyridazin-2-yl)cyclopropanecarboxamide

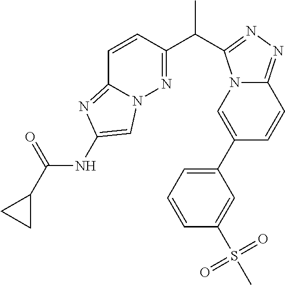 [SMILES: CC(c1ccc2nc(NC(=O)C3CC3)cn2n1)c1nnc2ccc(-c3cccc(S(C)(=O)=O)c3)cn12]

The title compound was synthesized using an analogous procedure to that described in the preparation of Compound 49 using 3-(methylsulfonyl)phenylboronic acid. The crude reaction mixture was purified by preparative LCMS, and the product was isolated as the TFA salt. ¹H NMR (400 MHz, DMSO-d6) δ ppm 0.74-0.85 (m, 5H) 1.83-1.98 (m, 5H) 5.34 (d, J=7.07 Hz, 1H) 7.32 (d, J=9.35 Hz, 1H) 7.73-7.85 (m, 1H) 7.89-8.03 (m, 5H) 8.05-8.14 (m, 2H) 8.27 (t, J=1.64 Hz, 1H) 8.93 (s, 1H) 11.13 (s, 1H). ESI-MS: m/z 502.2 (M+H)⁺.

### Compound 60: N-(6-(1-(6-(4-(methylsulfonyl)phenyl)-[1,2,4]triazolo[4,3-a]pyridin-3-yl)ethyl)imidazo[1,2-b]pyridazin-2-yl)cyclopropanecarboxamide

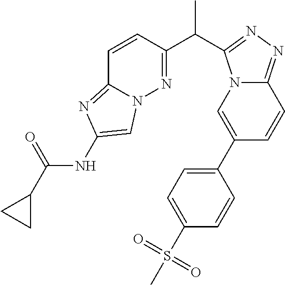 [SMILES: CC(c1ccc2nc(NC(=O)C3CC3)cn2n1)c1nnc2ccc(-c3ccc(S(C)(=O)=O)cc3)cn12]

The title compound was synthesized using an analogous procedure to that described in the preparation of Compound 49 using 4-(methylsulfonyl)phenylboronic acid. The crude reaction mixture was purified by preparative LCMS, and the product was isolated as the TFA salt. ¹H NMR (400 MHz, DMSO-d6) δ ppm 0.73-0.87 (m, 5H) 1.83-1.98 (m, 5H) 5.31 (d, J=7.07 Hz, 1H) 7.30 (d, J=9.35 Hz, 1H) 7.85-7.90 (m, 1H) 7.93-7.99 (m, 2H) 7.99-8.07 (m, 5H) 8.09 (s, 1H) 8.90 (s, 1H) 11.13 (s, 1H). ESI-MS: m/z 502.2 (M+H)⁺.

### Compound 61: N-(6-(1-(6-(3-methoxyphenyl)-[1,2,4]triazolo[4,3-a]pyridin-3-yl)ethyl)imidazo[1,2-b]pyridazin-2-yl)cyclopropanecarboxamide

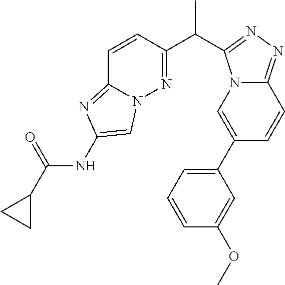 [SMILES: COc1cccc(-c2ccc3nnc(C(C)c4ccc5nc(NC(=O)C6CC6)cn5n4)n3c2)c1]

The title compound was synthesized using an analogous procedure to that described in the preparation of Compound 49 using 3-methoxyphenylboronic acid. The crude reaction mixture was purified by preparative LCMS, and the product was isolated as the TFA salt. ¹H NMR (400 MHz, DMSO-d6) δ ppm 0.74-0.85 (m, 4H) 1.90 (d, J=7.33 Hz, 4H) 3.61 (s, 3H) 5.32 (d, J=7.07 Hz, 1H) 7.02 (ddd, J=8.21, 2.40, 0.76 Hz, 1H) 7.21-7.34 (m, 3H) 7.37-7.49 (m, 1H) 7.87-8.00 (m, 3H) 8.09 (s, 1H) 8.77 (s, 1H) 11.14 (s, 1H). ESI-MS: m/z 454.2 (M+H)⁺.

### Compound 62: N-(6-(1-(6-(4-methylthiophen-2-yl)-[1,2,4]triazolo[4,3-a]pyridin-3-yl)ethyl)imidazo[1,2-b]pyridazin-2-yl)cyclopropanecarboxamide

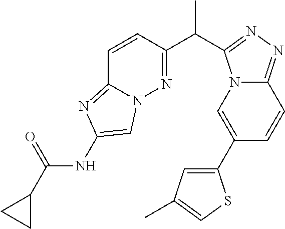 [SMILES: Cc1csc(-c2ccc3nnc(C(C)c4ccc5nc(NC(=O)C6CC6)cn5n4)n3c2)c1]

The title compound was synthesized using an analogous procedure to that described in the preparation of Compound 49 using 4-methylthiophen-2-ylboronic acid. The crude reaction mixture was purified by preparative LCMS, and the product was isolated as the TFA salt. ¹H NMR (400 MHz, DMSO-d6) δ ppm 0.75-0.87 (m, 4H) 1.82-1.98 (m, 4H) 2.24 (d, J=0.76 Hz, 3H) 5.24 (d, J=7.33 Hz, 1H) 7.17-7.30 (m, 2H) 7.45 (d, J=1.26 Hz, 1H) 7.71 (dd, J=9.60, 1.52 Hz, 1H) 7.86 (dd, J=9.47, 0.88 Hz, 1H) 7.95 (d, J=9.60 Hz, 1H) 8.13 (s, 1H) 8.64 (s, 1H) 11.15 (s, 1H). ESI-MS: m/z 444.2 (M+H)⁺.

### Compound 63: N-(6-(1-(6-(4-cyanophenyl)-[1,2,4]triazolo[4,3-a]pyridin-3-yl)ethyl)imidazo[1,2-b]pyridazin-2-yl)cyclopropanecarboxamide

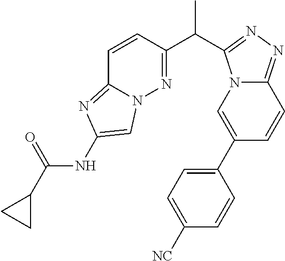 [SMILES: CC(c1ccc2nc(NC(=O)C3CC3)cn2n1)c1nnc2ccc(-c3ccc(C#N)cc3)cn12]

The title compound was synthesized using an analogous procedure to that described in the preparation of Compound 49 using 4-cyanophenylboronic acid. The crude reaction mixture was purified by preparative LCMS, and the product was isolated as the TFA salt. ¹H NMR (400 MHz, DMSO-d6) δ ppm 0.73-0.87 (m, 4H) 1.83-2.00 (m, 4H) 5.29 (d, J=7.07 Hz, 1H) 7.29 (d, J=9.35 Hz, 1H) 7.80-7.89 (m, 1H) 7.90-8.04 (m, 6H) 8.08 (s, 1H) 8.90 (s, 1H) 11.13 (s, 1H). ESI-MS: m/z 449.2 (M+H)⁺.

### Compound 64: N-(6-(1-(6-(1H-pyrazol-4-yl)-[1,2,4]triazolo[4,3-a]pyridin-3-yl)ethyl)imidazo[1,2-b]pyridazin-2-yl)cyclopropanecarboxamide

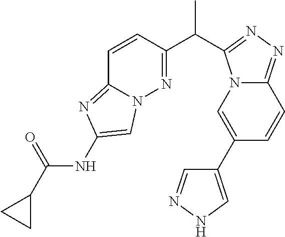 [SMILES: CC(c1ccc2nc(NC(=O)C3CC3)cn2n1)c1nnc2ccc(-c3cn[nH]c3)cn12]

The title compound was synthesized using an analogous procedure to that described in the preparation of Compound 49 using 1H-pyrazol-4-ylboronic acid. The crude reaction mixture was purified by preparative LCMS, and the product was isolated as the TFA salt. ¹H NMR (400 MHz, DMSO-d6) δ ppm 0.73-0.86 (m, 4H) 1.82-1.99 (m, 4H) 5.21 (d, J=7.33 Hz, 1H) 7.31 (d, J=9.60 Hz, 1H) 7.88 (d, J=6.06 Hz, 2H) 7.97 (d, J=9.35 Hz, 1H) 8.11 (s, 1H) 8.19 (s, 2H) 8.75 (s, 1H) 11.13 (s, 1H). ESI-MS: m/z 414.2 (M+H)⁺.

### Compound 65: N-(6-(1-(6-(3-fluorophenyl)-[1,2,4]triazolo[4,3-a]pyridin-3-yl)ethyl)imidazo[1,2-b]pyridazin-2-yl)cyclopropanecarboxamide

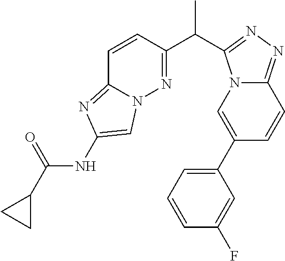 [SMILES: CC(c1ccc2nc(NC(=O)C3CC3)cn2n1)c1nnc2ccc(-c3cccc(F)c3)cn12]

The title compound was synthesized using an analogous procedure to that described in the preparation of Compound 49 using 4-fluorophenylboronic acid. The crude reaction mixture was purified by preparative LCMS, and the product was isolated as the TFA salt. ¹H NMR (400 MHz, DMSO-d6) δ ppm 0.72-0.87 (m, 4H) 1.83-1.97 (m, 4H) 5.29 (d, J=7.33 Hz, 1H) 7.51-7.61 (m, 2H) 7.64 (dt, J=10.29, 2.05 Hz, 1H) 7.81 (dd, J=9.73, 1.64 Hz, 1H) 7.87-7.92 (m, 1H) 7.94 (d, J=9.35 Hz, 1H) 8.09 (s, 1H) 8.80 (s, 1H) 11.08 (s, 1H). ESI-MS: m/z 442.2 (M+H)⁺.

### Compound 66: 3-(3-(1-(2-(cyclopropanecarboxamido)imidazo[1,2-b]pyridazin-6-yl)ethyl)-[1,2,4]triazolo[4,3-a]pyridin-6-yl)benzamide

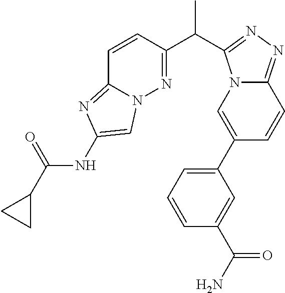 [SMILES: CC(c1ccc2nc(NC(=O)C3CC3)cn2n1)c1nnc2ccc(-c3cccc(C(N)=O)c3)cn12]

The title compound was synthesized using an analogous procedure to that described in the preparation of Compound 49 using 3-carbamoylphenylboronic acid. The crude reaction mixture was purified by preparative LCMS, and the product was isolated as the TFA salt. ¹H NMR (400 MHz, DMSO-d6) □ppm 0.71-0.89 (m, 4H) 1.83-2.00 (m, 4H) 7.31 (d, J=9.35 Hz, 1H) 7.46 (br. s., 1H) 7.60 (t, J=7.71 Hz, 1H) 7.82-8.03 (m, 5H) 8.04-8.14 (m, 2H) 8.22 (t, J=1.52 Hz, 1H) 8.86 (s, 1H) 11.09 (s, 1H). ESI-MS: m/z 467.2 (M+H)⁺.

### Compound 67: 4-(3-(1-(2-(cyclopropanecarboxamido)imidazo[1,2-b]pyridazin-6-yl)ethyl)-[1,2,4]triazolo[4,3-a]pyridin-6-yl)benzamide

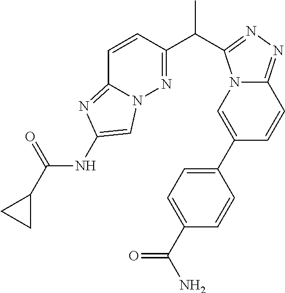 [SMILES: CC(c1ccc2nc(NC(=O)C3CC3)cn2n1)c1nnc2ccc(-c3ccc(C(N)=O)cc3)cn12]

The title compound was synthesized using an analogous procedure to that described in the preparation of Compound 49 using 4-carbamoylphenylboronic acid. The crude reaction mixture was purified by preparative LCMS, and the product was isolated as the TFA salt. ¹H NMR (400 MHz, DMSO-d6) δ ppm 0.73-0.89 (m, 4H) 1.83-1.99 (m, 4H) 5.29 (d, J=7.33 Hz, 1H) 7.28 (d, J=9.35 Hz, 1H) 7.77-7.86 (m, 3H) 7.87-7.92 (m, 1H) 7.94 (d, J=9.35 Hz, 1H) 7.97-8.06 (m, 3H) 8.10 (s, 1H) 8.79 (s, 1H) 11.08 (s, 1H). ESI-MS: m/z 467.2 (M+H)⁺.

### Compound 68: 4-(3-(1-(2-(cyclopropanecarboxamido)imidazo[1,2-b]pyridazin-6-yl)ethyl)-[1,2,4]triazolo[4,3-a]pyridin-6-yl)-N-methylbenzamide

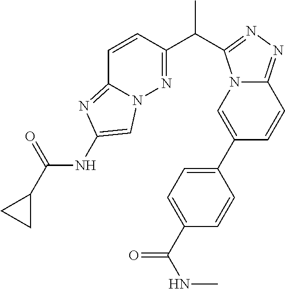 [SMILES: CNC(=O)c1ccc(-c2ccc3nnc(C(C)c4ccc5nc(NC(=O)C6CC6)cn5n4)n3c2)cc1]

The title compound was synthesized using an analogous procedure to that described in the preparation of Compound 49 using 4-(methylcarbamoyl)phenylboronic acid. The crude reaction mixture was purified by preparative LCMS, and the product was isolated as the TFA salt. ¹H NMR (400 MHz, DMSO-d6) δ ppm 0.72-0.88 (m, 4H) 1.83-1.99 (m, 4H) 2.81 (d, J=4.55 Hz, 3H) 5.30 (d, J=7.07 Hz, 1H) 7.28 (d, J=9.35 Hz, 1H) 7.78-7.87 (m, 3H) 7.87-8.00 (m, 4H) 8.10 (s, 1H) 8.49 (d, J=4.55 Hz, 1H) 8.81 (s, 1H) 11.09 (s, 1H). ESI-MS: m/z 481.2 (M+H)⁺.

### Compound 69: N-(6-(1-(6-(4-ethoxyphenyl)-[1,2,4]triazolo[4,3-a]pyridin-3-yl)ethyl)imidazo[1,2-b]pyridazin-2-yl)cyclopropanecarboxamide

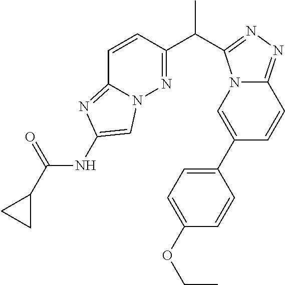 [SMILES: CCOc1ccc(-c2ccc3nnc(C(C)c4ccc5nc(NC(=O)C6CC6)cn5n4)n3c2)cc1]

The title compound was synthesized using an analogous procedure to that described in the preparation of Compound 49 using 4-ethoxyphenylboronic acid. The crude reaction mixture was purified by preparative LCMS, and the product was isolated as the TFA salt. ¹H NMR (400 MHz, DMSO-d6) δ ppm 0.72-0.87 (m, 4H) 1.34 (t, J=6.95 Hz, 3H) 1.82-1.98 (m, 4H) 5.27 (d, J=7.07 Hz, 1H) 6.99-7.10 (m, 2H) 7.26 (d, J=9.35 Hz, 1H) 7.57-7.69 (m, 2H) 7.74-7.83 (m, 1H) 7.84-7.91 (m, 1H) 7.94 (d, J=9.60 Hz, 1H) 8.10 (s, 1H) 8.64 (s, 1H) 11.09 (s, 1H). ESI-MS: m/z 468.2 (M+H)⁺.

### Compound 70: N-(6-(1-(6-(4-methoxy-3-methylphenyl)-[1,2,4]triazolo[4,3-a]pyridin-3-yl)ethyl)imidazo[1,2-b]pyridazin-2-yl)cyclopropanecarboxamide

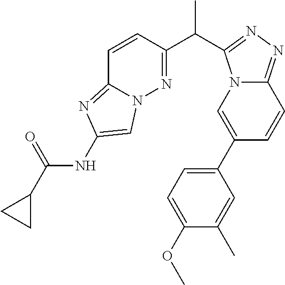 [SMILES: COc1ccc(-c2ccc3nnc(C(C)c4ccc5nc(NC(=O)C6CC6)cn5n4)n3c2)cc1C]

The title compound was synthesized using an analogous procedure to that described in the preparation of Compound 49 using 4-methoxy-3-methylphenylboronic acid. The crude reaction mixture was purified by preparative LCMS, and the product was isolated as the TFA salt. ¹H NMR (400 MHz, DMSO-d6) δ ppm 0.74-0.86 (m, 5H) 1.85-1.99 (m, 5H) 2.21 (s, 4H) 5.29 (d, J=7.33 Hz, 2H) 7.06 (d, J=8.59 Hz, 1H) 7.28 (d, J=9.35 Hz, 1H) 7.42-7.57 (m, 2H) 7.78-7.99 (m, 4H) 8.12 (s, 1H) 8.65 (s, 1H) 11.10 (s, 1H). ESI-MS: m/z 468.2 (M+H)⁺.

### Compound 71: N-(6-(1-(6-(3-cyano-4-fluorophenyl)-[1,2,4]triazolo[4,3-a]pyridin-3-yl)ethyl)imidazo[1,2-b]pyridazin-2-yl)cyclopropanecarboxamide

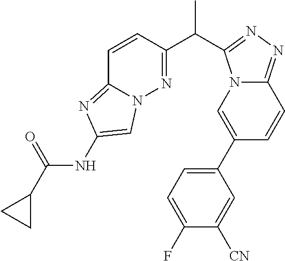 [SMILES: CC(c1ccc2nc(NC(=O)C3CC3)cn2n1)c1nnc2ccc(-c3ccc(F)c(C#N)c3)cn12]

The title compound was synthesized using an analogous procedure to that described in the preparation of Compound 49 using 3-cyano-4-fluorophenylboronic acid. The crude reaction mixture was purified by preparative LCMS, and the product was isolated as the TFA salt. ¹H NMR (400 MHz, DMSO-d6) δ ppm 0.73-0.86 (m, 4H) 1.84-1.99 (m, 5H) 5.26 (d, J=7.33 Hz, 1H) 7.29 (d, J=9.60 Hz, 1H) 7.68 (t, J=8.97 Hz, 1H) 7.75-7.84 (m, 1H) 7.87-7.98 (m, 2H) 8.08 (s, 1H) 8.12-8.22 (m, 1H) 8.38 (dd, J=6.19, 2.40 Hz, 1H) 8.86 (s, 1H) 11.08 (s, 1H). ESI-MS: m/z 467.2 (M+H)⁺.

### Compound 72: N-(6-(1-(6-(4-cyano-3-fluorophenyl)-[1,2,4]triazolo[4,3-a]pyridin-3-yl)ethyl)imidazo[1,2-b]pyridazin-2-yl)cyclopropanecarboxamide

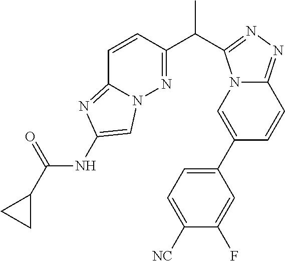 [SMILES: CC(c1ccc2nc(NC(=O)C3CC3)cn2n1)c1nnc2ccc(-c3ccc(C#N)c(F)c3)cn12]

The title compound was synthesized using an analogous procedure to that described in the preparation of Compound 49 using 4-cyano-3-fluorophenylboronic acid. The crude reaction mixture was purified by preparative LCMS, and the product was isolated as the TFA salt. ¹H NMR (400 MHz, DMSO-d6) δ ppm 0.73-0.86 (m, 4H) 1.82-2.00 (m, 4H) 5.29 (d, J=7.33 Hz, 1H) 7.29 (d, J=9.60 Hz, 1H) 7.82-7.88 (m, 2H) 7.89-7.97 (m, 2H) 8.02 (dd, J=10.99, 1.64 Hz, 1H) 8.05-8.10 (m, 2H) 8.95 (s, 1H) 11.08 (s, 1H). ESI-MS: m/z 467.2 (M+H)⁺.

### Compound 73: N-(6-(1-(6-(3-(dimethylamino)phenyl)-[1,2,4]triazolo[4,3-a]pyridin-3-yl)ethyl)imidazo[1,2-b]pyridazin-2-yl)cyclopropanecarboxamide

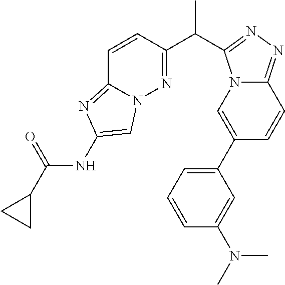 [SMILES: CC(c1ccc2nc(NC(=O)C3CC3)cn2n1)c1nnc2ccc(-c3cccc(N(C)C)c3)cn12]

The title compound was synthesized using an analogous procedure to that described in the preparation of Compound 49 using 3-(dimethylamino)phenylboronic acid. The crude reaction mixture was purified by preparative LCMS, and the product was isolated as the TFA salt. ¹H NMR (400 MHz, DMSO-d6) δ ppm 0.74-0.86 (m, 4H) 1.91 (d, J=7.07 Hz, 4H) 2.94 (s, 6H) 5.29 (d, J=7.33 Hz, 1H) 6.82 (dd, J=8.21, 2.15 Hz, 1H) 6.90 (s, 1H) 6.97 (d, J=7.83 Hz, 1H) 7.25-7.34 (m, 2H) 7.83-7.89 (m, 1H) 7.89-7.98 (m, 2H) 8.09 (s, 1H) 8.62 (s, 1H) 11.09 (s, 1H). ESI-MS: m/z 467.2 (M+H)⁺.

### Compound 74: N-(6-(difluoro(6-(4-fluorophenyl)-[1,2,4]triazolo[4,3-a]pyridin-3-yl)methyl)imidazo[1,2-b]pyridazin-2-yl)cyclopropanecarboxamide

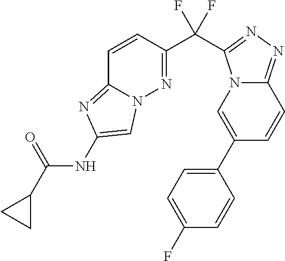 [SMILES: O=C(Nc1cn2nc(C(F)(F)c3nnc4ccc(-c5ccc(F)cc5)cn34)ccc2n1)C1CC1]

The title compound was synthesized using an analogous procedure to that described in the preparation of Compound 45 using 4-fluorophenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (quin, J=6.19 Hz, 1H) 7.37 (t, J=8.84 Hz, 2H) 7.71 (d, J=9.35 Hz, 1H) 7.79-7.85 (m, 2H) 7.96 (dd, J=9.73, 1.39 Hz, 1H) 8.12 (d, J=9.60 Hz, 1H) 8.24 (s, 1H) 8.28 (d, J=9.35 Hz, 1H) 8.74 (s, 1H) 11.35 (s, 1H). ESI-MS: m/z 464.2 (M+H)⁺.

### Compound 75: N-(6-(difluoro(6-(4-methoxyphenyl)-[1,2,4]triazolo[4,3-a]pyridin-3-yl)methyl)imidazo[1,2-b]pyridazin-2-yl)cyclopropanecarboxamide

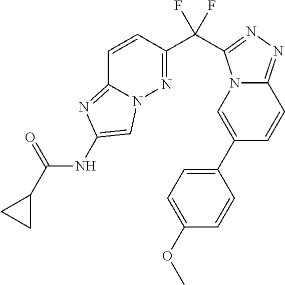 [SMILES: COc1ccc(-c2ccc3nnc(C(F)(F)c4ccc5nc(NC(=O)C6CC6)cn5n4)n3c2)cc1]

The title compound was synthesized using an analogous procedure to that described in the preparation of Compound 45 (Method B) using 4-methoxyphenylboronic acid. The crude reaction mixture was purified by preparative LCMS, and the product was isolated as the TFA salt. ¹H NMR (400 MHz, DMSO-d₆) δ ppm 0.83 (d, J=6.32 Hz, 4H) 1.92-2.00 (m, 1H) 3.82 (s, 3H) 7.09 (d, J=8.84 Hz, 2H) 7.70 (d, 2H) 7.72 (s, 1H) 7.95 (dd, J=9.60, 1.52 Hz, 1H) 8.08 (d, J=9.60 Hz, 1H) 8.25 (s, 1H) 8.27 (d, J=9.60 Hz, 1H) 8.66 (s, 1H) 11.35 (s, 1H). ESI-MS: m/z 476.2 (M+H)⁺.

### Compound 76: N-(6-(difluoro(6-(3-methoxyphenyl)-[1,2,4]triazolo[4,3-a]pyridin-3-yl)methyl)imidazo[1,2-b]pyridazin-2-yl)cyclopropanecarboxamide

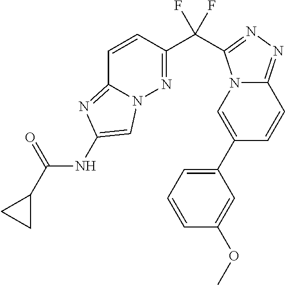 [SMILES: COc1cccc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 3-methoxyphenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.91-2.01 (m, 1H) 3.83 (s, 3H) 7.05 (dd, J=8.21, 1.89 Hz, 1H) 7.26 (d, J=2.02 Hz, 1H) 7.30 (d, J=7.58 Hz, 1H) 7.44 (t, 1H) 7.71 (d, J=9.35 Hz, 1H) 7.98 (dd, J=9.60, 1.52 Hz, 1H) 8.11 (d, J=8.84 Hz, 1H) 8.24 (s, 1H) 8.28 (d, J=9.35 Hz, 1H) 8.73 (s, 1H) 11.35 (s, 1H). ESI-MS: m/z 476.2 (M+H)⁺.

### Compound 77: N-(6-(difluoro(6-(3-fluorophenyl)-[1,2,4]triazolo[4,3-a]pyridin-3-yl)methyl)imidazo[1,2-b]pyridazin-2-yl)cyclopropanecarboxamide

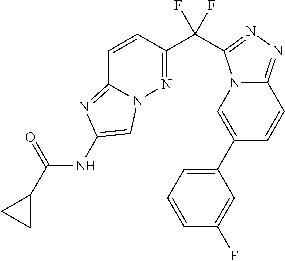 [SMILES: O=C(Nc1cn2nc(C(F)(F)c3nnc4ccc(-c5cccc(F)c5)cn34)ccc2n1)C1CC1]

The title compound was synthesized using an analogous procedure to that described in the preparation of Compound 45 using 3-fluorophenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (quin, J=6.19 Hz, 1H) 7.32 (dt, 1H) 7.54-7.59 (m, 1H) 7.62 (t, J=7.58 Hz, 1H) 7.68 (dt, 1H) 7.71 (d, 1H) 8.00 (dd, J=9.60, 1.52 Hz, 1H) 8.13 (d, J=9.60 Hz, 1H) 8.23 (s, 1H) 8.28 (d, J=9.60 Hz, 1H) 8.81 (s, 1H) 11.34 (s, 1H). ESI-MS: m/z 464.2 (M+H)⁺.

### Compound 78: N-(6-(difluoro(6-(pyridin-3-yl)-[1,2,4]triazolo[4,3-a]pyridin-3-yl)methyl)imidazo[1,2-b]pyridazin-2-yl)cyclopropanecarboxamide

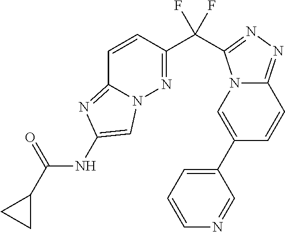 [SMILES: O=C(Nc1cn2nc(C(F)(F)c3nnc4ccc(-c5cccnc5)cn34)ccc2n1)C1CC1]

The title compound was synthesized using an analogous procedure to that described in the preparation of Compound 45 using pyridin-3-ylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (quin, J=6.13 Hz, 1H) 7.61 (dd, J=7.96, 4.93 Hz, 1H) 7.73 (d, J=9.35 Hz, 1H) 8.02 (dd, J=9.60, 1.52 Hz, 1H) 8.17 (d, J=9.09 Hz, 1H) 8.22 (s, 1H) 8.25 (s, 1H) 8.26-8.32 (m, 1H) 8.69 (d, J=4.29 Hz, 1H) 8.90 (s, 1H) 9.00 (br. s., 1H) 11.34 (s, 1H). ESI-MS: m/z 447.2 (M+H)⁺.

### Compound 79: N-(6-(difluoro(6-(pyridin-4-yl)-[1,2,4]triazolo[4,3-a]pyridin-3-yl)methyl)imidazo[1,2-b]pyridazin-2-yl)cyclopropanecarboxamide

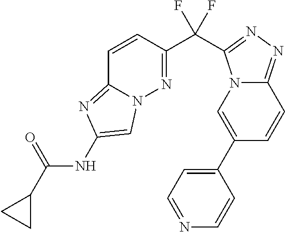 [SMILES: O=C(Nc1cn2nc(C(F)(F)c3nnc4ccc(-c5ccncc5)cn34)ccc2n1)C1CC1]

The title compound was synthesized using an analogous procedure to that described in the preparation of Compound 45 using pyridin-4-ylboronic acid. The crude reaction mixture was purified by preparative LCMS, and the product was isolated as the TFA salt. ¹H NMR (400 MHz, DMSO-d₆) δ ppm 0.83 (d, J=5.31 Hz, 4H) 1.96 (quin, J=6.13 Hz, 1H) 7.69-7.77 (m, 1H) 7.93 (d, J=5.05 Hz, 2H) 8.07 (dd, J=9.60, 1.52 Hz, 1H) 8.19 (d, J=9.85 Hz, 1H) 8.23 (s, 1H) 8.29 (d, J=9.35 Hz, 1H) 8.76 (br. s., 2H) 8.98 (s, 1H) 11.34 (s, 1H). ESI-MS: m/z 447.2 (M+H)⁺.

### Compound 80: N-(6-(difluoro(6-(4-isopropylphenyl)-[1,2,4]triazolo[4,3-a]pyridin-3-yl)methyl)imidazo[1,2-b]pyridazin-2-yl)cyclopropanecarboxamide

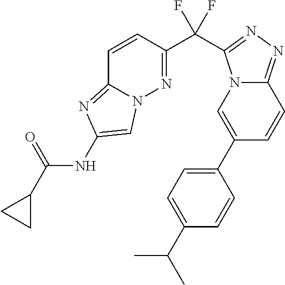 [SMILES: CC(C)c1ccc(-c2ccc3nnc(C(F)(F)c4ccc5nc(NC(=O)C6CC6)cn5n4)n3c2)cc1]

The title compound was synthesized using an analogous procedure to that described in the preparation of Compound 45 using 4-isopropylphenylboronic acid. The crude reaction mixture was purified by preparative LCMS, and the product was isolated as the TFA salt. ¹H NMR (400 MHz, DMSO-d₆) δ ppm 0.84 (d, J=6.06 Hz, 4H) 1.24 (d, J=6.82 Hz, 6H) 1.95 (q, 1H) 2.96 (spt, J=6.86 Hz, 1H) 7.40 (d, J=8.34 Hz, 2H) 7.67 (d, J=8.34 Hz, 2H) 7.71 (d, J=9.35 Hz, 1H) 7.96 (dd, J=9.60, 1.26 Hz, 1H) 8.11 (d, J=9.60 Hz, 1H) 8.24 (s, 1H) 8.28 (d, J=9.35 Hz, 1H) 8.70 (s, 1H) 11.36 (s, 1H). ESI-MS: m/z 488.2 (M+H)⁺.

### Compound 81: N-(6-((6-(3,5-difluorophenyl)-[1,2,4]triazolo[4,3-a]pyridin-3-yl)difluoromethyl)imidazo[1,2-b]pyridazin-2-yl)cyclopropanecarboxamide

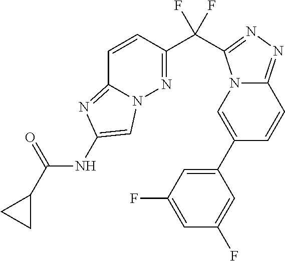 [SMILES: O=C(Nc1cn2nc(C(F)(F)c3nnc4ccc(-c5cc(F)cc(F)c5)cn34)ccc2n1)C1CC1]

The title compound was synthesized using an analogous procedure to that described in the preparation of Compound 45 using 3,5-difluorophenylboronic acid. The crude reaction mixture was purified by preparative LCMS, and the product was isolated as the TFA salt. ¹H NMR (400 MHz, DMSO-d₆) δ ppm 0.83 (d, J=5.81 Hz, 4H) 1.95 (quin, 1H) 7.32-7.42 (m, 1H) 7.56-7.66 (m, 2H) 7.73 (d, J=9.60 Hz, 1H) 8.01 (dd, J=9.73, 1.39 Hz, 1H) 8.14 (d, J=9.60 Hz, 1H) 8.21 (s, 1H) 8.28 (d, J=9.35 Hz, 1H) 8.88 (s, 1H) 11.34 (s, 1H). ESI-MS: m/z 482.2 (M+H)⁺.

### Compound 82: N-(6-((6-(3-cyanophenyl)-[1,2,4]triazolo[4,3-a]pyridin-3-yl)difluoromethyl)imidazo[1,2-b]pyridazin-2-yl)cyclopropanecarboxamide

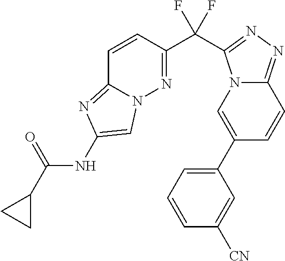 [SMILES: N#Cc1cccc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 3-cyanophenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (quin, J=6.13 Hz, 1H) 7.68-7.77 (m, 2H) 7.94 (d, J=7.83 Hz, 1H) 8.03 (dd, J=9.73, 1.14 Hz, 1H) 8.12 (d, J=8.34 Hz, 1H) 8.16 (d, J=9.60 Hz, 1H) 8.23 (s, 1H) 8.29 (d, J=9.35 Hz, 1H) 8.33 (s, 1H) 8.93 (s, 1H) 11.35 (s, 1H). ESI-MS: m/z 471.2 (M+H)⁺.

### Compound 83: N-(6-((6-(4-cyanophenyl)-[1,2,4]triazolo[4,3-a]pyridin-3-yl)difluoromethyl)imidazo[1,2-b]pyridazin-2-yl)cyclopropanecarboxamide

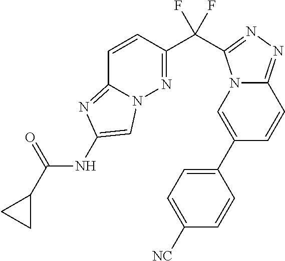 [SMILES: N#Cc1ccc(-c2ccc3nnc(C(F)(F)c4ccc5nc(NC(=O)C6CC6)cn5n4)n3c2)cc1]

The title compound was synthesized using an analogous procedure to that described in the preparation of Compound 45 using 4-cyanophenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (quin, J=6.19 Hz, 1H) 7.72 (d, J=9.60 Hz, 1H) 8.00 (s, 4H) 8.03 (d, J=9.60 Hz, 1H) 8.16 (d, J=9.60 Hz, 1H) 8.20-8.24 (m, 1H) 8.28 (d, J=9.60 Hz, 1H) 8.89 (s, 1H) 11.35 (s, 1H). ESI-MS: m/z 471.2 (M+H)⁺.

### Compound 84: N-(6-(difluoro(6-(4-methylthiophen-2-yl)-[1,2,4]triazolo[4,3-a]pyridin-3-yl)methyl)imidazo[1,2-b]pyridazin-2-yl)cyclopropanecarboxamide

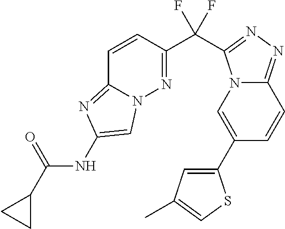 [SMILES: Cc1csc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 4-methylthiophen-2-ylboronic acid. The crude reaction mixture was purified by preparative LCMS, and the product was isolated as the TFA salt. ¹H NMR (400 MHz, DMSO-d₆) δ ppm 0.84 (d, J=6.32 Hz, 4H) 1.95 (q, 1H) 2.26 (s, 3H) 7.29 (s, 1H) 7.59 (s, 1H) 7.70 (d, J=9.35 Hz, 1H) 7.91 (dd, J=9.73, 1.64 Hz, 1H) 8.07 (d, J=9.60 Hz, 1H) 8.26 (s, 1H) 8.25-8.30 (m, 1H) 8.69 (s, 1H) 11.36 (s, 1H). ESI-MS: m/z 466.2 (M+H)⁺.

### Compound 85: N-(6-(difluoro(6-(4-(methylsulfonyl)phenyl)-[1,2,4]triazolo[4,3-a]pyridin-3-yl)methyl)imidazo[1,2-b]pyridazin-2-yl)cyclopropanecarboxamide

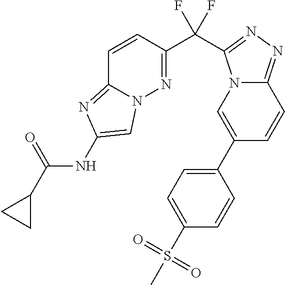 [SMILES: CS(=O)(=O)c1ccc(-c2ccc3nnc(C(F)(F)c4ccc5nc(NC(=O)C6CC6)cn5n4)n3c2)cc1]

The title compound was synthesized using an analogous procedure to that described in the preparation of Compound 45 using 4-(methylsulfonyl)phenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (q, 1H) 3.28 (s, 3H) 7.72 (d, J=9.35 Hz, 1H) 8.02 (dd, 1H) 8.05 (s, 4H) 8.17 (d, J=9.60 Hz, 1H) 8.23 (s, 1H) 8.28 (d, J=9.60 Hz, 1H) 8.88 (s, 1H) 11.35 (s, 1H). ESI-MS: m/z 524.2 (M+H)⁺.

### Compound 86: N-(6-(difluoro(6-(6-methoxypyridin-3-yl)-[1,2,4]triazolo[4,3-a]pyridin-3-yl)methyl)imidazo[1,2-b]pyridazin-2-yl)cyclopropanecarboxamide

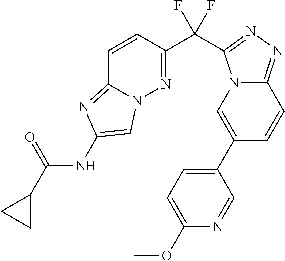 [SMILES: COc1ccc(-c2ccc3nnc(C(F)(F)c4ccc5nc(NC(=O)C6CC6)cn5n4)n3c2)cn1]

The title compound was synthesized using an analogous procedure to that described in the preparation of Compound 45 using 6-methoxypyridin-3-ylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (quin, J=6.19 Hz, 1H) 3.91 (s, 3H) 6.97 (d, J=8.59 Hz, 1H) 7.72 (d, J=9.35 Hz, 1H) 7.97 (dd, J=9.60, 1.52 Hz, 1H) 8.09-8.15 (m, 2H) 8.22 (s, 1H) 8.28 (d, J=9.35 Hz, 1H) 8.56 (d, J=2.53 Hz, 1H) 8.78 (s, 1H) 11.35 (s, 1H). ESI-MS: m/z 477.2 (M+H)⁺.

### Compound 87: N-(6-(difluoro(6-(pyrimidin-5-yl)-[1,2,4]triazolo[4,3-a]pyridin-3-yl)methyl)imidazo[1,2-b]pyridazin-2-yl)cyclopropanecarboxamide

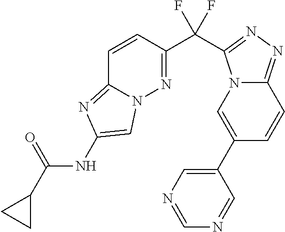 [SMILES: O=C(Nc1cn2nc(C(F)(F)c3nnc4ccc(-c5cncnc5)cn34)ccc2n1)C1CC1]

The title compound was synthesized using an analogous procedure to that described in the preparation of Compound 45 using pyrimidin-5-ylboronic acid. The crude reaction mixture was purified by preparative LCMS, and the product was isolated as the TFA salt. ¹H NMR (400 MHz, DMSO-d₆) δ ppm 0.83 (d, J=5.56 Hz, 4H) 1.90-2.00 (m, 1H) 7.74 (d, J=9.60 Hz, 1H) 8.06 (dd, J=9.60, 1.26 Hz, 1H) 8.17-8.25 (m, 2H) 8.29 (d, J=9.35 Hz, 1H) 9.03 (s, 1H) 9.21 (s, 2H) 9.24-9.29 (m, 1H) 11.34 (s, 1H). ESI-MS: m/z 448.2 (M+H)⁺.

### Compound 88: N-(6-(difluoro(6-(3-(methylsulfonyl)phenyl)-[1,2,4]triazolo[4,3-a]pyridin-3-yl)methyl)imidazo[1,2-b]pyridazin-2-yl)cyclopropanecarboxamide

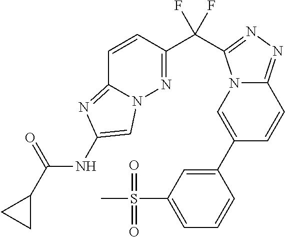 [SMILES: CS(=O)(=O)c1cccc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 3-(methylsulfonyl)phenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.91-2.01 (m, 1H) 3.32 (s, 3H) 7.72 (d, J=9.35 Hz, 1H) 7.81 (t, J=7.83 Hz, 1H) 8.02 (d, J=7.83 Hz, 1H) 8.06 (dd, J=9.60, 1.52 Hz, 1H) 8.13 (d, J=7.83 Hz, 1H) 8.18 (d, J=9.60 Hz, 1H) 8.23-8.31 (m, 3H) 8.92 (s, 1H) 11.35 (s, 1H). ESI-MS: m/z 524.2 (M+H)⁺.

Compound 89: 4-(3-((2-(cyclopropanecarboxamido)imidazo[1,2-b]pyridazin-6-yl)difluoromethyl)-[1,2,4]triazolo[4,3-a]pyridin-6-yl)benzamide

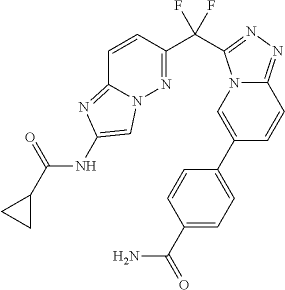 [SMILES: NC(=O)c1ccc(-c2ccc3nnc(C(F)(F)c4ccc5nc(NC(=O)C6CC6)cn5n4)n3c2)cc1]

The title compound was synthesized using an analogous procedure to that described in the preparation of Compound 45 using 4-carbamoylphenylboronic acid. The crude reaction mixture was purified by preparative LCMS, and the product was isolated as the TFA salt. ¹H NMR (400 MHz, DMSO-d₆) δ ppm 0.83 (d, J=6.32 Hz, 4H) 1.90-2.01 (m, 1H) 7.48 (br. s., 1H) 7.72 (d, J=9.35 Hz, 1H) 7.87 (d, J=8.34 Hz, 2H) 7.98-8.06 (m, 3H) 8.10 (br. s., 1H) 8.11-8.17 (m, 1H) 8.24 (s, 1H) 8.28 (d, J=9.35 Hz, 1H) 8.82 (s, 1H) 11.35 (s, 1H). ESI-MS: m/z 489.2 (M+H)⁺.

### Compound 90: 3-(3-((2-(cyclopropanecarboxamido)imidazo[1,2-b]pyridazin-6-yl)difluoromethyl)-[1,2,4]triazolo[4,3-a]pyridin-6-yl)benzamide

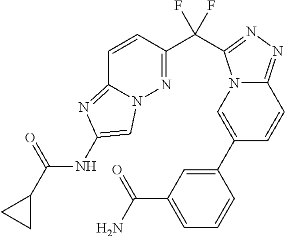 [SMILES: NC(=O)c1cccc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 3-carbamoylphenylboronic acid. The crude reaction mixture was purified by preparative LCMS, and the product was isolated as the TFA salt. ¹H NMR (400 MHz, DMSO-d₆) δ ppm 0.84 (d, J=6.06 Hz, 4H) 1.96 (quin, J=6.13 Hz, 1H) 7.53 (s, 1H) 7.62 (t, J=7.70 Hz, 1H) 7.72 (d, J=9.60 Hz, 1H) 7.90-7.98 (m, 2H) 8.04 (dd, J=9.73, 1.39 Hz, 1H) 8.16 (d, J=9.60 Hz, 1H) 8.19 (br. s., 1H) 8.22 (s, 1H) 8.25 (s, 1H) 8.28 (d, J=9.35 Hz, 1H) 8.85 (s, 1H) 11.35 (s, 1H). ESI-MS: m/z 489.2 (M+H)⁺.

### Compound 91: N-(6-(difluoro(6-(4-(2-methoxyethoxy)phenyl)-[1,2,4]triazolo[4,3-a]pyridin-3-yl)methyl)imidazo[1,2-b]pyridazin-2-yl)cyclopropanecarboxamide

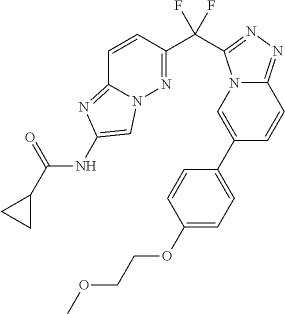 [SMILES: COCCOc1ccc(-c2ccc3nnc(C(F)(F)c4ccc5nc(NC(=O)C6CC6)cn5n4)n3c2)cc1]

The title compound was synthesized using an analogous procedure to that described in the preparation of Compound 45 using 4-(2-methoxyethoxy)phenylboronic acid. The crude reaction mixture was purified by preparative LCMS, and the product was isolated as the TFA salt. ¹H NMR (400 MHz, DMSO-d₆) δ ppm 0.84 (d, J=6.32 Hz, 4H) 1.96 (quin, J=6.13 Hz, 1H) 3.32 (s, 3H) 3.66-3.71 (m, 2H) 4.13-4.19 (m, 2H) 7.09 (d, J=8.59 Hz, 2 H) 7.70 (dd, J=9.09, 2.78 Hz, 3H) 7.95 (dd, J=9.60, 1.52 Hz, 1H) 8.08 (d, J=9.60 Hz, 1H) 8.25 (s, 1H) 8.28 (d, J=9.35 Hz, 1H) 8.67 (s, 1H) 11.36 (s, 1H). ESI-MS: m/z 520.2 (M+H)⁺.

### Compound 92: N-(6-((6-(1H-pyrazol-4-yl)-[1,2,4]triazolo[4,3-a]pyridin-3-yl)difluoromethyl)imidazo[1,2-b]pyridazin-2-yl)cyclopropanecarboxamide

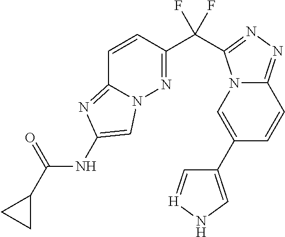

The title compound was synthesized using an analogous procedure to that described in the preparation of Compound 45 using 1H-pyrazol-4-ylboronic acid. The crude reaction mixture was purified by preparative LCMS, and the product was isolated as the TFA salt. ¹H NMR (400 MHz, DMSO-d₆) δ ppm 0.84 (d, J=6.06 Hz, 4H) 1.96 (quin, J=6.19 Hz, 1H) 7.71 (d, J=9.35 Hz, 1H) 7.97 (dd, 1H) 8.03-8.08 (m, 1H) 8.17-8.43 (m, 2H) 8.25 (s, 1H) 8.28 (d, J=9.35 Hz, 1H) 8.75 (s, 1H) 11.35 (s, 1H).). ESI-MS: m/z 436.2 (M+H)⁺.

### Compound 93: N-(6-(difluoro(6-(3-isopropoxyphenyl)-[1,2,4]triazolo[4,3-a]pyridin-3-yl)methyl)imidazo[1,2-b]pyridazin-2-yl)cyclopropanecarboxamide

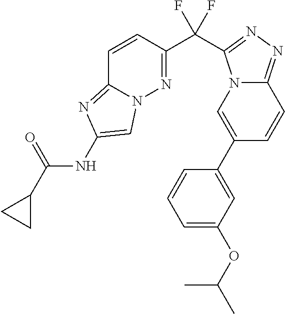 [SMILES: CC(C)Oc1cccc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 3-isopropoxyphenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.28 (d, J=6.06 Hz, 6H) 1.96 (quin, J=6.13 Hz, 1H) 4.65-4.76 (m, 1H) 7.02 (dd, J=8.21, 1.89 Hz, 1H) 7.21 (s, 1H) 7.26 (d, J=7.83 Hz, 1H) 7.38-7.46 (m, 1H) 7.71 (d, J=9.35 Hz, 1H) 7.96 (dd, J=9.60, 1.26 Hz, 1H) 8.10 (d, J=9.60 Hz, 1H) 8.24 (s, 1H) 8.28 (d, J=9.35 Hz, 1H) 8.71 (s, 1H) 11.36 (s, 1H). ESI-MS: m/z 504.2 (M+H)⁺.

### Compound 94: N-(6-(difluoro(6-(4-methylpyridin-3-yl)-[1,2,4]triazolo[4,3-a]pyridin-3-yl)methyl)imidazo[1,2-b]pyridazin-2-yl)cyclopropanecarboxamide

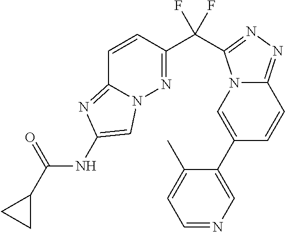 [SMILES: Cc1ccncc1-c1ccc2nnc(C(F)(F)c3ccc4nc(NC(=O)C5CC5)cn4n3)n2c1]

The title compound was synthesized using an analogous procedure to that described in the preparation of Compound 45 using 4-methylpyridin-3-ylboronic acid. The crude reaction mixture was purified by preparative LCMS, and the product was isolated as the TFA salt. ¹H NMR (400 MHz, DMSO-d₆) δ ppm 0.79-0.89 (m, 4H) 1.19-1.29 (m, 3H) 1.89-2.00 (m, 1H) 6.87 (s, 1H) 7.43 (br. s., 1H) 7.68-7.76 (m, 2H) 8.11 (d, J=8.84 Hz, 1H) 8.22 (s, 1H) 8.28 (d, J=9.35 Hz, 1H) 8.51 (br. s., 1H) 8.68 (s, 1H) 11.35 (s, 1H). ESI-MS: m/z 461.2 (M+H)⁺.

### Compound 95: 4-(3-((2-(cyclopropanecarboxamido)imidazo[1,2-b]pyridazin-6-yl)difluoromethyl)-[1,2,4]triazolo[4,3-a]pyridin-6-yl)-N-methylbenzamide

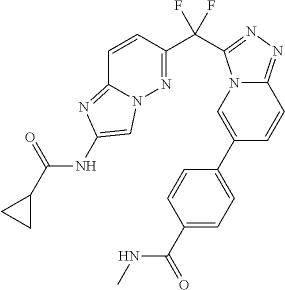 [SMILES: CNC(=O)c1ccc(-c2ccc3nnc(C(F)(F)c4ccc5nc(NC(=O)C6CC6)cn5n4)n3c2)cc1]

The title compound was synthesized using an analogous procedure to that described in the preparation of Compound 45 using 4-(methylcarbamoyl)phenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.96 (quin, J=6.13 Hz, 1H) 2.76-2.86 (m, 3H) 7.72 (d, J=9.60 Hz, 1H) 7.88 (d, J=8.34 Hz, 2H) 7.93-8.00 (m, 2H) 8.03 (dd, J=9.73, 1.39 Hz, 1H) 8.14 (d, J=9.35 Hz, 1H) 8.24 (s, 1H) 8.28 (d, J=9.35 Hz, 1H) 8.57 (d, J=4.55 Hz, 1H) 8.83 (s, 1H) 11.35 (s, 1H).). ESI-MS: m/z 503.2 (M+H)⁺.

### Compound 96: N-(6-((6-(3-(dimethylamino)phenyl)-[1,2,4]triazolo[4,3-a]pyridin-3-yl)difluoromethyl)imidazo[1,2-b]pyridazin-2-yl)cyclopropanecarboxamide

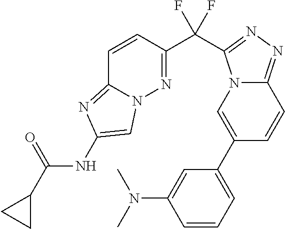 [SMILES: CN(C)c1cccc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 3-(dimethylamino)phenylboronic acid. The crude reaction mixture was purified by preparative LCMS, and the product was isolated as the TFA salt. ¹H NMR (400 MHz, DMSO-d₆) δ ppm 0.84 (d, J=6.06 Hz, 4H) 1.96 (quin, J=6.13 Hz, 1H) 2.96 (s, 6H) 6.85 (d, J=8.34 Hz, 1H) 6.94 (br. s., 1H) 6.98 (d, J=7.33 Hz, 1H) 7.33 (t, J=7.83 Hz, 1H) 7.68-7.74 (m, 1H) 7.93-7.99 (m, 1H) 8.09 (d, J=9.60 Hz, 1H) 8.25 (s, 1H) 8.28 (d, J=9.60 Hz, 1H) 8.65 (s, 1H) 11.32-11.40 (m, 1H). ESI-MS: m/z 489.2 (M+H)⁺.

### Compound 97: N-(6-(difluoro(6-(3-fluoro-5-methylphenyl)-[1,2,4]triazolo[4,3-a]pyridin-3-yl)methyl)imidazo[1,2-b]pyridazin-2-yl)cyclopropanecarboxamide

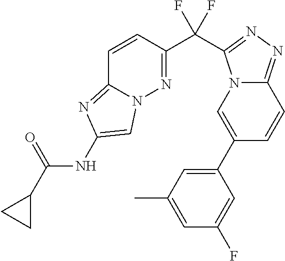 [SMILES: Cc1cc(F)cc(-c2ccc3nnc(C(F)(F)c4ccc5nc(NC(=O)C6CC6)cn5n4)n3c2)c1]

The title compound was synthesized using an analogous procedure to that described in the preparation of Compound 45 using 3-fluoro-5-methylphenylboronic acid. The crude reaction mixture was purified by preparative LCMS, and the product was isolated as the TFA salt. ¹H NMR (400 MHz, DMSO-d₆) δ ppm 0.83 (d, J=6.06 Hz, 4H) 1.95 (q, 1H) 2.41 (s, 3H) 7.15 (d, J=9.60 Hz, 1H) 7.42-7.50 (m, 2H) 7.72 (d, J=9.35 Hz, 1H) 7.98 (dd, J=9.73, 1.39 Hz, 1H) 8.12 (d, J=9.60 Hz, 1H) 8.24 (s, 1H) 8.28 (d, J=9.60 Hz, 1H) 8.80 (s, 1H) 11.36 (s, 1H). ESI-MS: m/z 478.2 (M+H)⁺.

### Compound 98: 4-(3-((2-(cyclopropanecarboxamido)imidazo[1,2-b]pyridazin-6-yl)difluoromethyl)-[1,2,4]triazolo[4,3-a]pyridin-6-yl)-2-fluoro-N-methylbenzamide

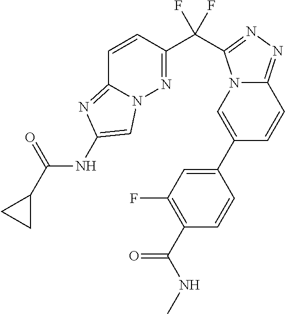 [SMILES: CNC(=O)c1ccc(-c2ccc3nnc(C(F)(F)c4ccc5nc(NC(=O)C6CC6)cn5n4)n3c2)cc1F]

The title compound was synthesized using an analogous procedure to that described in the preparation of Compound 45 using 3-fluoro-4-(methylcarbamoyl)phenylboronic acid. The crude reaction mixture was purified by preparative LCMS, and the product was isolated as the TFA salt. ¹H NMR (400 MHz, DMSO-d₆) δ ppm 0.83 (d, J=5.81 Hz, 4H) 1.90-2.00 (m, 1H) 2.80 (d, J=4.55 Hz, 3H) 7.68-7.78 (m, 2H) 7.81 (d, J=11.87 Hz, 1H) 8.00-8.07 (m, 1H) 8.11-8.18 (m, 1H) 8.22 (s, 1H) 8.28 (d, J=9.35 Hz, 1H) 8.33 (d, J=4.55 Hz, 1H) 8.88 (s, 1H) 11.35 (s, 1H).). ESI-MS: m/z 521.2 (M+H)⁺.

### Compound 99: 6-(difluoro(6-(1-methyl-1H-pyrazol-4-yl)-[1,2,4]triazolo[4,3-a]pyridin-3-yl)methyl)imidazo[1,2-b]pyridazin-2-amine

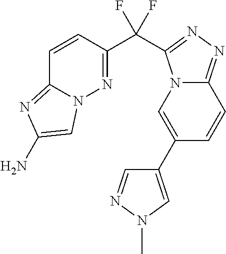 [SMILES: Cn1cc(-c2ccc3nnc(C(F)(F)c4ccc5nc(N)cn5n4)n3c2)cn1]

A solution of 45 (100 mg) in 3N HCl:50% MeOH:water was heated at 50° C. for 3 hrs. The reaction was concentrated and purified by preparative LCMS to provide the title compound, 6-(difluoro(6-(1-methyl-1H-pyrazol-4-yl)-[1,2,4]triazolo[4,3-a]pyridin-3-yl)methyl)imidazo[1,2-b]pyridazin-2-amine (30 mg) as the TFA salt. ¹H NMR (400 MHz, DMSO-d₆) δ ppm 3.89 (s, 3H) 7.46 (br. s., 1H) 7.51-7.58 (m, 1H) 7.90 (dd, J=9.47, 1.39 Hz, 1H) 7.93-7.99 (m, 1H) 8.04 (d, J=9.60 Hz, 1H) 8.08 (s, 1H) 8.39-8.43 (m, 2H) 8.67 (s, 1H). ESI-MS: m/z 382.2 (M+H)⁺.

### Compound 100: N-(6-(6-(5-methyl-1H-1,2,4-triazol-3-yl)-[1,2,4]triazolo[4,3-a]pyridin-3-ylthio)imidazo[1,2-b]pyridazin-2-yl)cyclopropanecarboxamide

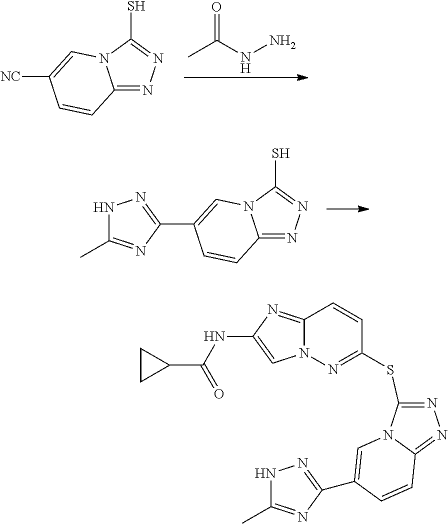

### 6-(5-methyl-1H-1,2,4-triazol-3-yl)-[1,2,4]triazolo[4,3-a]pyridine-3-thiol

A mixture of 3-mercapto-[1,2,4]triazolo[4,3-a]pyridine-6-carbonitrile (38A, 450 mg, 2.56 mmol), acetohydrazide (400 mg, 5.4 mmol), and sodium 2-methylpropan-2-olate (200 mg, 2.08 mmol) in propan-2-ol (2.5 ml) was heated at 180° C. under microwave condition for 2 hrs. The product was purified by preparative LCMS to provide the title compound, 6-(5-methyl-1H-1,2,4-triazol-3-yl)-[1,2,4]triazolo[4,3-a]pyridine-3-thiol.

N-(6-(6-(5-methyl-1H-1,2,4-triazol-3-yl)-[1,2,4]triazolo[4,3-a]pyridin-3-ylthio)imidazo[1,2-b]pyridazin-2-yl)cyclopropanecarboxamide: The title compound was synthesized using an analogous procedure to that described in the preparation of Compound 4 using 6-(5-methyl-1H-1,2,4-triazol-3-yl)-[1,2,4]triazolo[4,3-a]pyridine-3-thiol. The crude reaction mixture was purified by preparative LCMS, and the product was isolated as the TFA salt. ¹H NMR (DMSO-d₆,400 MHz): δ=11.26 (s, 1H), 9.25 (s, 1H), 8.19-8.29 (m, 2H), 8.00 (d, J=9.6 Hz, 1H), 7.85-7.93 (m, 1H), 7.40 (d, J=9.3 Hz, 1H), 2.43 (s, 3H), 1.96 (br. s., 1H), 0.75-0.93 ppm (m, 5H). ESI-MS: m/z 433.1 (M+H)⁺.

### Compound 101: N-(6-(6-(6-methoxypyridin-2-yl)-[1,2,4]triazolo[4,3-a]pyridin-3-ylthio)imidazo[1,2-b]pyridazin-2-yl)cyclopropanecarboxamide

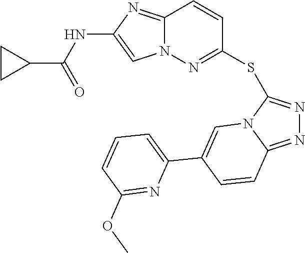 [SMILES: COc1cccc(-c2ccc3nnc(Sc4ccc5nc(NC(=O)C6CC6)cn5n4)n3c2)n1]

The title compound was synthesized using an analogous procedure to that described in the preparation of Compound 5 using 6-methoxypyridin-2-ylboronic acid. The crude reaction mixture was purified by preparative LCMS, and the product was isolated as the TFA salt. ¹H NMR (METHANOL-d₄,400 MHz): δ=9.09 (s, 1H), 8.35 (d, J=9.9 Hz, 1H), 7.95-8.04 (m, 2H), 7.84 (d, J=9.3 Hz, 1H), 7.71-7.78 (m, 1H), 7.55 (d, J=7.3 Hz, 1H), 7.23 (d, J=9.1 Hz, 1H), 6.79 (d, J=8.3 Hz, 1H), 3.85 (s, 3H), 1.84 (m, 1H), 0.82-0.96 ppm (m, 4H). ESI-MS: m/z 459.2 (M+H)⁺.

### Compound 102: N-(6-(6-(6-methoxypyridin-3-yl)-[1,2,4]triazolo[4,3-a]pyridin-3-ylthio)imidazo[1,2-b]pyridazin-2-yl)cyclopropanecarboxamide

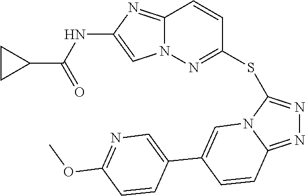 [SMILES: COc1ccc(-c2ccc3nnc(Sc4ccc5nc(NC(=O)C6CC6)cn5n4)n3c2)cn1]

The title compound was synthesized using an analogous procedure to that described in the preparation of Compound 5 using 6-methoxypyridin-3-ylboronic acid. The crude reaction mixture was purified by preparative LCMS, and the product was isolated as the TFA salt. ¹H NMR (METHANOL-d₄,400 MHz): δ=8.71 (br. s., 1H), 8.40 (br. s., 1H), 7.86-8.16 (m, 4H), 7.80 (br. s., 1H), 7.20 (br. s., 1H), 6.87 (d, J=8.8 Hz, 1H), 3.92 (s, 3H), 1.85 (br. s., 1H), 0.73-1.02 ppm (m, 4H). ESI-MS: m/z 459.2 (M+H)⁺.

### Compound 103: N-(6-(6-(6-morpholinopyridin-3-yl)-[1,2,4]triazolo[4,3-a]pyridin-3-ylthio)imidazo[1,2-b]pyridazin-2-yl)cyclopropanecarboxamide

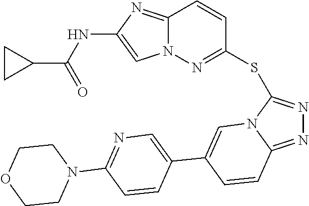 [SMILES: O=C(Nc1cn2nc(Sc3nnc4ccc(-c5ccc(N6CCOCC6)nc5)cn34)ccc2n1)C1CC1]

The title compound was synthesized using an analogous procedure to that described in the preparation of Compound 5 using 4-(5-(4,4,5,5-tetramethyl-1,3,2-dioxaborolan-2-yl)pyridin-2-yl)morpholine. The crude reaction mixture was purified by preparative LCMS, and the product was isolated as the TFA salt.

### Compound 104: N-(6-(6-(3-cyanophenyl)-[1,2,4]triazolo[4,3-a]pyridin-3-ylthio)imidazo[1,2-b]pyridazin-2-yl)cyclopropanecarboxamide

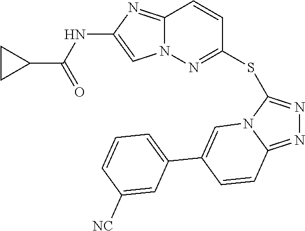 [SMILES: N#Cc1cccc(-c2ccc3nnc(Sc4ccc5nc(NC(=O)C6CC6)cn5n4)n3c2)c1]

The title compound was synthesized using an analogous procedure to that described in the preparation of Compound 5 using 3-cyanophenylboronic acid. The crude reaction mixture was purified by preparative LCMS, and the product was isolated as the TFA salt. ¹H NMR (METHANOL-d₄,400 MHz): δ=8.72 (s, 1H), 8.34 (d, J=2.3 Hz, 1H), 8.12 (dd, J=9.2, 2.4 Hz, 1H), 8.04 (d, J=9.6 Hz, 1H), 7.92 (d, J=8.8 Hz, 1H), 7.82 (s, 2H), 7.16-7.27 (m, 2H), 3.80-3.89 (m, 4H), 3.61-3.69 (m, 4H), 1.85 (br. s., 1H), 0.95 (m, 2H), 0.80-0.92 ppm (m, 2H). ESI-MS: m/z 514.2 (M+H)⁺.

### Compound 105: N-(6-(6-(1-methyl-6-oxo-1,6-dihydropyridin-3-yl)-[1,2,4]triazolo[4,3-a]pyridin-3-ylthio)imidazo[1, 2-1)]pyridazin-2-yl)cyclopropanecarboxamide

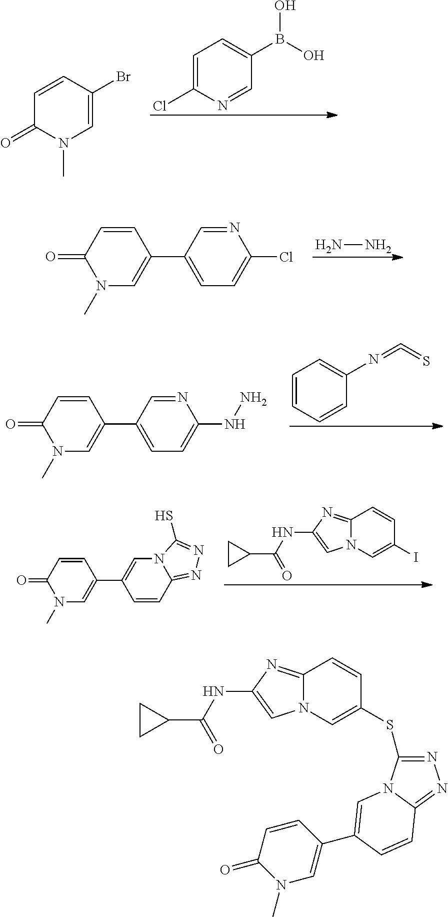

### 5-(6-Chloropyridin-3-yl)-1-methylpyridin-2(1H)-one

A mixture of 5-bromo-1-methylpyridin-2(1H)-one (2.2 g, 11.7 mmol), 6-chloropyridin-3-ylboronic acid (1.57 g, 9.98 mmol), Na₂CO₃ (2.2 g, 20.76 mmol), and tetrakis(triphenylphosphine)palladium (0) (500 mg, 0.05 mmol) in dioxane:water (15:1, 20 ml) was degassed and heated at 110° C. in a microwave for 1 hr. The reaction was diluted with EtOAc and washed with water. The organic layer was dried over Na₂SO₄, filtered, and concentrated to dryness. The resulting residue was purified by silica gel chromatography (AcOEt-Hexanes) to provide title compound, 5-(6-Chloropyridin-3-yl)-1-methylpyridin-2(1H)-one (1.6 g, 73% yield).

### 5-(6-hydrazinylpyridin-3-yl)-1-methylpyridin-2(1H)-one

A mixture of 5-(6-chloropyridin-3-yl)-1-methylpyridin-2(1H)-one (1.2 g, 5.44 mmol) and hydrazine (870 mg, 2.2 mmol) in 2-propanol (5 ml) was heated in a microwave at 140° C. for 5 hrs. The reaction mixture was co-evaporated with MeOH (3×) and then suspended in water (50 ml). Solid product was collected by filtration and dried under high vacuum for 24 hrs to provide the title compound, 5-(6-hydrazinylpyridin-3-yl)-1-methylpyridin-2(1H)-one (600 mg, 51% yield).

### 5-(3-mercapto-[1,2,4]triazolo[4,3-a]pyridin-6-yl)-1-methylpyridin-2(1H)-one

A mixture of 5-(6-hydrazinylpyridin-3-yl)-1-methylpyridin-2(1H)-one (600 mg, 2.78 mmol) and isothiocyanatobenzene (450 mg, 3.33 mmol) in NMP-1,3-dichlorobenzene (1:1, 10 mL) was heated in a microwave at 160° C. for 1 hr and then purified by preparative LCMS to provide the title compound, 5-(3-mercapto-[1,2,4]triazolo[4,3-a]pyridin-6-yl)-1-methylpyridin-2(1H)-one (310 mg, 43% yield).

N-(6-(6-(1-methyl-6-oxo-1,6-dihydropyridin-3-yl)-[1,2,4]triazolo[4,3-a]pyridin-3-ylthio)imidazo[1,2-b]pyridazin-2-yl)cyclopropanecarboxamide: The title compound was prepare from N-(6-iodoimidazo[1,2-b]pyridazin-2-yl)cyclopropanecarboxamide and 5-(3-mercapto-[1,2,4]triazolo[4,3-a]pyridin-6-yl)-1-methylpyridin-2(1H)-one following the procedure for the synthesis of compound 4. The crude reaction mixture was purified by preparative LCMS, and the product was isolated as the TFA salt. The product was then dissolved in MeOH, treated 4M HCl in dioxane, and concentrated to dryness to provide the HCl salt of the title compound. ¹H NMR (METHANOL-d₄,400 MHz): δ=8.83 (s, 1H), 8.18 (d, J=9.9 Hz, 2H), 8.11 (d, J=9.6 Hz, 1H), 7.98 (d, J=9.3 Hz, 1H), 7.77-7.92 (m, 2H), 7.55 (d, J=9.1 Hz, 1H), 6.58 (d, J=9.3 Hz, 1H), 3.52-3.59 (m, 3H), 1.73 (br. s., 1H), 0.80-0.90 ppm (m, 4H). ESI-MS: m/z 459.2 (M+H)⁺.

### Compound 106: N-(6-(6-(1-ethyl-6-oxo-1,6-dihydropyridin-3-yl)-[1,2,4]triazolo[4,3-a]pyridin-3-ylthio)imidazo[1,2-b]pyridazin-2-yl)cyclopropanecarboxamide

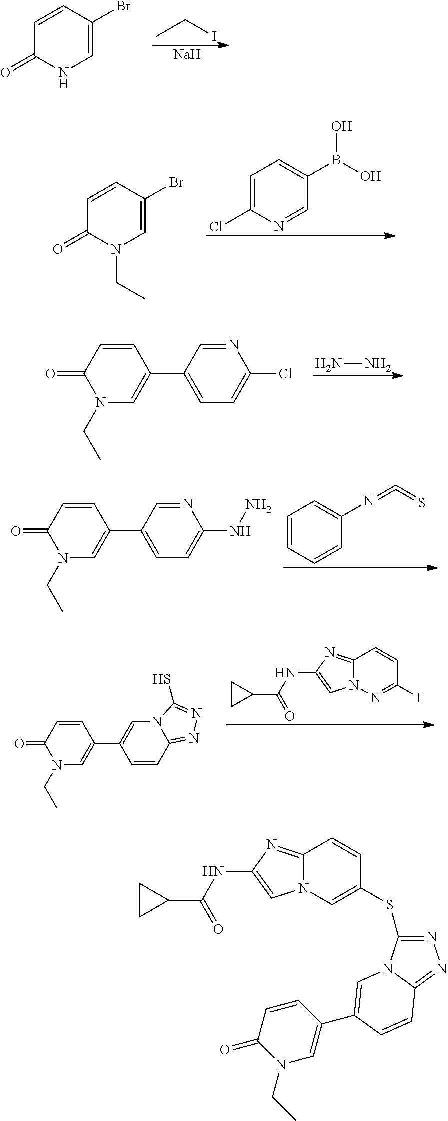

### 5-bromo-1-ethylpyridin-2(1H)-one

A solution of 5-bromopyridin-2-ol (1.74 g, 10.00 mmol) in DMF (20 ml) was treated with NaH at room temperature and then stirred for 30 min. Iodoethane (0.823 ml, 12.00 mmol) was added to the mixture and the reaction was stirred overnight at room temperature. The reaction was diluted with DCM, washed with water, dried over Na₂SO₄, and concentrated to dryness via rotary evaporation to provide the title compound as a crude product which was used without further purification.

### 5-(6-chloropyridin-3-yl)-1-ethylpyridin-2(1H)-one

A mixture of 5-bromo-1-ethylpyridin-2(1H)-one (1.9 g, 9.45 mmol), 6-chloropyridin-3-ylboronic acid (1.8 g, 11.46 mmol), Cs₂CO₃ (9.9 g, 30.37 mmol) and tetrakis(triphenylphosphine)palladium (0) (543 mg, 0.55 mmol) were suspended in dioxane:water (15:1, 20 ml). After degassing, the mixture was heated in a microwave at 140° C. for 30 min. The reaction was then diluted with EtOAc and washed with water. The organic layer was dried over Na₂SO₄, filtered, and concentrated to dryness via rotary evaporation. The resulting residue was purified by LCMS to provide the title compound, 5-(6-chloropyridin-3-yl)-1-ethylpyridin-2(1H)-one (1.2 g, 51% yield).

### 5-(6-hydrazinylpyridin-3-yl)-1-ethylpyridin-2(1H)-one

A mixture of 5-(6-chloropyridin-3-yl)-1-ethylpyridin-2(1H)-one (1.2 g, 5.44 mmol) and hydrazine (820 mg, 25.62 mmol) in 2-propanol (5 ml) was heated in a microwave at 140° C. for 4 hrs. The mixture was co-evaporated with MeOH (3×) and then suspended in water (50 ml). The resulting solid was collected by filtration and dried under high vacuum for one day to give crude title compound, 5-(6-hydrazinylpyridin-3-yl)-1-ethylpyridin-2(1H)-one (1.2 g, 95.9% yield).

### 5-(3-mercapto-[1,2,4]triazolo[4,3-a]pyridin-6-yl)-1-ethylpyridin-2(1H)-one

A mixture of 5-(6-hydrazinylpyridin-3-yl)-1-ethylpyridin-2(1H)-one (1.2 g, 5.22 mmol) and isothiocyanatobenzene (775 mg, 5.74 mmol) in NMP (3 ml) and DCM (10 ml) was stirred at room temperature for 30 min. The DCM was removed via rotary evaporation and the reaction was diluted with 1,3-dichlorobenzene (7 mL). The mixture was then heated in a microwave at 160° C. for 1 hr. Ether (10 ml) was added to the reaction and the resulting solid was collected by filtration and washed with ether to provide the title compound, 5-(3-mercapto-[1,2,4]triazolo[4,3-a]pyridin-6-yl)-1-ethylpyridin-2(1H)-one (600 mg, 42.3% yield).

N-(6-(6-(1-ethyl-6-oxo-1,6-dihydropyridin-3-yl)-[1,2,4]triazolo[4,3-a]pyridin-3-ylthio)imidazo[1,2-b]pyridazin-2-yl)cyclopropanecarboxamide: The title compound was prepare from N-(6-iodoimidazo[1,2-b]pyridazin-2-yl)cyclopropanecarboxamide and 5-(3-mercapto-[1,2,4]triazolo[4,3-a]pyridin-6-yl)-1-ethylpyridin-2(1H)-one followed the procedure of the synthesis of compound 4. The crude reaction mixture was purified by preparative LCMS, and the product was isolated as the TFA salt. The product was then dissolved in MeOH, treated 4M HCl in dioxane, and concentrated to dryness to provide the HCl salt of the title compound. ¹H NMR (DMSO-d₆,400 MHz): δ=11.15 (s, 1H), 8.66-8.72 (m, 1H), 8.24 (d, J=2.5 Hz, 1H), 8.08 (dd, J=9.6, 1.0 Hz, 1H), 7.93 (s, 2H), 7.90-7.92 (m, 1H), 7.87 (dd, J=9.5, 2.9 Hz, 1H), 7.06 (d, J=9.3 Hz, 2H), 6.47 (d, J=9.3 Hz, 1H), 3.96 (q, J=7.1 Hz, 2H), 1.92 (t, J=6.1 Hz, 1H), 1.24 (t, J=7.2 Hz, 3H), 0.75-0.82 ppm (m, 4H). ESI-MS: m/z 473.2 (M+H)⁺.

### Compound 107: N-(6-(6-(3-cyano-4-(2-hydroxyethoxy)phenyl)-[1,2,4]triazolo[4,3-a]pyridin-3-ylthio)imidazo[1,2-b]pyridazin-2-yl)cyclopropanecarboxamide

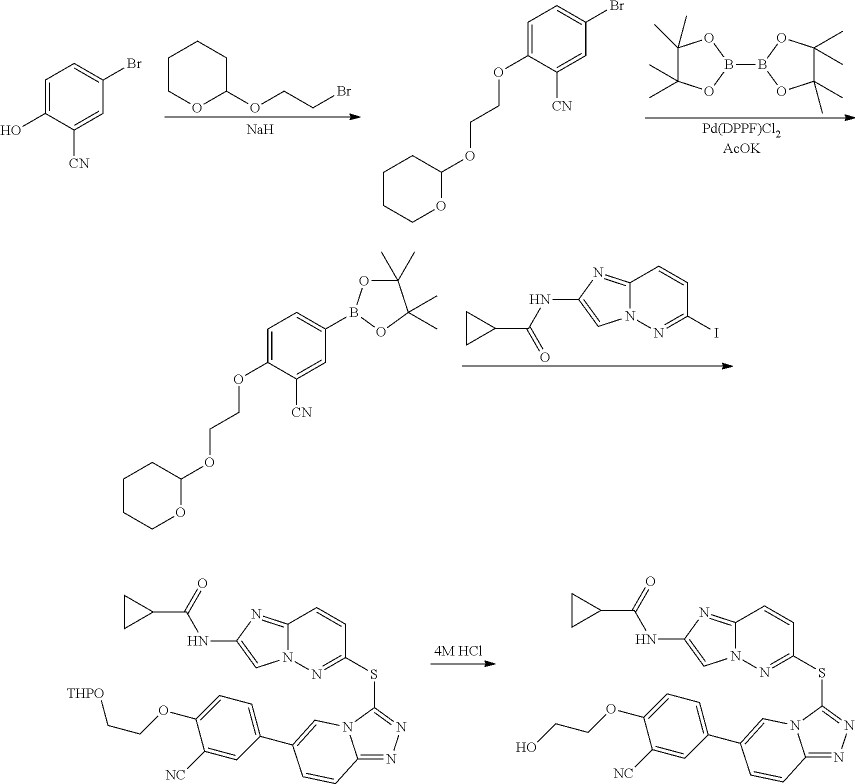

### 5-bromo-2-(2-(tetrahydro-2H-pyran-2-yloxy)ethoxy)benzonitrile

A solution of 5-bromo-2-hydroxybenzonitrile (1 g, 5.05 mmol) in DMF was treated with 95% NaH (242 mg, 9.6 mmol) for 20 min at room temperature. 2-(2-Bromoethoxy)tetrahydro-2H-pyran (0.839 ml, 5.56 mmol) was then added to the reaction and the mixture was stirred at room temperature for 5 hrs. The reaction was then diluted with DCM and washed with water. The organic phase was dried over Na₂SO₄, filtered, and concentrated to dryness to provide the crude title compound, 5-bromo-2-(2-(tetrahydro-2H-pyran-2-yloxy)ethoxy)benzonitrile (1.6 g, 9.75% yield) which was used without further purification.

### 2-(2-(tetrahydro-2H-pyran-2-yloxy)ethoxy)-5-(4,4,5,5-tetramethyl-1,3,2-dioxaborolan-2-yl)benzonitrile

A mixture of 5-bromo-2-(2-(tetrahydro-2H-pyran-2-yloxy)ethoxy)benzonitrile (1.6 g, 4.92 mmol), 4,4,4′,4′,5,5,5′,5′-octamethyl-2,2′-bi(1,3,2-dioxaborolane) (1.9 g, 7.48 mmol), AcOK (1.47 g, 15 mmol) and Pd(DPPF)Cl₂ (366 mg, 0.5 mmol) in dioxane (50 ml) was heated at 110° C. for 60 min. The reaction was evaporated to dryness via rotary evaporation and the resulting residue was purified by silica gel chromatography (DCM-Hexane) to provide the title compound, 2-(2-(tetrahydro-2H-pyran-2-yloxy)ethoxy)-5-(4,4,5,5-tetramethyl-1,3,2-dioxaborolan-2-yl)benzonitrile (1.7 g, 92.6% yield).

N-(6-(6-(3-cyano-4-(3-(tetrahydro-2H-pyran-2-yloxy)propyl)phenyl)-[1,2,4]triazolo[4,3-a]pyridin-3-ylthio)imidazo[1,2-b]pyridazin-2-yl)cyclopropanecarboxamide: The title compound was prepared from compound 4 and 2-(2-(tetrahydro-2H-pyran-2-yloxy)ethoxy)-5-(4,4,5,5-tetramethyl-1,3,2-dioxaborolan-2-yl)benzonitrile following the procedure for the synthesis of compound 5. The reaction mixture was used without further purification.

N-(6-(6-(3-cyano-4-(2-hydroxyethoxy)phenyl)-[1,2,4]triazolo[4,3-a]pyridin-3-ylthio)imidazo[1,2-b]pyridazin-2-yl)cyclopropanecarboxamide: As a crude reaction mixture, N-(6-(6-(3-cyano-4-(3-(tetrahydro-2H-pyran-2-yloxy)propyl)phenyl)-[1,2,4]triazolo[4,3-a]pyridin-3-ylthio)imidazo[1,2-b]pyridazin-2-yl)cyclopropanecarboxamide was treated with [HCl]:dioxane (1:5) at 70° C. for 30 min. The reaction was concentrated and purified by LCMS to provide the title compound as a TFA salt. The product was then dissolved in MeOH, treated 4M HCl in dioxane, and concentrated to dryness to provide the HCl salt of the title compound. ¹H NMR (METHANOL-d₄,400 MHz): δ=9.00 (br. s., 1H), 8.36 (br. s., 1H), 8.24 (br. s., 1H), 8.08 (br. s., 2H), 8.01 (br. s., 1H), 7.94 (br. s., 1H), 7.66-7.74 (m, 1H), 7.36 (br. s., 1H), 4.27 (br. s., 2H), 3.95 (br. s., 2H), 1.83 (br. s., 1H), 0.84-1.04 ppm (m, 4H). ESI-MS: m/z 513.4 (M+H)⁺.

### Compound 108: N-(6-(6-(5-cyano-6-hydroxypyridin-3-yl)-[1,2,4]triazolo[4,3-a]pyridin-3-ylthio)imidazo[1,2-b]pyridazin-2-yl)cyclopropanecarboxamide

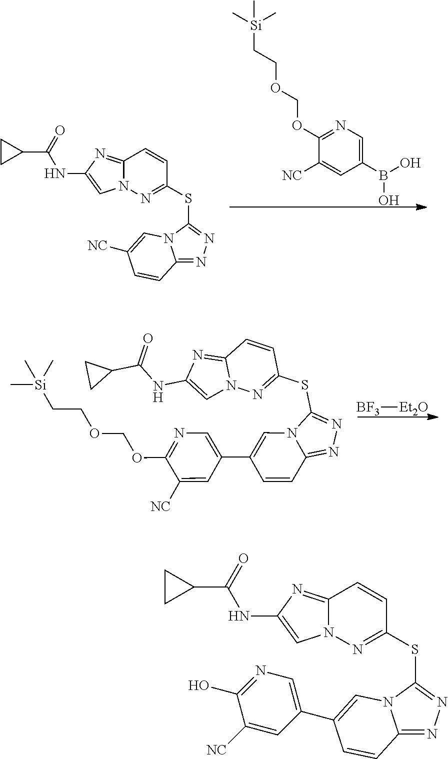

N-(6-(6-(5-cyano-6-((2-(trimethylsilyl)ethoxy)methoxy)pyridin-3-yl)-[1,2,4]triazolo[4,3-a]pyridin-3-ylthio)imidazo[1,2-b]pyridazin-2-yl)cyclopropanecarboxamide: The title compound was synthesized using an analogous procedure to that described in the preparation of Compound 5 using 5-cyano-6-((2-(trimethylsilyl)ethoxy)methoxy)pyridin-3-ylboronic acid. The crude reaction was used for the next step without further purification.

N-(6-(6-(5-cyano-6-hydroxypyridin-3-yl)-[1,2,4]triazolo[4,3-a]pyridin-3-ylthio)imidazo[1,2-b]pyridazin-2-yl)cyclopropanecarboxamide: As a crude reaction mixture, N-(6-(6-(5-cyano-6-((2-(trimethylsilyl)ethoxy)methoxy)pyridin-3-yl)-[1,2,4]triazolo[4,3-a]pyridin-3-ylthio)imidazo[1,2-b]pyridazin-2-yl)cyclopropanecarboxamide was refluxed in TFA:DCM (1:1, 5 mL) for 30 min. The reaction was then concentrated and purified by preparative LCMS to provide the title compound, N-(6-(6-(5-cyano-6-hydroxypyridin-3-yl)-[1,2,4]triazolo[4,3-a]pyridin-3-ylthio)imidazo[1,2-b]pyridazin-2-yl)cyclopropanecarboxamide as a TFA salt. The product was then dissolved in MeOH, treated 4M HCl in dioxane, and concentrated to dryness to provide the HCl salt of the title compound. ¹H NMR (METHANOL-d₄,400 MHz): δ=8.80 (br. s., 1H), 8.51 (d, J=2.5 Hz, 1H), 8.13 (d, J=2.8 Hz, 1H), 8.04 (d, J=9.6 Hz, 1H), 7.79-7.96 (m, 3H), 7.27 (d, J=7.8 Hz, 1H), 1.83 (br. s., 1H), 0.91 ppm (d, J=16.7 Hz, 4H). ESI-MS: m/z 513.4 (M+H)⁺.

### Compound 109: N-(6-(6-(2-(3-hydroxypropylamino)pyrimidin-5-yl)-[1,2,4]triazolo[4,3-a]pyridin-3-ylthio)imidazo[1,2-b]pyridazin-2-yl)cyclopropanecarboxamide

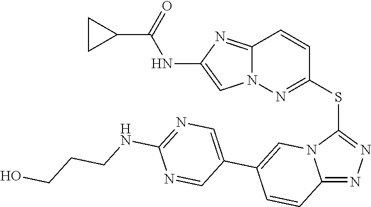 [SMILES: O=C(Nc1cn2nc(Sc3nnc4ccc(-c5cnc(NCCCO)nc5)cn34)ccc2n1)C1CC1]

The title compound was synthesized using an analogous procedure to that described in the preparation of Compound 5 using 2-(3-hydroxypropylamino)pyrimidin-5-ylboronic acid. The crude reaction mixture was purified by preparative LCMS, and the product was isolated as the TFA salt. ¹H NMR (ACETONITRILE-d₃,400 MHz): δ=8.50 (s, 2H), 8.44 (s, 1H), 7.89-8.00 (m, 2H), 7.67-7.78 (m, 2H), 7.62 (s, 3H), 7.01 (d, J=9.3 Hz, 1H), 3.55 (t, J=6.1 Hz, 2H), 3.45 (t, J=6.6 Hz, 2H), 1.70-1.79 (m, 3H), 0.85 ppm (d, J=16.2 Hz, 4H). ESI-MS: m/z 503.3 (M+H)⁺.

### Compound 110: N-(6-(6-(2-morpholinothiazol-4-yl)-[1,2,4]triazolo[4,3-a]pyridin-3-ylthio)imidazo[1,2-b]pyridazin-2-yl)cyclopropanecarboxamide

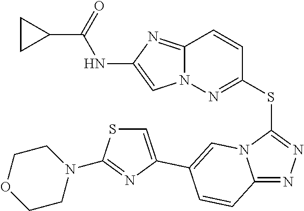 [SMILES: O=C(Nc1cn2nc(Sc3nnc4ccc(-c5csc(N6CCOCC6)n5)cn34)ccc2n1)C1CC1]

The title compound was synthesized using an analogous procedure to that described in the preparation of Compound 5 using 2-morpholinothiazol-4-ylboronic acid. The crude reaction mixture was purified by preparative LCMS, and the product was isolated as the TFA salt. ¹H NMR (ACETONITRILE-d₃,400 MHz): δ=8.74 (s, 1H), 8.03 (br. s., 1H), 7.91-7.97 (m, 1H), 7.83-7.90 (m, 1H), 7.71 (br. s., 1H), 7.15 (s, 1H), 7.03 (br. s., 1H), 3.75 (s, 4H), 3.39-3.45 (m, 4H), 1.78 (dt, J=5.0, 2.4 Hz, 1H), 0.81-0.92 ppm (m, 4H). ESI-MS: m/z 520.2 (M+H)⁺.

### Compound 111: N-(6-(6-(3-(3-hydroxypropyl)phenyl)-[1,2,4]triazolo[4,3-a]pyridin-3-ylthio)imidazo[1,2-b]pyridazin-2-yl)cyclopropanecarboxamide

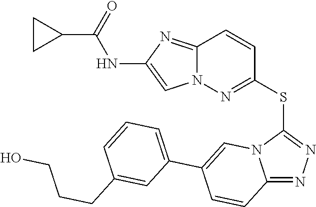 [SMILES: O=C(Nc1cn2nc(Sc3nnc4ccc(-c5cccc(CCCO)c5)cn34)ccc2n1)C1CC1]

The title compound was synthesized using an analogous procedure to that described in the preparation of Compound 5 using 3-(3-hydroxypropyl)phenylboronic acid. The crude reaction mixture was purified by preparative LCMS, and the product was isolated as the TFA salt. ¹H NMR (METHANOL-d₄,400 MHz): δ=8.54 (s, 1H), 7.84-7.94 (m, 3H), 7.72 (d, J=9.3 Hz, 1H), 7.33-7.39 (m, 2H), 7.25-7.32 (m, 1H), 7.18 (d, J=7.6 Hz, 1H), 7.10 (d, J=9.3 Hz, 1H), 3.46 (t, J=6.3 Hz, 2H), 2.58-2.67 (dd, J=8.0 and 7.6, 2 H), 1.67-1.79 (m, 3H), 0.72-0.87 ppm (m, 4H). ESI-MS: m/z 486.3 (M+H)⁺.

### Compound 112: N-(6-(6-((2-methoxyethoxy)methyl)-[1,2,4]triazolo[4,3-a]pyridin-3-ylthio)imidazo[1,2-b]pyridazin-2-yl)cyclopropanecarboxamide

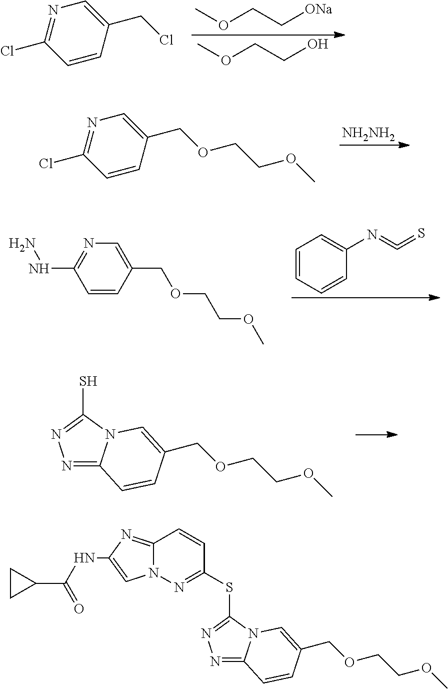

### 2-hydrazinyl-5-((2-methoxyethoxy)methyl)pyridine

To a solution of 2-chloro-5-(chloromethyl)pyridine (1620 mg, 10 mmol) in 2-methoxyethanol (10 mL) was added sodium 2-methoxyethanolate (5.9 g, 12.00 mmol). The mixture was stirred at room temperature for 10 min and then at 50° C. for 2 hrs. Hydrazine (1.6 g, 50.0 mmol) was then added to the mixture and the reaction was heated at 120° C. for 1 hr. The mixture was purified by preparative LCMS to provide the title compound, 2-hydrazinyl-5-((2-methoxyethoxy)methyl)pyridine (540 mg, 27.4% yield).

### 6-((2-methoxyethoxy)methyl)[1,2,4]triazolo[4,3-a]pyridine-3-thiol

The title compound was prepared from 2-hydrazinyl-5-((2-methoxyethoxy)methyl)pyridine and isothiocyanatobenzene following the procedure described in the synthesis of compound 4A.

N-(6-(6-((2-methoxyethoxy)methyl)-[1,2,4]triazolo[4,3-a]pyridin-3-ylthio)imidazo[1,2-b]pyridazin-2-yl)cyclopropanecarboxamide: The title compound was prepared from N-(6-iodoimidazo[1,2-b]pyridazin-2-yl)cyclopropanecarboxamide and 6-((2-methoxyethoxy)methyl)-[1,2,4]triazolo[4,3-a]pyridine-3-thiol following the procedure described for the synthesis of Compound 4. ¹H NMR (METHANOL-d₄,400 MHz): δ=8.75-8.78 (m, 1H), 8.15 (dd, J=9.3, 1.0 Hz, 1H), 8.04-8.11 (m, 1H), 8.02 (d, J=9.3 Hz, 1H), 7.94 (s, 1H), 7.56 (d, J=9.3 Hz, 1H), 4.71 (d, J=1.0 Hz, 2H), 3.61-3.67 (m, 2H), 3.47-3.53 (m, 2H), 3.29 (s, 3H), 1.80-1.87 (m, 1H), 0.88-1.01 ppm (m, 4H). ESI-MS: m/z 440.2 (M+H)⁺.

### Compound 113: N-(6-(6-fluoro-5-methyl-[1,2,4]triazolo[4,3-a]pyridin-3-ylthio)imidazo[1,2-b]pyridazin-2-yl)cyclopropanecarboxamide

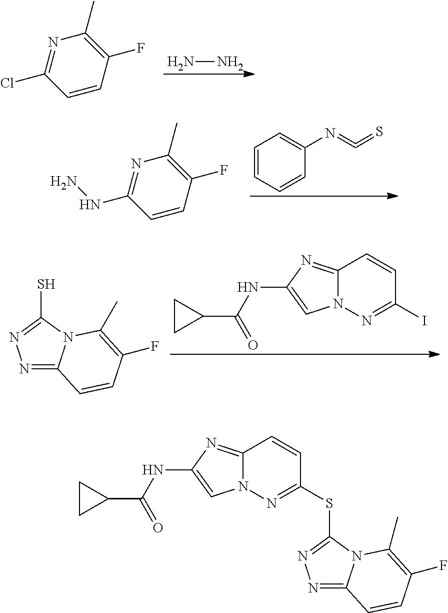

### 3-fluoro-6-hydrazinyl-2-methylpyridine

To a mixture of 6-chloro-3-fluoro-2-methylpyridine (4.7 g, 32.3 mmol) in isopropanol (100 mL) was added hydrazine hydrate (5 g, 100 mmol). After the mixture was refluxed for 5 hrs, additional hydrazine hydrate (5 g, 100 mmol) was added, and the reaction was refluxed for 3 days. The mixture was then concentrated to dryness and stored under high vacuum overnight. This material was used without further purification.

### 6-fluoro-5-methyl-[1,2,4]triazolo[4,3-a]pyridine-3-thiol

The title compound was prepared from 3-fluoro-6-hydrazinyl-2-methylpyridine and isothiocyanatobenzene following the procedure described for the synthesis of compound 4A.

### N-(6-(6-fluoro-5-methyl-[1,2,4]triazolo[4,3-a]pyridin-3-ylthio)inidazo[1,2-b]pyridazin-2-yl)cyclopropanecarboxamide

The title compound was prepared from N-(6-iodoimidazo[1,2-b]pyridazin-2-yl)cyclopropanecarboxamide and 6-fluoro-5-methyl-[1,2,4]triazolo[4,3-a]pyridine-3-thiol following the procedure described for the synthesis of compound 4. The crude reaction mixture was purified by preparative LCMS, and the product was isolated as the TFA salt. The product was then dissolved in MeOH, treated 4M HCl in dioxane, and concentrated to dryness to provide the HCl salt of the title compound. ¹H NMR (METHANOL-d₄,400 MHz): δ=8.00 (s, 1H), 7.83-7.89 (m, 3H), 7.63 (dd, J=9.9, 8.3 Hz, 1H), 7.22 (d, J=9.6 Hz, 1H), 2.96 (d, J=3.5 Hz, 4H), 1.81-1.91 (m, 1H), 0.96 (dt, J=4.5, 3.0 Hz, 2H), 0.86-0.92 (m, 2H). ESI-MS: m/z 384.3 (M+H)⁺.

### Compound 114: 6-([1,2,4]triazolo[4,3-a]pyridin-3-ylthio)imidazo[1,2-b]pyridazin-2-amine

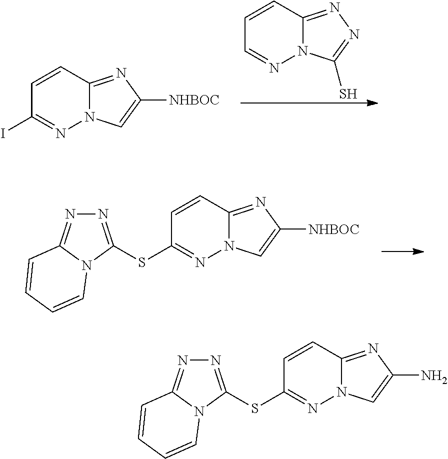

### tert-Butyl 6-([1,2,4]triazolo[4,3-a]pyridin-3-ylthio)imidazo[1,2-b]pyridazin-2-ylcarbamate

The title compound was prepared from tert-butyl 6-iodoimidazo[1,2-b]pyridazin-2-ylcarbamate and [1,2,4]triazolo[4,3-a]pyridine-3-thiol following the procedure for the synthesis of compound 4.

### 6-([1,2,4]triazolo[4,3-a]pyridin-3-ylthio)imidazo[1,2-b]pyridazin-2-amine

A solution of tert-butyl 6-([1,2,4]triazolo[4,3-a]pyridin-3-ylthio)imidazo[1,2-b]pyridazin-2-ylcarbamate (300 mg, 0.78 mmol) in dioxane (5 ml) was treated with 4M HCl in dioxane (5 ml) at 80° C. for 1 hr. The mixture was concentrated and purified by LCMS to give the title compound, 6-([1,2,4]triazolo[4,3-a]pyridin-3-ylthio)imidazo[1,2-b]pyridazin-2-amine (201 mg, 68% yield) as the TFA salt. ¹H NMR (400 MHz, DMSO-d₆) δ ppm 7.00 (d, J=9.35 Hz, 1H) 7.16 (t, J=6.44 Hz, 1H) 7.22 (s, 1H) 7.54-7.65 (m, 1H) 7.73 (d, J=9.35 Hz, 1H) 8.00 (d, J=9.09 Hz, 1H) 8.47 (d, J=6.82 Hz, 1H). ESI-MS: m/z 284.2 (M+H)⁺.

### Biological Testing

A. In Vivo Evaluation of Compound 45, Alone and in Combination with Anti-HGF mAb HuL2G7, in Human U87MG Glioblastoma Implanted Nude Mice

Female athymic nude mice (Harlan), 10 weeks of age, were fed ad libitum water (reverse osmosis, 1 ppm C12) and an irradiated standard rodent diet (NIH31 Modified and Irradiated) consisting of: 18% protein, 5% fat, and 5% fiber. Mice were housed on irradiated Enrich-o′-Cobs™ laboratory animal bedding in static microisolators on a 12-hour light cycle at 21-22° C. (70-72° F.) and 40-60% humidity.

The human U87MG glioblastoma was maintained in serial s.c. transplantation in nude mice. Mice were implanted subcutaneously in the flank with a 1 mm³ fragment of U87MG glioblastoma. Tumors were monitored twice weekly then subsequently daily as the neoplasms reached the desired size, approximately 190 mm³. At 13 days, when the tumors attained a size between 126-288 mm³ in calculated tumor volume, the animals were pair-matched into various treatment groups (the mean tumor volume in each group was 184-192 mm³). Estimated tumor volume was calculated using the formula:

$$Tumor Volume ( {mm}^{3} ) = \frac{{w}^{2} \times l}{2}$$

where w=width and l=length in mm of a U87MG glioblastoma.

Compound 45 was produced as described above. Compound 45 was then prepared for administration in 30% Captisol/25 mM citrate at pH 3.0. HuL2G7 was then provided as a 19.0 mg/ml stock solution in normal saline. The stock solution was diluted with normal saline to provide the formulation for injection.

The concentration of each compound was such that the desired dose was delivered in a volume of 5 μl per gram body weight. Dosing solutions of Compound 45 were prepared fresh weekly and stored at room temperature. Dosing solutions of HuL2G7 were prepared fresh prior to each treatment and were stored under refrigeration along with the stock solution.

The protocol design for this study is provided in Table 1. On Day 1, animals were pairmatched by tumor size into 16 groups of 10 mice each. Compound 45 was given p.o. at 10.8, 32.4 and 108 mg/kg qd×14. HuL2G7 was given i.v. at 1, 3 and 10 mg/kg qwk×2. Treatment with both agents either alone or in combination was initiated on Day 1. Mice treated with monotherapy received the vehicle used for the other agent. There was a vehicle control group which received the Captisol/citrate, pH 3.0 vehicle p.o. qd×14 as well as saline i.v. qwk×2. Treatment groups were compared for statistical significance to the vehicle control. Groups that received the combination were compared for statistical significance to the monotherapy groups that received the same dose of the two agents used in combination. The experiment was terminated on Day 44.

TABLE 1

| | | Treatment Regimen 1 | | | | Treatment Regimen 2 | | | |
|---|---|---|---|---|---|---|---|---|---|
| Group | n | Agent | mg/kg | Route | Schedule | Agent | mg/kg | Route | Schedule |
| 1 | 10 | Vehicle 1 | — | po | qd x 14 | Vehicle 2 | — | iv | qwk x 2 |
| 2 | 10 | Cmpd 45 | 10.81 | po | qd x 14 | Vehicle 2 | — | iv | qwk x 2 |
| 3 | 10 | Cmpd 45 | 32.43 | po | qd x 14 | Vehicle 2 | — | iv | qwk x 2 |
| 4 | 10 | Cmpd 45 | 108.11 | po | qd x 14 | Vehicle 2 | — | iv | qwk x 2 |
| 5 | 10 | Vehicle 1 | — | po | qd x 14 | HuL2G7 | 1 | iv | qwk x 2 |
| 6 | 10 | Vehicle 1 | — | po | qd x 14 | HuL2G7 | 3 | iv | qwk x 2 |
| 7 | 10 | Vehicle 1 | — | po | qd x 14 | HuL2G7 | 10 | iv | qwk x 2 |
| 8 | 10 | Cmpd 45 | 108.11 | po | qd x 14 | HuL2G7 | 1 | iv | qwk x 2 |
| 9 | 10 | Cmpd 45 | 108.11 | po | qd x 14 | HuL2G7 | 3 | iv | qwk x 2 |
| 10 | 10 | Cmpd 45 | 108.11 | po | qd x 14 | HuL2G7 | 10 | iv | qwk x 2 |
| 11 | 10 | Cmpd 45 | 32.43 | po | qd x 14 | HuL2G7 | 1 | iv | qwk x 2 |
| 12 | 10 | Cmpd 45 | 32.43 | po | qd x 14 | HuL2G7 | 3 | iv | qwk x 2 |
| 13 | 10 | Cmpd 45 | 32.43 | po | qd x 14 | HuL2G7 | 10 | iv | qwk x 2 |
| 14 | 10 | Cmpd 45 | 10.81 | po | qd x 14 | HuL2G7 | 1 | iv | qwk x 2 |
| 15 | 10 | Cmpd 45 | 10.81 | po | qd x 14 | HuL2G7 | 3 | iv | qwk x 2 |
| 16 | 10 | Cmpd 45 | 10.81 | po | qd x 14 | HuL2G7 | 10 | iv | qwk x 2 |

Vehicle 1 = 30% Captisol in 0.1M sodium citrate buffer, pH 3
Vehicle 2 = Saline

The tumor growth delay (TGD) method was used in this study; treatment-effected median increases in time to endpoint (TTE) of various treatment groups were compared to untreated and vehicle controls. In the TGD method, each animal was euthanized as a cancer death when its U87MG glioblastoma reached a size of 2000 mm³. TTE values were determined for all groups based on the calculated day reaching endpoint as given by the formula:

$$T T E = \frac{{log}_{10} ( endpoint volume ) - b}{m}$$

where TTE is expressed in days, endpoint volume is in mm³, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. Animals that do not reach the endpoint are assigned a TTE value equal to the last day of the study (44 days). Animals classified as treatment related deaths (TR) or non-treatment-related metastatic deaths (NTRm) are assigned a TTE value equal to the day of death. Animals classified as non-treatment-related deaths (NTRu or NTRa) are excluded from TTE calculations. Treatment efficacy was determined from tumor growth delay (TGD), which is defined as the increase in the median TTE for a treatment group compared to the control group:

TGD=T−C,

expressed in days, or as a percentage of the median TTE of the control group:

$$\% T G D = \frac{T - C}{C} \times 100$$

where T is the median TTE for a treatment group and C is the median TTE for control Group 1. Treatment can cause complete tumor regression (CR, defined as tumor shrinkage to below measurable size [3×3 mm] for 3 consecutive measurements), partial tumor regression (PR, defined as reduction to <50% of initial tumor volume for 3 consecutive measurements), or inhibit the neoplasm's growth such that the tumor does not reach the 800 mm³ endpoint by the time that the experiment is terminated. A CR that persists through the end of the experiment is a long-term tumor-free survivor (TFS). The duration of a CR or PR was recorded throughout the study.

Tables 2 and 3 present the TGD values and regressions, respectively.

TABLE 2
Comparison of Tumor Growth Delay (TGD) Values for Combination
Therapy vs. Monotherapy in U87MG Glioblastoma

| | | HuL2G7 (mg/kg, qwk x 2) | | | |
|---|---|---|---|---|---|
| | | 0 | 1 | 3 | 10 |
| Compound 45 (mg/kg, qd x 14) | 0 | — | 0.3 | 7 | 5.7 |
| | 10.8 | 0 | 2.7 | 4.3 | 9.9 |
| | 32.4 | 3.9 | 4.4 | 8.7 | 14.1 |
| | 108 | 3.1 | 7.2 | 11.2 | 20.8 |

TABLE 3
Comparison of Regressions for Combination Therapy vs. Monotherapy
in U87MG Glioblastoma

| | | HuL2G7 (mg/kg, qwk x 2) | | | |
|---|---|---|---|---|---|
| | | 0 | 1 | 3 | 10 |
| Compound 45 | 0 | — | 0/10 | 0/10 | 0/10 |
| (mg/kg, qd x 14) | 10.8 | 0/10 | 0/10 | 0/10 | 1 PR/10 |
| | 32.4 | 0/10 | 0/10 | 1 PR/10 | 2 PR/10 |
| | 108 | 0/10 | 0/10 | 2 PR/10 | 1 CR + 8 PR/10 |

Compound 45 showed tumor growth delays of 3-4 days (ca. 30%) upon daily treatment lasting for 14 days. The anti-HGF mAb HuL2G7 showed tumor growth delays of 6-7 days (ca. 52%). Neither agent was able to induce regressions in this brain tumor model. When these two therapies were combined, there was a dramatic therapeutic synergism. This was achieved in the absence of any evidence of increased toxicity. The combination was able to induce regressions in 9/10 treated animals with 1/10 TFS and a tumor growth delay that was significantly greater than that seen with either agent alone and which far exceeded the sum of the TGD values for the individual components of the combination.

B. In Vivo Evaluation of Compounds 3 and 45, Alone and in Combination with Anti-HGF mAb HuL2G7, in Human U87MG Glioblastoma Implanted Nude Mice

Female athymic nude mice (Harlan), 9 weeks of age, were fed ad libitum water (reverse osmosis, 1 ppm Cl₂) and an irradiated standard rodent diet (NIH31 Modified and Irradiated) consisting of: 18% protein, 5% fat, and 5% fiber. Mice were housed on irradiated Enrich-O'Cobs™ laboratory animal bedding in static microisolators on a 12-hour light cycle at 21-22° C. (70-72° F.) and 40-60% humidity.

The human U87MG glioblastoma utilized in the present study was maintained in serial s.c. transplantation in nude mice. Mice were implanted subcutaneously in the flank with a 1 mm³ fragment of U87MG glioblastoma. Tumors were monitored as the neoplasms reached the desired size, approximately 190 mm³. At 12 days, when the tumors attained a size between 172-256 mm³ in calculated tumor volume, the animals were pair-matched into various treatment groups (the mean tumor volume in each group was 190-198 mm³). Estimated tumor volume was calculated using the formula:

$$Tumor Volume ( {mm}^{3} ) = \frac{{w}^{2} \times l}{2}$$

where w=width and l=length in mm of a U87MG glioblastoma.

Compounds 3 and 45 were prepared by as described above. Compounds 3 and 45 were then prepared for administration as suspensions in 30% Captisol/100 mM citrate at pH 3.0. HuL2G7 was then provided as a 20.7 mg/ml stock solution in normal saline. The stock solution was diluted with normal saline to provide the formulation for injection.

The concentration of each compound was such that the desired dose was delivered in a volume of 5 μl per gram body weight. Dosing solutions of Compounds 3 and 45 were prepared fresh weekly and stored under refrigeration. Dosing solutions of HuL2G7 were prepared fresh prior to each treatment and were stored under refrigeration along with the stock solution.

The protocol design for this study is provided in Table 4. On Day 1, animals were pairmatched by tumor size into 8 groups of 10 mice each. Compounds 3 and 45 were given p.o. qd×14 at 136 and 200 mg/kg, respectively. HuL2G7 was given i.v. at 10 mg/kg qwk×2. Treatment with all agents, either alone or in combination, was initiated on Day 1. Mice treated with monotherapy received the vehicle used for the other agent. There was a vehicle control group which received the Captisol/citrate, pH 3.0 vehicle p.o. qd×14 as well as saline i.v. qwk×2. Treatment groups were compared for statistical significance to the vehicle control. Groups that received the combinations were compared for statistical significance to the monotherapy groups that received the two agents used in combination. The experiment was terminated on Day 43.

TABLE 4

| | | Treatment Regimen 1 | | | | Treatment Regimen 2 | | | |
|---|---|---|---|---|---|---|---|---|---|
| Group | n | Agent | mg/kg | Route | Schedule | Agent | mg/kg | Route | Schedule |
| 1 | 10 | Vehicle 1 | — | po | qd x 14 | Vehicle 2 | — | iv | qwk x 2 |
| 2 | 10 | Cmpd 45 | 136.05 | po | qd x 14 | Vehicle 2 | — | iv | qwk x 2 |
| 3 | 10 | Cmpd 3 | 200 | po | qd x 14 | Vehicle 2 | — | iv | qwk x 2 |
| 4 | 10 | Vehicle 1 | — | po | qd x 14 | HuL2G7 | 10 | iv | qwk x 2 |
| 5 | 10 | Cmpd 45 | 136.05 | po | qd x 14 | HuL2G7 | 10 | iv | qwk x 2 |
| 6 | 10 | Cmpd 3 | 200 | po | qd x 14 | HuL2G7 | 10 | iv | qwk x 2 |

Vehicle 1 = 30% Captisol in 0.1M sodium citrate buffer, pH 3
Vehicle 2 = Saline

The tumor growth delay (TGD) method was used in this study; treatment-effected median increases in time to endpoint (TTE) of various treatment groups were compared to untreated and vehicle controls. In the TGD method, each animal was euthanized as a cancer death when its U87MG glioblastoma reached a size of 2000 mm³. TTE values were determined for all groups based on the calculated day reaching endpoint as given by the formula:

$$T T E = \frac{{log}_{10} ( endpoint volume ) - b}{m}$$

where TTE is expressed in days, endpoint volume is in mm³, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. Animals that do not reach the endpoint are assigned a TTE value equal to the last day of the study (43 days). Animals classified as treatment related deaths (TR) or non-treatment-related metastatic deaths (NTRm) are assigned a TTE value equal to the day of death. Animals classified as non-treatment-related deaths (NTRu or NTRa) are excluded from TTE calculations.

Treatment efficacy was determined from tumor growth delay (TGD), which is defined as the increase in the median TTE for a treatment group compared to the control group:

TGD=T−C,

expressed in days, or as a percentage of the median TTE of the control group:

$$\% T G D = \frac{T - C}{C} \times 100$$

where T is the median TTE for a treatment group and C is the median TTE for control Group 1. Treatment can cause complete tumor regression (CR, defined as tumor shrinkage to below measurable size [3×3 mm] for 3 consecutive measurements), partial tumor regression (PR, defined as reduction to <50% of initial tumor volume for 3 consecutive measurements), or inhibit the neoplasm's growth such that the tumor does not reach the 800 mm³ endpoint by the time that the experiment is terminated. A CR that persists through the end of the experiment is a long-term tumor-free survivor (TFS). The duration of a CR or PR was recorded throughout the study.

A graph of median tumor volume as a function of time is shown in FIG. 4. The combination of Compound 45 with HuL2G7 was tolerated at the dose levels tested in this experiment with no weight loss but 1 TR/10 mice. There was clear-cut evidence of therapeutic synergism as reflected in tumor growth delay and regression of U87MG glioblastoma. The combination produced a tumor growth delay of 19.2 days (152%). This was significantly different from both of the drugs used as monotherapy (P<0.0001 and P=0.0002). This combination produced 3 PR/10 with durations of 8-19 days (median of 8 days). Transient reductions in tumor volume of >50% were seen in all of the other mice, but they did not last for three consecutive measurements and thus did not meet the rigorous criterion for regression.

Compounds 3 and 45 showed tumor growth delays of 3 days (ca. 23%) upon daily treatment lasting for 14 days. Neither of these agents was able to induce regressions in this brain tumor model. When c-Met receptor kinase inhibitors were combined with the anti-HGF mAb, there was a dramatic therapeutic synergism. This was achieved in the absence of any evidence of increased toxicity. The combination of Compound 45 plus HuL2G7 showed 3/10 regressions and a TGD of 152%. The combination of Compound 3 plus HuL2G7 showed 1/10 regression and TGD of 103%. The TGD for all three of the combinations was significantly greater than that seen with either agent alone and far exceeded the sum of the TGD values for the individual components of the combinations.

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A method of treating a disease state that is mediated by HGF/cMET comprising administering a cMET inhibitor, or a pharmaceutically acceptable salt or solvate thereof, and an antibody that inhibits the HGF/cMET signaling pathway to a patient.

2. The method according to claim 1 wherein the disease state is cancer.

3. The method according to claim 1 wherein the cancer is hepatocellular carcinoma.

4. The method according to claim 2 wherein the cancer is brain cancer.

5. The method according to claim 2 wherein the cancer is a glioblastoma.

6. The method according to claim 2 wherein the cancer is pancreatic cancer.

7. The method according to claim 1 wherein the cMET inhibitor is administered in the form of a pharmaceutical composition comprising the cMET inhibitor, or a pharmaceutically acceptable salt or solvate thereof; and one or more pharmaceutically acceptable excipients.

8. The method according to claim 1 wherein the antibody binds to the cMET receptor.

9. The method according to claim 1 wherein the antibody binds to a cMET ligand.

10. The method according to claim 1 wherein the antibody binds to HGF.

11. The method according to claim 1 wherein the antibody binds to human HGF.

12. The method according to claim 1 wherein the antibody is a chimeric L2G7 monoclonal antibody.

13. The method according to claim 1 wherein the antibody is a humanized L2G7 monoclonal antibody.

14. The method according to claim 1 wherein the antibody is a human L2G7 monoclonal antibody.

15. The method according to claim 1 wherein the cMET inhibitor has the formula: 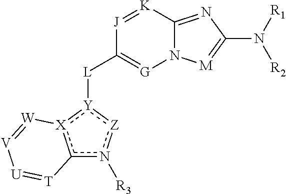
or a pharmaceutically acceptable salt thereof, wherein
G is selected from the group consisting of CR₄ and N;
J is selected from the group consisting of CR₅ and N;
K is selected from the group consisting of CR₆ and N;
M is selected from the group consisting of CR₇ and N;
L is absent or a linker providing 1, 2, 3, 4, 5 or 6 atom separation between the rings to which L is attached, wherein the atoms of the linker providing the separation are selected from the group consisting of carbon, oxygen, nitrogen, and sulfur;
T is selected from the group consisting of CR₈ and N;
U is selected from the group consisting of CR₉ and N;
V is selected from the group consisting of CR₁₀ and N;
W is selected from the group consisting of CR₁₁ and N;
X is selected from the group consisting of CR₁₂ and N;
Y is selected from the group consisting of CR₁₃ and N;
Z is selected from the group consisting of CR₁₄R₁₅ and NR₁₆;
R₁ is selected from the group consisting of hydrogen, carbonyloxy, (C₁₋₁₀)alkoxy, (C₄₋₁₂)aryloxy, hetero(C₁₋₁₀)aryloxy, carbonyl, oxycarbonyl, aminocarbonyl, (C₁₋₁₀)alkylcarbonyl, (C₃₋₁₂)cycloalkyl(C₁₋₅)carbonyl, hetero(C₃₋₁₂)cycloalkyl(C₁₋₁₀)carbonyl, aryl(C₁₋₁₀)carbonyl, hetero(C₁₋₁₀)aryl(C₁₋₅)carbonyl, (C₉₋₁₂)bicycloaryl(C₁₋₅)carbonyl, hetero(C₈₋₁₂)bicycloaryl(C₁₋₅)carbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicyclo aryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁ has the formula 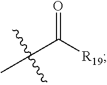
R₂ is hydrogen or a substituent convertible in vivo to hydrogen;
R₃ is selected from the group consisting of hydrogen, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₃ is absent when the nitrogen to which it is bound forms part of a double bond;
R₄ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₅ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₆ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₇ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₈ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₉ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, amido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, amino(C₁₋₁₀)alkyl, amido(C₁₋₁₀)alkylamino(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, carbonyl(C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₀ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, amido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, amino(C₁₋₁₀)alkyl, amido(C₁₋₁₀)alkylamino(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, carbonyl(C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₁ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted;
R₁₂ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₂ is absent when the carbon to which it is bound forms part of a double bond;
R₁₃ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₃ is absent when the carbon to which it is bound forms part of a double bond;
R₁₄ and R₁₅ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₅ is absent when the carbon to which it is bound forms part of a double bond;
R₁₆ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted, or R₁₆ is absent when the nitrogen to which it is bound forms part of a double bond; and
R₁₉ selected from the group consisting of hydrogen, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, aza(C₁₋₁₀)alkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₄₋₁₀)aryl, (C₉₋₁₂)bicycloaryl and hetero(C₄₋₁₂)bicycloaryl, each substituted or unsubstituted.

16. The method according to (claim 1) wherein the cMET inhibitor has the formula:

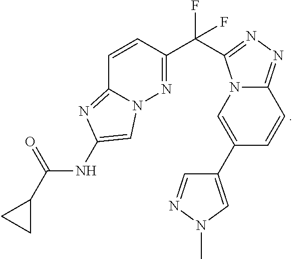 [SMILES: Cn1cc(-c2ccc3nnc(C(F)(F)c4ccc5nc(NC(=O)C6CC6)cn5n4)n3c2)cn1]

17. The method according to claim 1 wherein the cMET inhibitor has the formula:

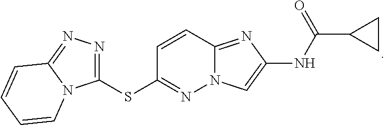 [SMILES: O=C(Nc1cn2nc(Sc3nnc4ccccn34)ccc2n1)C1CC1]

18. The method according to claim 1 wherein the cMET inhibitor has the formula:

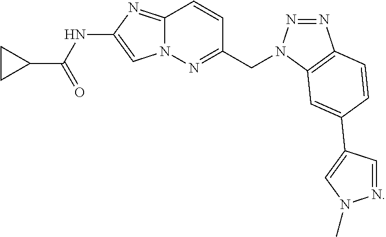 [SMILES: Cn1cc(-c2ccc3nnn(Cc4ccc5nc(NC(=O)C6CC6)cn5n4)c3c2)cn1]

19. The method according to claim 1 wherein the cMET inhibitor has the formula: 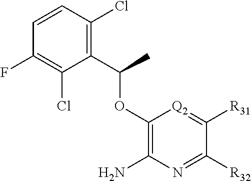
wherein
Q₂ is N or CR₄₂;
R₃₁ is selected from the group consisting of hydrogen, halogen, (C₆₋₁₂)aryl, 5-12 membered heteroaryl, (C₃₋₁₂)cycloalkyl, 3-12 membered heteroalicyclic,
O—(CR₃₆R₃₇)R₃₄, COR₃₄, C(O)OR₃₄, CN, NO₂, S(O)₁R₃₄, SO₂NR₃₄R₃₅, NR₃₄C(O)R₃₅, C(═NR₃₆)NR₃₄R₃₅, (C₁₋₈)alkyl, (C₂₋₈)alkenyl and (C₂₋₈)alkynyl, and each hydrogen in R₃₁ is optionally substituted by one or more R₃₃ groups;
R₃₂ is hydrogen, halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, O(CR₃₆R₃₇)_{q}R₃₄, NR₃₄C(O)R₃₅, (CR₃₆R₃₇)_{q}C(O)OR₃₄, (CR₃₆R₃₇)_{q}NCR₃₄R₃₅, C(═NR₃₆)NR₃₄R₃₅, NR₃₄C(O)NR₃₅R₃₆, NR₃₄S(O)ₚR₃₅ or C(O)NR₃₄R₃₅, and each hydrogen in R₃₂ is optionally substituted by R₃₈;
each R₃₃ is independently halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, O(CR₃₆R₃₇)R₃₄, NR₃₄C(O)R₃₅, (CR₃₆R₃₇)_{q}C(O)OR₃₄, (CR₃₆R₃₇)_{q}OR₃₄, (CR₃₆R₃₇)_{q}C(O)NR₃₄R₃₅, (CR₃₆R₃₇)_{q}NCR₃₄R₃₅, C(═NR₃₆)NR₃₄R₃₅, NR₃₄C(O)NR₃₅R₃₆, NR₃₄S(O)_{P}R₃₅ or C(O)NR₃₄R₃₅, each hydrogen in R₃₃ is optionally substituted by R₃₈, and R₃₃ groups on adjacent atoms may combine to form a (C₆₋₁₂)aryl, 5-12 membered heteroaryl, (C₃₋₁₂)cycloalkyl or 3-12 membered heteroalicyclic group;
each R₃₄, R₃₅, R₃₆ and R₃₇ is independently hydrogen, halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl; or any two of R₃₄, R₃₅, R₃₆ and R₃₇ bound to the same nitrogen atom may, together with the nitrogen to which they are bound, be combined to form a 3 to 12 membered heteroalicyclic or 5-12 membered heteroqaryl group optionally containing 1 to 3 additional heteroatoms selected from N, O and S; or any two of R₃₄, R₃₅, R₃₆ and R₃₇ bound to the same carbon atom may be combined to form a (C₃₋₁₂)cycloalkyl, (C₆₋₁₂)aryl, 3-12 membered heteroalicyclic or 5-12 memebered heteroaryl group; and each hydrogen in R₃₄, R₃₅, R₃₆ and R₃₇ is optionally substituted by R₃₈;
each R₃₈ is independently halogen, (C₁₋₁₂)alkyl, (C₂₋₁₂)alkenyl, (C₂₋₁₂)alkynyl, (C₃₋₁₂)cycloalkyl, (C₆₋₁₂)aryl, 3-12 membered heteroalicyclic, 5-12 membered heteroaryl, NH₂, CN, OH, O—(C₁₋₁₂)alkyl, O—(CH₂)_{q}(C₃₋₁₂)cycloalkyl, O—(CH₂)_{q}(C₆₋₁₂)aryl, O—(CH₂)_{q}(3-12 membered heteroalicyclic) or O—(CH₂)_{q}(5-12 membered heteroaryl); and each hydrogen in R₃₈ is optionally substituted by R₄₁;
each R₃₉ and R₄₀ is independently hydrogen, halogen, (C₁₋₁₂)alkyl, (C₃₋₁₂)cycloalkyl, (C₆₋₁₂)aryl, 3-12 membered heteroalicyclic, 5-12 membered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, OC(O)R₃₄, NR₃₄C(O)R₃₅, (CR₃₆R₃₇)^{q}C(O)OR₃₄, (CR₃₆R₃₇)_{q}NCR₃₄R₃₅; NR₃₄C(O)NR₃₅R₃₆, NR₃₄S(O)ₚR₃₅ or C(O)NR₃₄R₃₅; R₃₉ or R₄₀ may combine with a ring atom of A or a substitutent of A to form a (C₃₋₁₂)cycloalkyl, 3-12 memebered heteroalicyclic, (C₆₋₁₂)aryl or 5-12 memebered heteroaryl ring fused to A; and each hydrogen in R₃₉ and R₄₀ is optionally substituted by R₃₃;
each R₄₁ is independently halogen, (C₁₋₁₂)alkyl, (C₁₋₁₂)alkoxy, (C₃₋₁₂)cycloalkyl, (C₆₋₁₂) aryl, 3-12 membered heteroalicyclic, 5-12 membered heteroaryl, O—(C₁₋₁₂)alkyl, O—(CH2)₁(C₃₋₁₂)cycloalkyl, O—(CH₂)₄C₆₋₁₂)aryl, O—(CH₂)_{q}(3-12 membered heteroalicyclic, O—(CH₂)_{q}(5-12 membered heteroaryl or CN, and each hydrogen in R₄₁ is optionally substituted by halogen, OH, CN, (C₁₋₁₂)alkyl which may be partially or fully halogenated, O—(C₁₋₁₂)alkyl which may be partially or fully halogenated, CO, SO or SO₂;
R₄₂ is hydrogen, halogen (C₁₋₁₂)alkyl, (C₂₋₁₂)alkenyl, (C₂₋₁₂)alkynyl, (C₃₋₁₂)cycloalkyl, (C₆₋₁₂)aryl, 3-12 memebered heteroalicyclic, 5-12 memebered heteroaryl, S(O)₁R₃₄, SO₂NR₃₄R₃₅, S(O)₂OR₃₄, NO₂, NR₃₄R₃₅, (CR₃₆R₃₇)_{q}OR₃₄, CN, C(O)R₃₄, O—C(O)R₃₄, O—CR₃₆R₃₇)_{q}R₃₄, NR₃₄C(O)R₃₅, (CR₃₆R₃₇)_{q}C(O)OR₃₄, (CR₃₆R₃₇)_{q}NCR₃₄R₃₅, C(—NR₃₆)NR₃₄R₃₅, NR₃₄C(O)NR₃₅R₃₆, NR₃₄S(O)ₚR₃₅ or C(O)NR₃₄R₃₅, and each hydrogen in R₄₂ is optionally substituted by R₃₃;
each R₄₃ is independently halogen, (C₁₋₁₂)alkyl, (C₂₋₁₂)aleknyl, (C₂₋₁₂)alkynyl, (C₃₋₁₂)cycloalkyl, (C₆₋₁₂)aryl, 3-12 membered hertoalicyclic, 5-12 memebered heteroaryl, S(O)₁R₃₄, SO₂NR₃₄R₃₅, S(O)₂OR₃₄, NO₂, NR₃₄R₃₅, (CR₃₆R₃₇)_{q}OR₃₄; CN, C(O)R₃₄, O—C(O)R₃₄, O—(CR₃₆R₃₇)_{q}R₃₄, NR₃₄C(O)R₃₅, (CR₃₆R₃₇)₉C(O)OR₃₄, (CR₃₆R₃₇)_{q}OR₃₄, (CR₃₆R₃₇)_{q}C(O)NR₃₄R₃₅, (CR₃₆R₃₇)₉NCR₃₄R₃₅, C(═NR₃₆)NR₃₄R₃₅, NR₃₄C(O)NR₃₅R₃₆, NR₃₄S(O)ₚR₃₅, C(O)NR₃₄R₃₅; (CR₃₆R₃₇)_{q}(3-12 memebered heteroalicyclic), (CR₃₆R₃₇)_{q}(C₃₋₁₂)cycloalkyl, (CR₃₆R₃₇)_{q}(C₆₋₁₂)aryl, (CR₃₆R₃₇)_{q}(5-12 membered heteroaryl, (CR₃₆R₃₇)_{q}C(O)NR₃₄R₃₅ or (CR₃₆R₃₇)_{q}C(O)R₃₄, R₄₃ groups on adjacent atoms may combine to form a (C₆₋₁₂)aryl, 5-12 memebered heteroaryl, (C₃₋₁₂)cycloalkyl or 3-12 memebered heteroalicyclic group, and each hydrogen in R₄₃ is optionally substituted by R₃₃;
each 1 is independently 0, 1 or 2;
each q is independently 0, 1, 2, 3 or 4; and
each p is independently 1 or 2,
or a pharmaceutically acceptable salt, hydrate or solvate thereof.

20. The method according to claim 1 wherein the cMET inhibitor has the formula:

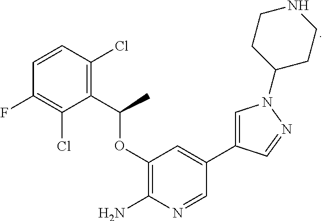 [SMILES: C[C@@H](Oc1cc(-c2cnn(C3CCNCC3)c2)cnc1N)c1c(Cl)ccc(F)c1Cl]

21. The method according to claim 1 wherein the cMET inhibitor has the formula: 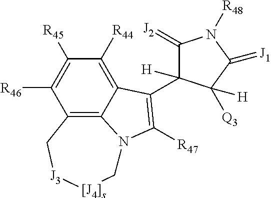
wherein
R₄₄, R₄₅ and R₄₆ are independently selected from the group consisting of H, F, Cl, Br, I, NR₅₀R₅₁, (C₁₋₆)alkyl, (C₁₋₆) substituted alkyl, (C₃₋₉)cycloalkyl, (C₃₋₉) substituted cycloalkyl, O—(C₁₋₆)alkyl, O—(C₃₋₉)cycloalkyl, O—(C₃₋₉) substituted cycloalkyl, aryl, heteroaryl and heterocyclyl;
R₄₇ is selected from the group consisting of H, (C₁₋₄)alkyl and (C₁₋₄) substituted alkyl;
R₄₈ is selected from the group consisting of H, (C₁₋₆)alkyl, CH₂R₄₉, CONHR₅₂, COR₅₃ and SO₂R₅₄;
R₄₉ is selected from the group consisting of O—P(═O)(OH)₂, O—P(═O)(OH)(O—(C₁₋₆)alkyl), O—P(═O)(O—(C₁₋₆)alkyl)₂, O—P(═O)(OH)(O—(CH₂)phenyl), O—P(═O)(O—(CH₂)phenyl)₂, a carboxylic acid group, an amino carboxylic acid group and a peptide;
R₅₀ and R₅₁ are independently selected from the group consisting of H and (C₁₋₆)alkyl;
R₅₂, R₅₃ and R₅₄ are independently selected from the group consisting of H, NHR₅₅, (C_{—}_{¹⁻⁶})alkyl, (C_{—}_{¹⁻⁶}) substituted alkyl, (C₃₋₉)cycloalkyl, (C₃₋₉) substituted cycloalkyl, aryl, heteroaryl and heterocyclyl;
Q₃ is selected from the group consisting of indolyl, substituted indolyl, aryl, heteroaryl, heterocyclyl and alkyl;
J₁ and J₂ are independently selected from the group consisting of O, S, H₂, where R₄₇ is (C₁₋₄)alkyl or (C₁₋₄) substituted alkyl when both J₁ and J₂ are O, and R₄₈ is H, (C₁₋₆)alkyl or CH₂R₄₉ when both J₁ and J₂ are not H₂;
J₃ is selected from the group consisting of —CH₂—, —NR₅₅—, S, O and a bond;
R₅₅ is selected from the group consisting of H, (C₁₋₆)alkyl, (C₁₋₆) substituted alkyl, (C₃₋₉)cycloalkyl, (C₃₋₉) substituted cycloalkyl, O—(C₁₋₆)alkyl, C(═O)—O—(C₁₋₆)alkyl and C(═O)—O—(C₁₋₆) substituted alkyl;
J₄ is selected from the group consisting of —CH₂—, CO and a bond; and
s is 0, 1 or 2.

22. The method according to claim 1 further comprising the step of administering one or more additional therapeutic agents.

23. The method according to claim 22 wherein the additional therapeutic agent comprises a Hedgehog inhibitor.

24. The method according to claim 22 wherein the additional therapeutic agent comprises an EGF inhibitor.

25. The method according to claim 22 wherein the additional therapeutic agent comprises a PTEN agonist.
